(19) United States
(12) Patent Application Publication
(10) Pub. No.: US 2004/0126834 A1
(43) Pub. Date: Jul. 1, 2004

(54) Compositions and methods for the modulation of sphingolipid metabolism and/or signaling

(75) Inventor: Julie D. Saba, Oakland, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Children's Hospital and Research Institute at Oakland, Oakland, CA

(21) Appl. No.: 10/622,011

(22) Filed: Jul. 16, 2003

Related U.S. Application Data
(63) Continuation-in-part of application No. 10/348,052, filed on Jan. 17, 2003.
(63) Continuation-in-part of application No. 10/053,510, filed on Jan. 17, 2002.
(60) Provisional application No. 60/349,582, filed on Jan. 17, 2002.

(51) Int. Cl. C12Q 1/44; C12N 1/18

(52) U.S. Cl. 435/19; 435/254.2

(57) ABSTRACT

Compositions, methods and kits for diagnosing and treating cancer and muscular disorders are provided. Therapeutic compositions may comprise agents that modulate sphingolipid metabolism and/or signaling pathways. Such compositions may be administered to a mammal afflicted with cancer. Diagnostic methods and kits may employ an agent suitable for detecting alterations in endogenous genes involved in sphingolipid metabolism. Such methods and kits may be used to detect the presence of a cancer or to evaluate the prognosis of a known disease. Screens for identifying agents that modulate sphingolipid metabolism and/or signaling pathways are also provided.

### CROSS REFERENCE TO RELATED APPLICATIONS

This application is a continuation-in-part of U.S. application Ser. No. 10/348,052, which claims the benefit of provisional application No. 60/349,582, filed Jan. 17, 2002 and U.S. application Ser. No. 10/053,510, filed Jan. 17, 2002, all applications incorporated herein by reference in their entirety.

### STATEMENT REGARDING FEDERALLY SPONSORED RESEARCH OR DEVELOPMENT

This invention was made with government support under Grant No. 1R01CA77528 awarded by the National Institutes of Health. The government has certain rights in this invention.

### BACKGROUND OF THE INVENTION

1. Field of the Invention

The present invention relates generally to cancer detection and therapy. The invention is more particularly related to polynucleotides encoding polypeptides involved in the metabolism of sphingolipids, polypeptides, and to agents that modulate the expression and/or activity of such polypeptides. Such agents may be used, for example, to diagnose and/or treat cancers such as breast, colon, uterus, stomach, ovary, lung, kidney and rectum cancer, the diagnosis and treatment of muscle developmental defects and cardiomyopathy, and diagnosis and treatment of hereditary sensory neuropathy type 1 and the sphingolipidoses. The present invention further relates to methods of screening agents that modulate the expression and/or activity of polynucleotides and/or polypeptides involved in sphingolipid metabolism.

2. Description of the Related Art

Breast cancer is a significant health problem for women in the United States and throughout the world. Although advances have been made in detection and treatment of the disease, breast cancer remains the most common form of cancer, and the second leading cause of cancer death, in American women. Among African-American women and women between 15 and 54 years of age, breast cancer is the leading cause of cancer death. One out of every eight women in the United States will develop breast cancer, a risk which has increased 52% during 1950-1990. In 1994, it is estimated that 182,000 new cases of female breast cancer were diagnosed, and 46,000 women died from the disease.

No vaccine or other universally successful method for the prevention or treatment of breast cancer is currently available. Management of the disease currently relies on a combination of early diagnosis (through routine breast screening procedures) and aggressive treatment, which may include one or more of a variety of treatments such as surgery, radiotherapy, chemotherapy and hormone therapy. The course of treatment for a particular breast cancer is often selected based on a variety of prognostic parameters, including an analysis of specific tumor markers. However, the use of established markers often leads to a result that is difficult to interpret.

With current therapies, tumor invasiveness and metastasis is a critical determinant in the outcome for breast cancer patients. Although the five year survival for women diagnosed with localized breast cancer is about 90%, the five year survival drops to 18% for women whose disease has metastasized. Present therapies are inadequate for inhibiting tumor invasiveness for the large population of women with this severe disease.

Colon cancer is the second most frequently diagnosed malignancy in the United States as well as the second most common cause of cancer death. The five-year survival rate for patients with colorectal cancer detected in an early localized stage is 92%; unfortunately, only 37% of colorectal cancer is diagnosed at this stage. The survival rate drops to 64% if the cancer is allowed to spread to adjacent organs or lymph nodes, and to 7% in patients with distant metastases.

The prognosis of colon cancer is directly related to the degree of penetration of the tumor through the bowel wall and the presence or absence of nodal involvement, consequently, early detection and treatment are especially important. Currently, diagnosis is aided by the use of screening assays for fecal occult blood, sigmoidoscopy, colonoscopy and double contrast barium enemas. Treatment regimens are determined by the type and stage of the cancer, and include surgery, radiation therapy and/or chemotherapy. Recurrence following surgery (the most common form of therapy) is a major problem and is often the ultimate cause of death. In spite of considerable research into therapies for the disease, colon cancer remains difficult to diagnose and treat. In spite of considerable research into therapies for these and other cancers, colon cancer remains difficult to diagnose and treat effectively. Accordingly, improvements are needed in the treatment, diagnosis and prevention of breast and colon cancer. The present invention fulfills this need and further provides other related advantages.

Mutations that result in failure or dysregulation of sphingolipid synthesis or catabolism are directly responsible for a number of human diseases, including hereditary sensory neuropathy type 1 and the group of lysosomal storage diseases called the sphingolipidoses (Bejaoui, K., Wu, C., Scheffler, M. D., Haan, G., Ashby, P., Wu, L., de Jong, P. and Brown, R. H., Jr. (2001). Nat Genet 27, 261-2.; Dawkins, J. L., Hulme, D. J., Brahmbhatt, S. B., Auer-Grumbach, M. and Nicholson, G. A. (2001). Nat Genet 27, 309-12.; Gable, K., Han, G., Monaghan, E., Bacikova, D., Natarajan, M., Williams, R. and Dunn, T. M. (2002). J Biol Chem 277, 10194-200.). A large body of evidence now indicates that sphingolipid metabolites and enzymes of sphingolipid metabolism play important roles in regulating cell migration, stress response, survival, differentiation, senescence, apoptosis, receptor signaling, and endocytosis in eukaryotic cells. These findings suggest molecular mechanisms by which sphingolipids may affect animal physiology and contribute to disease states.

Sphingosine-1-phosphate (S-1-P) is an endogenous sphingolipid metabolite present in most mammalian cells and in serum. Like other sphingolipid metabolites such as ceramide and sphingosine, S-1-P participates in specific signal transduction pathways. Many of the effects of S-1-P signaling, which include promotion of cellular proliferation, enhancement of migration, inhibition of apoptosis and stimulation of angiogenesis, influence the transformation, growth, drug resistance, vascularity and metastatic capacity of cancer cells. Several observations support the notion that sphingosine kinase (SK) and sphingosine-1-phosphate lyase (SPL) may be cancer related genes. First, the overexpression of SK in NIH3T3 fibroblasts leads to oncogenic transformation as determined by the ability of transfected cells to form foci in vitro and to form fibrosarcomas in NOD/SCID mice. Second, human SPL was cloned and mapped to 10q21, a chromosomal region frequently deleted in a variety of human cancers. Taken together, these observations raise the possibility that SK and SPL may be potentially effective targets for pharmacological intervention in the treatment of cancer. Accordingly, the present invention provides methods for screening agents that modulate sphingolipid metabolism. Further, the present invention provides methods for detecting and treating cancer.

Critical steps in the identification and development of new therapeutic agents are: (a) generation of candidate agents; and (b) screening of the candidate agents for efficacy and safety. With the advent of combinatorial chemistry protocols, large numbers of potential compounds, known as libraries, can be rapidly generated. Such libraries serve as collections of potential therapeutic agents. Following generation of a library of potential therapeutic agents, the library must be screened to identity the promising candidates.

For screening purposes, a number of in vitro high throughput screening protocols have been developed. However, these in vitro screening assays must be followed by in vivo screening assays. Since it is undesirable to immediately screen compounds that show promise from in vitro assays in humans, an important step in the identification of therapeutic agents for such cellular proliferative diseases is the screening of potential therapeutic compounds in non-human animal models. As such, non-human animal models of cancer and other cellular proliferative diseases play an important role in the discovery of therapeutic agents for such diseases.

One type of non-human animal model that can be used for screening purposes to identify therapeutic agents for use in treating cancer and other cellular proliferative diseases is a non-human mammalian model, e.g. mice, etc. However, mice are expensive, have a slow reproduction time, and generate small numbers of offspring. As such, they are less than ideal for many high throughput screening assays.

Accordingly, there is a need for additional cellular and animal models for the identification of therapeutic agents for cancer and other diseases associated with altered sphingolipid metabolism. Of particular interest would be the development of a model in a biological system having a relatively short life cycle and a rapid growth cycle characterized by the production of large numbers of progeny. Preferably, such a model system should also be relatively simple and economic to maintain.

### BRIEF SUMMARY OF THE INVENTION

As noted above, the present invention relates generally to cancer detection and therapy. The invention is more particularly related to polynucleotides encoding polypeptides involved in the metabolism and/or signaling of sphingolipids, polypeptides, and to agents that modulate the expression and/or activity of such polypeptides and/or the alter the levels of sphingolipid intermediates. Such agents may be used, for example, to diagnose and/or treat cancers such as breast, colon, uterus, stomach, ovary, lung, kidney and rectum cancer, the diagnosis and treatment of muscle developmental defects and cardiomyopathy, and diagnosis and treatment of hereditary sensory neuropathy type 1 and the sphingolipidoses. The present invention further relates to methods of screening agents that modulate the components and intermediates involved in sphingolipid metabolism and/or signaling.

It is an aspect of the present invention to provide a method for identifying an agent that modulates sphingolipid metabolism, comprising (a) culturing a homozygous null mutant Drosophila melanogaster in the absence and presence of a candidate agent under conditions and for a time sufficient to observe in the mutant Drosophila melanogaster an effect of the agent on a level of either (i) at least one sphingolipid intermediate, or (ii) activity of at least one component of a sphingolipid pathway, wherein the mutant Drosophila melanogaster comprises a P-element transposon insertion in a gene encoding a component of a sphingolipid pathway that results in at least one of an altered level of at least one sphingolipid intermediate and an altered activity level of at least one sphingolipid pathway component; and (b) comparing the level of either (i) the sphingolipid intermediate that is generated, or (ii) the activity of the sphingolipid pathway component, in the presence of the candidate agent to the level in the absence of the candidate agent, wherein an altered level indicates the agent modulates sphingolipid metabolism. In certain embodiments the altered level of a sphingolipid intermediate comprises an increase in C_{14/16} long chain bases, and in certain other embodiments the altered level of a sphingolipid intermediate comprises an increase in C_{14/16} phosphorylated long chain bases. In certain embodiments the gene encoding a component of a sphingolipid pathway comprises a polynucleotide sequence set forth in any one of SEQ ID NOS:15, 24 and 25. In certain embodiments the homozygous null mutant Drosophila melanogaster exhibits a flightless phenotype, and in certain other embodiments the homozygous null mutant Drosophila melanogaster comprises a tumor. In certain embodiments the homozygous null mutant Drosophila melanogaster comprises a T2 segment which comprises abnormal developmental patterning of thoracic muscles. In certain embodiments the altered level of the sphingolipid intermediate that is generated in the presence of the candidate agent comprises a decrease in sphingosine-1-phosphate and in certain embodiments the altered level of the sphingolipid intermediate that is generated in the presence of the candidate agent comprises an increase in sphingosine-1-phosphate.

In still other embodiments the altered level of the activity of the sphingolipid pathway component in the presence of the candidate agent comprises a decrease in sphingosine-1-phosphate lyase (SPL) activity, while in other embodiments the altered level of the activity of the sphingolipid pathway component in the presence of the candidate agent comprises an increase in sphingosine-1-phosphate lyase (SPL) activity in still other embodiments the altered level of the activity of the sphingolipid pathway component in the presence of the candidate agent comprises a decrease in sphingosine kinase (SK) activity, while in other embodiments the altered level of the activity of the sphingolipid pathway component in the presence of the candidate agent comprises an increase in sphingosine kinase (SK) activity. In certain embodiments the agent inhibits SK activity, and in certain other embodiments the agent inhibits SPL activity. In certain embodiments the agent comprises a 1-aryl-2-dimethylaminopropane-1,3-diol derivative, and in certain other embodiments the derivative comprises a substitution of a fatty acid amide group. In certain further embodiments the substitution comprises two N-methyl groups. In another embodiment the agent increases activity of serine palmitoyltransferase.

Turning to another aspect, the present invention provides a method for identifying an agent that modulates sphingolipid metabolism, comprising (a) culturing a homozygous null mutant Drosophila melanogaster in the absence and presence of a candidate agent under conditions and for a time sufficient to observe in said mutant Drosophila melanogaster an effect of the agent on a level of either (i) at least one sphingolipid intermediate, or (ii) activity of at least one component of a sphingolipid pathway, wherein the mutant Drosophila melanogaster comprises a P-element transposon insertion in a gene encoding a component of a sphingolipid pathway that results in an altered activity level of at least one sphingolipid pathway component, and wherein the mutant Drosophila melanogaster exhibits a flightless phenotype that results from said insertion; and (b) comparing flight performance of the mutant Drosophila that is cultured in the presence of the candidate agent to the flight performance of the mutant Drosophila that is cultured in the absence of the candidate agent, wherein an increased flight performance of the mutant Drosophila cultured in the presence of the agent indicates the agent modulates sphingolipid metabolism. In certain embodiments the mutant Drosophila melanogaster comprises a homozygous mutation in a gene encoding a sphingosine-1-phosphate lyase (SPL), and in certain embodiments the homozygous null mutant Drosophila melanogaster comprises a T2 segment which comprises abnormal developmental patterning of thoracic muscles. In certain embodiments the agent that modulates sphingolipid metabolism inhibits sphingosine kinase activity.

In yet another embodiment there is provided a method for identifying an agent that modulates sphingolipid signaling, comprising (a) culturing a homozygous null mutant Drosophila melanogaster in the absence and presence of a candidate agent under conditions and for a time sufficient to observe in said mutant Drosophila melanogaster an effect of the agent on a level of at least one sphingolipid intermediate, wherein the mutant Drosophila melanogaster comprises a P-element transposon insertion in a gene encoding a component of a sphingolipid pathway that results in an altered level of at least one sphingolipid intermediate; and (b) comparing the level of the sphingolipid intermediate that is generated in the presence of the candidate agent to the level in the absence of the candidate agent, wherein an altered level indicates the agent modulates sphingolipid signaling. It is also an aspect of the invention to provide an agent identified by the method of any one of the above described methods, which in certain embodiments is a composition comprising such agent in combination with a physiologically acceptable excipient. In certain embodiments there is provided a composition comprising an agent that increases flight performance in a homozygous null mutant Drosophila melanogaster, wherein the mutant Drosophila melanogaster comprises a P-element transposon insertion in a gene encoding a sphingosine-1-phosphate lyase (SPL) polypeptide that comprises the amino acid sequence set forth in SEQ ID NO:16, and wherein the mutant Drosophila melanogaster exhibits a flightless phenotype that results from said insertion, and in certain further embodiments the agent inhibits sphingosine kinase activity.

According to certain other embodiments of the present invention there is provided a method for preparing a sphingosine-1-phosphate lyase (SPL) polypeptide, comprising culturing a host cell transformed or transfected with a nucleic acid construct comprising a promoter operably linked to a polynucleotide comprising the nucleotide sequence set forth in SEQ ID NO:15; and recovering a sphingosine-1-phosphate lyase polypeptide.

In still other embodiments there is provided a method for identifying an agent that modulates sphingosine-1-phosphate lyase activity, comprising (a) contacting a candidate agent with an isolated polypeptide that comprises an amino acid sequence selected from an amino acid sequence set forth in SEQ ID NO:16 and an amino acid sequence having at least 90% identity to the sequence set forth in SEQ ID NO:16, wherein the polypeptide has sphingosine-1-phosphate lyase activity, and wherein the step of contacting is carried out under conditions and for a time sufficient to allow the candidate agent to interact with said polypeptide; and (b) determining degradation by the polypeptide of sphingosine-1-phosphate or a sphingosine-1-phosphate derivative thereof in the presence of the candidate agent, relative to degradation by said polypeptide of sphingosine-1-phosphate or a sphingosine-1-phosphate derivative thereof in the absence of the candidate agent, and therefrom identifying an agent that modulates sphingosine-1-phosphate lyase activity.

In another embodiment there is provided a method for identifying an agent that modulates sphingosine-1-phosphate lyase activity, comprising (a) contacting a candidate agent with a biological sample that comprises a cell which expresses a polypeptide that comprises an amino acid sequence selected from an amino acid sequence set forth in SEQ ID NO:16 and an amino acid sequence having at least 90% identity to the sequence set forth in SEQ ID NO:16, wherein said polypeptide has sphingosine-1-phosphate lyase activity, and wherein the step of contacting is carried out under conditions and for a time sufficient to allow the candidate agent to interact with the polypeptide; and (b) determining degradation by said polypeptide of sphingosine-1-phosphate or a sphingosine-1-phosphate derivative thereof in the presence of the candidate agent, relative to degradation by said polypeptide of sphingosine-1-phosphate or a sphingosine-1-phosphate derivative thereof in the absence of the candidate agent, and therefrom identifying an agent that modulates sphingosine-1-phosphate lyase activity. In certain embodiments the step of determining comprises an in vitro assay of an extract from the cell.

In certain embodiments the invention provides a composition comprising an agent that modulates sphingosine-1-phosphate lyase activity of a polypeptide, said polypeptide comprising a sequence set forth in SEQ ID NO:16, in combination with a pharmaceutically acceptable carrier. In certain further embodiments the agent comprises a polynucleotide. In certain other further embodiments the agent comprises an antibody or an antigen-binding fragment thereof that specifically binds a sphingosine phosphate lyase (SPL) polypeptide comprising the sequence set forth in SEQ ID NO:16, and wherein the antibody increases the ability of the SPL polypeptide to degrade sphingosine-1-phosphate. In certain embodiments the invention provides a method for inhibiting growth of a cancer cell, comprising contacting the cancer cell with an agent that increases sphingosine-1-phosphate lyase activity of a polypeptide comprising a sequence set forth in SEQ ID NO:16. In certain further embodiments the agent increases expression of an endogenous sphingosine-1-phosphate lyase gene, and in certain other further embodiments the cancer cell is a breast cancer cell.

According to another embodiment there is provided a method for inhibiting development of cancer, metastasis, or both development of cancer and metastasis in a mammal, comprising administering to said mammal an agent that increases sphingosine-1-phosphate lyase activity of a polypeptide comprising a sequence set forth in SEQ ID NO:16. In certain further embodiments the agent increases expression of an endogenous sphingosine-1-phosphate lyase gene, and in certain still further embodiments the agent is linked to a targeting component, which in certain still further embodiments is an anti-tumor antibody and in certain other still further embodiments binds to an estrogen receptor. In certain embodiments the mammal is afflicted with breast cancer.

It is another aspect of the present invention to provide a method for determining the presence of cancer in a patient, comprising the steps of (a) contacting a first biological sample comprising at least one polynucleotide and being obtained from a patient suspected of having cancer with at least one oligonucleotide that is specific for a polynucleotide which comprises a nucleic acid sequence as set forth in SEQ ID NO:23; (b) detecting an amount of the olignucleotide that hybridizes to the polynucleotide in the first sample; and (d) comparing the amount of oligonucleotide that hybridizes to the polynucleotide in the first sample to an amount of oligonucleotide that hybridizes to a polynucleotide in a second biological sample obtained from a normal control subject known to be free of cancer, wherein a statistically significant decrease in the amount of olignucleotide that hybridizes to the polynucleotide in the first biological sample relative to the amount of oligonucleotide that hybridizes to the polynucleotide in the second sample signifies the presence of a cancer in said patient.

It is another aspect of the present invention to provide a method for diagnosing a disease associated with altered sphingolipid metabolism comprising (a) contacting a first biological sample comprising at least one polynucleotide and being obtained from a patient suspected of having a disease associated with altered sphingolipid metabolism with at least one oligonucleotide that is specific for a polynucleotide which comprises a nucleic acid sequence as set forth in SEQ ID NO:23; (b) detecting an amount of the olignucleotide that hybridizes to the polynucleotide in the first sample; and (d) comparing the amount of oligonucleotide that hybridizes to the polynucleotide in the first sample to an amount of oligonucleotide that hybridizes to a polynucleotide in a second biological sample obtained from a normal control subject known to be free of a disease associated with altered sphingolipid metabolism, wherein a statistically significant decrease in the amount of olignucleotide that hybridizes to the polynucleotide in the first biological sample relative to the amount of oligonucleotide that hybridizes to the polynucleotide in the second sample signifies the presence of a disease associated with altered sphingolipid metabolism in said patient.

It is another aspect of the present invention to provide a method for determining the presence of a cancer in a patient, comprising the steps of (a) contacting a first biological sample comprising at least one polynucleotide and being obtained from a patient suspected of having cancer with at least one oligonucleotide that is specific for a polynucleotide which comprises a nucleic acid sequence as set forth in SEQ ID NO:22; (b) detecting an amount of the olignucleotide that hybridizes to the polynucleotide in the first sample; and (d) comparing the amount of oligonucleotide that hybridizes to the polynucleotide in the first sample to an amount of oligonucleotide that hybridizes to a polynucleotide in a second biological sample obtained from a normal control subject known to be free of cancer, wherein a statistically significant increase in the amount of olignucleotide that hybridizes to the polynucleotide in the first biological sample relative to the amount of oligonucleotide that hybridizes to the polynucleotide in the second sample signifies the presence of a cancer in said patient.

It is another aspect of the present invention to provide a method for diagnosing a disease associated with altered sphingolipid metabolism comprising (a) contacting a first biological sample comprising at least one polynucleotide and being obtained from a patient suspected of having a disease associated with altered sphingolipid metabolism with at least one oligonucleotide that is specific for a polynucleotide which comprises a nucleic acid sequence as set forth in SEQ ID NO:22; (b) detecting an amount of the olignucleotide that hybridizes to the polynucleotide in the first sample; and (d) comparing the amount of oligonucleotide that hybridizes to the polynucleotide in the first sample to an amount of oligonucleotide that hybridizes to a polynucleotide in a second biological sample obtained from a normal control subject known to be free of a disease associated with altered sphingolipid metabolism, wherein a statistically significant increase in the amount of olignucleotide that hybridizes to the polynucleotide in the first biological sample relative to the amount of oligonucleotide that hybridizes to the polynucleotide in the second sample signifies the presence of a disease associated with altered sphingolipid metabolism in said patient. It is another aspect of the present invention to provide a method for treating a disease associated with altered sphingolipid metabolism in a patient, comprising administering to said patient an agent identified according to any of the above described methods. In certain further embodiments the disease is colon cancer, breast cancer, uterine cancer, stomach cancer, ovarian cancer, lung cancer, kidney cancer, adenocarcinoma of the rectum, hereditary sensory neuropathy type 1, or any one of the sphingolipidoses.

Another embodiment of the present invention provides a method for identifying an agent that modulates sphingolipid metabolism, comprisingculturing a mutant yeast strain with sphingosine in the absence and presence of a candidate agent under conditions and for a time sufficient to observe in said mutant yeast strain an altered level of either (i) at least one sphingolipid intermediate, or (ii) activity of at least one component of a sphingolipid pathway, wherein the mutant yeast strain comprises a null allele of at least one gene encoding a component of a sphingolipid pathway that results in an altered activity level of at least one sphingolipid pathway component, and wherein said mutant strain of yeast has been genetically altered to express at least one nonendogenous sphingolipid pathway component and wherein the mutant yeast strain exhibits growth inhibition in the presence of sphingosine; and comparing growth of the mutant yeast strain in the presence of the candidate agent to the growth of the mutant yeast strain in the absence of the candidate agent, wherein an increase in growth of the mutant yeast strain in the presence of the agent indicates the agent modulates sphingolipid metabolism. In one embodiment, the at least one gene comprises DPL1. In another embodiment the at least one gene comprises DPL1 and LCB4. In another embodiment, the nonendogenous sphingolipid pathway component comprises human SPHK1. In certain embodiments, the altered level of the sphingolipid intermediate in the presence of the candidate agent comprises a decrease in at least one LCBP. In one embodiment, the LCBP comprises sphingosine-1-phosphate. In another embodiment, the altered level of the activity of the sphingolipid pathway component in the presence of the candidate agent comprises a decrease in the human SPHK1 activity.

In certain embodiments, the present invention provides a method for identifying an inhibitor of SK, comprising culturing a null mutant yeast strain with sphingosine in the absence and presence of a candidate agent under conditions and for a time sufficient to observe in said mutant yeast strain an altered ability of said yeast mutant strain to grow, wherein the mutant yeast strain comprises a null allele of at least DPL1 and LCB4, and wherein said mutant strain of yeast has been genetically altered to express nonendogenous SK and wherein the mutant yeast strain exhibits growth inhibition in the presence of sphingosine; and comparing growth of the mutant yeast strain in the presence of the candidate agent to the growth of the mutant yeast strain in the absence of the candidate agent, wherein an increase in growth of said mutant yeast strain in the presence of the agent indicates the agent is an inhibitor of SK.

In certain embodiments, the present invention provides a method for identifying an inhibitor of SK, comprising culturing a null mutant yeast strain with an inducer in the absence and presence of a candidate agent under conditions and for a time sufficient to observe altered growth in said mutant yeast strain, wherein the mutant yeast strain comprises a null allele of at least DPL1, LCB4, and SPP, and wherein said mutant strain of yeast has been genetically altered to express nonendogenous SK under the control of a promoter that is induced by the inducer and wherein the mutant yeast strain exhibits growth inhibition in the presence of the inducer; and comparing growth of the mutant yeast strain in the presence of the candidate agent to the growth of the mutant yeast strain in the absence of the candidate agent, wherein an increase in growth of said mutant yeast strain in the presence of the candidate agent indicates the candidate agent is an inhibitor of SK. In one embodiment, the inducible promoter is a galactose-inducible promoter. In certain embodiment, the inducer includes but is not limited to galactose.

The present invention also provides a method for identifying an agent that modulates sphingolipid metabolism, comprising culturing a mutant yeast strain with sphingosine in the absence and presence of a candidate agent under conditions and for a time sufficient to observe in said mutant yeast strain an altered level of either (i) at least one sphingolipid intermediate, or (ii) activity of at least one component of a sphingolipid pathway, wherein the mutant yeast strain comprises a null allele of at least one gene encoding a component of a sphingolipid pathway that results in an altered activity level of at least one sphingolipid pathway component, and wherein said mutant strain of yeast has been genetically altered to express at least one nonendogenous sphingolipid pathway component and wherein the mutant yeast strain exhibits growth inhibition in the presence of sphingosine; and comparing the level of either (i) or (ii) in the mutant yeast strain cultured in the presence of the candidate agent to the level of either (i) or (ii) in the mutant yeast strain cultured in the absence of the candidate agent, wherein an altered level in the presence of the agent indicates the agent modulates sphingolipid metabolism. In one embodiment, the altered level of said at least one sphingolipid intermediate comprises a decrease in S-1-P. In certain embodiments, the altered level of said activity of at least one component of a sphingolipid pathway comprises a decrease in the activity of said at least one nonendogenous sphingolipid pathway component. In one embodiment, the nonendogenous sphingolipid pathway component comprises human SPHK1.

In another embodiment, the present invention provides a method for identifying an agent that modulates sphingolipid metabolism, comprising culturing a null mutant yeast strain with sphingosine in the absence and presence of a candidate agent under conditions and for a time sufficient to observe altered growth of said mutant yeast strain, wherein the mutant yeast strain comprises a null allele of at least one gene encoding a component of a sphingolipid pathway that results in an altered activity level of at least one sphingolipid pathway component, and wherein said mutant strain of yeast has been genetically altered to express at least one nonendogenous sphingolipid pathway component, and wherein the mutant yeast strain exhibits growth inhibition in the presence of sphingosine; and comparing growth of the mutant yeast strain in the presence of the candidate agent to growth of the mutant yeast strain in the absence of the candidate agent, wherein an increase in growth of said mutant yeast strain in the presence of the candidate agent indicates the agent modulates sphingolipid metabolism. In one embodiment the null allele of at least one gene encoding a component of a sphingolipid pathway comprises DPL1. In another embodiment, the at least one gene comprises DPL1 and LCB4. In certain embodiments, the at least one nonendogenous sphingolipid pathway component comprises human SPHK1.

The present invention also provides a method for identifying an agent that modulates sphingolipid metabolism, comprising culturing a mutant yeast strain with an inducer in the absence and presence of a candidate agent under conditions and for a time sufficient to observe in said mutant yeast strain an altered level of either (i) at least one sphingolipid intermediate, or (ii) activity of at least one component of a sphingolipid pathway, wherein the mutant yeast strain comprises a null allele of at least one gene encoding a component of a sphingolipid pathway that results in an altered activity level of at least one sphingolipid pathway component, and wherein said mutant strain of yeast has been genetically altered to express at least one nonendogenous sphingolipid pathway component under the control of a promoter that is induced by the inducer and wherein the mutant yeast strain exhibits growth inhibition in the presence of the inducer; and comparing the level of either (i) or (ii) in the mutant yeast strain cultured in the presence of the candidate agent to the level of either (i) or (ii) in the mutant yeast strain cultured in the absence of the candidate agent, wherein an altered level in the presence of the agent indicates the agent modulates sphingolipid metabolism. In certain embodiments, the altered level of said at least one sphingolipid intermediate comprises a decrease in LCBPs. In another embodiment the altered level of said activity of at least one component of a sphingolipid pathway comprises a decrease in the activity of said at least one nonendogenous sphingolipid pathway component. In yet another embodiment, the at least one nonendogenous sphingolipid pathway component comprises human SPHK1. In certain embodiments the at least one gene comprises DPL1. In another embodiment, the at least one gene comprises DPL1 and LCB4. In yet another embodiment, the at least one gene comprises DPL1, LCB4, and YSR2.

The present invention also provides a method for identifying an agent that modulates sphingolipid metabolism, comprising culturing a null mutant yeast strain with an inducer in the absence and presence of a candidate agent under conditions and for a time sufficient to observe altered growth of said mutant yeast strain, wherein the mutant yeast strain comprises a null allele of at least one gene encoding a component of a sphingolipid pathway that results in an altered activity level of at least one sphingolipid pathway component, and wherein said mutant strain of yeast has been genetically altered to express at least one nonendogenous sphingolipid pathway component under the control of a promoter that is induced by the inducer and wherein the mutant yeast strain exhibits growth inhibition in the presence of the inducer; and comparing growth of the mutant yeast strain in the presence of the candidate agent to growth of the mutant yeast strain in the absence of the candidate agent, wherein an increase in growth of said mutant yeast strain in the presence of the candidate agent indicates the agent modulates sphingolipid metabolism. In one embodiment, the at least one gene comprises DPL1. In another embodiment, the at least one gene comprises DPL1 and LCB4. In yet another embodiment, the at least one gene comprises DPL1, LCB4, and YSR2. In certain embodiments, the at least one nonendogenous sphingolipid pathway component comprises human SPHK1.

These and other aspects of the present invention Will become apparent upon reference to the following detailed description and attached drawings. All references (including websites) disclosed herein are hereby incorporated by reference in their entireties as if each was incorporated individually.

### BRIEF DESCRIPTION OF THE FIGURES AND SEQUENCE IDENTIFIERS

FIG. 1 shows the amino acid sequence of 2 potential Drosophila melanogaster SK proteins aligned with the amino acid sequence of a human SK protein. (DSK1747 set forth in SEQ ID NO:19; DSK2159 set forth in SEQ ID NO:20).

FIG. 2 shows a first chemical synthesis scheme.

FIG. 3 shows a second chemical synthesis scheme.

FIG. 4 is a schematic diagram showing a first yeast screen for inhibitors of SK.

FIG. 5 is a schematic diagram showing a second yeast screen for inhibitors of SK.

SEQ ID NO:1 is the determined cDNA sequence of S. cerevisiae SPL

SEQ ID NO:2 is the amino acid sequence of S. cerevisiae SPL encoded by the polynucleotide sequence set forth in SEQ ID NO:1

SEQ ID NO:3 is the determined cDNA sequence of C. elegans SPL

SEQ ID NO:4 is the amino acid sequence of C. elegans SPL encoded by the polynucleotide sequence set forth in SEQ ID NO:3

SEQ ID NO:5 is the determined cDNA sequence of the mouse SPL

SEQ ID NO:6 is the amino acid sequence of mouse SPL encoded by the polynucleotide sequence set forth in SEQ ID NO:5

SEQ ID NO:7 is the determined cDNA sequence of the full-length human SPL

SEQ ID NO:8 is the amino acid sequence of human SPL encoded by the polynucleotide sequence set forth in SEQ ID NO:7

SEQ ID NO:9 is the determined cDNA sequence of a human SPL with a deletion

SEQ ID NO:10 is the amino acid sequence of a human SPL with a deletion, encoded by the polynucleotide sequence set forth in SEQ ID NO:9.

SEQ ID NO:11 is the amino acid sequence of C. elegans SPL encoded by the polynucleotide sequence set forth in SEQ ID NO:12

SEQ ID NO:12 is the determined cDNA sequence of a C. elegans SPL

SEQ ID NO:13 is a PCR primer

SEQ ID NO:14 is a PCR primer

SEQ ID NO:15 is the determined cDNA sequence encoding the Drosophila melanogaster SPL

SEQ ID NO:16 is the amino acid sequence of the Drosophila melanogaster SPL, encoded by the cDNA sequence set forth in SEQ ID NO:15

SEQ ID NO:17 is the determined cDNA sequence of a human SPL as set forth in Genbank Accession No: AF144638.

SEQ ID NO:18 is the amino acid sequence of a human SPL encoded by the polynucleotide sequence provided in SEQ ID NO:17.

SEQ ID NO:19 is the amino acid sequence of a first Drosophila melanogaster SK protein.

SEQ ID NO:20 is the amino acid sequence of a second Drosophila melanogaster SK protein.

SEQ ID NO:21 is the amino acid sequence of a human SK protein.

SEQ ID NO:22 is the cDNA encoding the human SK protein set forth in SEQ ID NO:21.

SEQ ID NO:23 is a cDNA sequence of human SPL, encoding the amino acid sequence set forth in SEQ ID NO:18.

SEQ ID NO:24 is the full length cDNA sequence for a first Drosophila melanogaster SK1, GI:21429173, encoding the amino acid sequence set forth in SEQ ID NO:19 and 28.

SEQ ID NO:25 is the full length cDNA sequence for a second Drosophila melanogaster SK2, GI:17862169, encoding the amino acid sequence set forth in SEQ ID NO:20 and 29.

SEQ ID NO:26 is the full length cDNA sequence for Drosophila melanogaster SPL, clone GH13783, GI:15292460.

SEQ ID NO:27 is the full length cDNA sequence for Drosophila melanogaster SPL, clone LP04413, GI:15292460.

SEQ ID NO:28 is the full length amino acid sequence of Drosophila melanogaster SK1 CG1747.

SEQ ID NO:29 is the full length amino acid sequence of Drosophila melanogaster CG2159.

SEQ ID NO:30 is the polynucleotide coding region for the Saccharomyces cerevisiaie LCB4 protein as available from yeastgenome.org.

SEQ ID NO:31 is the amino acid sequence of the protein encoded by the polynucleotide of SEQ ID NO:30, the yeast LCB4 protein.

SEQ ID NO:32 is the polynucleotide coding region for the Saccharomyces cerevisiaie LCB5 protein as available from yeastgenome.org.

SEQ ID NO:33 is the amino acid sequence of the protein encoded by the polynucleotide of SEQ ID NO:32, the yeast LCB5 protein.

SEQ ID NO:34 is the polynucleotide coding region for the Saccharomyces cerevisiaie YSR2 protein (also referred to as LCB3) as available from yeastgenome.org.

SEQ ID NO:35 is the amino acid sequence of the protein encoded by the polynucleotide of SEQ ID NO:34, the yeast YSR2 protein (also referred to as LCB3).

SEQ ID NO:36 is the polynucleotide coding region for the Saccharomyces cerevisiaie YSR3 protein as available from yeastgenome.org.

SEQ ID NO:37 is the amino acid sequence of the protein encoded by the polynucleotide of SEQ ID NO:36, the yeast YSR3 protein.

SEQ ID NO:38 is the polynucleotide coding region for the Saccharomyces cerevisiaie SUR2 protein as available from yeastgenome.org.

SEQ ID NO:39 is the amino acid sequence of the protein encoded by the polynucleotide of SEQ ID NO:38, the yeast SUR2 protein.

SEQ ID NO:40 is a PCR primer as described in Example 17.

SEQ ID NO:41 is a PCR primer as described in Example 17.

### DETAILED DESCRIPTION OF THE INVENTION

The present invention provides compositions and methods, including screening assays, for agents that modulate sphingolipid metabolism and/or signaling wherein the agents have an effect on a level of sphingolipid intermediates and/or the activity of one or more components of a sphingolipid metabolic and/or signaling pathway and further provides methods for screening for said agents. Agents of the present invention have utility in the detection, diagnosis, and therapy of cancer and other diseases associated with altered sphingolipid metabolism and/or signaling.

Generally, the present invention relates to involvement of sphingolipid intermediates, and components involved in sphingolipid metabolism and/or signaling pathways, in numerous human diseases, including a variety of cancers (e.g. colon, breast, uterus, stomach, ovary, lung, kidney and adenocarcinoma of the rectum). In particular, the present invention derives from the unexpected observation that SPL expression is reduced in colloid cancer of the colon and adenocarcinoma of the colon. Also according to the present invention as disclosed below in greater detail, reduced SPL expression is observed in adenocarcinoma of the uterus, and SK expression is increased in a variety of tumor tissues as compared to normal tissue (e.g. breast, uterus, stomach, ovary, lung, kidney and adenocarcinoma of the rectum). Other components involved in sphingolipid metabolism and/or signaling pathways are also associated with other human diseases. In particular, failure and/or dysregulation of sphingolipid synthesis and/or catabolism are directly responsible for a number of human diseases, including hereditary sensory neuropathy type 1 and the group of lysosomal storage diseases called the sphingolipidoses (Bejaoui, K., Wu, C., Scheffler, M. D., Haan, G., Ashby, P., Wu, L., de Jong, P. and Brown, R. H., Jr. (2001). Nat Genet 27, 261-2.; Dawkins, J. L., Hulme, D. J., Brahmbhatt, S. B., Auer-Grumbach, M. and Nicholson, G. A. (2001). Nat Genet 27, 309-12.; Gable, K., Han, G., Monaghan, E., Bacikova, D., Natarajan, M., Williams, R. and Dunn, T. M. (2002). J Biol Chem 277, 10194-200.).

The present invention further relates in certain embodiments to the unanticipated observation that Drosophila melanogaster SPL and SK mutants demonstrate altered sphingolipid metabolism. Surprisingly, SPL mutant flies have a flightless phenotype that can be restored by growing such mutant flies in the presence of an agent that modifies a component of the sphingolipid metabolic and/or signaling pathway. Thus, the present invention provides mutant and/or transgenic Drosophila melanogaster that have altered sphinoglipid metabolism and/or signaling that can be used to screen agents useful for the detection, diagnosis, and treatment of the human diseases described herein.

In certain other embodiments, the present invention relates to the surprising discovery that components of yeast and mammalian sphingolipid pathways can be functionally combined in a yeast mutant expression system. Unexpectedly, such yeast systems exhibit cell growth inhibition in response to elevated intracellular phosphorylated long chain sphingoid bases (LCBP) (e.g., S-1-P), unlike many mammalian cells that proliferate in response to excessive accumulations of LCBPs. These and related embodiments advantageously provide mutant and/or recombinant yeast strains having sphingolipid pathway defects that permit the use of such strains as indicator cells in screening assays for agents which modulate sphingolipid metabolism, and in particular, for SK inhibitors.

Components of Sphingolipid Metabolism and/or Signaling

Any component of the sphingolipid metabolic and/or signaling pathway may be regarded as within the context of the present invention. As such, components of the sphingolipid metabolic and/or signaling pathway include but are not limited to, enzymes involved in these pathways (and the polynucleotides encoding said enzymes), such as, SPL, SK, ceramidase, S-1-PP, serine palmitoyltransferase (SPT), 3-keto dihydrosphingosine reductase, ceramide synthase, sphingosine desaturase, ceramide kinase, phosphoethanolamine cytidylyltransferase, CDP-ethanolamine phosphotransferase, acid sphingomyelinase, sphingomyelin synthase, neutral sphingomyelinase, oxosphinanine reductase, and glucosylceramide synthase. Components of the sphingolipid metabolic and/or signaling pathway further include intracellular or cell surface receptors, and the polynucleotides encoding said receptors, such as EDG receptors (e.g. EDG1, EDG3, EDG5, EDG6, EDG8) and CFTR.

Generally sphingolipid metabolism can be viewed as all synthetic and catabolic pathways involving any sphingolipid or sphingolipid intermediate as described herein. Sphingolipid signaling pathways are known in the art and can generally be viewed herein as any signaling pathway activated by a sphingolipid, such as the signaling pathways of sphingosine-1-phosphate such as those described in Pyne, S., and N. J. Pyne. 2000 Biochem. J. 349:385-402 and Pyne, S., and N. J. Pyne, 2000 Pharmacology and Therapeutics 88:115-131. However, the skilled artisan would recognize that other sphingolipid signaling pathways fall within the scope of the present invention and are contemplated herein.

The present invention therefore provides polypeptides involved in sphingolipid metabolism and/or signaling, and polynucleotides encoding such polypeptides. As used herein, the term “polypeptide” encompasses amino acid chains of any length, including full length endogenous (i.e., native) proteins and variants of endogenous sequences that are involved in sphingolipid metabolism and/or signaling. Illustrative polypeptides of the present invention are set forth in SEQ ID NOs:2, 4, 6, 8, 10,11, 16, 18-21, and 28-29. Particularly illustrative polypeptides are set forth in SEQ ID NOs:16, 18-21, and 28-29. “Variants” are polypeptides that differ in sequence from the polypeptides of the present invention only in substitutions, deletions and/or other modifications, such that the variant retains ability to modulate sphingolipid metabolism and/or signaling, for example by effecting the levels of one or more sphingolipid intermediates, such as intracellular S-1-P, ceramide, sphingosine, or other LCB or LCBP levels, which may be determined using a representative method described herein. Polypeptide variants generally encompassed by the present invention will typically exhibit at least about 70%, 75%, 80%, 85%, 86%, 87%, 88%, 89%, 90%, 91%, 92%, 93%, 94%, 95%, 96%, 97%, 98%, or 99% or more identity along its length, to a polypeptide sequence set forth herein. Within a polypeptide variant, amino acid substitutions are preferably made at no more than 50% of the amino acid residues in the native polypeptide, and more preferably at no more than 25% of the amino acid residues. Such substitutions are preferably conservative. A conservative substitution is one in which an amino acid is substituted for another amino acid that has similar properties, such that one skilled in the art of peptide chemistry would expect the secondary structure and hydropathic nature of the polypeptide to be substantially unchanged. In general, the following amino acids represent conservative changes: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. Substitutions, deletions and/or amino acid additions may be made at any location(s) in the polypeptide, provided that the modification does not diminish the ability of the variant to modulate intracellular S-1-P levels. Thus, a variant may comprise only a portion of a native polypeptide sequence as provided herein. In addition, or alternatively, variants may contain additional amino acid sequences (such as, for example, linkers, tags and/or ligands), preferably at the amino and/or carboxy termini. Such sequences may be used, for example, to facilitate purification, detection or cellular uptake of the polypeptide.

When comparing polypeptide sequences, two sequences are said to be “identical” if the sequence of amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Saitou, N. Nei, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

Preferred examples of algorithms that are suitable for determining percent sequence identity and sequence similarity include the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

An “isolated” polypeptide is one that is removed from its original environment. For example, a naturally-occurring protein or polypeptide is isolated if it is separated from some or all of the coexisting materials in the natural system. Preferably, such polypeptides are also purified, e.g., are at least about 90% pure, more preferably at least about 95% pure and most preferably at least about 99% pure.

In one embodiment of the present invention, a polypeptide comprises a fusion protein comprising a component of a sphingolipid metabolic and/or signaling pathway. The present invention further provides, in other aspects, fusion proteins that comprise at least one polypeptide as described above, as well as polynucleotides encoding such fusion proteins, typically in the form of pharmaceutical compositions, e.g., vaccine compositions, comprising a physiologically acceptable carrier or excipient. The fusion proteins may comprise multiple polypeptides or portions/variants thereof, as described herein, and may further comprise one or more polypeptide segments for facilitating the expression, purification, detection, and/or activity of the polypeptide(s).

In general, polypeptide components of a sphingolipid metabolic and/or signaling pathway, and polynucleotides encoding such polypeptides as described herein, may be prepared using any of a variety of techniques that are well known in the art. For example, a DNA sequence encoding native SK, SPL or SPT may be prepared by amplification from a suitable cDNA or genomic library using, for example, polymerase chain reaction (PCR) or hybridization techniques. Libraries may generally be prepared and screened using methods well known to those of ordinary skill in the art, such as those described in Sambrook et al., Molecular Cloning: A Laboratory Manual, Cold Spring Harbor Laboratories, Cold Spring Harbor, N.Y., 1989. cDNA libraries may be prepared from any of a variety of sources known to contain enzymes involved in sphingolipid metabolism. For example, SPL activity is ubiquitous with regard to species and mammalian tissues, with the exception of platelets, in which SPL activity is notably absent. In rat tissues, the highest levels of activity have been demonstrated in intestinal mucosa, liver and Harderian gland, with low activity in skeletal muscle and heart. Activity has also been demonstrated in a number of human (hepatoma cell line HB 8065, cervical carcinoma HeLa), mouse (hepatoma line BW1, mouse embryo 3T3-L1, Swiss 3T3 cells) and other cell lines, as well as in human cultured fibroblasts. Preferred cDNA libraries may prepared from human liver, intestine or brain tissues or cells. Other libraries that may be employed will be apparent to those of ordinary skill in the art. Primers for use in amplification may be readily designed based on the polynucleotide sequence of a native SPL, SK, SPT, S-1-PP or other polynucleotide as provided herein or known to the skilled artisan and available on any number of public databases.

A polynucleotide encoding a polypeptide component involved in a sphingolipid pathway (metabolic and/or signaling), such as a polynucleotide encoding SPL, SK, SPT, and S-1-PP, are also provided by the present invention. A polynucleotide as used herein may be single-stranded (coding or antisense) or double-stranded, and may be DNA (genomic, cDNA or synthetic) or RNA molecules. Thus, within the context of the present invention, a polynucleotide encoding a polypeptide may also be a gene. A gene is a segment of DNA involved in producing a polypeptide chain; it includes regions preceding and following the coding region (leader and trailer) as well as intervening sequences (introns) between individual coding segments (exons). Additional coding or non-coding sequences may, but need not, be present within a polynucleotide of the present invention, and a polynucleotide may, but need not, be linked to other molecules and/or support materials. “Isolated,” as used herein, means that a polynucleotide is substantially away from other coding sequences, and that the DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment by the hand of man.

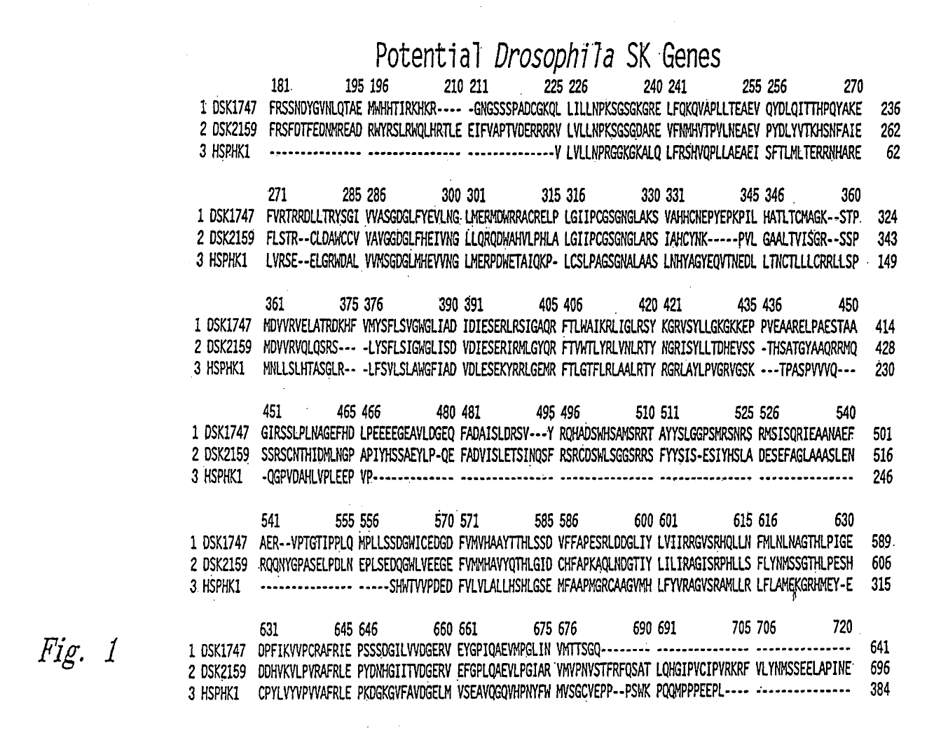

Polynucleotides of the present invention may comprise a native sequence (i.e., an endogenous polynucleotide, for instance, a native or non-artificially engineered or naturally occurring gene as provided herein) encoding SPL, SK, SPT, or other components of the sphingolipid metabolic or signaling pathways, alternate form sequence, or a portion or splice variant thereof or may comprise a variant of such a sequence. Polynucleotide variants may contain one or more substitutions, additions, deletions and/or insertions such that the activity of the encoded polypeptide is not substantially diminished, as described herein. The effect on the activity of the encoded polypeptide may generally be assessed as described herein. Variants preferably exhibit at least about 70% identity, more preferably at least about 80%, 85%, 86%, 87%, 88%, 89%, identity and most preferably at least about 90%, 91%, 92%, 93%, 94%, 95%, 96%, 97%, 98%, or 99% identity to a polynucleotide sequence that encodes a native polypeptide involved in sphingolipid metabolism or signaling, such as the polynucleotides set forth in SEQ ID NOs:1, 3, 5, 7, 9, 12, 15, 17, and 22-27 or an alternate form or a portion thereof, and the polynucleotides that encode a polypeptide sequence as recited in any one of SEQ ID NOs:2, 4, 6, 8, 10, 11, 16, and 18-21, or a portion thereof. Particularly illustrative polynucleotides of the present invention comprise polynucleotides encoding a polypeptide comprising an amino acid sequence shown in FIG. 1, such as the amino acid sequences set forth in SEQ ID NOs:18-21 and 28-29. The percent identity may be readily determined by comparing sequences using computer algorithms well known to those having ordinary skill in the art and described herein.

Polynucleotides that are substantially homologous to a sequence complementary to a polynucleotide as described herein are also within the scope of the present invention. “Substantial homology,” as used herein refers to polynucleotides that are capable of hybridizing under moderately stringent conditions to a polynucleotide complementary to an SK, SPL, SPT, S-1-PP or other polynucleotide sequence provided herein, provided that the encoded polypeptide variant retains enzymatic or signaling activity. Suitable moderately stringent conditions include prewashing in a solution of 5×SSC, 0.5% SDS, 1.0 mM EDTA (pH 8.0); hybridizing at 50-65° C., 5×SSC, overnight; followed by washing twice at 65° C. for 20 minutes with each of 2×, 0.5× and 0.2×SSC containing 0.1% SDS. Nucleotide sequences that, because of code degeneracy, encode a polypeptide encoded by any of the above sequences are also encompassed by the present invention.

A polynucleotide as described herein may be identified using standard yeast genetics known to the skilled artisan. A cDNA expression library may be generated using a regulatable yeast expression vector (e.g., pYES, which is available from Invitrogen, Inc.) and standard techniques. A yeast mutant strain may then be transformed with the cDNA library, and endogenous cDNAs having the ability to functionally complement the yeast sphingolipid metabolism defect (i.e., restore the ability to grow in the presence of D-erythro-sphingosine or other appropriate sphingolipid intermediate) may be isolated.

A polynucleotide encoding a polypeptide affecting sphingolipid metabolism and/or signaling may also be identified based on cross-reactivity of the protein product with antibodies that react to SPL, SK, SPT, and other polypeptides involved in sphingolipid metabolism or signaling, which may be prepared as described herein. Such screens may generally be performed using standard techniques (see Huynh et al., “Construction and Screening cDNA Libraries in λgt11,” in D. M. Glover, ed., DNA Cloning: A Practical Approach, 1:49-78, 1984 (IRL Press, Oxford)).

Polypeptides of the present invention may be prepared by expression of recombinant DNA encoding the polypeptide in cultured host cells. Preferably, the host cells are bacteria, yeast, insect or mammalian cells, and preferably the host cells are S. cerevisiae bst1Δ cells. The recombinant DNA may be cloned into any expression vector suitable for use within the host cell and transfected into the host cell using techniques well known to those of ordinary skill in the art. A suitable expression vector contains a promoter sequence that is active in the host cell. A tissue-specific or conditionally active promoter may also be used. Preferred promoters express the polypeptide at high levels. As is readily appreciated by the skilled artisan, the polynucleotide encoding the polypeptide of interest is cloned into the expression vector such that it is operably linked to the promoter such that the polypeptide of interest is properly translated. Thus, in certain embodiments, the ligated DNA sequences are operably linked to suitable transcriptional or translational regulatory elements. The regulatory elements responsible for expression of DNA are generally located only 5′ to the DNA sequence encoding the first polypeptides. Similarly, stop codons required to end translation and transcription termination signals are only present 3′ to the DNA sequence encoding the second polypeptide.

Optionally, the construct may contain an enhancer, a transcription terminator, a poly(A) signal sequence, a bacterial or mammalian origin of replication and/or a selectable marker, all of which are well known in the art. Enhancer sequences may be included as part of the promoter region or separately. Transcription terminators are sequences that stop RNA polymerase-mediated transcription. The poly(A) signal may be contained within the termination sequence or incorporated separately. A selectable marker includes any gene that confers a phenotype on the host cell that allows transformed cells to be identified. Such markers may confer a growth advantage under specified conditions. Suitable selectable markers for bacteria are well known and include resistance genes for ampicillin, kanamycin and tetracycline. Suitable selectable markers for mammalian cells include hygromycin, neomycin, genes that complement a deficiency in the host (e.g., thymidine kinase and TK-cells) and others well known in the art. For yeast cells, one suitable selectable marker is URA3, which confers the ability to grow on medium without uracil.

DNA sequences expressed in this manner may encode a native polypeptide (e.g., human) involved in sphingolipid metabolism or signaling, such as SK, SPL, SPT, or may encode portions or other variants of a native polypeptide involved in sphingolipid metabolism or signaling, such as SK, SPL, SPT or other polypeptides of the present invention described herein. DNA molecules encoding variants of a native polynucleotide may generally be prepared using standard mutagenesis techniques, such as oligonucleotide-directed site-specific mutagenesis, and sections of the DNA sequence may be removed to permit preparation of truncated polypeptides.

To generate cells that express a polynucleotide encoding a polypeptide, such as SPL, SPT, SK, involved in sphingolipid metabolism, cells may be transfected, transformed or transduced using any of a variety of techniques known in the art. Any number of transfection, transformation, and transduction protocols known to those in the art may be used, for example those outlined in Current Protocols in Molecular Biology, John Wiley & Sons, New York. N.Y., or in numerous kits available commercially (e.g., Invitrogen Life Technologies, Carlsbad, Calif.). Such techniques may result in stable transformants or may be transient. One suitable transfection technique is electroporation, which may be performed on a variety of cell types, including mammalian cells, yeast cells and bacteria, using commercially available equipment. Optimal conditions for electroporation (including voltage, resistance and pulse length) are experimentally determined for the particular host cell type, and general guidelines for optimizing electroporation may be obtained from manufacturers. Other suitable methods for transfection will depend upon the type of cell used (e.g., the lithium acetate method for yeast), and will be apparent to those of ordinary skill in the art. Following transfection, cells may be maintained in conditions that promote expression of the polynucleotide within the cell. Appropriate conditions depend upon the expression system and cell type, and will be apparent to those skilled in the art.

Polypeptides involved in sphingolipid metabolism may be expressed in transfected cells by culturing the cell under conditions promoting expression of the transfected polynucleotide. Appropriate conditions will depend on the specific host cell and expression vector employed, and will be readily apparent to those of ordinary skill in the art. For commercially available expression vectors, the polypeptide may generally be expressed according to the manufacturer's instructions. For certain purposes, expressed polypeptides of this invention may be isolated in substantially pure form. Preferably, the polypeptides are isolated to a purity of at least 80% by weight, more preferably to a purity of at least 95% by weight, and most preferably to a purity of at least 99% by weight. In general, such purification may be achieved using, for example, the standard techniques of ammonium sulfate fractionation, SDS-PAGE electrophoresis, and/or affinity chromatography.

Sphingolipid Intermediates

As noted herein above, the present invention provides agents that modulate the activity of one or more components of a sphingolipid metabolic and/or signaling pathway. The agents of the present invention also may alter the levels (e.g., relative or absolute amounts, concentrations, stability, or the like) of at least one sphingolipid intermediate. Sphingolipid intermediates of the present invention include any sphingolipid intermediate in the sphingolipid metabolic pathway. As such, the sphingolipid intermediates of the present invention include, but are not limited to, long chain bases (LCBs) and phosphorylated long chain bases (LCBPs) comprising sphingoid backbone structures of between C₁₀ and C₂₀. In one embodiment, the backbone structure comprises C₁₄, C₁₅, C₁₆, C₁₇, C₁₈, C₁₉, or C₂₀. In a further embodiment, the sphingolipid intermediates of the present invention include endogenous free sphingoid bases isolated from Drosophila melanogaster, including C₁₄ and C₁₆ sphingosine and C₁₄ and C₁₆ dihydrosphingosine. In another embodiment, a sphingolipid intermediate comprises any one or more of S-1-P, hexadecanal, phosphoethanolamine, ceramide, sphingosine, 3-keto-dihydrosphingosine, dihydrosphingosine, sphingomyelin, dihydroceramide, ceramide-1-phosphate, dihydrosphingosine-1-phosphate, ethanolamine phosphate, long chain unsaturated aldehyde, and long chain saturated aldehyde. The skilled artisan would readily appreciate that any sphingolipid intermediate species that is affected or generated by any one or more components of the sphingolipid metabolic and/or signaling pathway fall within the scope of the present invention and can be identified using a variety of assays known in the art and further described herein.

Agents that Modulate Sphingolipid Intermediates and/or Components of Sphingolipid Metabolism and/or Signaling

Agents for use according to the present invention are defined as any composition, compound, substance, molecule, material, product or the like, whether artificial or naturally derived, as described herein in further detail, that modulate sphingolipid metabolism and/or signaling. An agent that modulates sphingolipid metabolism and/or signaling is an agent that alters (e.g., increases or decreases in a statistically significant manner) the level of at least one sphingolipid intermediate or the activity of at least one component of a sphingolipid metabolic and/or signaling pathway. Alteration of a level or activity comprises any statistically significant change, e.g. increase or decrease, in the level of one or more intermediates or in the activity of one or more components of sphingolipid metabolism and/or signaling as described herein, when an isolated component, or a host cell or an animal comprising an intermediate or component is contacted with the agent as compared to an isolated component, a host cell or animal comprising an intermediate or component that is not contacted with the agent. As such, in one embodiment, modulation comprises an altered level, e.g. a statistically significant decrease or increase in, a polynucleotide encoding a protein involved in sphingolipid metabolism and/or signaling as described herein. Numerous methods for detecting polynucleotide levels (e.g. gene expression) are known in the art and are useful in the context of the instant invention. Illustrative methods are described in Ausubel et al. (1993 Current Protocols in Molecular Biology, Greene Publ. Assoc. Inc. & John Wiley & Sons, Inc., Boston, Mass.); Sambrook et al. (1989 Molecular Cloning, Second Ed., Cold Spring Harbor Laboratory, Plainview, N.Y.); Maniatis et al. (1982 Molecular Cloning, Cold Spring Harbor Laboratory, Plainview, N.Y.) and elsewhere.

In a further embodiment, modulation comprises an altered activity level, that is a statistically significant decrease or increase in enzymatic activity of any enzyme involved in sphingolipid metabolism and/or signaling, such as SPL, SK, SPT, S-1-PP, and the like. Numerous methods for detecting and measuring enzymatic activity are known in the art and can be used in the context of the present invention (see e.g. Current Protocols in Protein Science, John Wiley & Sons, Inc., Boston, Mass.). Certain illustrative methods are described in, e.g., Saba, J. D., Nara, F., Bielawska, A., Garrett, S. and Hannun, Y. A. (1997). J Biol Chem 272, 26087-26090, and Van Veldhoven, P. P. and Mannaerts, G. P. (1991). J Biol Chem 266, 12502-7, Williams, R, Wang E and Merrill A, 1984., Arch Biochem Biophys 228:282-291., Caligan, T B, Peters K, Ou J, Wang E, Saba J and Merrill A H, Jr., 2000. Analytical Biochemistry 281:36-44.

In certain embodiments, modulation comprises a statistically significant decrease or increase in the levels of (i.e. altered level of) one or more sphingolipid intermediates as described herein, such as S-1-P, ceramide, sphingosine, or other LCBs or LCBPs. A variety of methods for measuring sphingolipid intermediates (e.g., sphingosine-1-phosphate or its degradation products, ceramide, sphingosine, etc.) is known in the art and may be useful in the context of the present invention. Illustrative methods are described in the following references: Bose, R and Kolesnick R, 2000. Methods in Enzymology 322:373-378; Fyrst, H, et al., 1999, Biochemistry 38:5864-5871; Fyrst, H, et al., 1996, Biochemistry 35:2644-2650.

In certain embodiments modulation of sphingolipid metabolism and/or signaling comprises an increase or decrease in cellular proliferation, apoptosis, angiogenesis, drug resistance and cell motility. A variety of assays are known in the art to measure these activities, including those described in Current Protocols in Immunology, or Current Protocols in Cell Biology, both published by John Wiley & Sons, Inc., Boston, Mass.

Candidate agents of the present invention include polynucleotides encoding polypeptide components of the sphingolipid metabolic and/or signaling pathways such as any of said polypeptide components described herein. Agents of the present invention further include a polypeptide comprising an enzyme involved in sphingolipid metabolism or signaling such as those described herein.

Candidate agents further include any of the sphingolipid intermediates described herein, such as, but not limited to, S-1-P, hexadecanal, phosphoethanolamine, ceramide, sphingosine, 3-keto-dihydrosphingosine, dihydrosphingosine, sphingomyelin, dihydroceramide, ceramide-1-phosphate, dihydrosphingosine-1-phosphate, ethanolamine phosphate, long chain unsaturated aldehyde, and long chain saturated aldehyde. In one embodiment, an agent of the present invention comprises LCBs and LCBPs such as C₁₄ and C₁₆ sphingosine and C₁₄ and C₁₆ dihydrosphingosine identified in the Drosophila melanogaster as described herein.

In one particular embodiment, agents of the present invention decrease the level of endogenous S-1-P. Such modulating agents may be identified using methods described herein and used, for example, in cancer therapy and treatment of muscle developmental defects and cardiomyopathy. It has also been found, within the context of the present invention, that the detection of alterations in endogenous S-1-P levels can be used to diagnose cancer and defects in muscle developmental and cardiomyopathy, and to assess the prognosis for recovery. The present invention further provides such diagnostic methods and kits.

Agents which inhibit or block SK activity or expression are also provided in the present invention. In one aspect of the invention, such drugs may be effective treatment for at least some kinds of cancer, especially those in which a dominant Ras mutation is involved. In one embodiment of the invention, such drugs are effective in the treatment of inflammatory and immune disorders. In further embodiments, such drugs may be used as antithrombotic therapy, such as for the therapy of pathologic thrombus formation associated with ischemic heart disease, stroke, vascular disease, angioplasty, and stent deployment. Methods for the identification of new and effective pharmacological agents which inhibit SK activity, as well as drug targets downstream of S-1-P signaling are also provided in the present invention. As used herein, inhibition of SK activity means to decrease the level of SK enzymatic activity as measured using any number of assays known in the art, or certain illustrative assays described herein. Preferably, the decrease in enzymatic SK activity is a statistically significant decrease in enzymatic activity as compared to an appropriate control. Likewise inhibition may apply to the activity of any component of a sphingolipid metabolic and/or signaling pathway, such as SPL, SPT, and the like. In certain embodiments, inhibition of SK activity is assessed by measuring levels of LCBPs, as described herein in particular in the Examples below.

In certain embodiments, inhibition of SK activity is determined by the ability of an agent to protect or “rescue” a mutant yeast strain from otherwise lethal, toxic and/or growth inhibitory intracellular accumulations of phosphorylated long chain bases (such as S-1-P).

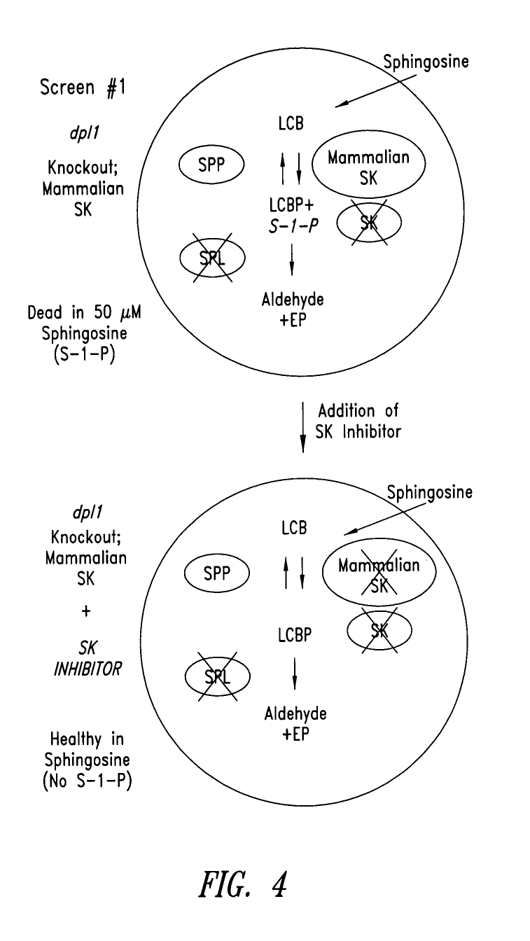

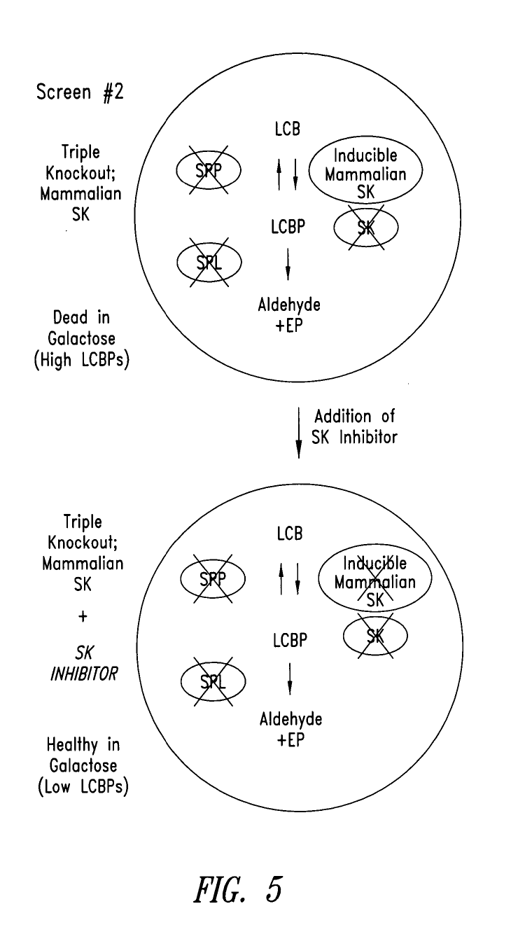

One embodiment of the present invention thus provides agents that inhibit the activity of SK, and related methods and compositions (including mutant and/or recombinant yeast strains) for identifying such agents, for example as determined using one or more yeast screens described herein. In this regard, an agent that rescues certain herein described yeast strains from growth inhibition in the presence of sphingosine (as shown, for example, in FIG. 4 and FIG. 5) may be a useful agent within the context of the present invention. Thus, an increase in growth of said mutant yeast strain in the presence of sphingosine would be observed in the presence of an illustrative agent. Illustrative agents rescue the mutant yeast strain from growth inhibition in the presence of sphingosine when the growth is altered (e.g., restored, increased) in the presence of sphingosine in a statistically significant manner relative to the growth of the mutant yeast strains in the absence of the agent. Illustrative agents rescue the mutant yeast strain from growth inhibition in the presence of sphingosine when growth is restored in the presence of sphingosine by at least 1.5, 2, 2.5, 3, 3.5, 4, 4.5, 5, 5.5, 6, 6.5, 7, 7.5, 8, 8.5, 9, 9.5, or 10 fold or higher as compared to mutant yeast strains grown in the absence of the agent. Growth curves can be determined according to methodologies described herein and known to the art, for example, by inoculating synthetic complete media with logarithmically growing cells (of a mutant yeast strain of interest as described herein) to an O.D._{600 nm}=0.1, then following increase in optical density as a function of time. Controls include mutant cells grown in the absence of sphingosine, mutant cells grown in medium containing sphingosine of various concentrations, mutant cells grown in medium containing sphingosine of various concentrations plus one of two available known SK inhibitors, N,N-dimethylsphingosine or D,L-threo-dihydrosphingosine, and wild type cells in the presence/absence of the inhibitor.

Agents of the present invention that modulate sphingolipid metabolism and/or signaling may be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or may be readily produced. Additionally, natural or synthetically produced libraries and compounds can be readily modified through conventional chemical, physical and biochemical means, and may be used to produce combinatorial libraries. Known pharmacological agents may also be subjected to directed or random chemical modifications, such as acylation, alkylation, esterification, amidification, etc. to produce structural analogs. New potential therapeutic agents may also be created using methods such as rational drug design or computer modeling.

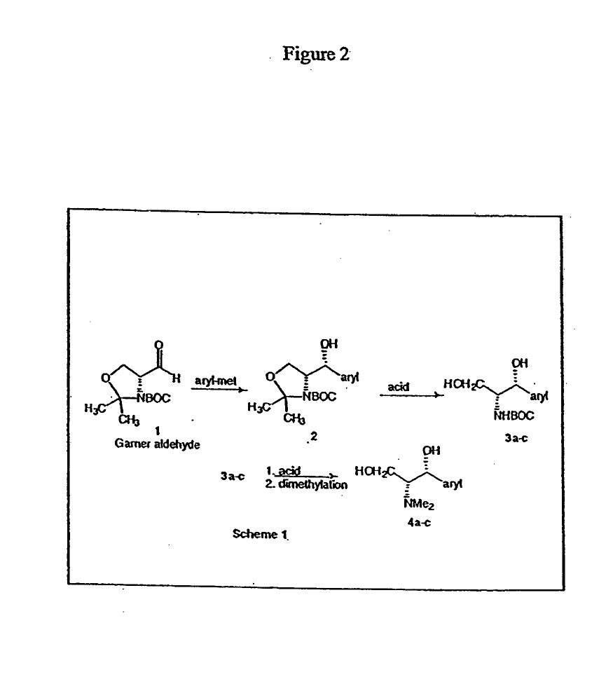

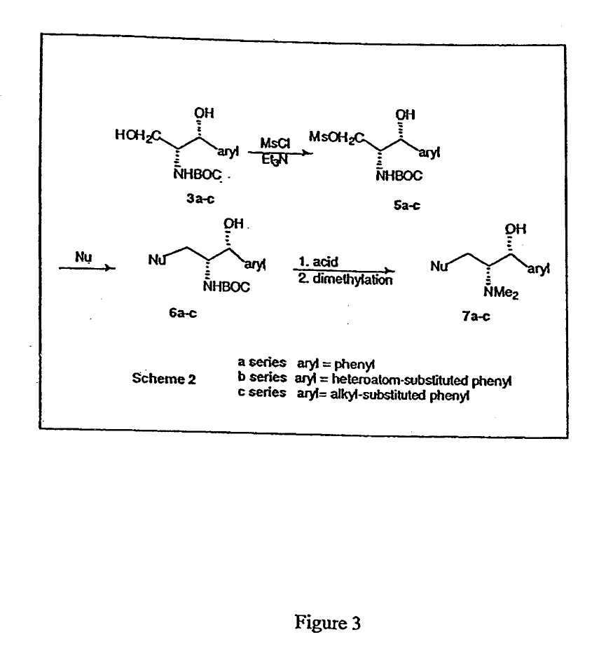

Illustrative agents of the present invention include arrays of rationally designed chemicals with homology to sphingolipids. In particular, synthetic analogs are created that modulate sphingolipid metabolic and/or signaling pathways. In one embodiment, a rationally designed chemical library includes 1-aryl-2-dimethylaminopropane-1,3-diol derivatives. Derivative is a term understood by the ordinarily skilled artisan. For example, derivative means a compound that can be imagined to arise from a parent compound by replacement of one atom with another atom or group of atoms. Within the context of this invention, these derivatives may be rationally designed. Four diastereomers (D or L, erythro or threo) are possible for each member of the library. In certain embodiments, the 1-aryl-2 dimethylaminopropane-1,3-diol derivative is derivitized by modifying the amine, the fatty acid amide and the benzene ring of PDMP. In certain embodiments, the fatty acid amide group is replaced with two N-methyl groups. The skilled artisan would readily appreciate that similar variation can be made in the polar and aromatic substituents to provide other illustrative candidate agents within the context of the instant invention. In another embodiment of the present invention, a 1-aryl-2-dimethylaminopropane-1,3-diol derivative is designed such that lipophilic alkyl groups attached to the arene ring would more closely mimic the character of sphingosine. In one particular embodiment, the synthetic plan makes use of the well-known Garner aldehyde (See 1 in FIG. 2) as starting material, since 1 is readily available in either enantiomeric form. In one embodiment, the D- or L-enantiomer of 1 is used as starting material, and pure erythro stereoisomers of each library member are prepared. In an additional embodiment, a novel and flexible route for assembling the corresponding threo analogues (4a-c, FIG. 2) is followed using a straightforward extension of methodology for making PDMP analogues. The strategy relies on the syn-selective addition to 1 of arylmetal compounds (Aryl-Met) in the presence of certain sulfide and phosphine additives. In this embodiment, both the erythro and threo synthetic routes are modified to prepare substituted variations at the primary carbon atom. A representative synthetic procedure is shown in FIG. 3 for the preparation of 7a-c. Thus, a wide range of nitrogen, oxygen, and carbon nucleophiles could react with mesylates like 5a-c and furnish new libraries of dimethylated PDMP analogues and homologues for use as candidates in the context of the present invention.

Candidate agents for use in a method of screening for a modulator of sphingolipid metabolism and/or signaling according to the present invention may be provided as “libraries” or collections of compounds, compositions or molecules. Such molecules typically include compounds known in the art as “small molecules” and having molecular weights less than 10⁵ daltons, preferably less than 10⁴ daltons and still more preferably less than 10³ daltons. For example, members of a library of test compounds can be contacted with or administered to a mutant or transgenic yeast strain, or a mutant or transgenic Drosophila melanogaster as described herein, and then assayed for their effects on yeast cell growth and survival, or for their ability to restore the wild type phenotype to mutant and/or transgenic Drosophila melanogaster. Compounds so identified as capable of influencing components of the sphingolipid metabolic or signaling pathway (e.g., by altering levels of a sphingolipid intermediate such as S-1-P, ceramide, or sphingosine) may be valuable for therapeutic and/or diagnostic purposes, since they permit treatment and/or detection of diseases associated with sphingolipid metabolism and/or signaling.

Candidate agents further may be provided as members of a combinatorial library, which preferably includes synthetic agents prepared according to a plurality of predetermined chemical reactions performed in a plurality of reaction vessels. For example, various starting compounds may be prepared employing one or more of solid-phase synthesis, recorded random mix methodologies and recorded reaction split techniques that permit a given constituent to traceably undergo a plurality of permutations and/or combinations of reaction conditions. The resulting products comprise a library that can be screened followed by iterative selection and synthesis procedures, such as a synthetic combinatorial library of peptides (see e.g., PCT/US91/08694, PCT/US91/04666) or other compositions that may include small molecules as provided herein (see e.g., PCT/US94/08542, EP 0774464, U.S. Pat. No. 5,798,035, U.S. Pat. No. 5,789,172, U.S. Pat. No. 5,751,629). Those having ordinary skill in the art will appreciate that a diverse assortment of such libraries may be prepared according to established procedures, and tested using the screening methods according to the present disclosure.

Candidate agents of the present invention may also include antibodies that bind to a polypeptide involved in sphingolipid metabolism or signaling. Antibodies may function as modulating agents (as discussed further below) to inhibit or block activity of the polypeptides of the present invention in vivo. Alternatively, or in addition, antibodies may be used within screens for endogenous activity of the polypeptides of the present invention, e.g., SK, SPL, SPT, or modulating agents, for purification of said polypeptides, for assaying the level of activity of said polypeptides within a sample and/or for studies of expression of said polypeptides. Such antibodies may be polyclonal or monoclonal, and are generally specific for one or more polypeptides involved in sphingolipid metabolism and/or one or more variants thereof. Within certain preferred embodiments, antibodies are polyclonal.

Antibodies may be prepared by any of a variety of techniques known to those of ordinary skill in the art (see, e.g., Harlow and Lane, Antibodies: A Laboratory Manual, Cold Spring Harbor Laboratory, 1988). In one such technique, an immunogen comprising an SPL polypeptide or antigenic portion thereof is initially injected into a suitable animal (e.g., mice, rats, rabbits, sheep and goats), preferably according to a predetermined schedule incorporating one or more booster immunizations. The use of rabbits is preferred. To increase immunogenicity, an immunogen may be linked to, for example, glutaraldehyde or keyhole limpet hemocyanin (KLH). Following injection, the animals are bled periodically to obtain post-immune serum containing polyclonal antibodies that bind to a polypeptide involved in sphingolipid metabolism, such as SK, SPL, SPT, S-1-PP. Polyclonal antibodies may then be purified from such antisera by, for example, affinity chromatography using a polypeptide of the present invention, such as SK or SPL, or antigenic portion thereof coupled to a suitable solid support. Such polyclonal antibodies may be used directly for screening purposes and for Western blots.

More specifically, an adult rabbit (e.g., NZW) may be immunized with 10 μg purified (e.g., using a nickel-column) SK or SPL polypeptide emulsified in complete Freund's adjuvant (1:1 v/v) in a volume of 1mL. Immunization may be achieved via injection in at least six different subcutaneous sites. For subsequent immunizations, 5 μg of an SK, SPL, or SPT polypeptide may be emulsified in complete Freund's adjuvant and injected in the same manner. Immunizations may continue until a suitable serum antibody titer is achieved (typically a total of about three immunizations). The rabbit may be bled immediately before immunization to obtain pre-immune serum, and then 7-10 days following each immunization.

For certain embodiments, monoclonal antibodies may be desired. Monoclonal antibodies may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction.

An antibody that specifically binds to a component of a sphingolipid metabolic and/or signaling pathway may interact with said polypeptide component via specific binding if the antibody binds the polypeptide with a Kₐ of greater than or equal to about 10⁴ M⁻¹, preferably of greater than or equal to about 10⁵ M⁻¹, more preferably of greater than or equal to about 10⁶ M⁻¹ and still more preferably of greater than or equal to about 10⁷ M⁻¹ to 10⁹ M⁻¹. Affinities of binding partners such as antibodies and the polypeptides that they bind to can be readily determined using conventional techniques, for example those described by Scatchard et al., Ann. N. Y. Acad. Sci. 51:660 (1949) and in Current Protocols in Immunology, or Current Protocols in Cell Biology, both published by John Wiley & Sons, Inc., Boston, Mass.

As noted above, the present invention provides agents that alter the expression (transcription or translation), stability and/or activity of a polypeptide involved in sphingolipid metabolism. To identify such a modulating agent, any of a variety of screens may be performed. Candidate modulating agents may be obtained using well known techniques from a variety of sources, such as plants, fungi or libraries of chemicals, small molecules or random peptides. Antibodies that bind to a polypeptide of the present invention, and anti-sense polynucleotides that hybridize to a polynucleotides that encodes a protein involved in sphingolipid metabolism, may be candidate modulating agents. Preferably, a modulating agent has a minimum of side effects and is non-toxic. For some applications, agents that can penetrate cells are preferred.

The subject methods find use in the screening of a variety of different potentially therapeutic candidate agents. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents of the present invention further include agents that restore wild type phenotype to mutant or transgenic flies as described herein, in particular in the Examples. In one embodiment, modulating agents are screened by culturing or otherwise contacting the agent with a Drosophila melanogaster null mutant for a time sufficient to observe in said mutant Drosophila melanogaster an effect of the agent on a level of either at least one sphingolipid intermediate, or the activity of at least one component of sphingosine metabolism and/or signaling pathway. The term “null allele” is a term of art meaning an allele that makes no gene product or whose product has no activity of any kind. Thus a null mutant is a mutant comprising a null allele. In one embodiment, the Drosophila melanogaster null mutant has a flightless phenotype caused by abnormal development of indirect flight muscles (IFM) during metamorphosis. This phenotype provides a novel schema by which to elucidate sphingolipid metabolism and signaling, identify genetic suppressors and identify chemicals which modulate sphingolipid metabolism and/or signaling through their effect on key components in the sphingolipid metabolic and/or signaling pathway. Agents that result in a statistically significant alteration in the level of a sphingolipid intermediate or alteration in the level of activity of a component of a sphingolipid metabolic or signaling pathway is an agent that modulates sphingolipid metabolism and/or signaling. Agents that result in a statistically significant restoration in the phenotype of a mutant or transgenic fly grown or otherwise cultured in the presence of said agent as compared to a mutant or transgenic fly grown or otherwise cultured in the absence of the agent as described herein is an agent that modulates sphingolipid metabolism and/or signaling.

As mentioned above, the subject mutant and transgenic flies find particular utility in screening assays designed to identify diagnostic and therapeutic compounds for a variety of human diseases as described herein, such as numerous cancers including breast, colon, uterus, stomach, ovary, lung, kidney and rectal cancer, and diagnosis and treatment of hereditary sensory neuropathy type 1 and the sphingolipidoses. Through use of the subject transgenic flies (or cells derived therefrom depending on the particular screening assay), one can identify compounds that have activity with respect to sphingolipid metabolism and/or signaling and therefore, the diseases associated with modulation of sphingolipid metabolism and/or signaling. Compounds have activity with respect to sphingolipid metabolism and/or signaling if they modulate or have an effect on at least one parameter or symptom of the disease, such as tumor development, etc., where the modulatory activity may be to reduce or enhance the magnitude of the symptom. Tumors comprise abnormal masses of tissue and can be benign or cancerous. As would be readily appreciated by the skilled artisan, there are dozens of different types of tumors and their identification and diagnosis are known in the art and can be determined by a qualified clinician.

Thus, the screening methods of subject invention can be used to identify compounds that modulate the progression of disease, e.g. by binding to, modulating, enhancing or repressing the activity of a protein or peptide involved in the sphingolipid metabolism and/or signaling, and/or compounds that ameliorate, alleviate or even remove the phenotypic symptoms of the disease, where such activity may or may not be the result of activity with respect to the underlying mechanism of the disease.

Assays of the invention make it possible to identify compounds which ultimately: (1) have a positive affect with respect to diseases associated with sphingolipid metabolism and/or signaling and as such are therapeutics, e.g., agents which arrest or reverse development of tumors or ameliorate or alleviate the symptoms of such a condition; or (2) have an adverse affect with respect to the disease and as such should be avoided as therapeutic agents.

In certain preferred screening methods of the subject invention, a quantity of a candidate agent is generally orally administered to the fly. Following oral administration, the affect of the candidate agent on phenotype of the fly is determined, typically by comparison with a control (i.e. a mutant or transgenic fly to which the candidate agent has not been administered). The effect of the candidate agent is determined by determining whether one or more of the phenotypic characteristics of the mutant or transgenic fly as described herein are exacerbated or ameliorated in the test fly as compared to the control fly, where characteristics that are monitored include levels of sphingolipid intermediates, flight behavior, flight muscle developmental defects, and the like. The candidate agent is generally orally administered to the fly by mixing the agent into the fly nutrient medium and placing the medium in the presence of the fly, (either the larva or adult fly) such that the fly feeds on the medium. Generally a plurality of assay mixtures are run in parallel with different candidate agent concentrations (or no candidate agent) to obtain a differential response to the various concentrations of the candidate agent. Typically, one of these test groups serves as a negative control, i.e., no candidate agent is present. In a preferred embodiment, a high throughput screening protocol is employed, in which a large number of candidate agents are tested in parallel using a large number of flies. By “large number” is meant a plurality, where plurality means at least 50, usually at least 100, and more usually at least 1000, where the number of may be 10,000 or 50,000 or more, but in many instances will not exceed 5000.

A modulating agent may additionally comprise, or may be associated with, a targeting component that serves to direct the agent to a desired tissue or cell type. As used herein, a “targeting component” may be any substance (such as a compound or cell) that, when linked to a compound enhances the transport of the compound to a target tissue, thereby increasing the local concentration of the compound. Targeting components include antibodies or fragments thereof, (e.g. anti-tumor antibodies) receptors, ligands and other molecules that bind to cells of, or in the vicinity of, the target tissue. Known targeting components include hormones, antibodies against cell surface antigens, lectins, adhesion molecules, tumor cell surface binding ligands, steroids, cholesterol, lymphokines, fibrinolytic enzymes and other drugs and proteins that bind to a desired target site. In particular, anti-tumor antibodies and compounds that bind to an estrogen receptor may serve as targeting components. An antibody employed in the present invention may be an intact (whole) molecule, a fragment thereof, or a functional equivalent thereof (e.g. antigen-binding fragments). Examples of antibody fragments are F(ab′)2, -Fab′, Fab and F[v] fragments, which may be produced by conventional methods or by genetic or protein engineering. Linkage may be via any suitable covalent bond using standard techniques that are well known in the art. Such linkage is generally covalent and may be achieved by, for example, direct condensation or other reactions, or by way of bi- or multi-functional linkers.

Assays for Detecting Modulation of Components of Sphingolipid Metabolism and/or Signaling and/or Sphinqolipid Intermediates

Numerous assays for detecting modulation of components of sphingolipid metabolism and/or signaling are available in the art. Illustrative assays are described further herein, for example as described in the Example section.

Numerous methods for detecting polynucleotides of the present invention are known in the art and are useful in the context of the instant invention. Illustrative methods are described in Ausubel et al. (1993 Current Protocols in Molecular Biology, Greene Publ. Assoc. Inc. & John Wiley & Sons, Inc., Boston, Mass.); Sambrook et al. (1989 Molecular Cloning, Second Ed., Cold Spring Harbor Laboratory, Plainview, N.Y.); Maniatis et al. (1982 Molecular Cloning, Cold Spring Harbor Laboratory, Plainview, N.Y.), and elsewhere. In one embodiment, polynucleotide expression is measured using any number of hybridization techniques. In further embodiments, polynucleotide expression is measure using amplification techniques, such as RT-PCR, PCR, quatitative-competitive (QC) PCR, and real-time PCR.

Numerous methods for detecting and measuring enzymatic activity of components involved in sphingolipid metabolism and/or signaling are known in the art and can be used in the context of the present invention (see e.g. Current Protocols in Protein Science, John Wiley & Sons, Inc., Boston, Mass.). Certain illustrative methods are described in Saba, J. D., Nara, F., Bielawska, A., Garrett, S. and Hannun, Y. A. (1997). J Biol Chem 272, 26087-26090, and Van Veldhoven, P. P. and Mannaerts, G. P. (1991). J Biol Chem 266, 12502-7, Williams, R, Wang E and Merrill A, 1984., Arch Biochem Biophys 228:282-291., Caligan, T B, Peters K, Ou J, Wang E, Saba J and Merrill A H, Jr., 2000. Analytical Biochemistry 281:36-44.

In one embodiment, SK activity of an SK polypeptide or variant thereof may generally be assessed using an in vitro assay that detects the production of labeled substrate (i.e., sphingosine-1-phosphate, or a derivative thereof. SK is responsible for the phosphorylation of sphingosine to generate S-1-P. In one embodiment of the present invention, an in vitro assay for SK requires both ATP and a divalent cation (magnesium, calcium or manganese) for the phosphorylation of the hydroxyl group on the first carbon of sphingosine. SK activity may be assayed in tissues from a variety of species, including human and porcine platelets, bovine brain and kidney, rat liver, the yeast Hansenula ciferrii, and Tetrahymena pyriformis. In one embodiment, the assay requires a fixed ratio of magnesium to ATP of 5:1 and a neutral pH (between 7.2-7.5). SK is found in the cytoplasm of platelets and is associated with membranes in rat brain and several other tissues. D-erythro-sphingosine, the naturally occurring isomer of sphingosine and most abundant sphingoid base in most mammalian cells, serves as a substrate for SK from all sources. Sphingosine inhibits the activity of protein kinase C, and stereospecificity for the erythro conformation has been demonstrated in mixed micellar assays using human platelet and rat brain-derived enzyme. A variety of long chain bases can also serve as substrates for SK, including erythro-dihydrosphingosine and phytosphingosine. SK activity increases with the carbon chain length of a D-erythro-dihydrosphingosine substrate. In one embodiment, stimulation of Swiss 3T3 cells with some inducers of proliferation (fetal calf serum or PDGF) can be used to assay an increase in both sphingosine levels and SK activity. Illustrative stimuli which can be used to activate SK include nerve growth factor, muscarinic acetylcholine agonists, TNFα, and cross-linking of the FcεRI and FcγRI immunoglobulin receptors. Additional mitogens such as the b subunit of the cholera toxin and 12-O-tetradecanoyl phorbol-13-acetate may also be used to increase SK enzyme activity.

Within certain embodiments, an in vitro assay for SK activity may be performed using cellular extracts prepared from cells that express a polypeptide of interest. Preferably, in the absence of a polynucleotide encoding an SK polypeptide, such cells do not produce a significant amount of endogenous SK (i.e., a cellular extract should not contain a detectable increase in the level of SK, as compared to buffer alone without extract). Illustrative assays for detection of SK activity are known in the art, such as those described herein in the Examples.

Screens for modulating agents that alter expression or stability of a polypeptide of the present invention may be readily performed using well known techniques that detect the level of protein or mRNA. Suitable assays include RNAse protection assays, in situ hybridization, ELISAs, Northern blots and Western blots. Such assays may generally be performed using standard methods (see Sambrook et al., Molecular Cloning: A Laboratory Manual, Cold Spring Harbor Laboratories, Cold Spring Harbor, N.Y., 1989). For example, to detect mRNA encoding SK, SPL or other polynucleotides involved in the metabolism of sphingolipids, a nucleic acid probe complementary to all or a portion of the gene sequence of interest may be employed in a Northern blot analysis of mRNA prepared from suitable cells. Additionally, In situ hybridization may be performed as described in Blair, S. (Blair S., 2000. Imaginal discs. In Drosophila Protocols. W. Sullivan, M. Ashburner, and R. Hawley, editors. Cold Spring Harbor Laboratory Press, Cold Spring Harbor, N.Y. 159-175).

Alternatively, real-time PCR can also be used to detect levels of mRNA encoding SPL, SK, or other polypeptides involved in sphingolipid metabolism as described herein (see Gibson et al., Genome Research 6:995-1001, 1996; Heid et al., Genome Research 6:986-994, 1996). The first-strand cDNA to be used in the quantitative real-time PCR is synthesized from 20 μg of total RNA that is first treated with DNase I (e.g., Amplification Grade, Gibco BRL Life Technology, Gaitherburg, Md.), using Superscript Reverse Transcriptase (RT) (e.g., Gibco BRL Life Technology, Gaitherburg, Md.). Real-time PCR is performed, for example, with a GeneAmp™ 5700 sequence detection system (PE Biosystems, Foster City, Calif.). The 5700 system uses SYBR™ green, a fluorescent dye that only intercalates into double stranded DNA, and a set of gene-specific forward and reverse primers. The increase in fluorescence is monitored during the whole amplification process. The optimal concentration of primers is determined using a checkerboard. The PCR reaction is performed in 25 μl volumes that include 2.5 μl of SYBR green buffer, 2 μl of cDNA template and 2.5 μl each of the forward and reverse primers for the SPL gene, or other gene of interest. The cDNAs used for RT reactions are diluted approximately 1:10 for each gene of interest and 1:100 for the β-actin control. In order to quantitate the amount of specific cDNA (and hence initial mRNA) in the sample, a standard curve is generated for each run using the plasmid DNA containing the gene of interest. Standard curves are generated using the Ct values determined in the real-time PCR which are related to the initial cDNA concentration used in the assay. Standard dilution ranging from 20−2×10⁶ copies of the SPL gene or other gene of interest are used for this purpose. In addition, a standard curve is generated for β-actin ranging from 200 fg-2000 fg. This enables standardization of the initial RNA content of a sample to the amount of β-actin for comparison purposes. The mean copy number for each sample tested is normalized to a constant amount of β-actin, allowing the evaluation of the observed expression levels of SPL or other genes of interest.

To detect a protein of the present invention, a reagent that binds to the protein (typically an antibody, as described herein) may be employed within an ELISA or Western assay. Following binding, a reporter group suitable for direct or indirect detection of the reagent is employed (i.e., the reporter group may be covalently bound to the reagent or may be bound to a second molecule, such as Protein A, Protein G, immunoglobulin or lectin, which is itself capable of binding to the reagent). Suitable reporter groups include, but are not limited to, enzymes (e.g., horseradish peroxidase), substrates, cofactors, inhibitors, dyes, radionuclides, luminescent groups, fluorescent groups and biotin. Such reporter groups may be used to directly or indirectly detect binding of the reagent to a sample component using standard methods known to those of ordinary skill in the art.

Alternatively, or in addition, a candidate modulating agent may be tested for the ability to alter enzymatic activity, such as SPL or SK activity, using an in vitro assay as described herein (see Van Veldhoven and Mannaerts, J. Biol. Chem. 266:12502-07, 1991) that detects the degradation of labeled substrate (i.e., sphingosine-1-phosphate, or a derivative thereof). Briefly, a solution (e.g., a cellular extract) containing an SK or SPL polypeptide (e.g., 10 nM to about 10 mM) may be incubated with a candidate modulating agent (typically 1 nM to 10 mM, preferably 10 nM to 1 mM) and a substrate (e.g., 40 μM) at 37° C. for 1 hour in the presence of, for example, 50 mM sucrose, 100 mM K-phosphate buffer pH 7.4, 25 mM NaF, 0.1% (w/v) Triton X-100, 0.5 mM EDTA, 2 mM DTT, 0.25 mM pyridoxal phosphate. Reactions may then be terminated and analyzed by thin-layer chromatography to detect the formation of labeled fatty aldehydes and further metabolites. A modulating agent (e.g., an antibody or other modulating agent as described herein) that alters SK or SPL activity results in a statistically significant increase or decrease in the degradation of sphingosine-1-phosphate, relative to the level of degradation in the absence of modulating agent. Such modulating agents may be used to increase or decrease SK or SPL activity in a cell culture or a mammal, as described herein.

Modulating agents that alter the SPL activity of an SPL polypeptide or variant thereof may generally be assessed using an in vitro assay that detects the degradation of labeled substrate (i.e., sphingosine-1-phosphate, or a derivative thereof. Within such assays, pyridoxal 5′-phosphate is normally a requirement for SPL activity. In addition, the reaction generally proceeds optimally at pH levels around 7.4-7.6 and requires chelators due to sensitivity toward heavy metal ions. PH levels may be from 6.5, 6.7, 6.9, 7.0, 7.1, 7.2, 7.3, 7.7, 7.8, 7.9, 8.0, 8.1, 8.2, 8.3, 8.4, or 8.5. The substrate should be a D-erythro isomer, but in derivatives of sphingosine-1-phosphate the type and chain length of sphingoid base may vary. In general, an assay as described by Van Veldhoven and Mannaerts, J. Biol. Chem. 266:12502-07, 1991 may be employed. Briefly, a solution (e.g., a cellular extract) containing the polypeptide may be incubated with about 40 μM substrate at 37° C. for about 1 hour in the presence of, for example, 50 mM sucrose, 100 mM K-phosphate buffer pH 7.4, 25 mM NaF, 0.1% (w/v) Triton X-100, 0.5 mM EDTA, 2 mM DTT, 0.25 mM pyridoxal phosphate. Reactions may then be terminated and analyzed by thin-layer chromatography to detect the formation of labeled fatty aldehydes and further metabolites. A modulating agent as described herein that alters SPL activity of an SPL polypeptide or variant thereof will result in a statistically significant increase or decrease in SPL activity as assayed herein as compared to the activity in the absence of said modulating agent.

Within certain embodiments, an in vitro assay for SPL activity may be performed using cellular extracts prepared from cells that express the polypeptide of interest. Preferably, in the absence of a gene encoding an SPL polypeptide, such cells do not produce a significant amount of endogenous SPL (i.e., a cellular extract should not contain a detectable increase in the level of SPL, as compared to buffer alone without extract). It has been found, within the context of the present invention, that yeast cells containing deletion of the SPL gene (BST1) are suitable for use in evaluating the SPL activity of a polypeptide. bst1Δ cells can be generated from S. cerevisiae using standard techniques, such as PCR, as described herein. A polypeptide to be tested for SPL activity may then be expressed in bst1Δ cells, and the level of SPL activity in an extract containing the polypeptide may be compared to that of an extract prepared from cells that do not express the polypeptide. For such a test, a polypeptide is preferably expressed on a high-copy yeast vector (such as pYES2, which is available from Invitrogen) yielding more than 20 copies of the gene per cell. In general, a polypeptide has SPL activity if, when expressed using such a vector in a bst1Δ cell, a cellular extract results in a two-fold increase in substrate degradation over the level observed for an extract prepared from cells not expressing the polypeptide.

A further test for SPL activity may be based upon functional complementation in the bst1Δ strain. It has been found, within the context of the present invention, that bst1Δ cells are highly sensitive to D-erythro-sphingosine. In particular, concentrations as low as 10 μM sphingosine completely inhibit the growth of bst1Δ cells. Such a level of sphingosine has no effect on the growth of wildtype cells. A polypeptide having SPL activity as provided above significantly diminishes (i.e., by at least two fold) the sphingosine sensitivity when expressed on a high-copy yeast vector yielding more than 20 copies of the gene per cell.

The present invention also provides yeast assays for the identification of agents that modulate sphingolipid metabolism and/or signaling. In one embodiment, the present invention provides yeast assays for the identification of agents that modulate SK activity. In one embodiment, the growth of yeast mutant strains lacking any one or more of SPL, SK and SPP activity, and/or activity of one or more other components of the sphingolipid metabolic and/or signaling pathway as described herein, is inhibited by the presence of sphingosine. In certain embodiments, the yeast mutant strain comprises a null allele in DPL1 and LCB4. (As noted above, the term “null allele” is a term of art meaning an allele that makes no gene product or whose product has no activity of any kind. Thus a null mutant is a mutant comprising a null allele). In another embodiment, the yeast mutant strain comprises a null allele in DPL1, LCB4, and YSR2. In a further embodiment, the yeast mutant strain comprises a null allele in YSR2 and SUR2. In yet a further embodiment, the yeast mutant strain comprises a null allele in at least one or more of DPL1, LCB4, YSR2, and SUR2. In yet another embodiment, the mutant yeast strain for use in screening agents that modulate sphingolipid metabolism and/or signaling is further transformed or transfected to express nonendogenous SK (e.g., from an exogenous source, a source other than from yeast, such as human, mouse, Drosophila melanogaster, C. elegans) such as SK derived from human, mouse, or from other sources, such as Drosophila melanogaster, C. elegans, etc. In certain related embodiments, a mutant yeast strain is genetically modified to express nonendogenous SPL, SPP, SPT, or one or more other components of the sphingolipid metabolic and/or signaling pathway. In one embodiment, the nonendogenous component of the sphingolipid metabolic and/or signaling pathway, such as SK, SPL, SPP, or SPT, is expressed under the control of an inducible promoter, such as a galactose inducible promoter. In this context, the assay includes the appropriate concentration of galactose in the growth media. In one embodiment, inhibition of SK activity by a candidate agent leads to the rescue of the phenotype from sensitivity to sphingosine or to an inducer (e.g., galactose) that induces expression of a nonendogenous component of the sphingolipid metabolic and/or signaling pathway, and allows for growth of the yeast strain in the presence of sphingosine or the inducer.

In one particular embodiment, the screen for agents that modulate sphingolipid metabolism and/or signaling, such as inhibitors of SK, is performed on agar plates containing between about 10 to about 200 μM D-erythro-sphingosine. In one embodiment, the agar plates contain between about 20, 21, 22, 23, 24, 25, 26, 27, 28, 29, 30, 31, 32, 33, 34,35, 36, 37, 38, 39, 40, 41, 42, 43, 44, 45, 50, 51, 52, 53, 54, 55, 56, 57, 58, 59, 60, 65, or 70 μM D-erythro-sphingosine, or appropriate concentration of any other long chain sphingoid base that inhibits growth of the mutant yeast strains described herein. Agar plates may further contain an appropriate concentration of dispersant, such as NP40 at about 0.0015%. In one embodiment the screen for agents that modulate sphingolipid metabolism and/or signaling, such as agents that inhibit SK activity, is performed in a 96-well plate. In this context, small, equal numbers of mutant yeast cells are placed into each well, and an array of candidate agents as described herein are diluted into the wells prior to incubation. As would be recognized by the skilled artisan, cell numbers and the concentration of candidate agents can be optimized experimentally. Generally, concentration of sphingosine and cells are kept constant. In certain embodiments, growth curves are determined by inoculating the appropriate growth medium, such as synthetic complete medium with logarithmically growing cells to an O.D.₆₀₀ₙₘ of about 0.1, then following increase in optical density as a function of time. As noted above, in certain embodiments, the appropriate growth medium may include galactose or the appropriate inducer for an inducible promoter. The lowest inhibitory concentration of sphingosine is determined. Controls for optimization include mutant yeast strain in sphingosine of various concentrations plus known SK inhibitors, such as N,N-dimethylsphingosine or D,L-threo-dihydrosphingosine, and wild type cells in the presence and absence of these inhibitors.

Generally, illustrative agents rescue the growth inhibition of a mutant yeast strain in the presence of any long chain sphingoid base that inhibits growth of a mutant yeast strain, such as D-erythro-sphingosine, when the mutant strain growth is restored in the presence of a growth inhibitory long chain sphingoid base in a statistically significant manner as compared to mutant yeast strains grown in the absence of the agent. In one embodiment, the long chain sphingoid base that inhibits growth of a mutant yeast strain as described herein is D-erythro-sphingosine. In certain embodiments, the growth of a mutant yeast strain in the presence of sphingosine in the appropriate growth medium is restored in the presence of a candidate agent, by at least 1.5, 2, 2.5, 3, 3.5, 4, 4.5, 5, 5.5, 6, 6.5, 7, 7.5, 8, 8.5, 9, 9.5, or 10 fold or higher as compared to mutant yeast strains grown in the absence of the agent.

In certain embodiments illustrative agents rescue the growth inhibition of a mutant yeast strain in the presence of an inducer that induces expression of a nonendogenous gene (e.g., a nonendogenous component of the sphingolipid metabolic and/or signaling pathway, such as SK, SPL, SPP, or SPT) from an inducible promoter. Thus, in certain embodiments, expression of the nonendogenous gene results in growth inhibition of a mutant yeast strain (e.g. even in the absence of sphingosine in the growth media). Illustrative inducible promoters include but are not limited to galactose-inducible promoters and thus, an illustrative inducer in this context is galactose. Illustrative candidate agents include agents that restore mutant strain growth in the presence of the appropriate inducer (e.g., galactose) in a statistically significant manner as compared to mutant yeast strains grown in the absence of the candidate agent.

The skilled artisan would readily appreciate that any variety of inducible promoters and their cognate inducers can be used in the context of the present invention. A variety of inducible systems can be used, including galactose-inducible promoters, pXyI-xyIR-inducible promoter, the pSpac-IacI system, the arabinose-inducible PBAD promoter and a tetracycline-inducible promoter system using, for example, the xyl/tetO promoter. Other illustrative inducible promoters that can be used in the context of the present invention are activated by physical means such as the heat shock promoter. Others are activated by (i.e., the inducers are) chemicals such as IPTG or Tetracycline. Thus, in certain embodiment of the invention the nucleic acid molecule encoding a desired protein such as SK, SPL, SPP, or SPT is under the control of an inducible control element (e.g., a promoter) such that expression of the desired protein can be turned on or off (or intermediate levels in between) using an inducer which affects the inducible control element (e.g., expression can be modulated by modulating the concentration of the inducer in the presence of the yeast strain). This allows for switching on or off of the expression of the protein. A variety of inducible promoters and their cognate inducers are commercially available, for example, from Invitrogen Life Technologies (Carlsbad, Calif.) or Stratagene (La Jolla, Calif.). Other illustrative inducible or otherwise regulated expression systems include but are not limited to those controlled by heavy metals (Mayo et al. 1982 Cell 29:99-108), RU486 (a progesterone antagonist) (Wang et al. 1994 Prxoc. Natl. Acad. Sci. USA 91:8180-8184), steroids (Mader and White, 1993 Proc. Natl. Acad. Sci. USA 90:5603-5607), and tetracycline (Gossen and Bujard 1992 Proc. Natl. Acad. Sci. USA 89:5547-5551; U.S. Pat. No. 5,464,758).

In certain embodiments, the plate assay utilizes two available SK inhibitors, N,N-dimethylsphingosine and D,L-threo-dihydrosphingosine (Yatomi, Y., et al., Biochemistry 1996, 35(2), 626-633; Stoffel, W. & Bister, K. Hoppe-Seyler's Z Physiol Chem 1973, 354, 169-181.) Concentrations of D-erythro-sphingosine that inhibit cell growth overnight (at 30° C. with shaking) can be determined by measuring turbidity at OD₆₀₀ₙₘ, or by any other appropriate means, such as counting cells on a hemacytometer. D-erythro-sphingosine concentrations that inhibit growth of a specific number of inoculated cells except in the presence of SK inhibitors are chosen as conditions for screening.

In certain embodiments, the assay to screen for modulators of sphingolipid metabolism and/or signaling can be further validated in a dpl1 Icb4 Icb5 strain containing murine SphK1 cDNA (GenBank Accession No. AF068748) (Kim, S., Fyrst, H. & Saba, J. Genetics 2000, 156, 1519-1529). Chemicals promoting growth of this strain in sphingosine can be validated using survival assays on agar plates containing the chemical. The efficacy and biochemical characteristics of candidate compounds may be quantified by using any number of assays, such as 1) determining the IC₅₀ of the inhibitor on purified SK and recombinant human SK, 2) analyzing the kinetics of inhibition using classical Michaelis-Menten methods, 3) determining the reversibility of the inhibition and/or 4) by comparing the LCBP accumulation of a dpl1 human SK strain treated with no inhibitor versus treatment with a known SK inhibitors versus treatment with the new compound. In vitro SK assays are performed using an HPLC based assay as described in Caligan, et al., (supra).

In certain embodiments, in order to validate a yeast mutant strain screen for modulators of sphingolipid metabolism and/or signaling, the mutant yeast strains as described herein are inoculated to a density of about 1×10⁴ cells per plate onto agar plates containing about 100 μM D-erythro-sphingosine and about 0.0015% NP40 (used as a dispersant). At these concentrations of sphingosine and this cell density, the dpl1Δ yeast strains do not grow. Similarly, plates containing D-erythro-sphingosine plus known SK inhibitors may be incubated for 3-5 days, and plates inspected for sphingosine resistance. If it appears that the strain containing human SK can survive sphingosine in the presence of SK inhibitors, a dilutional assay provides semi-quantitative information on the degree of protection afforded by the known inhibitors. For the sphingosine dilutional assay, cells are grown to saturation for about 2-3 days in liquid cultures and then resuspended in minimal medium, placed in the first row of a 96 well plate and diluted serially from about 1:2 to about 1:4000 across the plate. The cultures are then template-inoculated onto a control plate (rich medium) and a plate containing 50 μM sphingosine and 0.0015% NP40, plus or minus SK inhibitor (At this concentration of NP40, no effects on cell viability are observed.) Plates are then incubated at 30° C. for about 2 days and assessed visually for differences in growth.

Assays to detect and measure sphingolipid intermediates include solid phase extraction. In certain embodiments, a Strata C18-E solid phase extraction column (50 mg/ml) (Phenomenex, Torrance, Calif.) can be used. In this context the column is initially wetted with 200 μl of methanol, followed by equilibration with 1 ml of solvent A. Fly extracts or LCB standards in solvent A may be applied to the equilibrated Strata C18-E column, followed by a wash with 1 ml of solvent A. A second wash of the column is performed by the addition of 600 μl of methanol. LCBs are then eluted from the column with 600 μl of methanol:10 mM ammonium acetate, 9:1 (v/v) and dried down in a speed vac. The skilled artisan would readily appreciate that the above parameters can be optimized and changed according to extracts and LCBs being used.

High-performance liquid chromatography analysis (HPLC) can also be used within the context of the present invention. HPLC can be carried out as described for example in Lester, R. L., and R. C. Dickson. 2001. Anal. Biochem. 298: 283-292. Briefly, LCBs are derivatized with, for example, ortho-phthalaldehyde (OPA) (Sigma St. Louis, Mo.) as described in Caligan, T. B., K. Peters J. Ou, E. Wang, J. Saba, and A. H. Jr. Merrill. 2000. Anal. Biochem. 281: 36-44. The OPA-derivatized LCBs are separated on a reverse-phase column with the mobile phase methanol/10 mM ammonium acetate, pH 5.2, 82:18 (v/v). Numerous reverse-phase columns are known in the art. Illustrative reverse-phase columns include but are not limited Luna RP-18, 3μ, 4.6×75 mm (Phenomenex, Torrance, Calif.). Flow rate is generally in the range of 1 ml/min. The skilled artisan would appreciate that flow rates can range from 0.2 ml/min to 3 ml/min and include any integer in between. Any number of HPLC systems can be used. Illustrative systems include a Beckman System Gold with a 125 solvent module. Fluorescent LCBs can be detected using a variety of systems. In one particular embodiment, fluorescent LCBs are detected and quantified using a Spectra-Physics fluorescence detector (SP 8410).

Mass Spectrometry may also used in the context of the present invention to detect and measure sphingolipid intermediates as described herein. In one particular embodiment, a Strata C18-E column-purified lipid extract from a desired source, and a C₁₄ sphingolipid standard are analyzed on a Micromass Quattro LCZ instrument following direct injection of 10 μl of sample. Mobile phase is generally in the range of 80 percent methanol containing 0.1 percent formic acid. The skilled artisan would appreciate that the mobile phase can be optimized. Flow rate is generally in the range of 0.2 ml/min. Structural confirmation of LCBs is obtained by positive electrospray ionization (ESI+) mass spectrometry. LCBs can be detected by precursor ion scans of structurally distinct ion fragments as described in the art, in particular as described in Sullards, M. C., and A. H. Jr. Merrill. 2001. Sci. STKE. 67: 1-11. Generally, 3.5 kV is applied to the capillary to start the spray and the collision-induced decomposition spectra, at a cone voltage of 20 V, are recorded at a collision energy of 15 eV with argon as collision gas. The skilled artisan would readily understand that any of the above parameters can change according to different samples and desired intermediates being measured as is known in the art.

Thus, LCBs can be identified through their patterns of collision- induced dissociation and precursor ion scans using positive ion electrospray mass spectrometry (ESI+) as described in Sullards, M. C., and A. H. Jr. Merrill. 2001. Sci. STKE. 67: 1-11. Based on their unique molecular structures, typical decomposition products arise from the loss of two water molecules. For example, the precursor ion spectrum of m/z 208 (C₁₄ sphingosine minus two water molecules) shows parents as m/z 244 (C₁₄ sphingosine) and m/z 226 (C₁₄ sphingosine minus one water molecule). In order to verify the existence of, for example C₁₄dihydrosphingosine in Drosophila melanogaster, a Strata C18-E column purified lipid extract may be analyzed by ESI+. In addition, precursor ion scans of m/z 236 and m/z 238 identify C₁₆ sphingosine and C₁₆ dihydrosphingosine in a sample.

Lipid extracts for analysis in the context of the present invention can be prepared using any number of procedures known in the art. For example, to prepare Drosophila melanogaster lipid extracts, samples containing 25 mg of frozen intact fly material are placed in a homogenizer, for example a 7 ml Potter Elvehjem homogenizer. 20 μl of a mixture of internal LCB standards, (commercially available from, for example Matreya Inc., Pleasant Gap, Pa.) containing 250 to 500 pmol of each LCB are then added. Flies are homogenized in 2 ml of ice cold methanol/water, 1:1 (v/v) with a loose pestle followed by a tight pestle until it moved smoothly. Extracts are further homogenized with a tip sonicator (3×20 sec.) while on ice, then transferred to a glass tube and centrifuged at 1500×g for 10 minutes. Supernatants are recovered and dried down in a speed vac. Extracts are resuspended in 200 μl of methanol containing 0.1 M ammonium hydroxide, followed by vortexing, bath sonication and incubation at 37° C. for 1 hr to allow hydrolysis of esterified acyl chains. Following hydrolysis, the samples are cooled to room temperature, dried down in a speed vac and resuspended in 500 μl of methanol/water, 2:3 (v/v) containing 0.1% glacial acetic acid (solvent A). The skilled artisan would recognize that the above procedure may be modified accordingly to prepare lipid extracts from other samples including mammalian cells, yeast, bacteria or any other desired source of sphingolipid intermediate.

As noted herein, sphingolipid signaling contributes to specific pathways for biological signal transduction, including those associated with cell division, cell survival, apoptosis, proliferation and differentiation and “biological signal transduction pathways” or “inducible signaling pathways” in the context of the present invention include transient or stable associations or interactions among molecular components involved in the control of these and similar processes in cells. Depending on the particular sphingolipid signaling pathway of interest, such as a pathway induced by S-1-P binding to an EDG receptor and the like, an appropriate parameter for determining induction of such pathway may be selected. Signaling pathways associated with cell proliferation, there is available a variety of well known methodologies for quantifying proliferation, including, for example, incorporation of tritiated thymidine into cellular DNA, monitoring of detectable (e.g., fluorimetric or calorimetric) indicators of cellular respiratory activity, or cell counting, or the like. Similarly, in the cell biology arts there are known multiple techniques for assessing cell survival (e.g., vital dyes, metabolic indicators, etc.) and for determining apoptosis (e.g., annexin V binding, DNA fragmentation assays, caspase activation, etc.). Other signaling pathways will be associated with particular cellular phenotypes, for example specific induction of gene expression (e.g., detectable as transcription or translation products, or by bioassays of such products, or as nuclear localization of cytoplasmic factors), altered (e.g., statistically significant increases or decreases) levels of intracellular mediators (e.g., activated kinases or phosphatases, altered levels of cyclic nucleotides or of physiologically active ionic species, etc.), or altered cellular morphology, and the like, such that cellular responsiveness to a particular stimulus as provided herein can be readily identified to determine whether a particular cell responds to a particular sphingolipid signaling pathway.

Methods for Detecting Cancer

Within other aspects, the present invention provides methods and kits for diagnosing cancer and/or identifying individuals with a risk for developing cancer or with a risk for metastasis that is higher or lower than average. It has been found, within the context of the present invention, that certain human tumor cells contain an altered SK and SPL expression. In particular, decrease SPL expression was observed in certain tumor tissues as compared to corresponding normal tissue from the same individual, as described further in the Examples. Further, increase SK expression was observed in numerous tumor tissues as compared to corresponding normal tissue from the same individual. In other words, such polynucleotides or the proteins encoded by these polynucleotides, may be used as markers to indicate the presence or absence of a cancer in a patient.

Thus, one aspect of the present invention provides methods for detecting cancer by detecting alterations in expression level of polynucleotides encoding components of a sphingolipid metabolic and/or signaling pathway, in particular SK and SPL. In this regard, an individual demonstrating a statistically significant decrease in expression of SPL as compared to a control is considered to be afflicted with a cancer. In particular, a 50% to 60%, 61%, 62%, 63%, 64%, or 65% reduction in SPL expression in a cancer sample as compared to a corresponding normal tissue indicates the presence of cancer in a patient. In one embodiment, a 20%, 30%, 35%, 40%, 45%, 46%, 47%, 48%, or 49% reduction in SPL expression in a cancer sample as compared to a corresponding normal tissue indicates the presence of cancer in a patient. Likewise, an individual demonstrating a statistically significant increase in expression of SK as compared to a control is considered to be afflicted with a cancer. In particular, a 50% to 60%, 61%, 62%, 63%, 64%, or 65% increase in SK expression in a cancer sample as compared to a corresponding normal tissue indicates the presence of cancer in a patient. In one embodiment, a 20%, 30%, 35%, 40%, 45%, 46%, 47%, 48%, or 49% increase in SK expression in a cancer sample as compared to a corresponding normal tissue indicates the presence of cancer in a patient.

A cancer may be detected based on the level of mRNA encoding a protein involved in sphingolipid metabolism an/or signaling in a biological sample obtained from an individual suspected of having a cancer as compared to the level of mRNA detected in a biological sample obtained from a normal control subject known to be free of cancer. In certain embodiments, biological samples which contain cDNA pairs representing tumor tissue and corresponding normal tissue from the same patient can be used to determine the presence of cancer, for example as described in the examples. By utilizing sample (e.g., cell, tissue or biological fluid) pairs from one patient, differences between gene expression in tumor and normal tissue which might be due to person-to-person variability should not confound the interpretation of results. In certain other embodiments, samples may be obtained both from a subject suspected of having or being at risk for having cancer (e.g., a patient) and from a normal, control subject known to be free of the presence and/or risk for having cancer (e.g., a malignancy). Those familiar with the art will appreciate that for the described or characterized cancers, clinical criteria have been established for ascertaining when one or more signs or symptoms are apparent at levels upon which a suspicion that cancer is present may be based. A biological sample may include, but is not limited to, blood, sera, urine, cells or tissue of any type such as breast, lung, colon and the like, biopsy, tumor, lymph node, and the like. For example, at least two oligonucleotide primers may be employed in a polymerase chain reaction (PCR) based assay (e.g. RT-PCR, QC-RT-PCR, real-time PCR, etc.) to amplify a portion of a cDNA derived from a biological sample, wherein at least one of the oligonucleotide primers is specific for (i.e., hybridizes to specifically as determined using any one of a variety of techniques and controls known in the art) a polynucleotide encoding the protein. The amplified cDNA is then separated and detected using techniques well known in the art, such as gel electrophoresis. Generally, the oligonucleotide primers used in this context can be generated using guidelines known in the art. In particular, oligonucleotide primers are designed such that they are specific for a polynucleotide of interest. The PCR conditions used can be optimized in terms of temperature, annealing times, extension times and number of cycles depending on the oligonucleotide and the polynucleotide to be amplified. Such techniques are well know in the art and are described in for example, Mullis et al., Cold Spring Harbor Symp. Quant. Biol., 51:263, 1987; Erlich ed., PCR Technology, Stockton Press, NY, 1989). Oligonucleotide primers can be anywhere from 8, 9, 10, 11, 12, 13, 14, 15, 16, 17, 18, 19, 20, 21, 22, 23, 24, 25, 26, 27, 28, 29, or 30 nucleotides in length. In certain embodiments, the oligonucleotide primers of the present invention are 35, 40, 45, 50, 55, or 60 nucleotides in length. In one embodiment, the oligonucleotides comprise a sequence described herein, such as those set forth in SEQ ID NOs:1, 3, 5, 7, 9, 12, 15, 17, and 22-27, or the complement thereof.

Similarly, oligonucleotide probes that specifically hybridize to a polynucleotide encoding a protein involved in sphingolipid metabolism an/or signaling may be used in a hybridization assay to detect the presence of polynucleotide encoding said protein in a biological sample as described herein (biological sample may include, but is not limited to, blood, tissue, biopsy, tumor, lymph node, and the like) obtained from a patient suspected of having cancer. Oligonucleotide probes can be of the lengths as described above. In certain embodiments, a probe may comprise the entire sequence as set forth in SEQ ID NOs: 1, 3, 5, 7, 9, 12, 15, 17, and 22-27, or the complement thereof.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding a protein of the invention that is at least 10 nucleotides, and preferably at least 20 nucleotides, in length. Preferably, oligonucleotide primers and/or probes hybridize to a polynucleotide encoding a polypeptide described herein under moderately stringent conditions, as defined above. Oligonucleotide primers and/or probes which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule having a sequence as disclosed herein. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, NY, 1989).

One preferred assay employs RT-PCR, in which PCR is applied in conjunction with reverse transcription. Typically, RNA is extracted from a biological sample, such as biopsy tissue, and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a cancer. The amplification reaction may be performed on several dilutions of cDNA spanning two orders of magnitude. A statistically significant increase or decrease in expression in several dilutions of the test patient sample as compared to the same dilutions of the non-cancerous sample is typically considered positive. Alternatively, levels of polynucleotide in corresponding normal tissues from the same test patient may be used as controls.

One aspect of the present invention provides methods for monitoring the progression of a cancer by detecting alterations in expression level of polynucleotides encoding components of a sphingolipid metabolic and/or signaling pathway, in particular SK and SPL. In this embodiment, assays as described above for the diagnosis of a cancer may be performed over time, and the change in the level of reactive polypeptide(s) or polynucleotide(s) evaluated. For example, the assays may be performed every 24-72 hours for a period of 6 months to 1 year, and thereafter performed as needed. In general, a cancer is progressing in those patients in whom the level of polypeptide or polynucleotide detected shows a statistically significant increase (such as for SK) or decrease (such as for SPL) over Lime. In contrast, the cancer is not progressing when the level of reactive polypeptide or polynucleotide either remains constant with time.

Specific alterations present in the genes encoding the polypeptides of the present invention involved in sphingolipid metabolism in other tumor cells, such as breast, colon, uterine, or other tumor cells, may be readily identified using standard techniques, such as PCR. Alterations that may be associated with a particular tumor include amino acid deletions, insertions, substitutions and combinations thereof. Methods in which the presence or absence of such an alteration is determined may generally be used to detect cancer and to evaluate the prognosis for a patient known to be afflicted with cancer.

To detect an altered gene, any of a variety of well-known techniques may be used including, but not limited to, PCR and hybridization techniques, using polynucleotides of the present invention, or variants thereof. Any sample that may contain cancerous cells may be assayed. In general, suitable samples are tumor biopsies. Within a preferred embodiment, a sample is a breast tumor biopsy.

Kits for diagnosing or evaluating the prognosis of a cancer generally comprise reagents for use in the particular assay to be employed. In general, a kit of the present invention comprises one or more containers enclosing elements, such as primers, probes, reagents or buffers, to be used in an assay. For example, a kit may contain one or more polynucleotide primers or probes comprising at least 15 nucleotides complementary to a polynucleotide encoding a polypeptide involved in sphingolipid metabolism. In a preferred embodiment, said polypeptide is SK. In certain embodiments, the primers or probes comprise at least 10, 25, 30, 35, 40, 45, 50, 55, 60, 65, 70, 75, 80, 85, 90, 95 or 100 nucleotides, and preferably at least 150 or 200 nucleotides, complementary to an mRNA or to a polynucleotide encoding a polypeptide involved in sphingolipid metabolism. Such probe(s) may be used to detect, for example, an altered SK gene by hybridization. For example, a kit may contain one probe that hybridizes to a region of an SK or SPL gene that is not generally altered in tumors (a control) and a second probe that hybridizes to a region commonly deleted in breast cancer. A sample that contains mRNA that hybridizes to the first probe, and not to the second (using standard techniques) contains an altered SK or SPL gene. Suitable control probes include probes that hybridize to a portion of the SK or SPL gene outside of a deleted region. Alternatively, a kit may comprise one or more primers for PCR analyses, which may be readily designed based upon the sequences provided herein by those of ordinary skill in the art. Optionally, a kit may further comprise one or more solutions, compounds or detection reagents for use within an assay as described above.

In a related aspect of the present invention, kits for detecting a polypeptide involved in sphingolipid metabolism are provided. Such kits may be designed for detecting the level of protein or nucleic acid encoding a protein, e.g. SK or SPL, within a sample, or may detect the level of SK or SPL activity as described herein. A kit for detecting the level of SK or SPL, or nucleic acid encoding SK or SPL, or other component of sphingolipid metabolism and/or signaling as described herein, typically contains a reagent that binds to the protein, DNA or RNA. To detect nucleic acid encoding SK, SPL or other protein, the reagent may be a nucleic acid probe or a PCR primer. To detect SK, SPL, or other protein, the reagent is typically an antibody. The kit may also contain a reporter group suitable for direct or indirect detection of the reagent as described above.

Generation of Mutant and Transgenic Drosophila melanogaster

The invention further provides mutant and/or transgenic Drosophila melanogaster. In one embodiment, a mutant Drosophila melanogaster comprises a P-element transposon insertion in a coding region of a gene encoding a component of a sphingolipid metabolic and/or signaling pathway. In certain embodiments, the P-element transposon insertion results in an altered level (e.g., a statistically significant increase or decrease) of at least one sphingolipid intermediate as described herein. In a further embodiment, the P-element transposon results in altered activity level of at least one sphingolipid pathway component as described herein. In further embodiments, the mutant Drosophila melanogaster of the present invention comprise a P-element insertion in the coding region of more than one gene encoding a component of a sphingolipid metabolic and/or signaling pathway. Mutants can be generated comprising any number of insertions in any number of genes encoding components of a sphingolipid metabolic and/or signaling pathway. In certain embodiments, 1, 2, 3, 4, or 5 genes encoding components of a sphingolipid metabolic and/or signaling pathway contain P-element insertions.

Illustrative lines of Drosophila melanogaster include Wild type Canton S, lace²/lace⁰⁵³⁰⁵ and Sply mutant lines. Flies can be obtained from the Drosophila Genome Project Stock Center (Bloomington, Ind.). General fly husbandry is known in the art and is described for example, in Ashburner, M and Roote J, 2000. Laboratory culture of Drosophila, Drosophila Protocols 585-600. Analysis of Drosophila melanogaster anatomical structures may be carried out by the skilled artisan using a variety of techniques in the art, including those described in (O'Donnell, P. T. and Bernstein, S. I. (1988). J Cell Biol 107, 2601-12.; Fyrberg, E. A., Bernstein, S. I. and VijayRaghavan, K. (1994). Methods Cell Biol 44, 237-58.).

The invention further provides Drosophila melanogaster mutants that exhibit a flightless phenotype, where the phenotype results from the disruption of an endogenous gene involved in sphingolipid metabolism and/or signaling, for example, the SPL, SK, SPT, or other gene as described in detail herein. In one embodiment, flightless phenotype is meant that the subject non-mammalian organism models spontaneously develop a reduced number of muscle fibers comprising the dorsal longitudinal muscles (DLM) and have compensatory hypertrophy in the remaining fibers. Analysis of DLM formation is carried using markers specific for different stages of differentiation, as well as GFP markers which distinguish myoblasts emerging from imaginal discs versus larval template muscles. Expression of a series of markers of muscle development can also be used in evaluating embryonic muscle differentiation. For example, Dmef2 is expressed in migrating myoblasts, allowing analysis of early steps in embryonic myogenesis. These myoblasts divide, forming two myocytes that express αMHC and ultimately fuse to form embryonic muscles. Thus, by evaluating αMHC, embryonic muscle fusion can be evaluated.

In certain aspects, the Drosophila melanogaster mutant of the present invention may also demonstrate abnormal developmental patterning of thoracic muscles of the T2 segment. Identification of Drosophila melanogaster anatomy is readily carried out by the skilled artisan using a variety of techniques knows in the art, including those described in the Examples herein, or, for example, Developmental Biology, 6th Edition, Scott F. Gilbert, Sinauer Associates, Inc., Sunderland, Mass. In a preferred embodiment, the above phenotypes result in an inability to fly or otherwise reduced flight performance as described in the Examples or as described in Vigoreaux, et al., 1993 J. Cell Biol. May; 121(3):587-98. The subject Drosophila melanogaster, within a preferred embodiment, demonstrate altered activity of at least one component of a sphingolipid metabolic and/or signaling pathway, such as SPL, SK, SPT, ceramidase or other component as described herein. In a particularly illustrative embodiment, said Drosophila melanogaster has decreased activity of endogenous SPL and/or increased or decreased activity of SK.

In a preferred embodiment, the strain contains a mutation in any one or more of the genes encoding a component of the sphingolipid metabolism and/or signaling, such as SPL, SK, SPT, S-1-PP, ceramidase, or any combination thereof. In a further embodiment of the present invention the D. melanogaster strain are heterozygous for a P-element transposon which sits in any region of the gene encoding the SPL protein set forth in SEQ ID NO:16. In a certain embodiment, the P-element transposon sits in a regulatory region of the gene. In a preferred embodiment, the flies are homozygous insertional mutants in the coding region of the gene encoding the SPL protein set forth in SEQ ID NO:16. In a further embodiment of the present invention the D. melanogaster strain are heterozygous for a P-element transposon which sits in the coding region of the gene encoding the SK protein set forth in any one of or all of SEQ ID NOs:18, 19, 20, 28, and 29. In a preferred embodiment, the flies are homozygous insertional mutants in the coding region of the gene encoding the SK protein set forth in any one or more of SEQ ID NOs:18, 19, 20, 28, and 29. In yet a further embodiment of the present invention, the homozygous mutant strain of fly has a flightless phenotype. In certain embodiments, the mutant flies have a reduced number of muscle fibers comprising the dorsal longitudinal muscles and have compensatory hypertrophy in the remaining fibers. In certain aspects, the mutant flies of the present invention may also demonstrate abnormal developmental patterning of thoracic muscles of the T2 segment, for example as described herein in the Examples. Identification of normal and abnormal anatomy of the Drosophila melanogaster can be carried out using techniques known to the skilled artisan and described herein, and for example, in Developmental Biology, 6th Edition, Scott F. Gilbert, Sinauer Associates, Inc., Sunderland, Mass. Illustrative mutant flies have altered sphingolipid metabolism.

Flies heterozygous for a P-element transposon which sits in a gene encoding a component of sphingolipid metabolism and/or signaling may be obtained from the Drosophila Genome Project. Homozygous insertional mutants can be made, using techniques known in the art, by genetically crossing and evaluating progeny for the presence of homozygous insertional mutants (for example, based on presence of rosy eye color, encoded by a recessive marker carried on the P-element). Expression of the SPL or other gene involved in sphingolipid metabolism, can be evaluated using any number of assays known to the skilled artisan, for example, by Northern blot analysis. To determine the SPL function of each genotype, +/+, +/− and −/− flies may be homogenized using standard techniques and whole extracts can be assayed for SPL activity using assays as described herein. The transposon can be mobilized by crossing SPL mutant flies with flies carrying an actively transcribed transposase gene, which should cause the P-element to be excised in the chromosomes of both somatic cells and in the germline. Germline transposon loss is heritable and can be identified in progeny by virtue of eye color or other relevant marker. Progeny which lost both the transposase gene and the P-element can then be isolated and the restored allele can be homozygosed.

Mutations in Drosophila melanogaster as described herein which permanently block expression of a functional protein can be created in several ways, such as with P-element transposon insertions or chemical or radiation induced mutagenesis. Exemplary strains of mutant flies are available through the Drosophila Genome Project, at the University of California at Berkeley (Adams, M. et al 2000. The genome sequence of Drosophila melanogaster. Science. 287:2185-2195.). Alternatively, insertional mutant of interest may be obtained by using local hop strategies essentially as described in Tower, J. et al (Tower, J., et al. 1993. Preferential transposition of Drosophila P elements to nearby chromosomal sites. Genetics. 133:347-359.). Transposons can be mobilized by crossing in a transposase gene, followed by crossing the transposase back out (reintroducing genetic stability). Mutant flies can be identified using techniques know to those of skill in the art. For example, mutant flies can be identified by probing Southern blots prepared from extracts from flies generated in the screen using the target gene as probe. Subsequently, crosses can be performed to introduce a mutant allele of interest, (e.g. SPL, SK, SPT or other component of sphingolipid metabolism and/or signaling) and generate homozygosity at both mutant alleles (e.g. SPL and new transposon integration sites). Mutants can be screened for a phenotype of interest, for example the ability to restore flight to an SPL mutant when the mutated allele is homozygous (predicting a recessive phenotype).

In one aspect of the present invention, fly genetic manipulation may entail mating or “crossing” of flies and selection for or against progeny expressing various phenotypic markers. Exemplary techniques for fly genetic manipulation of the present invention are know in the art and are described, for example in, Ashburner, M., and J. Roote. 2000. Laboratory culture of Drosophila. In Drosophila Protocols. W. Sullivan, M. Ashburner, and R. Hawley, editors. Cold Spring Harbor Laboratory Press, Cold Spring Harbor, N.Y. 585-600. Phenotypic markers may be used to identify the inheritance of chromosomes, engineered transposable elements, or transposase genes used to facilitate their mobilization. Marker mutations affecting eye color, bristle shape, wing morphology and cuticle pigmentation, for example, may be employed in the crosses for the mutant flies of the present invention. Within one aspect of the present invention, it may be desirable to select the individuals which contain a collection of markers indicating the desired genotype. In another aspect of the present invention, balancer chromosomes may be used to create the ability to identify recessive mutations present in the heterozygous state. Balancer chromosomes may be employed to prevent homologous recombination during meiotic prophase in females. The presence of both dominant and recessive lethal markers allows one to determine the presence or absence of the balancer chromosomes and simultaneously to follow the homologous chromosomes, which may themselves not contain a dominant marker. One particularly illustrative cross of the present invention is to eliminate the P-element insertion in the Drosophila melanogaster SPL gene and establish phenotypic reversion, as described herein in the Examples.

The genetics required to create the mutant flies described herein may involve several successive steps. For example, lines homozygous for the Sply⁰⁵⁰⁹¹ allele and the lace⁰⁵³⁰⁵ allele can to be generated by meiotic recombination. Sply⁰⁵⁰⁹¹ and lace⁰⁵³⁰⁵ mutations can be introduced in trans and balanced in the next generation. Flies carrying the lace⁰⁵³⁰⁵ allele can be selected by the presence of w⁺. Presence of ply⁰⁵⁰⁹¹ and other mutation of the present invention can be verified by PCR. Similar strategies are employed to create other strategic crosses envisioned by the present invention. For example, lines containing null alleles for both SK genes will be generated. Lines containing null alleles for one or more components of a sphingolipid metabolic and/or signaling pathway are envisioned by the present invention. For example, lines containing null alleles for SK and SPL are envisioned. Lines containing null alleles for SPT and SK are envisioned as are other double, triple, and quadruple mutant lines of virtually any component in a sphingolipid metabolic and/or signaling pathway.

Selective markers to allow for selection of mutant flies is provided for in the present invention. Exemplary selective markers of the present invention may comprise a wild type rosy (ry⁺) allele carried on the transposon to allow for selection for or against the stable transposon. Introduction of an active transposase is selected for by presence of, for example, the dominant marker, Stubble (short bristle phenotype) in the first cross, and is selected against to identify progeny which have lost the transposase, restoring genetic stability in the second cross. Other illustrative markers include Curly O (CyO) which is lethal when present in two copies, allowing selection for heterozygotes containing the CyO balancer and another allele of interest originally containing the transposon (e.g., SPL). By selecting against rosy eye color, progeny in which the transposon has been excised from the locus of interest, e.g., SPL, SK, or other components of sphingolipid metabolism and/or signaling can be identified. Expansion of this “reverted” allele in the population can be achieved in the third cross, and the desired allele can be homozygosed in the final cross, to determine whether restoration of the intact allele of interest, for example SPL and/or SK, is associated with a desired phenotype of interest, such as restoration of flight.

Transgenic Drosophila melanogaster are also provided in the present invention. Relevant methods of preparing transgenic Drosophila melanogaster are disclosed in: Spradling, A. C., and Rubin, G. M. (1982). Science 218, 341-347; Brand & Perrimon, Development (1993) 118: 401-415; and Phelps & Brand, Methods (April 1998) 14:367-379. See also, Spradling A C, P Element Mediated Transformation in Drosophila: A Practical Approach (ed. D. D. Roberts, IRL Press, Oxford)(1986) pp 175-179; and U.S. Pat. No. 6,316,690.

The subject transgenic flies can be prepared using any convenient protocol that provides for stable integration of the transgene in to the fly genome in a manner sufficient to provide for the requisite spatial expression of the transgene. A number of different strategies can be employed to obtain the integration of the transgene with the requisite expression pattern. Generally, methods of producing the subject transgenic flies involve stable integration of the transgene into the fly genome. Stable integration is achieved by first introducing the transgene into a cell or cells of the fly, e.g. a fly embryo. The transgene is generally present on a suitable vector, such as a plasmid. Transgene introduction may be accomplished using any convenient protocol, where suitable protocols include: electroporation, microinjection, vesicle delivery, e.g. liposome delivery vehicles, and the like. Following introduction of the transgene into the cell(s), the transgene is stably integrated into the genome of the cell. Stable integration may be either site specific or random, but is generally random.

Where integration is random, the transgene is typically integrated with the use of transposase. In such embodiments, the transgene is introduced into the cell(s) within a vector that includes the requisite P element, terminal 31 base pair inverted repeats. Where the cell into which the transgene is to be integrated does not comprise an endogenous transposase, a vector encoding a transposase is also introduced into the cell, e.g. a helper plasmid comprising a transposase gene, such as PTURBO (as disclosed in Steller & Pirrotta, “P Transposons Controlled by the Heat Shock Promoter,” Mol. Cell. Biol. (1986) 6:1640-1649). Methods of random integration of transgenes into the genome of a target Drosophila melanogaster cell(s) are disclosed in U.S. Pat. No. 4,670,388.

In those embodiments in which the transgene is stably integrated in a random fashion into the fly genome, means are also provided for selectively expressing the transgene at the appropriate time during development of the fly. In other words, means are provided for obtaining targeted expression of the transgene. To obtain the desired targeted expression of the randomly integrated transgene, integration of particular promoter upstream of the transgene, as a single unit in the P element vector may be employed. Alternatively, a transactivator that mediates expression of the transgene may be employed. Of particular interest is the GAL4 system described in Brand & Perrimon, supra.

In one aspect of the present invention, transgenic flies can be created using P-elements to express, overexpress or misexpress proteins of interest, such as SPL, SK, SPT, S-1-PP, ceramidase, or any combination thereof. The transgenes for use in the context of the present invention may include SPL, SK, SPT, S-1-PP, ceramidase, or any other protein involved in sphingolipid metabolism and/or signaling from human, mouse, yeast, or C. elegans. In further embodiments of the present invention, the transgene of interest comprises a polynucleotide encoding a fusion protein. For example, a polynucleotide encoding a polypeptide component of the sphingolipid metabolic pathway, such as SK, SPL, SPT, S-1-PP and the like, can be engineered to fuse the protein of interest to a marker protein such as GFP for use as a transgene in the context of this invention. The skilled artisan would readily recognize that any number of marker proteins can be used in fusion proteins of the present invention.

In one embodiment of the invention, GAL4-mediated ectopic gene expression is employed, essentially as described (van Roessel, P., and A. Brand. 2000. GAL4-mediated ectopic gene expression in Drosophila. In Drosophila Protocols. W. Sullivan, M. Ashburner, and R. Hawley, editors. Cold Spring Harbor Laboratory Press, Cold Spring Harbor, N.Y. 439-448.). Other illustrative inducible promoter may be used as well, such as those described in Rubin, G, Hong L, Brokstein P, Evans-Holm M, Frise E, Stapleton M and Harvey D, 2000. A Drosophila complementary DNA resource, Science 287:2222-2224. The GAL4 protein is a yeast transcription factor capable of activating transcription of Drosophila melanogaster genes which have been engineered to contain upstream sequences recognized by the GAL4 protein. Various mutants can be created with a gene of interest expressed in specific tissue distributions, a construct containing the gene of interest (reporter) under regulation of a GAL4 containing promoter is introduced into embryos, and a genetic marker allows identification of progeny containing this construct. Illustrative GAL4 containing promoters include, but are not limited to, pUAS. The skilled artisan would readily appreciate that other inducible systems can be used in the context of the present invention. The use of embryos of a strain containing an active P-transposase increases the efficiency of transgene integration, although many of the embryos die. These progeny can then be crossed to various available lines containing GAL4 transgenes (driver) expressed under control of tissue-specific promoters. In one embodiment of the present invention, GAL4 driver constructs which allow expression during embryogenesis may be used.

Various methods to identify the etiology of the SPL, SPT and other mutant phenotypes are known to those of skill in the art and are also provided herein. The present invention provides for mutant Drosophila melanogaster with defective or overexpressed SPL, SK, SPT and S-1-P phosphatase genes. In a particular embodiment, abnormal sphingolipid metabolism is a phenotype of the mutant Drosophila melanogaster of the present invention. In a further embodiment, the abnormal sphingolipid metabolism affects developmental programs in the mutant flies. In certain embodiments, the mutant and/or transgenic Drosophila melanogaster of the present invention develop one or more tumors. Tumors can be identified and measured using a variety of techniques known to the skilled artisan. Particularly illustrative techniques are described in, for example, De Lorenzo, C., B. M. Mechler, and P. J. Bryant. 1999. Cancer Metastasis Rev. 18:295-311.; Watson, K., R. Justice, and P. Bryant. 1994. J Cell Sci Supp. 18:19-33.; Gateff, E., and H. Schneiderman. 1967. Amer Zool. 7:760.; Gateff, E. 1994. Int J Dev Biol. 4:565-590, or any references cited therein.

Quantitative analysis and characterization of sphingolipids in normal and mutant and/or transgenic flies throughout development may be carried out using any number of techniques known to the skilled artisan. Such techniques are described in, for example, Ashburner, M., and J. Roote. 2000, Laboratory culture of Drosophila. In Drosophila Protocols, W. Sullivan, M. Ashburner, and R. Hawley, editors, Cold Spring Harbor Laboratory Press, Cold Spring Harbor, N.Y. Pp. 585-600; Blair, S. 2000, Imaginal discs, In Drosophila Protocols, W. Sullivan, M. Ashburner, and R. Hawley, editors. Cold Spring Harbor Laboratory Press, Cold Spring Harbor, N.Y. 159-175, and Stern, D., and E. Sucena. 2000. Preparation of larval and adult cuticles for light microscopy. In Drosophila Protocols. W. Sullivan, M. Ashburner, and R. Hawley, editors. Cold Spring Harbor Laboratory Press, Cold Spring Harbor, N.Y. 601-616.

Further, identification of lipids which are not within normal concentration ranges in various mutants of the present invention, especially those demonstrating developmental defects, can be done using standard techniques. In certain embodiments, the embryonic, larval, pupal and adult stages of development of Drosophila melanogaster models harboring single or multiple sphingolipid metabolic defects are examined using techniques described herein, in particular in the Examples. Light and electron microscopy and in situ hybridization with appropriate tissue-specific markers of differentiation should identify both gross and subtle defects associated with altered sphingolipid metabolism.

Within further aspects, the present invention provides other transgenic organisms in which sphingolipid metabolism is altered, compared to wild-type organisms. Within the context of the present invention, organisms may include but are not limited to mice, rats, and C. elegans, and other species of Drosophila. Such organisms may contain an alteration, insertion or deletion in an endogenous gene involved in sphingolipid metabolism and/or signaling, or may contain DNA encoding a modulating agent that modulates expression or activity of a gene involved in sphingolipid metabolism. In one embodiment the altered endogenous gene comprises SK. In certain aspects, such organisms may contain DNA encoding a modulating agent that increases expression or activity of an SK or an SPL gene. Transgenic organisms may be generated using techniques that are known to those of ordinary skill in the art. For example, a transgenic organism containing an insertion or deletion in the coding region for the SK or SPL gene may be generated from embryonic stem cells, using standard techniques. Such stem cells may be generated by first identifying the full genomic sequence of the gene encoding the SK or SPL, and then creating an insertion or deletion in the coding region in embryonic stem cells. Alternatively, appropriate genetically altered embryonic stem cells may be identified from a bank. Using the altered stem cells, hybrid organisms may be generated with one normal SK or SPL gene and one marked, abnormal gene. These hybrids may be mated, and homozygous progeny identified.

Transgenic organisms may be used for a variety of purposes, which will be apparent to those of ordinary skill in the art. For example, such organisms may be used to prepare cell lines from different tissues, using well known techniques. Such cell lines may be used, for example, to evaluate the effect of the alteration, and to test various candidate modulators.

In addition to their use as animal models for screening candidate therapeutic agents, the subject mutant and transgenic flies also find use in the identification of gene targets involved in sphingolipid metabolism and/or signaling, i.e. genes whose expression can be beneficially modulated to treat diseases associated with sphingolipid metabolism and/or signaling. Gene based therapies can be identified by doing traditional enhancer/suppressor analyses in the subject mutant and transgenic flies. In these analyses, genes in the subject mutant and/or transgenic flies are mutated to identify ones that either exacerbate or alleviate the mutant or transgenic phenotype. Methods of mutating genes and carrying out enhancer/suppressor analyses are well known to those of skill in the art (Hays, T S et al., Molecular and Cellular Biology (March 1989) 9(3):875-84; Deuring, R; Robertson, B; Prout, M; and Fuller, M T. Mol. Cell. Biol., 1989 9:875-84.; Fuller, M T et al., Cell Mot. Cyto. (1989) 14 :128-35; Rottgen G, Wagner T, Hinz U Mol. Gen. Genet. 1998 257:442-51).

Genes that mutate to enhance the phenotype of mutant and/or transgenic flies of the present invention in a recessive manner yield potential protein therapeutics for conditions associated with sphingolipid metabolism and/or signaling, since elevating the normal gene product level of such genes potentially alleviates such condition. Genes that mutate to suppress the adult onset neurodegeneration phenotype in a recessive manner yield gene targets for disruption to alleviate the diseases associated with sphingolipid metabolism or signaling, where disruption of these genes can be achieved using a variety of methods, ranging from deleting the DNA for the target gene to inhibiting its transcription, translation, or protein activity. For screening candidate agents, small molecule antagonists to these genes can be constructed and evaluated for efficacy in the fly model through oral administration. Alternatively, large molecular antagonists can be delivered by gene therapy, as described infra.

Generation of Mutant Yeast Strains

As noted above, according to certain embodiments, the present invention provides mutant strains of yeast, in particular Saccharomyces cerevisiae, having one or more mutated and/or transformed components of a sphingolipid metabolic and/or signaling pathway, and methods of using such yeast strains to identify agents that modulate sphingolipid metabolism and/or signaling. Other yeast which can be used in practicing the present invention are Neurospora crassa, Aspergillus niger, Aspergillus nidulans, Pichia pastoris, Candida tropicalis, and Hansenula polymorpha. The term “yeast”, as used herein, includes not only yeast in a strictly taxonomic sense, i.e., unicellular organisms, but also yeast-like multicellular fungi or filamentous fungi. In particular, according to certain preferred embodiments, the present invention provides mutant strains of yeast comprising at least one mutation in at least one gene encoding any component of the sphingolipid metabolic and/or signaling pathway. In this regard a mutation may comprise an insertion, a deletion, a knockout allele, a null allele, a dominant negative mutation, or any other mutation that modifies the native sequence of the gene so as to modulate the activity of the component of sphingolipid metabolic and/or signaling pathway. In one embodiment, a mutant strain of yeast comprises a disruption mutant in one or more genes encoding a component of sphingolipid metabolism and/or signaling. In this context, the mutant yeast strains of the present invention include strains containing null alleles of one or more of the genes encoding any component of the sphingolipid metabolic and/or signaling pathway including, but not limited to, SPL (DPL1), SK (LCB4 and/or LCB5), SPP (YSR2 and/or YSR3), a gene that catalyzes the conversion of dihydrosphingosine to phytosphingosine (SUR2), ceramidase, SPT, and the like. In one embodiment, the mutant yeast strain comprises dpl1Δ. In a further embodiment, the mutant yeast strain comprises a dpl1, ysr2 double mutant. In a further embodiment, the mutant yeast strain comprises a dpl1, ysr2, Icb4 triple mutant. In one embodiment, the mutant yeast strains have a phenotype characterized by sensitivity to long chain sphingoid bases, such as D-erythro-sphingosine. In certain embodiments, the mutant yeast strain is characterized by intracellular accumulation of LCBPs.

In certain embodiments, the mutant yeast strains are transformed with one or more polynucleotides encoding one or more components of the sphingolipid metabolic and/or signaling pathway. In one particular embodiment, the mutant yeast strains are transformed with one or more polynucleotides encoding one or more nonendogenous (e.g., of non yeast origin or derived from a source different from the mutant yeast strain) components of the sphingolipid metabolic and/or signaling pathway in this regard, the polynucleotides may be derived from human, mouse, C. elegans, D. melanogaster, or other species. In certain embodiments, a mutant yeast strain is transformed with a polynucleotide encoding human SK (SPHK1), mouse SK, C. elegans SK, or D. melanogaster SK. In another embodiment, a mutant yeast strain of the present invention is transformed with a polynucleotide encoding human SPL, ceramidase, SPP, SPT, 3-keto dihydrosphingosine reductase, ceramide synthase, sphingosine desaturase, ceramide kinase, phosphoethanolamine cytidylyltransferase, CDP-ethanolamine phosphotransferase, acid sphingomyelinase, sphingomyelin synthase, neutral sphingomyelinase, oxosphinanine reductase, and/or glucosylceramide synthase. In yet a further embodiment, a mutant yeast strain of the present invention is transformed with a polynucleotide encoding a human intracellular or cell surface receptor, such as any one or more of the EDG receptors (e.g. EDG1, EDG3, EDG5, EDG6, EDG8) or CFTR. As noted above, the murine, C. elegans, and D. melanogaster homologs of the above polynucleotides may also be included within the context of this invention.

Polynucleotides for use in transforming mutant yeast strains as described herein may be cloned using standard techniques into any expression vector suitable for use within the yeast strain and transformed into the host cell using techniques well known to those of ordinary skill in the art, such as the lithium acetate method (Ito, H., Fukuda, Y., Murata, K. and Kimura, A. (1983). J Bacteriol 153, 163-8.). As is readily appreciated by the skilled artisan, the polynucleotide encoding the polypeptide of interest is cloned into the expression vector such that it is operably linked to the promoter such that the polypeptide of interest is properly translated and expressed as a functional protein. In certain embodiments, the promoter used is an inducible promoter, e.g. a galactose inducible promoter. A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al. (1983) in Experimental Manipulation of Gene Expression, ed. M. Inouye Academic Press, p. 83). These vectors can replicate in E. coli due the presence of the pBR322 ori, and in S. cerevisiae due to the replication determinant of the yeast 2 micron plasmid. In addition, drug resistance markers such as ampicillin may be used. Moreover, if yeast are used as a host cell, it will be understood that the expression of a gene in a yeast cell may require a promoter which is functional in yeast. Suitable promoters include the promoters for metallothionein, 3-phosphoglycerate kinase (Hitzeman et al., J. Biol. Chem. 255, 2073 (1980) or other glycolytic enzymes (Hess et al., J. Adv. Enzyme Req. 7, 149 (1968); and Holland et al. Biochemistry 17, 4900 (1978)), such as enolase, glyceraldehyde-3-phosphate dehydrogenase, hexokinase, pyruvate decarboxylase, phospho-fructokinase, glucose-6-phosphate isomerase, 3-phosphoglycerate mutase, pyruvate kinase, triosephosphate isomerase, phosphoglucose isomerase, and glucokinase. Suitable vectors and promoters for use in yeast expression are further described in R. Hitzeman et al., EPO Publication No. 73,657. Other promoters, which have the additional advantage of transcription controlled by growth conditions, are the promoter regions, alcohol dehydrogenase 2, isocytochrome C, acid phosphatase, degradative enzymes associated with nitrogen metabolism, and the aforementioned metallothionein and glyceraldehyde-3-phosphate dehydrogenase, as well as enzymes responsible for maltose and galactose utilization. Illustrative expression vectors for use in the present invention include pYES (Invitrogen, Carlsbad, Calif.). Finally, promoters that are active in only one of the two haploid mating types may be appropriate in certain circumstances. Among these haploid-specific promoters, the pheromone promoters MFa1 and MF.alpha.1 are of particular interest.

In certain embodiments, the ligated DNA sequences are operably linked to suitable transcriptional or translational regulatory elements. The regulatory elements responsible for expression of DNA are generally located only 5′ to the DNA sequence encoding the first polypeptides. Similarly, stop codons required to end translation and transcription termination signals are only present 3′ to the DNA sequence encoding the second polypeptide.

Optionally, the construct may contain an enhancer, a transcription terminator, a poly(A) signal sequence, a bacterial or mammalian origin of replication and/or a selectable marker, all of which are well known in the art. Enhancer sequences may be included as part of the promoter region or separately. Transcription terminators are sequences that stop RNA polymerase-mediated transcription. The poly(A) signal may be contained within the termination sequence or incorporated separately. A selectable marker includes any gene that confers a phenotype on the host cell that allows transformed cells to be identified. Such markers may confer a growth advantage under specified conditions. Suitable selectable markers for yeast cells include but are not limited to URA3 which confers the ability to grow on medium without uracil, and LEU2.

Illustrative methods for generating and growing mutant yeast strains are described, for example, in Sherman, F., et al. 1986, Methods in Yeast Genetics. Cold Spring Harbor Laboratory Press, Cold Spring Harbor, N.Y.; Kim and Saba, 2000 Genetics 156:1519-1529; Saba, J., et al., 1997. J. Biol. Chem. 272:26087-26090; Skrzypek, M., et al., 1999 J. Bacteriol. 181:1134-1140; and Lanterman and Saba, 1998 Biochem. J. 332:525-531. In particular, disruption or deletion procedures to mutate genes of interest with PCR-amplified disruption constructs are described for example in Baudin, A. et al., 1993 Nucleic Acids Res. 21:3329-3330; Lorenz, M. C., et al., 1995 Gene 158:113-117; and Wach, A., et al., 1994 Yeast 10:1793-1808. In certain embodiments, transposon mutagenesis is employed in which cells are transformed with a plasmid containing a transposable element, and the genomic location of the gene into which the transposon had inserted is determined using any number of assays including genetic suppressor screens as described herein.

Methods of Use and Pharmaceutical Compositions

The agents that modulates a component of sphingolipid metabolism and/or signaling and/or a sphingolipid intermediate as described herein are useful for the detection, diagnosis and treatment of any disease associated with altered sphingolipid metabolism and/or signaling. Illustrative diseases include but are not limited to a variety of cancers (e.g. breast, colon, uterus, stomach, ovary, lung, kidney and rectum cancer), diseases that result from muscle developmental defects, cardiomyopathy, and hereditary sensory neuropathy type 1 and the sphingolipidoses. Thus, the compositions of the present invention may be used to inhibit the development of cancer, metastasis, or both development of cancer and metastasis in an individual afflicted with a cancer. The compositions of the present invention may also be used in the treatment of inflammatory and immune disorders. In further embodiments, the compositions of the present invention may be used as antithrombotic therapy, such as for the therapy of pathologic thrombus formation associated with ischemic heart disease, stroke, vascular disease, angioplasty, and stent deployment.

The compositions of the present invention may be administered to an individual afflicted with a disease associated with altered sphingolipid metabolism and/or signaling. For in vivo use for the treatment of human disease, an agent that modulates a component of sphingolipid metabolism and/or signaling and/or a sphingolipid intermediate as described herein is generally incorporated into a pharmaceutical composition prior to administration. A pharmaceutical composition comprises one or more modulating agents in combination with a physiologically acceptable carrier or excipient. To prepare a pharmaceutical composition, an effective amount of one or more modulating agents is mixed with any pharmaceutical carrier(s) or excipient known to those skilled in the art to be suitable for the particular mode of administration. A pharmaceutical carrier may be liquid, semi-liquid or solid. Solutions or suspensions used for parenteral, intradermal, subcutaneous or topical application may include, for example, a sterile diluent (such as water), saline solution, fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously, suitable carriers include physiological saline or phosphate buffered saline (PBS), and solutions containing thickening and solubilizing agents, such as glucose, polyethylene glycol, polypropylene glycol and mixtures thereof. In addition, other pharmaceutically active ingredients (including other anti-cancer agents) and/or suitable excipients such as salts, buffers and stabilizers may, but need not, be present within the composition.

A modulating agent may be prepared with carriers that protect it against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

Administration may be achieved by a variety of different routes, including oral, parenteral, nasal, intravenous, intradermal, subcutaneous or topical. Preferred modes of administration depend upon the nature of the condition to be treated or prevented. An amount that, following administration, inhibits, prevents or delays the progression and/or metastasis of a cancer is considered effective. Preferably, the amount administered is sufficient to result in regression, as indicated by 50% mass or by scan dimensions. The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

In certain embodiments, particularly where the modulating agent comprises a polynucleotide, a polynucleotide encoding a modulating agent may be administered. Such a polynucleotide may be present in a pharmaceutical composition within any of a variety of delivery systems known to those of ordinary skill in the art, including nucleic acid, bacterial and viral expression systems, and colloidal dispersion systems such as liposomes. Appropriate nucleic acid expression systems contain the necessary DNA sequences for expression in the patient (such as a suitable promoter and terminating signal, as described above). The DNA may also be “naked,” as described, for example, in Ulmer et al., Science 259:1745-49, 1993.

Various viral vectors that can be used to introduce a nucleic acid sequence into the targeted patient's cells include, but are not limited to, vaccinia or other pox virus, herpes virus, retrovirus, or adenovirus. Techniques for incorporating DNA into such vectors are well known to those of ordinary skill in the art. Another delivery system for polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. The preparation and use of liposomes is well known to those of ordinary skill in the art.

Within certain aspects of the present invention, one or more modulating agents may be used to modulate expression and/or activity of a component of a sphingolipid metabolic and/or signaling pathway, in a cell or in a mammal. In vitro, a polypeptide that is involved in sphingolipid metabolism may be contacted with a modulating agent that increases or decreases it's activity (e.g., certain antibodies, chemicals, or small molecules). In one embodiment, activity can be measured through levels of sphingolipid intermediates using assays as described herein. In a further embodiment, the modulating agent increases or decreases activity of a component of sphingolipid metabolism and/or signaling and can be assayed using methods as described herein. For use within a cell or a mammal, such modulation may be achieved by contacting a target cell with an effective amount of a modulating agent, as described herein. Administration to a mammal may generally be achieved as described herein.

As noted above, altered expression and/or activity provides a method for inhibiting the growth (i.e., proliferation) of a cancer cell, either in culture or in a mammal afflicted with cancer. In vivo, such alteration or modulation may also be used to inhibit cancer development, progression and/or metastasis. Accordingly, one or more modulating agents as provided herein may be administered as described above to a mammal in need of anti-cancer therapy. Patients that may benefit from administration of a modulating agent are those afflicted with cancer. Such patients may be identified based on standard criteria that are well known in the art. Within preferred embodiments, a patient is afflicted with breast cancer, as identified based on tissue biopsy and microscopic evaluation, using techniques well known in the art. In particular, patients whose tumor cells contain a tissue-specific deletion and/or alteration within an endogenous gene encoding a component of a sphingolipid metabolic and/or signaling pathway may benefit from administration of a modulating agent, as provided herein. In further embodiments, the patient may be afflicted with cancer of the breast, uterus, stomach, ovary, lung, kidney and rectum.

The following Examples are offered by way of illustration and not by way of limitation.

### EXAMPLES

Materials and Methods used in some of the Examples below are described herein as follows. (see also D. Herr, H. Fyrst, et al. 2003, Development).

Saccharomyces Cerevisiae Strains and Growth Conditions

Wild type yeast used herein were of strain JK9-3d (leu2-3, 112 ura3-52 rme1 trp1 his4 HMLa) (Heitman, J., Movva, N. R., Hiestand, P. C. and Hall, M. N. (1991). FK 506-binding protein proline rotamase is a target for the immunosuppressive agent FK 506 in Saccharomyces cerevisiae. Proc Natl Acad Sci USA 88, 1948-52.) The yeast strain JSK386 (dpl1Δ) is an isogenic derivative of strain JK9-3d in which the DPL1 gene has been replaced by a G418-resistant marker (Kim, S., Fyrst, H. and Saba, J. (2000). Accumulation of phosphorylated sphingoid long chain bases results in cell growth inhibition in Saccharomyces cerevisiae. Genetics 156, 1519-29.). Strains JS204 and JS205 are derivatives of JSK386 which contain the Drosophila melanogaster ESTs LP04413 and GH3783 respectively in expression vector, pYES2 (Invitrogen, Inc., Carlsbad, Calif.). pYES2 is a yeast expression vector containing the URA3 gene (which provides transformants the ability to grow in media without uracil), and an Ampicillin resistance marker and origin of replication functional in Escherischia coli. Genes expressed using this system are regulated under the control of the GAL1, 10 promoter, which allows expression in the presence of galactose and not in the presence of glucose. Cells were grown in minimal or uracil⁻ media containing either 20 g glucose or galactose per liter, as indicated.

Functional Complementation in Yeast

Strains of interest were grown to saturation in liquid culture for 2-3 days. They were then resuspended in minimal medium, placed in the first row of a 96-well plate and diluted serially from 1:2 to 1:4000 across the plate. The cultures were normalized for O.D.₆₀₀=2 and template inoculated onto a control plate and a plate containing 50 μM sphingosine, obtained from Sigma Chemical Company (St. Louis, Mo.). Sphingosine enriched plates were made with minimal media containing 0.0015% NP40 and 50 μM D-erythro-sphingosine. At this concentration of NP40, no effects on cell viability are observed. Plates were incubated at 30° C. for two days and assessed visually for differences in growth.

SPL Assays

SPL assays of yeast extracts from strains expressing Drosophila melanogaster sequences LP04413 and GH3783 were performed as previously described using a [³H] labeled C₁₈ dihydrosphingosine substrate, obtained from American Radiolabeled Chemicals, Inc. (St. Louis, Mo.) (Saba, J. D., Nara, F., Bielawska, A., Garrett, S. and Hannun, Y. A. (1997). The BST1 gene of Saccharomyces cerevisiae is the sphingosine-1-phosphate lyase. J Biol Chem 272, 26087-26090. Van Veldhoven, P. P. and Mannaerts, G. P. (1991). Subcellular localization and membrane topology of sphingosine-1-phosphate lyase in rat liver. J Biol Chem 266, 12502-7.). In this method, SPL activity is measured by determining the conversion of radiolabeled C₁₈-dihydrosphingosine substrate to long chain aldehyde product. To assess the ability of homozygous Sply⁰⁵⁰⁹¹ versus wild type flies to degrade endogenous LCBPs, an HPLC method was developed and employed to examine extracts of wild type and homozygous Sply⁰⁵⁰⁹ adults. Endogenous LCBPs were first isolated as described under ‘Analysis of Drosophila melanogaster Sphingolipids,’ and the lipid extract from 15 mg of homozygous Sply⁰⁵⁰⁹¹ flies were dried down using nitrogen gas. Lipids were resuspended in SPL reaction buffer and incubated for various time points @ 37° C. Lipids were reisolated, derivatized with o-phthalaldehyde and analyzed by HPLC, as described below. Activity was determined by measuring the percent degradation of endogenous LCBPs in comparison to standards incubated in the absence of protein extracts.

Developmental Expression of Sply

For Northern analysis, full-length probes were labeled by random priming with [γ-³²P] dGTP. Hybridization was carried out under standard conditions against an RNA blot prepared from total RNA of flies harvested at different stages of development (embryos at hours 0-4, 4-8, 8-12, 12-24, larval instars 1^{st}, 2^{nd}, 3^{rd} and adults). RpL32 is a constitutively expressed ribosomal gene used as a loading control.

In situ hybridization was performed with a digoxygenin-labeled probe (Roche cat# 1 175 025) and hybridized to fixed embryos at various stages essentially as described (Tautz, D. and Pfeifle, C. (1989). Chromosoma 98, 81-5.).

Analysis of Drosophila sphingolipids

100 mg of flies were homogenized in 6 ml of ice cold methanol/water 1:1 (vol: vol) with a Potter-Elvehjem homogenizer with a loose pestle followed by a tight pestle until the pestle moved smoothly. Extract was further homogenized by tip sonication for 3 times 20 sec. Extract was spun at low speed and supernatant was removed and dried down in speed vac. Extract was resuspended in 500 μl of methanol containing 0.1 M ammonium hydroxide and incubated for 1 hour at 37° C. Following incubation the extract was dried down in speed vac. Extract was resuspended in 500 μl of 50% methanol containing 0.1% glacial acetic acid and applied to a C18E STRATA solid phase extraction column. C18E STRATA column was washed with 50% methanol containing 0.1% glacial acetic acid followed by a wash with 100% methanol containing 0.1% glacial acetic acid. Lipids of interest were eluted with methanol/10 mM ammonium acetate, 9:1 (vol: vol). Lipids were dried down in speed vac. and o-pthaladehyde labeled for HPLC analysis as previously described (Kim, S., Fyrst, H. and Saba, J. (2000). Genetics 156, 1519-29.).

Lethal Phase Analysis

100 embryos from the indicated lines were collected and observed at each developmental stage. Viability is expressed as the percentage of flies that survived through the indicated stage.

Adult Flight Performance

2-7 day old adult flies were released into a top-lit Plexiglas chamber. Flight behavior was scored as follows: upward flight=3, lateral flight=2, downward flight=1, flightless=0 (Vigoreaux, et al., 1993 J. Cell Biol. May; 121(3):587-98). Average flight scores were compared using a two-tailed student t-test.

Adult and Larval Microscopy

Preparation of tissue, staining, mounting and visualization was performed using standard techniques (Sullivan, W., Ashburner, M. and Hawley, R. S. (2000). Drosophila protocols. Cold Spring Harbor, N.Y.: Cold Spring Harbor Laboratory Press.). Thoraces from adult flies were dissected, fixed with formaldehyde and osmium tetroxide, and embedded in EPON. These blocks were then cut into 1 μm thick sections, stained with methylene blue and azure II, and visualized with a Lieca DMIRBE microscope.

Larvae were filleted during the third instar, pinned with the dorsal cuticle down, and eviscerated to allow an unobstructed view of the body wall muscles. The tissue was fixed with 4% formaldehyde, permeabilized in 100% acetone, and stained with fluorescein-conjugated phalloidin. (Molecular Probes cat# F-432).

Electron microscopic analysis of DLMs was performed on adults essentially as described (O'Donnell, P. T. and Bernstein, S. I. (1988). J Cell Biol 107, 2601-12.).

Hemithoraces were visualized essentially as described (Fyrberg, E. A., Bernstein, S. I. and VijayRaghavan, K. (1994). Methods Cell Biol 44, 237-58.). Briefly, adult flies were frozen in liquid nitrogen, bisected with a razor blade, and dehydrated in an ethanol series. The cuticles were then cleared with methyl salicylate to allow visualization of the muscles with a Lieca DMIRBE microscope under polarized light.

Fluorescent Microscopy

0-24 hour embryos were prepared and fixed using standard techniques (Rubin Manual) and stained with the indicated primary antibody or assayed for apoptosis using a TUNEL-based staining method (In situ cell death detection kit, Roche 1 684 795). Incorporation of fluorescein was assessed with a Leica DMIRBE epifluorescence microscope and an upright Leica TCS-NT confocal laser scanning microscope.

Antibodies and fluorescent reagents were as follows: polyclonal rabbit anti-Drosophila myosin heavy chain (Kiehart, D. P. and Feghali, R. (1986). Cytoplasmic myosin from Drosophila melanogaster. J Cell Biol 103, 1517-25.) 1:1,000. Polyclonal rabbit anti-DMEF2 (Lilly et al., 1995) 1:10,000. Secondary antibody was a fluorescein-conjugated goat anti-rabbit IgG (Jackson ImmunoResearch Laboratories, Inc.) 1:1,000.

Genetics

The precise excision of the ry⁺ PZ P-element was performed by introducing transposase allele Δ2-3 into insertion line BL-11393. In the subsequent generation the transposase was removed and the second chromosome was balanced over CyO. Offspring of these flies that lacked the P-element were selected by scoring for loss of ry⁺. Homozygous lines were generated, assayed for restoration of flight behavior, and assessed for precise excision by PCR if indicated. Lines homozygous for the Sply⁰⁵⁰⁹¹ allele the lace^{k05305} allele were generated by meiotic recombination. Sply⁰⁵⁰⁹¹ and lace^{k05305} mutations were introduced in trans and balanced in the next generation. Flies carrying the lace^{k05305} allele were selected by presence of w⁺. Presence of Sply⁰⁵⁰⁹¹ was verified by PCR.

Preparation of Transgenic Drosophila melanogaster

Relevant methods of preparing transgenic Drosophila melanogaster are disclosed in: Spradling, A. C., and Rubin, G. M. (1982). Science 218, 341-347; Brand & Perrimon, Development (1993) 118: 401-415; and Phelps & Brand, Methods (April 1998) 14:367-379. See also, Spradling A C, P Element Mediated Transformation in Drosophila: A Practical Approach (ed. D. D. Roberts, IRL Press, Oxford)(1986) pp 175-179.

Generally, Drosophila melanogaster stocks used in the experiments described herein are as follows: Wild type Canton-S (BL-1), Sply⁰⁵⁰⁹¹ (BL-11393), lace² (BL-3156) and lace^{k05305} (BL-12176) lines, obtained from the Bloomington Drosophila Stock Center (Indiana University, Bloomington, Ind.). General fly husbandry was performed as described (Sullivan, W., Ashburner, M. and Hawley, R. S. (2000). Drosophila protocols. Cold Spring Harbor, N.Y.: Cold Spring Harbor Laboratory Press).

Other techniques useful in generating mutant and/or transgenic flies are described in Rubin, G, Hong L, Brokstein P, Evans-Holm M, Frise E, Stapleton M and Harvey D, 2000. A Drosophila complementary DNA resource, Science 287:2222-2224.

### Example 1

### Isolation and Characterization of SPL cDNA from Drosophila Melanogaster (Sply)

In order to seek out the Drosophila melanogaster SPL cDNA and genomic sequence, the D. melanogaster genomic database was searched for sequences which demonstrated significant homology to human SPL cDNA. The Drosophila melanogaster genomic database (http://flybase.bio.indiana.edu) was searched for predicted proteins using mouse (accession number AAH26135; amino acid sequence set forth in SEQ ID NO:6) and human (accession number XP_{—}166113; amino acid sequence set forth in SEQ ID NO:18) SPL sequences. DNA homology searches were performed via the Berkeley Drosophila Genome Project web site using the BLAST search program (http://www.ncbi.nlm.nih.gov). One computed gene (CG8946) was identified that corresponded to a predicted SPL gene. Subsequently, two ESTs (LP04413, cDNA set forth in SEQ ID NO:27 and GH13783, cDNA set forth in SEQ ID NO:26) were identified which contained open reading frames that corresponded to the two predicted splice variants. The two clones are predicted based on alternative 5′ exon usage and may be expressed in different subcellular locations.

The predicted Drosophila melanogaster SPL is located on the right arm of chromosome 11, position 53F8-12. The cDNA sequence for the coding region of the Drosophila melanogaster SPL is set forth in SEQ ID NO:15 and encodes the SPL protein set forth in SEQ ID NO:16. The Drosophila SPL predicted protein sequence set forth in SEQ ID NO:16 is 49%, 49% and 43% identical to human, mouse and yeast SPL protein sequences, respectively.

In order to evaluate whether these clones contained a functional SPL gene, they were recloned into the yeast expression vector, pYES2 in which gene expression is driven by a galactose-inducible promoter. The open reading frame contained in LP04413 (polynucleotide sequence set forth in SEQ ID NO:) was amplified using primers LPEcoRI5=5′-TGGMTTCGATGCGTCCGTTCTCCGGCAGC-3′ and

LPXhoI3′=5′-CTCCTCGAGTCTATTTCTGGCTGGGAGT-3′ and was cloned into the yeast expression vector, pYES2, at EcoRI and XhoI restriction sites. This construct was transformed into a dpl1Δ strain using the lithium acetate method (Ito, H., Fukuda, Y., Murata, K. and Kimura, A. (1983). J Bacteriol 153, 163-8.). These constructs were transformed into a dpl1Δ strain in which the sole endogenous Saccharomyces cerevisiae SPL gene has been deleted (Saba, J. D., Nara, F., Bielawska, A., Garrett, S. and Hannun, Y. A. (1997). J Biol Chem 272, 26087-26090.). The dpl1Δ strain is unable to catabolize LCBPs, and it cannot proliferate on media containing low concentrations of D-erythro-sphingosine.

Expression of clones containing the potential Drosophila melanogaster SPL fully complement the dpl1Δ strain's sensitivity to 50 μM D-erythro-sphingosine. Further, whole cell extracts of dpl1 strains containing either pYES2-LP04413 or pYES2-GH3783 demonstrate restoration of SPL enzyme activity to wild type levels or greater, although not as high as a DPL1 overexpressing strain (DPL OE). Further, whole cell extracts of dpl1Δ strains overexpressing Sply demonstrate restoration of SPL enzyme activity.

Northern analysis of wild type Drosophila melanogaster embryos and larvae indicates that Sply expression is developmentally regulated, with the onset of expression occurring by 8-12 hours of embryogenesis. Embryonic expression was largely localized to the gut primordium as indicated by in situ hybridization.

Thus, this example describes Sply, the sphingosine-1-phosphate lyase gene in Drosophila melanogaster.

### Example 2

### Generation and Characterization of SPL Transposon Mutant D. Melanogaster

Drosophila melanogaster stocks used in the experiments described herein are as follows: Wild type Canton-S (BL-1), Sply⁰⁵⁰⁹¹ (BL-11393), lace² (BL-3156) and lace^{k05305} (BL-12176) lines, obtained from the Bloomington Drosophila Stock Center (Indiana University, Bloomington, Ind.). General fly husbandry was performed as described (Sullivan, W., Ashburner, M. and Hawley, R. S. (2000). Drosophila protocols. Cold Spring Harbor, N.Y.: Cold Spring Harbor Laboratory Press)

Flies from the Berkeley Drosophila Genome Project gene disruption project (Spradling, A. C., Stern, D. M., Kiss, I., Roote, J., Laverty, T. and Rubin, G. M. (1995). Gene disruptions using P transposable elements: an integral component of the Drosophila genome project. Proc Natl Acad Sci USA 92, 10824-30.) were identified that harbor a transposon within the Sply open reading frame (designated Sply⁰⁵⁰⁹¹). The transposon is located at nucleotide +269 relative to the start site of the larger transcript, LP04413, cDNA set forth in SEQ ID NO:27.

These flies were genetically crossed using techniques well known to ordinarily skilled artisans, and progeny were evaluated for the presence of homozygous insertional mutants (based on presence of rosy eye color, encoded by a recessive marker carried on the P-element). Northern analysis of total RNA obtained from Sply⁰⁵⁰⁹¹ homozygotes confirmed an absence of Sply expression.

To determine the SPL function of each genotype, +/+, +/− and −/− flies were homogenized and whole extracts assayed for SPL activity. It was observed that SPL genotype corresponded with SPL activity with +/+>+/−>−/−. Initial evaluation of homozygous mutants indicated that adult SPL mutants were flightless, suggesting a potential defect in either muscle development or energetics of the adult fly. Flight analysis was carried out essentially as described (Vigoreaux, J., J. Saide, K. Valgeirdottir, and M. Pardue. 1993. Flightin, a novel myofibrillar protein of Drosophila stretch-activated muscles. J Cell Biol. 121:587-598) by determining the percentage of flies that were flightless or exhibited downward, upward, or lateral flight capabilities in control Canton-S flies as compared to mutant flies as follows: 2-7 day old adult flies were released into a top-lit Plexiglas chamber. Flight behavior was scored as follows: upward flight=3, lateral flight=2, downward flight=1, flightless=0 (Vigoreaux, et al., 1993). Average flight scores were compared using a two-tailed student t-test.

### Example 3

### Further Characterization of the Sply P-element Insertional Mutant Drosophila Melanogaster

The sphingolipids of Drosophila melanogaster contain C₁₄ and C₁₆ sphingosine and dihydrosphingosine LCBs (see Example 4). Extracts of wild type and mutant flies were compared for their ability to degrade endogenous Drosophila melanogaster LCBPs in vitro. Extracts of Sply⁰⁵⁰⁹¹. mutants failed to catabolize endogenous LCBPs, whereas extracts of wild type flies degraded endogenous Drosophila melanogaster LCBPs, indicating that the Sply gene product is responsible for LCBP catabolism in this organism.

To determine whether loss of Sply expression affects the levels of Drosophila melanogaster endogenous LCBs and corresponding LCBPs, the sphingolipid profile of homozygous Sply⁰⁵⁰⁹¹ flies was evaluated and compared to wild type controls. Homozygous Sply⁰⁵⁰⁹¹ adults demonstrated an eight-fold increase in LCBs and a 20-fold increase in LCBPs when compared to wild type (Table 1), indicating significant derangement of sphingolipid metabolism. This accumulation of LCBs and LCBPs was observed in homozygous Sply⁰⁵⁰⁹¹ mutants as early as hours 12-18 of embryogenesis, correlating with the onset of Sply expression.

TABLE 1
Biochemical and Biological Characteristics of Mutant Models of Sphingolipid Metabolism

| | | | | | | | Sply⁰⁵⁰⁹¹ + 1 mM |
|---|---|---|---|---|---|---|---|
| | | | | | lace^{k05305/+}, | lace^{k05305}, | D,L- |
| A. Characteristic | Canton-S | Sply⁰⁵⁰⁹¹ | Sply^{14a} | lace^{k05305/2} | Sply⁰⁵⁰⁹¹ | Sply⁰⁵⁰⁹¹ | threo-DHS |
| C_{14/16} LCBs | 2.71 ± 0.28 | 24.22 ± 1.73 | 5.30 ± 0.59 | 0.15 ± 0.01 | 12.67 ± 1.93 | 5.75 ± 0.42 | 136%* |
| (nmol/100 mg) | | | | | | | |
| C_{14/16} LCBPs | 0.30 ± 0.09 | 6.38 ± 0.44 | 1.02 ± 0.33 | 0.08 ± 0.04 | 4.06 ± 0.64 | 1.88 ± 0.17 | 81%* |
| (nmol/100 mg) | | | | | | | |
| Average flight | 2.60 ± 0.032 | 0.40 ± 0.036 | 1.70 ± 0.074 | 1.62 ± 0.14 | 1.41 ± 0.063 | 0.56 ± 0.13 | 0.62 ± 0.057 |
| score | | | | | | | |
| # of DLM | 6.00 ± 0.00 | 4.15 ± 0.21 | 5.97 ± 0.089 | 5.94 ± 0.030 | 5.13 ± 0.26 | 5.81 ± 0.14 | N.D. |
| fibers/hemithorax | | | | | | | |
| Average # of | 44.5 ± 3.28 | 15.8 ± 2.98 | 43.4 ± 3.43 | N/A | 52.9 ± 4.03 | N/A | N.D. |
| eggs/day | | | | | | | |
| Developmental | 20% | 66.5% | 27% | N.D. | 20% | N.D. | N.D. |
| lethality | | | | | | | |
| Adult wild type flies and the indicated models of sphingolipid metabolism were analyzed for total phosphorylated (LCBPs) and unphosphorylated (LCBs) long chain base levels, flight performance, number of DLM per hemithorax, fecundity (egg-laying), and % mortality prior to completion of metamorphosis. Flight performance and LCB/LCBP levels were also determined in Sply⁰⁵⁰⁹¹ homozygous flies treated with the sphingosine kinase inhibitor, D,L-threo-DHS. LCB/LCBP levels in | | | | | | | |
| # inhibitor-treated flies are given as percentage of untreated controls; these determinations were obtained in a separate experiment, and baseline sphingolipid levels were not comparable between the two experiments. | | | | | | | |

Canton-S is wild type.
Sply⁰⁵⁰⁹¹ indicates the homozygous Sply null mutant.
lace² and lace^{k05305} are recessive lethal alleles of serine palmitoyltransferase.
Sply^{14a} indicates the homozygous
Sply⁰⁵⁰⁹¹ revertant.
All biochemical, flight, and fiber count data were obtained from mixed-age adults. Values are as indicated, ±s.e.m.

Homozygous and heterozygous Sply⁰⁵⁰⁹¹ flies were examined for evidence of anatomical, developmental, and functional abnormalities. Flies heterozygous for Sply⁰⁵⁰⁹¹ were indistinguishable from wild type. Initial evaluation of flies homozygous for the Sply⁰⁵⁰⁹¹ allele revealed no obvious defects in external anatomical structures at embryonic, larval or adult stages. However, adult mutants were almost uniformly flightless, with 91% of the mutant population scoring zero (in comparison to 4% wild type flies) in a standard flight performance assay (Table 1). Despite the severity of the flight defect in Sply⁰⁵⁰⁹¹ homozygotes, the function of other muscle groups, including the jump and leg muscles did not appear to be affected. Moreover, evaluation of the giant fiber neuromuscular pathway by electrophysiological analysis indicated that this pathway remained functionally intact and was not responsible for the observed flight defect.

Sply⁰⁵⁰⁹¹ Homozygotes Demonstrate Abnormal Flight Muscle Morphology.

To investigate further the etiology of Sply⁰⁵⁰⁹¹ flight defects, adult mutants were sectioned through the thoracic region, and muscles were examined by light microscopy.

These studies revealed a reduction in the number of muscle fibers comprising the DLMs required for flight. Whereas the thoraces of wild type flies invariably contained 6 symmetrical pairs of fibers, Sply⁰⁵⁰⁹¹ homozygotes exhibited a general pattern of missing fibers, asymmetry, and hypertrophy of remaining fibers. Quantitative analysis of DLM fibers revealed a reduction from 6 per hemithorax in wild type to an average of 4.15 per hemithorax in the mutants (Table 1). Microscopic analysis of hemithoraces illuminated with polarized light confirmed the abnormal muscle configuration while demonstrating that muscle insertions were not affected.

Sply⁰⁵⁰⁹¹ Mutation Does Not Disrupt Muscle Ultrastructure, Template Formation or Embryonic Muscle Fusion

To determine the origin of the DLM defect, adult myocyte ultrastructure and larval and embryonic muscle development were investigated. Examination of Dmef2 expression in myoblast nuclei of nascent muscle fibers of early wild type and mutant embryos revealed no appreciable differences in muscle organization. Thus, myoblasts appear to successfully migrate from somites to correct sites in mutant embryonic segments. Similarly, analysis of myosin heavy chain expression in 0-24 hour wild type and mutant embryos revealed no gross changes in the organization of the developing mutant muscle fibers as compared to wild type indicating that myocyte fusion was not impaired.

To determine whether the DLM defect observed in Sply⁰⁵⁰⁹¹ adult homozygotes occurred due to lack of template structures required for their formation during metamorphosis, T2 dorsal oblique muscles (DOMs) were evaluated in mutant larvae. Late-stage mutant larvae exhibited no alterations in number and/or size of DOMs. Therefore, it appears that the mutant muscle defect is restricted to DLMs and affects the adult muscle configuration subsequent to myoblast fusion events during metamorphosis. Despite this defect, the ultrastructure of the DLMs that are present in the Sply⁰⁵⁰⁹¹ mutants generally appear to be intact as evidenced by transmission electron microscopy.

Sply⁰⁵⁰⁹¹ Homozygotes Demonstrate Decreased Fecundity, Semi-lethality and Increased Apoptosis in Embryos

The number of offspring resulting from homozygous Sply⁰⁵⁰⁹¹ crosses was about 10% of the number observed in wild type crosses. This loss of progeny could result from diminished egg-laying and/or diminished survival of embryos and larvae. Analysis of egg-laying indicated that fecundity of the mutants was about one third that of control flies (Table 1). This outcome could be the result of diminished male and/or female fertility. To distinguish between these possibilities, both male and female Sply⁰⁵⁰⁹¹ homozygotes were mated to wild type flies, and egg-laying was measured in comparison to wild type pairs and homozygous mutant pairs. Numbers of eggs produced were significantly diminished in crosses of both male and female mutant flies with wild type mates (data not shown), indicating that the effect on fecundity was not gender-specific. Additionally, crosses between Sply⁰⁵⁰⁹¹ homozygous males and females resulted in progeny with an overall survival (from egg to adulthood) of 33.5%, compared to an 80% survival rate in wild type flies. Lethality in the sply⁰⁵⁰⁹¹ mutants was high during larval stages (46%, compared to 3% in wild type), with the majority of larval death occurring during the first larval instar. Less severe effects were observed during pupation (22% lethality, compared to 1% in wild type), and no appreciable differences in survival were noted during embryogenesis. Sply⁰⁵⁰⁹¹ mutant embryos were examined by in situ TUNEL assay, and patterns of apoptosis were compared to those of wild type controls. Sply⁰⁵⁰⁹¹ mutant embryos demonstrated a pronounced enhancement of apoptosis compared to wild type controls, especially in a specific region of the posterior pole near the developing genital disc.

### Example 4

### Characterization of Sphingolipid Species in the Drosophila Melanogaster

Without being bound by theory, it is hypothesized that the phenotype of the SPL mutant Drosophila melanogaster is caused by an abnormal level of S-1-P during development. Further, without being bound by theory, it is our hypothesis that phosphorylated sphingoid base species are responsible for regulating cell proliferation, migration and other events required for both tumor formation and normal developmental processes in this model organism. Therefore, characterization of sphingolipid species in Drosophila melanogaster was determined.

Method

Wild type (Canton S) whole fly extracts were prepared by mechanical disruption. Lipids were isolated by two-phase extraction and derivatized with the fluorescent molecule o-pthalaldehyde essentially as described in Caligan, et al. hereby incorporated by reference in its entirety (Caligan, T. B., K. Peters, J. Ou, E. Wang, J. Saba, and A. H. Merrill, Jr. 2000. A high-performance liquid chromatographic method to measure sphingosine 1-phosphate and related compounds from sphingosine kinase assays and other biological samples. Analytical Biochemistry. 281:36-44). Derivatized lipid extracts were separated by HPLC using a C₁₈ ODS column (LUNA 4.6×250 mm) and mobile phase MeOH/H₂O/1M TBAP 82:17:0.9, pH 4.8. Standards included commercially available C₁₀, C₁₂, C₁₄, C₁₆, C₁₈ and C₂₀ sphingosines, as well as the phosphorylated forms of these standards, prepared by incubation of sphingosine standards with extract from a yeast strain which overexpresses the major yeast sphingosine kinase, LCB4.

Results

Drosophila melanogaster extracts contained only sphingolipid species which comigrated with C₁₄ sphingosine and C₁₄ sphingosine-1-phosphate (S-1-P) standards under the stated conditions. To verify the identity of the peaks in fly extracts which comigrated with C₁₄ sphingosine and C₁₄S-1-P standards, extracts and standards were compared in four different mobile phase buffers. The peak identified as C₁₄ sphingosine comigrated with the C₁₄ sphingosine standard under all four conditions (Table 2). However, the peak identified as C₁₄S-1-P demonstrated a slight difference from the C₁₄S-1-P standard under conditions which exploit differences in charge (MeOH/10 mM KP/1 M TBAP, pH 7.2, 81:18:1).

TABLE 2
Sphingolipid Identification

| | C₁₄S | C₁₄S | C₁₄S-1-P | C₁₄S-1-P |
|---|---|---|---|---|
| Mobile Phase | std | in extract | std | in extract |
| MeOH/H₂O/1 M | 19.1 min | 19.0 min | 14.8 min | 14.8 min |
| TBAP pH 4.8 | | | | |
| 82.1:17:0.9 | | | | |
| MeOH/H₂O/1 M | 27.3 min | 27.1 min | 22.5 min | 22.1 min |
| TBAP pH 4.8 | | | | |
| 79.1:20:0.9 | | | | |
| MeOH/10 mM KP/ | 21.9 min | 22.0 min | 18.3 min | 17.2 min |
| 1 M TBAP pH 5.5 | | | | |
| 81:18.1 | | | | |
| MeOH/10 mM KP/ | 21.4 min | 21.8 min | 15.0 min | 17.1 min |
| 1 M TBAP pH 7.2 | | | | |
| 81:18.1 | | | | |

This finding is likely to be due to a chemical modification of the phosphate group, since a phosphatase capable of dephosphorylating the C₁₄S-1-P standard does not recognize this substrate.
Mass spectroscopy is being utilized to identify the phosphate group modification of this S-1-P species.
Herein, this sphingolipid is referred to as “modified C₁₄S-1-P.”

### Example 5

### Genetic Reversion of the Sply⁰⁵⁰⁹¹ Mutation Restores Normal Muscle Configuration

To verify the importance of Sply in mediating the semi-lethality, egg-laying defects and flight muscle phenotype of the mutant line, the transposon in the Sply⁰⁵⁰⁹¹ locus was mobilized Genetic reversion of the Sply⁰⁵⁰⁹¹ mutation restores normal muscle configuration in Sply⁰⁵⁰⁹¹ homozygotes following introduction of an active transposase. Precise excision of the transposon was subsequently confirmed by PCR and DNA sequence analysis. A homozygous revertant line (Sply^{14a}) was generated as described in Materials and Methods and was found to express Sply mRNA at levels equivalent to wild type. Sply^{14a} demonstrated reversion of the muscle fiber morphology defect, and flight performance was largely restored (Table 1). Additionally, apoptosis in the revertant embryo was diminished in comparison to Sply mutants. The appearance of the specific cluster of TUNEL-positive cells was <1% (n=197), 48% (n=160) and 72% (n=324) in Canton-S, Sply^{14a}, and Sply⁰⁵⁰⁹¹ in stage 12-15 embryos, respectively. Phenotypic reversion correlated with normalization of LCB and LCBP levels in revertant extracts (Table 1).

### Example 6

### The Sply⁰⁵⁰⁹¹ Muscle Defect is Suppressed by Reducing Sphingolipid Intermediates

To investigate the possibility that the Sply⁰⁵⁰⁹¹ muscle phenotype was caused by accumulation of LCBPs, an inhibitor of sphingosine kinase, D, L-threo-DHS, was introduced to the growth media of mutant and wild type flies. Flies were grown on the supplemented media, and F2 progeny were examined. When wild type flies were grown on media supplemented with 10 μM D,L-threo-DHS, no deleterious effects were observed. Sply⁰⁵⁰⁹¹ mutants grown on this media demonstrated a slight but significant improvement in flight performance. To determine whether the flight improvement coincided with a restoration of LCBP levels, LCB/LCBP levels were analyzed in mutants and controls grown on D,L-threo-DHS. LCBP levels in Sply⁰⁵⁰⁹¹ homozygotes grown in the presence of sphingosine kinase inhibitor were reduced by approximately 20% (Table 1). Similarly, LCBP levels in wild type flies were reduced to 20% of normal levels.

Assuming that the mutant phenotypes are caused by an accumulation of LCB/LCBPs, we predicted that diminishing SPT activity in the Sply⁰⁵⁰⁹¹ homozygote would suppress the Sply⁰⁵⁰⁹¹ phenotype by reducing production of sphingolipid intermediates. Toward that end, a lace^{k05305} null allele was introduced onto the Sply⁰⁵⁰⁹¹ chromosome by genetic recombination, thus generating a Sply⁰⁵⁰⁹¹, lace^{k05305}/+ line. Sply⁰⁵⁰⁹¹, lace^{k05305}/Sply⁰⁵⁰⁹¹, lace⁺ flies exhibited reversion of the abnormal muscle patterning, and flight performance was substantially improved (Table 1). Additionally, the pattern of embryonic apoptosis appeared similar to that of the wild type. Phenotypic reversion correlated with a marked reduction of the LCBs and LCBPs (Table 1).

### Example 7

### Loss of Sply Expression Suppresses the Lace Null Phenotype

Inheritance of two lace^{k05305} hypomorphic alleles was reported to be almost completely lethal, whereas a heterozygous allelic combination (lace^{k05305}/lace²) yields flies that frequently survive but manifest severe developmental phenotypes leading to eye, bristle and wing abnormalities (Adachi-Yamada, T., Gotoh, T., Sugimura, I., Tateno, M., Nishida, Y., Onuki, T. and Date, H. (1999). Mol Cell Biol 19, 7276-7286.). We predicted that the lace mutant phenotype is due to diminished levels of sphingolipid intermediates. Further, we reasoned that inhibiting sphingolipid catabolism in lace mutants might allow sufficient accumulation of trace sphingolipids obtained through the diet to ameliorate developmental defects induced by the lack of critical sphingolipid intermediates. To address this possibility, a Drosophila melanogaster line homozygous for both the Sply⁰⁵⁰⁹¹ and lace^{k05305} alleles was generated. Significantly, the presence of the Sply⁰⁵⁰⁹¹ allele increased the recovery of lace homozygotes from 9% to 39% of that expected by independent assortment. Furthermore, the introduction of Sply⁰⁵⁰⁹¹ fully suppressed the eye, bristle and wing phenotypes in the resulting flies. In accordance, sphingolipid intermediates were substantially increased in this line, in comparison to lace²/lace^{k05305} heterozygotes, which are the only available lace mutants with sufficient viability for comparison (Table 1).

### Example 8

### Human SPL and SK Expression Patterns in Cancer

To determine if SK and/or SPL expression is altered in human tumors, we utilized a cancer profiling array which contains more than 240 cDNA pairs representing tumor tissue and corresponding normal tissue from the same patient. By utilizing tissue pairs from one patient, differences between gene expression in tumor and normal tissue which might be due to person to person variability should not confound the interpretation of results. Additionally, each blot was normalized for loading using four separate housekeeping genes.

Standard hybridization techniques known in the art were utilized to probe this cDNA blot with the full length human SPHK1 cDNA (set forth in SEQ ID NO:22), which was obtained by RT-PCR of human umbilical vein endothelial cell total RNA. Analysis of the array indicates that SK expression appears to be significantly increased in numerous human cancers including tumors of breast, uterus stomach, ovary, lung, kidney and rectum. Additionally, some tumors demonstrated increased SK expression in metastatic lesions compared to tumor tissues. None of the SK overexpressing tumors demonstrate loss of SPL expression. Thus, altered SK expression is observed in primary human tumors. Therefore, modulating the activity of SK protein either by altering gene expression or through direct action on the protein may provide a useful treatment for individuals afflicted with an SK-related cancer. Furthermore, SK expression serves as a useful diagnostic marker of cancer in humans.

Standard hybridization techniques were utilized to probe the cDNA blot with a 300 nucleotide 3′ fragment of human SPL cDNA (SEQ ID NO:23), which was obtained as described in U.S. Pat. No. 6,423,527. Analysis of the array indicated that, whereas human SPL expression is matched closely in most tissue pairs, it is significantly reduced in colon cancer specimens, with a 50% reduction in expression in colloid cancer of the colon and 61% reduction in adenocarcinoma of the colon. Reduced SPL expression was also seen in adenocarcinaom of the uterus. None of the tumors in which SPL expression is diminished demonstrates SK overexpression. Thus, altered SPL expression is observed in primary human tumors. Therefore, modulating the activity of SPL protein either by altering gene expression or through direct action on the protein may provide a useful treatment for individuals afflicted with an SPL-related cancer. Furthermore, SPL expression serves as a useful diagnostic marker of cancer in humans.

### Example 9

### Isolation of Drosophila Melanogaster Genes Involved in Sphingolipid Metabolism

Drosophila melanogaster genes that are involved in sphingolipid metabolism were identified. Using the human SK protein sequence set forth in SEQ ID NO:21 as probe, we identified two homologous Drosophila melanogaster sequences which could potentially encode fly SK proteins. These two Drosophila melanogaster SK protein sequences are set forth SEQ ID NOs: 19 and 20 and are shown in FIG. 1. The annotation FBan0001747 for gene CG1747, which has FlyBase accession number FBgn0030300, is located on chromosome arm X, and has a transcription unit length of 2020 nucleotides. This gene has the transcript CT5088. The function of this gene has been categorized as enzyme/diacylglycerol kinase, based upon a conserved lipid kinase domain. The annotation FBan0002159 for gene CG2159, which has FlyBase accession number FBgn0035391, is located on chromosome arm 3L, and has a transcription unit length of 4431 nucleotides. This gene has the transcript CT2650. These two sequences have not been cloned, and neither functional data nor ESTs are available. (DSK1747 amino acid sequence is set forth in SEQ ID NO:19; DSK2159 amino acid sequence is set forth in SEQ ID NO:20).

### Example 10

### Characterization of 2 Drosophila Melanogaster SK Genes Invalved in Sphingolipid Metabolism

In order to identify potential SK genes in Drosophila melanogaster, the Drosophila melanogaster genomic database was searched using a tBLASTn enquiry for sequences that demonstrated significant homology to human SK cDNA. This led to the identification of two candidate SK as described above. Gene CG1747 is located on chromosome X and has been categorized as enzyme/diacylglycerol kinase, based upon a conserved lipid kinase domain. Gene CG2159 is located on chromosome arm 3L, and has a transcription unit length of 4431 nucleotides. ESTs corresponding to these two loci were obtained, their integrity confirmed by sequence and restriction analysis (sequences of the full length amino acids of SK1 and SK2 are set forth in SEQ ID NOs:28 and 29; full length cDNAs for SK1 and SK2 are set forth in SEQ ID NOs:24 and 25). These CG1747 and CG2159 cDNA clones were then re-cloned into yeast expression vector pYES2, under regulation of a galactose-inducible promoter (Rubin, G, Hong L, Brokstein P, Evans-Holm M, Frise E, Stapleton M and Harvey D, 2000. A Drosophila complementary DNA resource, Science 287:2222-2224.). These constructs were transformed into yeast strain JSK392 (Kim, S, Fyrst H and Saba J, 2000. Genetics 156:1519-1529.), in which the endogenous SPL, SK and S-1-PP genes (DPL1, LCB4 and YSR2 respectively) have been deleted. This strain can survive in the absence of LCBP synthesis, but expression of a functional SK gene in this background is lethal, due to severe accumulation of LCBPs which cannot be degraded. When CG1747 and CG2159 expression was induced in this background in the presence of galactose, no yeast growth occurred, indicating that these cDNAs encode functional SK enzymes. Finally, a CG2159 transposon mutant was obtained and demonstrates lack of expression from this locus (Rosemann, R, Johnson E, Rodesch C, Bjerke M, Nagoshi R and Geyer P, 1995. Drosophila melanogaster, Genetics 141:1061-1074.). This mutant (Sphk2^{KG05894}) was created by the insertion of a P-element into the 5′ UTR of CG2159, as previously described. Preliminary studies indicate this mutant has a mild defect in DLMs, similar to that seen in Sply mutants.

### Example 11

### Characterization of the Chemical Structures and Concentration of Sphingolipid Metabolites in Wild Type Flies and those with Defects of Sphingolipid Metabolism

Materials and Methods

Drosophila melanogaster Lines

The lace gene encodes one subunit of a Drosophila serine palmitoyltransferase. Inheritance of two lace^{k05305} null alleles is reported to be uniformly lethal, whereas the heterozygous allelic combination used in these experiments, lace^{k05305}/lace², leads to severe developmental phenotypes and a low percentage of viable progeny (Adachi-Yamada, T., T. Gotoh, I. Sugimura, M. Tateno, Y. Nishida, T. Onuki, and H. Date. 1999. Mol. Cell. Biol. 19: 7276-7286.). A Drosophila line homozygous for a null allele of one of two putative sphingosine kinase (SK) genes was also utilized in these experiments. This mutant (Sphk2^{KG05894}) was created by the insertion of a P-element into the 5′ UTR of CG2159, as previously described. The product of this gene functionally complements a yeast SK mutant. Wild type Canton-S (BL-1), lace² (BL-3156), lace (BL-12176), and Sphk2^{KG05894} (BL-14133) lines were obtained from the Bloomington Drosophila Stock Center (Indiana University, Bloomington, Ind.).

Flies were reared on standard fly media at room temperature. In all cases, control and mutant flies were reared in parallel under identical conditions. For developmental analysis, adult flies were allowed to deposit embryos on grape juice agar plates. After the collection period, plates were removed from the collection chamber, covered, and aged at room temperature to obtain appropriately staged embryos. For example, to collect 6-12 hour embryos, adults were exposed to plates for 6 hours, plates were removed and aged for an additional 6 hours before embryos were collected. Embryos were removed from the plates by washing with 0.7% sodium chloride/0.03% TritonX-100, rinsed extensively with water and frozen at −70° C. for storage.

Preparation of Drosophila Lipid Extracts

Samples containing 25 mg of frozen intact fly material were placed in a 7 ml Potter Elvehjem homogenizer. 20 μl of a mixture of internal LCB standards (Matreya Inc., Pleasant Gap, Pa.) containing 250 to 500 pmol of each LCB were then added. Flies were homogenized in 2 ml of ice cold methanol/water, 1:1 (v/v) with a loose pestle followed by a tight pestle until it moved smoothly. Extracts were further homogenized with a tip sonicator (3×20 sec.) while on ice, then transferred to a glass tube and centrifuged at 1500×g for 10 minutes. Supernatants were recovered and dried down in a speed vac. Extracts were resuspended in 200 μl of methanol containing 0.1 M ammonium hydroxide, followed by vortexing, bath sonication and incubation at 37° C. for 1 hr to allow hydrolysis of esterified acyl chains. Following hydrolysis, the samples were cooled to room temperature, dried down in a speed vac and resuspended in 500 μl of methanol/water, 2:3 (v/v) containing 0.1% glacial acetic acid (solvent A).

Solid phase extraction on a Strata C18-E column.

The Strata C18-E solid phase extraction column (50 mg/ml) (Phenomenex, Torrance, Calif.) was initially wetted with 200 μl of methanol, followed by equilibration with 1 ml of solvent A. Fly extracts or LCB standards in solvent A were applied to the equilibrated Strata C18-E column, followed by a wash with 1 ml of solvent A. A second wash of the column was performed by the addition of. 600 μl of methanol. LCBs were eluted from the column with 600 μl of methanol:10 mM ammonium acetate, 9:1 (v/v) and dried down in a speed vac.

HPLC Analysis

LCBs were derivatized with ortho-phthalaldehyde (OPA) (Sigma St. Louis, Mo.) as previously described (Caligan, T. B., K. Peters J. Ou, E. Wang, J. Saba, and A. H. Jr. Merrill. 2000. Anal. Biochem. 281: 36-44.). The OPA-derivatized LCBs were separated on a reverse-phase column (Luna RP-18, 3μp, 4.6×75 mm) (Phenomenex, Torrance, Calif.) with the mobile phase methanol/10 mM ammonium acetate, pH 5.2, 82:18 (v/v). Flow rate was 1 ml/min. The HPLC system used was a Beckman System Gold with a 125 solvent module. The fluorescent LCBs were detected and quantified using a Spectra-Physics fluorescence detector (SP 8410).

Mass Spectrometry Analysis of Drosophila LCBs.

A Strata C18-E column-purified lipid extract from adult Sphk2^{KG05894} flies or a C₁₄ So standard were analyzed on a Micromass Quattro LCZ instrument following direct injection of 10 μl of sample. Mobile phase was 80 percent methanol containing 0.1 percent formic acid. Flow rate was 0.2 ml/min. Structural confirmation of LCBs was obtained by positive electrospray ionization (ESI+) mass spectrometry. LCBs were detected by precursor ion scans of structurally distinct ion fragments as described (Sullards, M. C., and A. H. Jr. Merrill. 2001. Sci. STKE. 67: 1-11.). Applying 3.5 kV to the capillary started the spray and the collision-induced decomposition spectra, at a cone voltage of 20 V, were recorded at a collision energy of 15 eV with argon as collision gas.

Abbreviations

So: sphingosine; Sa: didydrosphingosine, LCB: free long chain sphingoid bases; LCBP: phosphorylated free long chain sphingoid bases; SPT: serine palmitoyltransferase; OPA: ortho-phthalaldehyde.

Results:

HPLC Separation of Sphingoid Bases and Solid Phase Extraction of Sphingoid Bases

An HPLC method was developed for the separation of LCBs with a carbon number of 14 to 18. Initial HPLC separation of crude methanol/water lipid extracts from adult flies were complicated by high content of contaminating fluorescent material. Consequently, a solid phase extraction step using a Strata C18-E column prior to HPLC analysis was introduced. When methanol was employed as the eluting solvent, recovery of LCB standards was less than 2 percent. This inadequate recovery of the LCB standards from the Strata C18-E column was completely overcome by addition of 10 percent by volume of a 10 mM ammonium acetate solution to the methanol elution solvent. By employing this elution system, recovery in the range of 60 to 95 percent was obtained for the C₁₄ and C₁₆ sphingoid base standards (Table 3).

TABLE 3
Recovery of Sphingoid Base Standards Following Solid Phase
Extraction on a STRATA C18-E column

| | Sphingoid base | | | | |
|---|---|---|---|---|---|
| | C₁₄ So | C₁₆ So | C₁₆ Sa | C₁₈ So | C₁₈ Sa |
| Recovery | 95.4 ± 3.3 | 77.9 ± 7.1 | 60.6 ± 4.2 | 39.3 ± 6.5 | 16.3 ± 4.0 |
| (%) | | | | | |

Values are shown as mean ± standard deviation for at least three independent measurements.

HPLC Analysis of LCBs from Drosophila

Elution of adult fly lipids from the Strata C18-E column with methanol/10 mM ammonium acetate 9:1 (v/v) still resulted in an HPLC spectrum with significant unwanted background fluorescence. This background was minimized with the addition of a methanol wash prior to elution with methanol/10 mM ammonium acetate 9:1 (v/v) (see Materials and Methods for details). Adult flies of three different lines were analyzed. The lipid profile of wild type flies was compared to that of a sphingosine kinase (Sphk2) mutant and a serine palmitoyltransferase (SPT, lace) mutant (See Materials and Methods above). The Sphk2 mutants would be predicted to manifest a reduced capacity to phosphorylate LCBs and as a consequence should demonstrate increased levels of LCBs. In contrast, the hypomorphic lace mutants are defective in the first step of sphingolipid de novo biosynthesis and would be predicted to exhibit diminished levels or complete absence of LCBs. Three peaks demonstrating the same retention times as the C₁₄ SO, C₁₆ So and C₁₆ Sa standards were identified in wild type fly extracts. In addition, a major peak that eluted with a retention time between that of C₁₄ So and C₁₆ So was identified. All four peaks mentioned above were increased in the Sphk2 mutant and decreased in the lace mutant, consistent with the likelihood that these peaks represented LCBs. No peaks that eluted with retention times corresponding to the C₁₈ LCB standards were observed.

Following isocratic elution from a C18 reverse phase HPLC column, a plot of the carbon length of a derivatized sphingoid base standard against the log of the retention time shows a linear correlation between sphingoid bases belonging to the same molecular class (Lester, R. L., and R. C. Dickson. 2001. Anal. Biochem. 298: 283-292.). This can be useful for the identification of an unknown sphingoid base. A linear correlation exists between the retention time of the unknown peak 2 and the two Sa standards in this plot. This finding strongly suggests that peak 2 is C₁₄ Sa.

Mass Spectrometry Analysis of LCBs from Drosophila

LCBs can be identified through their patterns of collision-induced dissociation and precursor ion scans using positive ion electrospray mass spectrometry (ESI+). Based on their unique molecular structures, typical decomposition products arise from the loss of two water molecules. The precursor ion spectrum of m/z 208 (C₁₄ So minus two water molecules) shows parents as m/z 244 (C₁₄ So) and m/z 226 (C₁₄ So minus one water molecule). In order to verify the existence of C₁₄ Sa in Drosophila, we analyzed a Strata C18-E column purified lipid extract by ESI+. A lipid extract from the Sphk2 mutant was chosen for the analysis since it demonstrated elevated levels of LCBs. Initially we sought the presence of endogenous C₁₄ So. A precursor ion spectrum of m/z 208 identified C₁₄ So (m/z 244) in the extract. Subsequently, we sought the presence of C₁₄ Sa. A precursor ion spectrum of m/z 210 identified endogenous C₁₄ Sa (m/z 246). In addition, precursor ion scans of m/z 236 and m/z 238 identified endogenous C₁₆ So and C₁₆ Sa in the fly extract. Precursor ion scans of m/z 264 and m/z 266 failed to identify C₁₈ LCBs in the fly extract supporting the results obtained from the HPLC analysis.

C₁₄ and C₁₆ Sphingoid Bases in Drosophila Models of Sphingolipid Metabolism

Endogenous Drosophila LCBs were quantified by performing HPLC separation of Strata C 18-E column purified extracts either with or without the addition of a defined amount of C₁₄ So, C₁₆ So and C₁₆ Sa standard. Separation was followed by comparison of the integrated area obtained for each fluorescent LCB peak (Table 4). Interestingly, lace and Sphk2 mutant flies differed appreciably from wild type flies in both the total amount and composition of LCBs, as determined by analysis of lipid extracts from each line. The total amount of LCBs in the wild type was approximately 1.5 nmol/100 mg of whole flies. The Sphk2 mutants exhibited a 3.3 fold increase and the lace mutants, a 2.5 fold decrease in the total amount of LCBs in comparison to wild type flies. C₁₄ So accounted for approximately 42 percent of the total amount of LCBs in the wild type flies, whereas C₁₄ Sa accounted for approximately 47 percent. Therefore, the molar ratio of C₁₄ So to C₁₄ Sa was approximately 1:1. In the Sphk2 mutant, the 5 corresponding values were 26 percent C₁₄ So and 67 percent C₁₄ Sa resulting in a molar ratio of approximately 1:2.5, whereas in the lace mutant the corresponding values were 16 percent C₁₄ So and 81 percent C₁₄ Sa resulting in a molar ratio of approximately 1:5.

TABLE 4
HPLC Analysis of Endogenous LCBs
in Various Adult Drosophila Lines

| | Wild type | Sphk2 | lace |
|---|---|---|---|
| c₁₄ So (nmol/ | 0.637 ± 0.132 | 1.282 ± 0.144 | 0.100 ± 0.019 |
| 100 mg of flies) | | (201.3) | (15.7) |
| c₁₄ Sa ^{(a)} (nmol/ | 0.718 ± 0.097 | 3.282 ± 0.361 | 0.496 ± 0.055 |
| 100 mg of flies) | | (457.1) | (69.1) |
| C₁₆ So (nmol/ | 0.055 ± 0.007 | 0.160 ± 0.011 | 0.018 ± 0.002 |
| 100 mg of flies) | | (290.9) | (37.7) |
| C₁₆ Sa (nmol/ | 0.051 ± 0.009 | 0.198 ± 0.026 | n.d. |
| 100 mg of flies) | | (388.2) | |
| Total LCBs | 1.461 ± 0.245 | 4.922 ± 0.542 ^{b} | 0.614 ± 0.076 ^{c} |
| (nmol/100 mg | | (336.9) | (42.0) |
| of flies) | | | |
| C₁₄ So/C₁₄ Sa | 0.89 | 0.39 | 0.20 |
| (mol: mol) | | | |

Values are shown as mean ± standard deviation for three independent measurements.
Value n.d. means there was no detectable LCB at the amount of fly material analyzed.
Numbers in parentheses represent percent of wild type.
^{(a)} An estimated value for the recovery of C₁₄ Sa from the Strata C18-E column was determined using the following formula; % recovery of C₁₄ Sa = % recovery of C₁₆ Sa × (% recovery of C₁₄ So/% recovery of C₁₆ So).
^{b} Significantly different from wild type; P < 0.001.
^{c} Significantly different from wild type; P < 0.005.

C₁₄ Sphingoid Bases in Drosophila Development.

Genetic studies have implicated a role for sphingolipid intermediates in the process of development. However, quantification of these molecules throughout development has not been performed. To investigate whether a biochemical basis for the potential role of sphingolipid intermediates exists, we evaluated the endogenous C₁₄ LCBs at different stages of Drosophila development (Table 5). The total amount of C₁₄ LCBs remained fairly constant throughout the development of the wild type. However, developmental progress from early embryos to pupae was associated with a seven fold increase in the molar ratio of C₁₄ So to C₁₄ Sa.

TABLE 5
HPLC Analysis of Endogenous C₁₄ LCBs in Different Stages of
Wild Type Drosophila development

| | | | | | |
|---|---|---|---|---|---|
| Embryo | Embryo | Embryo | Embryo | | |
| (0-6 hr) | (6-12 hr) | (12-18 hr) | (18-24 hr) | Larvae | Pupae |

| | | | | | | |
|---|---|---|---|---|---|---|
| C₁₄ So (nmol/100 mg | 1.210 ± 0.173 | 2.132 ± 0.359 | 1.616 ± 0.261 | 1.713 ± 0.072 | 3.328 ± 0.318 | 2.094 ± 0.126 |
| of material) | | | | | | |
| C₁₄ Sa (nmol/100 mg | 1.156 ± 0.122 | 1.010 ± 0.098 | 0.461 ± 0.051 | 0.387 ± 0.012 | 0.722 ± 0.033 | 0.309 ± 0.047 |
| of material | | | | | | |
| Total C₁₄ LCBs | 2.366 ± 0.295 | 3.142 ± 0.457 | 2.077 ± 0.312 | 2.100 ± 0.084 | 4.050 ± 0.351 | 2.403 ± 0.173 |
| (nmol/100 mg of | | | | | | |
| material) | | | | | | |
| C₁₄ So/C₁₄ Sa | 0.97 | 2.11 | 3.51 | 4.43 | 4.61 | 6.78 |
| (mol: mol) | | | | | | |

Values are shown as mean ± standard deviation for three independent measurements.

### Example 12

### Analysis of Gross and Microscopic Tumor Development in Drosophila Melanogaster Strains with Altered Sphingolipid Metabolism

Some Drosophila melanogaster sphingolipid metabolic mutants may develop tumors, especially those which demonstrate significant accumulation of S-1-P. Previously characterized Drosophila melanogaster tumors associated with Igl and dlg mutations possess characteristics associated with neoplastic growth in vertebrates, including lethality to the host, repeated transplantability, invasiveness, absence of terminal differentiation, rapid growth in vivo and in tissue culture, and defective cell-cell interactions and communication. Evaluation of each of these characteristics in grossly or microscopically visible tumors arising in mutants is performed. Overgrowth of imaginal discs is sought in all mutants even in the absence of gross tumors. Lethal mutations may suggest the presence of tumor formation during early developmental stages. The ability of cells of imaginal disc tumors or imaginal disc overgrowth to respond to normal differentiation signals during metamorphosis is evaluated by transplanting mutant imaginal discs into wild type larvae and following their fate. The ability of tumors or hyperproliferative imaginal discs to be serially cultured in vitro and in the abdomens of adult females for numerous transfer generations is assessed. Evidence of tumor invasiveness and metastatic capacity will be determined by determining the presence or absence of invasion into tissues surrounded by an epithelial sheath using histological analysis.

### Example 13

### Assessment of Gross and Microscopic Anatomic Defects in Drosophila Melanogaster Strains with Altered Sphingolipid Metabolism

Certain illustrative experimental methods are described herein, for example the above section before Example 1. Further illustrative techniques that can be used to further define the present invention, for example general techniques for assessment of gross and microscopic anatomical defects of Drosophila melanogaster, are known in the art and are further described herein. Lifespan of each line is determined and compared to wild type flies to assess effects on viability. Gross anatomy of tracheal and muscular systems is viewed in larvae with polarized light and in adults by viewing standard thick sections under light microscopy, in order to identify gross defects affecting trachea or muscle number, size, location or proper attachment. Preparation for light microscopy involves storing flies in a glycerol/ethanol solution, followed by 70% ethanol, dissecting away unnecessary structures, rehydrating and mounting or embedding the specimen for sectioning. Visualization is be performed using bright-field, DIC illumination, phase contrast or dark-field illumination. Electron microscopic analysis of the IFMs is performed on adults, larvae and pupae. The early Drosophila melanogaster embryo is amenable to structural analysis using any of a large number of highly specific antibodies which is detected using fluorescent secondary or, in some cases, primary antibodies. Large numbers of staged embryos can be collected from normal and mutant stocks and prepared for fluorescent analysis. This process involves dechorionation using a bleach solution, permeabilizing the vitelline membrane, fixation (without methanol) and vitelline membrane removal, staining, washing and mounting. Since lace mutants demonstrate abnormal apoptosis of imaginal wing discs, we plan to investigate any changes in imaginal disc structure and eversion in all mutants. Dissection of imaginal discs is performed in a saline solution and involves tearing the third instar larva in half, inverting the body wall and pinching clusters of dorsal or ventral discs away from the body. Discs may be left attached to the body wall for in situ hybridization studies, or they may be removed and fixed for other studies. Pupal imaginal discs can also be isolated by removing the cuticle after fixation overnight.

### Example 14

### Identification of Pharmacologic Suppressors of SPL Mutant's Inability to Fly by Screening an Array of Rationally Designed Chemicals with Homology to Sphingolipids for their Ability to Restore Flight to SPL Mutant Progeny

Pharmacologic suppressors of the Sply mutant Drosophila melanogaster's inability to fly are identified by screening an array of rationally designed chemicals with homology to sphingolipids for their ability to restore flight to the Sply mutant Drosophila melanogaster. Mutant SPL flies are grown at 18° C. in media supplemented with either vehicle control or micromolar concentrations of inhibitor. The ability of various inhibitors to restore flight will be measured using a standard scoring method, also as described elsewhere herein (see also Vigoreaux, et al., 1993, J Cell Biol 121:587-598). The efficacy and biochemical characteristics of interesting compounds will then be quantified by 1) determining the IC₅₀ of the inhibitor on purified SK, SPL and other enzymes involved in sphingolipid metabolism, such as serine palmitoyltransferase, ceramide synthase, sphingosine desaturase, ceramidase, ceramide kinase, phosphoethanolamine cytidylyltransferase, CDP-ethanolamine phosphotransferas, acid sphingomylelinase and neutral sphingomyelinase. The IC50 of the inhibitor is also determined on recombinant human SK, SPL and other enzymes involved in sphingolipid metabolism as listed above; 2) analyzing the kinetics of inhibition using classical Michaelis-Menten methods, and 3) determining the reversibility of the inhibition.

Synthetic analogs are created for the screen. A library of 1-aryl-2-dimethylaminopropane-1,3-diol derivatives for screening as potential SK inhibitors are synthesized. Four diastereomers (D or L, erythro or threo) are synthesized for each member of the library. In the library the fatty acid amide group is replaced with two N-methyl groups and make similar variations in the polar and aromatic substituents. The synthetic plan makes use of the well-known Garner aldehyde (See 1 in FIG. 2) as starting material, since 1 is readily available in either enantiomeric form. A recent and exhaustive review of organometallic additions to 1 summarizing the effects of metal, solvent, and added Lewis acid catalyst has been published, and indicates that the erythro-product is usually favored in most reactions. Thus, by choosing the D- or L-enantiomer of 1 as starting material, pure erythro stereoisomers of each library member are prepared. A novel and flexible route for assembling the corresponding threo analogues (4a-c, FIG. 2) is carried out using a straightforward extension of methodology for making PDMP analogues. The parent compound, 4a, is already known and readily available. The strategy relies on the syn-selective addition to 1 of arylmetal compounds (Aryl-Met) in the presence of certain sulfide and phosphine additives. Both the erythro and threo synthetic routes are modified to prepare substituted variations at the primary carbon atom. A representative synthetic procedure is shown in FIG. 3 for the preparation of 7a-c. A wide range of nitrogen, oxygen, and carbon nucleophiles could react with mesylates like 5a-c to furnish new libraries of dimethylated PDMP analogues and homologues.

### Example 15

### Further Evaluation of Candidate Drugs by Testing their Ability to Inhibit Human SK in a Yeast Screen Devised Such that Inhibition of SK Confers Cell Survival

Compounds identified in the fly screen as described in Example 14 are further evaluated for their ability to inhibit human SK in a yeast model. Compounds which block the activity of human SK should restore growth on galactose to yeast strain dpl1ysr2Icb4 (Gal1, 10p:human SPHK1). This strain cannot catabolize S-1-P or endogenous yeast S-1-P analogs due to the deletion of genes encoding SPL and S-1-P phosphatases. The endogenous yeast SK gene, LCB4 has also been deleted, so that no endogenous S-1-P analogs are made under baseline conditions. This strain has been transformed with a plasmid containing the human SPHK1 gene under regulation of a galactose inducible promoter, such that expression of an active SK in the presence of galactose is lethal to this strain. Inhibition of human SK protects cells from galactose-induced lethality and confirms the efficacy of the compound in question. Activity of the human SPHK1 gene in this strain has been verified by two methods, first HPLC analysis of SK activity in whole extracts of cells grown (for a limited time) in the presence of galactose, and second, the inability of this strain to form colonies on galactose-containing plates.

D-erythro-sphingosine, N,N-dimethylsphingosine, and D,L-threo-dihydrosphingosine are obtained from Biomol Research Inc. (Plymouth Meeting, Pa.). The morpholine, piperadine and erythro series of PDMP analogues is obtained. Other analogues are prepared as described above and according to previously described methods (Srivastava, M., L. Bubendorf, V. Srikantan, L. Fossom, L. Nolan, M. Glasman, X. Leighton, W. Fehrle, S. Pittaluga, M. Raffeld, P. Koivisto, N. Willi, T. C. Gasser, J. Kononen, G. Sauter, O. P. Kallioniemi, S. Srivastava, and H. B. Pollard. 2001. ANX7, a candidate tumor suppressor gene for prostate cancer. Proc Natl Acad Sci USA. 98:4575-4580.; Bockmuhl, U., S. Schmidt, S. Petersen, and 1. Petersen. 2000. [Deletion of chromosome 10q—a marker for metastasis of head-neck carcinomas?]. Laryngorhinootologie. 79:81 -85.; Morita, R., S. Saito, J. Ishikawa, O. Ogawa, O. Yoshida, K. Yamakawa, and Y. Nakamura. 1991. Common regions of deletion on chromosomes 5q, 6q, and 10q in renal cell carcinoma. Cancer Res. 51:5817-5820.; Jenkins, R. B., I. D. Hay, J. F. Herath, C. G. Schultz, J. L. Spurbeck, C. S. Grant, J. R. Goellner, and G. W. Dewald. 1990. Frequent occurrence of cytogenetic abnormalities in sporadic nonmedullary thyroid carcinoma. Cancer. 66:1213-1220.; Shen, W. P., R. F. Young, B. N. Walter, B. H. Choi, M. Smith, and J. Katz. 1990. Molecular analysis of a myxoid chondrosarcoma with rearrangements of chromosomes 10 and 22. Cancer Genet Cytogenet. 45:207-215.; Simpson, N. E., K. K. Kidd, P. J. Goodfellow, H. McDermid, S. Myers, J. R. Kidd, C. E. Jackson, A. M. Duncan, L. A. Farrer, K. Brasch, and et al. 1987. Assignment of multiple endocrine neoplasia type 2A to chromosome 10 by linkage. Nature. 328:528-530.; Ichimura, K., E. Schmidt, A. Miyakawa, H. Goike, and V. Collins. 1998. Distinct patterns of deletion on 10p and 10q suggest involvement of multiple tumor suppressor genes in the development of astrocytic gliomas of different malignancy grades. Genes Chromosomes Cancer. 22:9-15.; Kim, S., H. Fyrst, and J. Saba. 2000. Accumulation of phosphorylated sphingoid long chain bases results in cell growth inhibition in Saccharomyces cerevisiae. Genetics. 156:1519-1529.).

Lipid extraction is carried out by Ion-exchange chromatography and HPLC analysis. SK assays are performed essentially as described (Taylor, M. V. 2000. Muscle development: molecules of myoblast fusion. Curr Biol. 10:R646-648.). Purification of native and recombinant SK is performed essentially as described Mao, C., M. Wadleigh, G. Jenkins, Y. Hannun, and L. Obeid. 1997. Identification and characterization of Saccharomyces cerevisiae dihydrosphingosine-1-phosphate phosphatase. J Biol Chem. 272:28690-28694).

### Example 16

### Generation of a Transgenic Drosophila Melanogaster Expression Human SPL and Human SPL-GFP Fusions

Transgenic Drosophila melanogaster were generated that overexpress human SPL (cDNA set forth in SEQ ID NO:23; amino acid sequence set forth in SEQ ID NO:18) and human SPL-GFP fusion proteins using standard techniques as described herein. The transgenes were introduced into wild type Canton-S (BL-1), and Sply⁰⁵⁰⁹¹ (BL-11393), mutant fly backgrounds.

### Example 17

### Development of a Yeast Model System for Screening SK Inhibitors

A yeast model system was developed that can be used as a screen to identify inhibitors of SK. A collection of yeast mutants demonstrating defects of long chain phosphorylated sphingoid bases (LCBPs) synthesis and metabolism was created. These mutants provide a novel schema by which to elucidate LCBP metabolism and identify genetic suppressors. These same mutant yeast strains can be used to screen chemicals which influence LCBP metabolism through their effect on key enzymes in the LCBP metabolic pathway.

Yeast strains which contain a null allele of DPL1, the yeast sphingosine phosphate lyase (SPL) gene, are unable to grow in the presence of even very low concentrations of D-erythro-sphingosine added to growth media. Not to be bound by theory, this is presumed to be caused by the fact that sphingosine is rapidly phosphorylated to S-1-P, which subsequently accumulates due to the failure of dpl1 mutant strain to catabolize the phosphorylated sphingoid base. This phenotype can be utilized to isolate mutants which cannot accumulate S-1-P and, therefore, are resistant to D-erythro-sphingosine. Mutations in the SK gene leading to diminished SK activity would reduce or eliminate production of S-1-P. Therefore, sphingosine resistant suppressors of dpl1Δ were selected in order to isolate SK-deficient mutants.

To ensure that deletion of DPL1 has no effect on SK activity, the SK activity present in wild type and dpl1Δ strains was evaluated. No significant differences in the SK activity of these two strains were observed, indicating that S. cerevisiae SK was not subject to end product inhibition in vivo.

A population of dpl1Δ cells was then subjected to a selection on D-erythro-sphingosine-enriched plates in order to isolate spontaneous suppressors of sphingosine sensitivity. Utilizing this approach, a number of suppressor mutants were isolated that exhibited significantly greater resistance to the growth-inhibitory effects of sphingosine than the original dpl1Δ cells. To determine whether these putative SK mutants were capable of phosphorylating a sphingoid base substrate, several clones were incubated with 100 μM D-erythro-sphingosine and extracts from these clones were subjected to S-1-P measurements using an HPLC-based assay essentially as described in Caligan, T. B., et al., 2000, Analytical Biochemistry 281:36-44. The strain in which the selection was performed (dpl1Δ) is unable to catabolize S-1-P and, therefore, accumulated S-1-P when exposed to exogenous sphingosine. Sphingosine resistant mutants of dpl1Δ synthesized no detectable S-1-P, indicating that SK activity was absent. Subsequent to the genetic screen described above, transposon mutagenesis was employed in a new screen in which dpl1 cells were transformed with a plasmid containing a transposable element, and the genomic location of the suppressor gene into which the transposon had inserted was determined. This approach led to the isolation of LCB4 as the major yeast sphingosine kinase gene (Saccharomyces cerevisiae gene and protein sequences are available at www.yeastgenome.org). In further experiments, LCB5, a homologue of LCB4 was identified as a minor yeast sphingosine kinase gene.

Subsequent to the isolation of DPL1 as the yeast SPL gene and LCB4 and its homologue LCB5 as the yeast sphingosine kinase genes (Nagiec, M., et al., J Biol Chem 1998, 273(31), 19437-19442; Lanterman, M. & Saba, J. Biochemical J. 1998, 332(2), 525-531.) two additional genes of sphingolipid metabolism were identified and characterized in yeast (Mao, C., et al., J Biol Chem 1997, 272(45), 28690-28694; Mao, C., et al., 1999, 342, 667-675; Mandala, S. M., et al., Proc Natl Acad Sci USA. 2000 Jul. 5;97(14):7859-64). These genes, YSR2 and YSR3, encode the major and minor sphingosine phosphate phosphatases, which catalyze the dephosphorylation of LCBPs to LCBs. These genes were identified through their ability, when overexpressed, to allow the DPL1 strain to survive in the presence of sphingosine. SUR2 catalyzes conversion of dihydrosphingosine to phytosphingosine and was also recently cloned using yeast genetic approaches.

To further explore the biological roles of LCBPs, disruption mutants for LCB4, LCB5, DPL1, YSR2, YSR3, and SUR2 were created, essentially as described in Kim, S., et al., 2000 Genetics. 156:1519-1529. An HPLC method was adapted to measure intracellular concentrations of the phosphorylated long chain bases in a variety of yeast mutants carrying single or multiple null alleles in the genes described above (Caligan, T. B., et al., 2000, supra). Mutants were grouped according to those which either accumulated or lacked LCBPs. Double mutants of dpl1 and ysr2 were inviable, while triple mutants of dpl1, ysr2, and Icb4 were viable. Further, growth inhibition associated with accumulated phosphorylated long chain bases was observed in the triple mutant dpl1 ysr2 Icb4 overexpressing LCB4, utilizing the pYES2 plasmid, in which gene expression was activated in the presence of galactose but inhibited in the presence of glucose. These results indicated that phosphorylated long chain bases inhibited cell growth. Mutants defective in both YSR2 and SUR2, which accumulated dihydrosphingosine 1-phosphate only, grew poorly. The phenotypes of the ysr2 sur2 mutants were suppressed by overexpression of DPL1.

These results suggested according to non-limiting theory that elevated levels of phosphorylated long chain bases had an antiproliferative effect in yeast and that this phenomenon could be regulated by the presence or absence of SK activity within the yeast cell.

To help elucidate the mechanism of growth inhibition induced by LCBP accumulation, growth in the presence of galactose was then utilized as a method for selecting suppressor strains in order. This screen was successful in identifying several interesting genetic suppressors of LCBP accumulation. To discern such interesting suppressors and avoid repeatedly identifying SK mutants as suppressors of LCBP degradation mutants, a LCB4-LacZ fusion aliele was used, which allowed the determination of which suppressors maintained SK expression as shown by blue colonies in the presence of IPTG and X-gal in the media. Colonies which gave a blue color in the presence of IPTG and X-gal, which grew on galactose-containing plates (which induced LCB4 expression) and which were prototrophic for leucine (the marker present in the transposon used in the screen) were considered interesting isolates which had not lost the SK gene.

Yeast SK activity demonstrated biochemical characteristics quite distinct from those of human SPHK1. Although yeast SK was able to phosphorylate both yeast and mammalian sphingoid bases, yeast SK was not inhibited by the mammalian SK inhibitor D,L-threo-dihydrosphingosine. Therefore, in order to validate two yeast genetic screens designed for the identification of inhibitors of human SK, it was necessary to create yeast strains which expressed human SPHK1 and which lacked yeast SK activity. Human SK cDNA (the human SK cDNA is set forth in SEQ ID NO:22, encoding the amino acid sequence set forth in SEQ ID NO:21) was synthesized by reverse transcription from total RNA obtained from human umbilical vein endothelial cells (HUVECs), and this fragment was cloned into the yeast expression vector pYES2 (Invitrogen, Carlsbad, Calif.), in which genes were expressed in a regulated fashion under control of a galactose inducible promoter as follows: Total RNA was extracted from HUVECs using the RNeasy Kit (Qiagen, Inc., Santa Clarita, Calif.), according to the manufacturer's instructions. Total RNA was utilized to create cDNA using reverse transcriptase and an oligo dT primer at 42° C. for 50 minutes. This cDNA was used as a template for amplification of the human SPHK1 cDNA sequence using primers 5′-ATATTAGCTTATGGATCCAGCGGGCGGCCCC-3′ (SEQ ID NO:40) and 5′-AGATCTAGATCATAAGGG-CTCTTCTGGCGG-3′ (SEQ ID NO:41), corresponding to the first 21 nucleotides and the last 19 nucleotides of the SPHK1 cDNA. Taq polymerase and the following PCR cycling conditions were used for amplification: 40 cycles of 94° C. for 30 seconds; 550 C for 1 minute; 680 C for 2 minutes; 68° C. for 7 minutes. The resulting DNA fragment was linearized by digestion with Eco RI and Xba I and cloned into the pYES2 yeast expression vector (Invitrogen Inc., Carlsbad, Calif.), in which genes cloned into the multiple cloning site were expressed under the regulation of the GAL4 glucose-repressible/galactose inducible promoter. This expression vector contained a URA3 gene useful in the selection of transformants on the basis of uracil prototrophy.

This plasmid was used to transform two yeast strains, the first of which contained a null allele for DPL1 and LCB4 and was therefore SPL and SK deficient, and a second strain which contained the aforementioned two null alleles and, in addition, a null allele for YSR2, therefore being additionally deficient in sphingosine phosphate phosphatase activity. The latter strain did not survive in the presence of SK activity, due to the massive accumulation of LCBPs. Transformants in each background were selected for uracil prototrophy.

These strains were analyzed and it was found that the SPHK1 gene was expressed and functional in yeast. First, whole cell extracts of the strains overexpressing mammalian SPHK1 genes demonstrated SK activity. Second, the triple knockout strain was streaked onto galactose plates and evaluated for its ability to survive. Lethality on galactose containing plates and normal growth on glucose plates were considered evidence for the expression of an active SK gene under galactose inducible regulation.

In summary, in this example, two important yeast mutant strains were created:

1. the first strain contained a null allele for DPL1 and LCB4 (and was therefore SPL and SK deficient) and was further transformed with either the mouse or human SPHK1 gene (see FIG. 4). This strain, like the dpl1 mutant, was highly sensitive to exogenous sphingosine and did not grow in culture medium containing 50 μM sphingosine unless SK activity was blocked.

2. a second strain which contained the aforementioned two null alleles of strain 1 and, in addition, a null allele for YSR2, therefore being additionally deficient in sphingosine phosphate phosphatase activity (see FIG. 5). This strain was further transformed to express human SPHK1 gene under the regulation of an inducible galactose promoter. Thus, this strain survived in glucose but did not survive in the presence of galactose (even in the absence of media containing sphingosine), unless SK activity was blocked.

The yeast strains identified herein provide a powerful tool for the isolation of genes involved in sphingolipid metabolism and signaling and a useful model with which to explore the role of phosphorylated sphingoid bases in eukaryotic cells. Further, these strains afford a novel approach to the identification of mammalian SK inhibitors.

In summary, the experiments described herein show that significant accumulation of the S-1-P analogues (LCBPs) in S. cerevisiae caused cell death. This lethality in yeast was the opposite of the growth stimulatory effect of S-1-P in mammalian cells, permitting the creation of screens in which SK inhibition allows for the survival of mutants containing defects in LCBP catabolism.

Methods

For the experiments described herein, yeast media and genetic methods were standard, as described in Adams, A., et al., 1998. Methods in Yeast Genetics. Cold Spring Harbor Laboratory Press, Plainview, N.Y. 177 pp. Yeast transformations were performed by the lithium acetate procedure (Ito, H., et al., 1983. J. Bact. 153:163-168). One step gene disruption or deletion procedures were employed to replace the endogenous genes with selectable PCR-amplified disruption constructs as described in Baudin, A., et al., 1993. Nucleic Acids Res. 21:3329-3330; Lorenz, M., et al., 1995. Gene. 158:113-117; Wach, A., et al., 1994. Yeast. 10:1793-1808. Yeast strains are isogenic derivatives of JK9-3d (leu2-3, 112 ura3-52 rme1 trp1 his4 HMLa). Most of the strains are meiotic segregants of diploid JK9-3d made singly or multiply heterozygous for disruption constructs dpl1Δ::KanMX, ysr2Δ::KanMX, ysr3Δ::KanMX, Icb4Δ::KanMX, Icb5Δ::KanMX, and sur2Δ::KanMX.

For HPLC analysis of LCBPs, strains containing a dpl1 allele can be grown for a short time periods in sphingosine before dying. To determine the S-1-P concentration of dpl1, human SK yeast in the presence of sphingosine with or without SK inhibitors, yeast cultures are incubated at a cell density of 5×10⁷ cells/ml for 2 hours in the presence of sphingosine at 30° C. Cultures are washed twice in sterile water and once in YP prior to lipid extraction. Lipids are extracted essentially as described in Kim, S., et al., 2000. Genetics. 156:1519-1529. Briefly, lipids are extracted by adding 5 ml of ice-cold MeOH followed by vortexing for 1 minute and tip sonication for 20 seconds. Extracts are centrifuged for 5 minutes in a tabletop centrifuge and the supernatant collected. A second extraction is performed on the pellet by adding an additional 5 ml ice-cold methanol followed by vortexing and sonication. The combined lipid extracts are then dried down in a speed vac, resuspended in 200 ml of 0.1M NH₄OH in MeOH, sonicated for 30 seconds in a bath sonicator and incubated for 1 hour at 37° C. to allow for the hydrolysis of esterified acyl chains. Following hydrolysis, the samples are cooled to room temperature, dried down with a flow of N₂ and resuspended in 1 ml of EtOH:H2O:diethylether:pyridine(15:15:5:1).

### Example 18

### Development of Two Screens for SK Inhibitors Using Mutant Yeast Strains

As described above in Example 17, two yeast mutant strains were developed (see FIG. 4 and FIG. 5). In this Example, two screens using the above described mutant strains to identify inhibitors of SK are described.

Screen 1: (FIG. 4)

Previous screens for suppressors of the dpl1 strain's sensitivity have been performed on agar plates containing 50 μM D-erythro-sphingosine ( Saba, J., et al., J Biol Chem 1997, 272(42), 26087). Since lipids disperse among cell membranes, the toxicity of sphingosine to this strain is dependent upon the number of cells plated, and therefore the concentration of sphingosine and cell concentration are both kept constant. A 96 well plate assay is used in which small, equal numbers of dpl1 cells are placed into each well, and an array of candidate chemicals (as described herein, for example in the section entitled, AGENTS THAT MODULATE SPHINGOLIPID INTERMEDIATES AND/OR COMPONENTS OF SPHINGOLIPID METABOLISM AND/OR SIGNALING and essentially as described in Example 14) are diluted into the wells prior to incubation. Growth curves are determined by inoculating synthetic complete media with logarithmically growing cells to an O.D._{600nm.}32 0.1, then following increase in optical density as a function of time. The lowest inhibitory concentration of sphingosine is determined. Controls for optimization include dpl1 in sphingosine of various concentrations plus N,N-dimethylsphingosine or D,L-threo-dihydrosphingosine, and wild type cells in the presence and absence of these inhibitors.

This approach is then compared to the standard plate assay, utilizing two available SK inhibitors, N,N-dimethylsphingosine and D,L-threo-dihydrosphingosine (Yatomi, Y., et al., Biochemistry 1996, 35(2), 626-633; Stoffel, W. & Bister, K. Hoppe-Seyler's Z Physiol Chem 1973, 354,169-181.) Concentrations of D-erythro-sphingosine that inhibit cell growth overnight (at 30° C. with shaking) are determined by measuring turbidity at OD₆₀₀ₙₘ. D-erythro-sphingosine concentrations that inhibit growth of a specific number of inoculated cells except in the presence of SK inhibitors are chosen as conditions for the screen. The assay is then validated in a dpl1 Icb4 Icb5 strain containing mouse SphK1 cDNA (GenBank Accession No. AF068748) (Kim, S., Fyrst, H. & Saba, J. Genetics 2000, 156, 1519-1529). Candidate agents promoting growth of this strain in sphingosine are validated using survival assays on agar plates containing the agent. The efficacy and biochemical characteristics of interesting compounds are quantified by 1) determining the IC₅₀ of the inhibitor on purified SK and recombinant human SK, 2) analyzing the kinetics of inhibition using classical Michaelis-Menten methods, 3) by determining the reversibility of the inhibition and 4) by comparing the LCBP accumulation of a dpl1 human SK strain treated with no inhibitor vs. treatment with a known SK inhibitors vs. treatment with the new compound. In vitro SK assays are performed using an HPLC based assay as described in Caligan, et al., (supra).

Screen 2: (FIG. 5)

Screen 2 is carried out as described above for Screen 1 except for the use of media containing 2% galactose in place of 2% glucose as a carbon source during screening, and lacking sphingosine.

In order to validate the dpl1Δ screen on agar plates the strains created above are inoculated to a density of 1×10⁴ cells per plate onto agar plates containing 100 μM D-erythro-sphingosine and 0.0015% NP40 (used as a dispersant). At these concentrations of sphingosine and this cell density, the dpl1Δ strains do not grow. Similarly, plates containing D-erythro-sphingosine plus known SK inhibitors are incubated for 3-5 days, and plates are inspected for sphingosine resistance. If it appears that the strain containing human SK can survive sphingosine in the presence of SK inhibitors, a dilutional assay provides semi-quantitative information on the degree of protection afforded by the known inhibitors. For the sphingosine dilutional assay, cells are grown to saturation for 2-3 days in liquid cultures and then resuspended in minimal medium, placed in the first row of a 96 well plate and diluted serially from 1:2 to 1:4000 across the plate. The cultures are then template-inoculated onto a control plate (rich medium) and a plate containing 50 μM sphingosine and 0.0015% NP40, plus or minus SK inhibitor (At this concentration of NP40, no effects on cell viability are observed.) Plates are then incubated at 30° C. for 2 days and assessed visually for differences in growth.

Methods for yeast growth and genetics and measuring LCBPs are as described in the preceding examples.

### Example 19

### Biochemical Analysis of SK Inhibitors

The biochemical characteristics of the SK inhibitors identified in the above described yeast screens are evaluated as follows.

IC₅₀ on recombinant human SK and SK obtained from a variety of sources including human platelets, leukemia cell lines, breast cancer cell lines, normal human lymphocytes and crude yeast extracts expressing human, mouse, Drosophila and C. elegans SK cDNA is carried out using standard assays. Kinetics of inhibition using classical Michaelis-Menten methods are determined. The presence or absence of inhibitor phosphorylation is determined using standard assays. The effect on SK activity at baseline and in response to known SK inhibitors is determined using SK assays as described herein and in the art. The effect of inhibitor on intracellular levels of sphingosine, dihydrophingosine, S-1-P, DHS-1-P, ceramide and total phospholipids is determined using an HPLC-based assay (In vitro SK assays and all sphingolipid measurements are performed using an HPLC-based assay essentially as described in Caligan, et al., 2000 Analytical Biochemistry 281:36-44). In addition to determining specificity for SK from the sources mentioned above, specificity for SK is determined by evaluating inhibitor effects on other lipid kinases (diacylglycerol kinase and P13 kinase) and protein kinase C, which is inhibited by sphingosine. Mass measurements of sphingolipids are determined using a radioactive uptake thin-layer chromatography (TLC) assay. Activity against other enzymes in the sphingolipid metabolic pathway, including SPP, SPL, serine palmitoyl transferase (SPT) and ceramide synthase are evaluated using standard assays. Methods used for the above analyses are essentially as described in Caligan, et al., 2000 Supra; Skrzypek, M, et al., 1999 J. Bacteriol. 181:1134-1140; Kim S., et al., 2000 Genetics, 156:1519-1529; Lanterman, M and Saba, J., 1998 Biochem J, 332:525-531.

### Example 20

### Biological Analysis of SK Inhibitors

The biological characteristics of the SK inhibitors identified in the above described yeast screens are evaluated as follows.

SK inhibitors identified using the screens as described herein are evaluated for dosage-dependent effects on the morphology, proliferation, apoptosis and stress response in cancer cell lines, including lines harboring dominant Ras mutations or SK overexpression (ovarian, colon, and breast cancer.) SK inhibitors are investigated for potential synergy with SPL overexpression in reducing S-1-P levels and signaling in a HEK293 cell line stably overexpressing a human SPL-GFP fusion protein. This cell line demonstrates very high SPL activity and increased apoptosis under stress conditions compared to a cell line stably expressing a catalytically inactive human SPL. Apoptosis in 0-10% serum is determined by caspase 3 activation. Information gained from these studies provides tools necessary to identify human SK inhibitors from any chemical array or library that might potentially contain them. In addition, compounds are tested for their ability to inhibit Drosophila SK function and to correct the physiological, anatomical, and reproductive defects of the Drosophila Sply mutant as described herein which lacks SPL expression.

From the foregoing, it will be appreciated that, although specific embodiments of the invention have been described herein for the purpose of illustration, various modifications may be made without deviating from the spirit and scope of the invention.

```
# SEQUENCE LIS
#TING
<160> NUMBER OF SEQ ID NOS: 41
<210> SEQ ID NO 1
<211> LENGTH: 1770
<212> TYPE: DNA
<213> ORGANISM: S. cerevisiae
<220> FEATURE:
<221> NAME/KEY: CDS
<222> LOCATION: (1)...(1770)
<400> SEQUENCE: 1
atg agt gga gta tca aat aaa aca gta tca at
#t aat ggt tgg tat ggc 48
Met Ser Gly Val Ser Asn Lys Thr Val Ser Il
#e Asn Gly Trp Tyr Gly
  1 5
# 10
# 15
atg cca att cat tta cta agg gaa gaa ggc ga
#c ttt gcc cag ttt atg 96
Met Pro Ile His Leu Leu Arg Glu Glu Gly As
#p Phe Ala Gln Phe Met
                          20
# 25
# 30
att cta acc atc aac gaa tta aaa ata gcc at
#a cat ggt tac ctc aga 144
Ile Leu Thr Ile Asn Glu Leu Lys Ile Ala Il
#e His Gly Tyr Leu Arg
                  35
# 40
# 45
aat acc cca tgg tac aac atg ttg aag gat ta
#t ttg ttt gtg atc ttt 192
Asn Thr Pro Trp Tyr Asn Met Leu Lys Asp Ty
#r Leu Phe Val Ile Phe
          50
# 55
# 60
tgt tac aag cta ata agt aat ttt ttt tat ct
#g ttg aaa gtt tat ggg 240
Cys Tyr Lys Leu Ile Ser Asn Phe Phe Tyr Le
#u Leu Lys Val Tyr Gly
  65
# 70
# 75
# 80
ccg gtg agg tta gca gtg aga aca tac gag ca
#t agt tcc aga aga ttg 288
Pro Val Arg Leu Ala Val Arg Thr Tyr Glu Hi
#s Ser Ser Arg Arg Leu
                                  85
# 90
# 95
ttt cgt tgg tta ttg gac tca cca ttt ttg ag
#g ggt acc gta gaa aag 336
Phe Arg Trp Leu Leu Asp Ser Pro Phe Leu Ar
#g Gly Thr Val Glu Lys
                        100
# 105
# 110
gaa gtc aca aag gtc aaa caa tcg atc gaa ga
#c gaa cta att aga tcg 384
Glu Val Thr Lys Val Lys Gln Ser Ile Glu As
#p Glu Leu Ile Arg Ser
                115
# 120
# 125
gac tct cag tta atg aat ttc cca cag ttg cc
#a tcc aat ggg ata cct 432
Asp Ser Gln Leu Met Asn Phe Pro Gln Leu Pr
#o Ser Asn Gly Ile Pro
        130
# 135
# 140
cag gat gat gtt att gaa gag cta aat aaa tt
#g aac gac ttg ata cca 480
Gln Asp Asp Val Ile Glu Glu Leu Asn Lys Le
#u Asn Asp Leu Ile Pro
145 1
#50 1
#55 1
#60
cat acc caa tgg aag gaa gga aag gtc tct gg
#t gcc gtt tac cac ggt 528
His Thr Gln Trp Lys Glu Gly Lys Val Ser Gl
#y Ala Val Tyr His Gly
                                165
# 170
# 175
ggt gat gat ttg atc cac tta caa aca atc gc
#a tac gaa aaa tat tgc 576
Gly Asp Asp Leu Ile His Leu Gln Thr Ile Al
#a Tyr Glu Lys Tyr Cys
                        180
# 185
# 190
gtt gcc aat caa tta cat ccc gat gtc ttt cc
#t gcc gta cgt aaa atg 624
Val Ala Asn Gln Leu His Pro Asp Val Phe Pr
#o Ala Val Arg Lys Met
                195
# 200
# 205
gaa tcc gaa gtg gtt tct atg gtt tta aga at
#g ttt aat gcc cct tct 672
Glu Ser Glu Val Val Ser Met Val Leu Arg Me
#t Phe Asn Ala Pro Ser
        210
# 215
# 220
gat aca ggt tgt ggt acc aca act tca ggt gg
#t aca gaa tcc ttg ctt 720
Asp Thr Gly Cys Gly Thr Thr Thr Ser Gly Gl
#y Thr Glu Ser Leu Leu
225 2
#30 2
#35 2
#40
tta gca tgt ctg agc gct aaa atg tat gcc ct
#t cat cat cgt gga atc 768
Leu Ala Cys Leu Ser Ala Lys Met Tyr Ala Le
#u His His Arg Gly Ile
                                245
# 250
# 255
acc gaa cca gaa ata att gct ccc gta act gc
#a cat gct ggg ttt gac 816
Thr Glu Pro Glu Ile Ile Ala Pro Val Thr Al
#a His Ala Gly Phe Asp
                        260
# 265
# 270
aaa gct gct tat tac ttt ggc atg aag cta cg
#c cac gtg gag cta gat 864
Lys Ala Ala Tyr Tyr Phe Gly Met Lys Leu Ar
#g His Val Glu Leu Asp
                275
# 280
# 285
cca acg aca tat caa gtg gac ctg gga aaa gt
#g aaa aaa ttc atc aat 912
Pro Thr Thr Tyr Gln Val Asp Leu Gly Lys Va
#l Lys Lys Phe Ile Asn
        290
# 295
# 300
aag aac aca att tta ctg gtc ggt tcc gct cc
#a aac ttt cct cat ggt 960
Lys Asn Thr Ile Leu Leu Val Gly Ser Ala Pr
#o Asn Phe Pro His Gly
305 3
#10 3
#15 3
#20
att gcc gat gat att gaa gga ttg ggt aaa at
#a gca caa aaa tat aaa 1008
Ile Ala Asp Asp Ile Glu Gly Leu Gly Lys Il
#e Ala Gln Lys Tyr Lys
                                325
# 330
# 335
ctt cct tta cac gtc gac agt tgt cta ggt tc
#c ttt att gtt tca ttt 1056
Leu Pro Leu His Val Asp Ser Cys Leu Gly Se
#r Phe Ile Val Ser Phe
                        340
# 345
# 350
atg gaa aag gct ggt tac aaa aat ctg cca tt
#a ctt gac ttt aga gtc 1104
Met Glu Lys Ala Gly Tyr Lys Asn Leu Pro Le
#u Leu Asp Phe Arg Val
                355
# 360
# 365
ccg gga gtc acc tca ata tca tgt gac act ca
#t aaa tat gga ttt gca 1152
Pro Gly Val Thr Ser Ile Ser Cys Asp Thr Hi
#s Lys Tyr Gly Phe Ala
        370
# 375
# 380
cca aaa ggc tcg tca gtt ata atg tat aga aa
#c agc gac tta cga atg 1200
Pro Lys Gly Ser Ser Val Ile Met Tyr Arg As
#n Ser Asp Leu Arg Met
385 3
#90 3
#95 4
#00
cat cag tat tac gta aat cct gct tgg act gg
#c ggg tta tat ggc tct 1248
His Gln Tyr Tyr Val Asn Pro Ala Trp Thr Gl
#y Gly Leu Tyr Gly Ser
                                405
# 410
# 415
cct aca tta gca ggg tcc agg cct ggt gct at
#t gtc gta ggt tgt tgg 1296
Pro Thr Leu Ala Gly Ser Arg Pro Gly Ala Il
#e Val Val Gly Cys Trp
                        420
# 425
# 430
gcc act atg gtc aac atg ggt gaa aat ggg ta
#c att gag tcg tgc caa 1344
Ala Thr Met Val Asn Met Gly Glu Asn Gly Ty
#r Ile Glu Ser Cys Gln
                435
# 440
# 445
gaa ata gtc ggt gca gca atg aag ttt aaa aa
#a tac atc cag gaa aac 1392
Glu Ile Val Gly Ala Ala Met Lys Phe Lys Ly
#s Tyr Ile Gln Glu Asn
        450
# 455
# 460
att cca gac ctg aat ata atg ggc aac cct ag
#a tat tca gtc att tca 1440
Ile Pro Asp Leu Asn Ile Met Gly Asn Pro Ar
#g Tyr Ser Val Ile Ser
465 4
#70 4
#75 4
#80
ttt tct tca aag acc ttg aac ata cac gaa ct
#a tct gac agg ttg tcc 1488
Phe Ser Ser Lys Thr Leu Asn Ile His Glu Le
#u Ser Asp Arg Leu Ser
                                485
# 490
# 495
aag aaa ggc tgg cat ttc aat gcc cta caa aa
#g ccg gtt gca cta cac 1536
Lys Lys Gly Trp His Phe Asn Ala Leu Gln Ly
#s Pro Val Ala Leu His
                        500
# 505
# 510
atg gcc ttc acg aga ttg agc gct cat gtt gt
#g gat gag atc tgc gac 1584
Met Ala Phe Thr Arg Leu Ser Ala His Val Va
#l Asp Glu Ile Cys Asp
                515
# 520
# 525
att tta cgt act acc gtg caa gag ttg aag ag
#c gaa tca aat tct aaa 1632
Ile Leu Arg Thr Thr Val Gln Glu Leu Lys Se
#r Glu Ser Asn Ser Lys
        530
# 535
# 540
cca tcc cca gac gga act agc gct cta tat gg
#t gtc gcc ggg agc gtt 1680
Pro Ser Pro Asp Gly Thr Ser Ala Leu Tyr Gl
#y Val Ala Gly Ser Val
545 5
#50 5
#55 5
#60
aaa act gct ggc gtt gca gac aaa ttg att gt
#g gga ttc cta gac gca 1728
Lys Thr Ala Gly Val Ala Asp Lys Leu Ile Va
#l Gly Phe Leu Asp Ala
                                565
# 570
# 575
tta tac aag ttg ggt cca gga gag gat acc gc
#c acc aag tag
#1770
Leu Tyr Lys Leu Gly Pro Gly Glu Asp Thr Al
#a Thr Lys *
                        580
# 585
<210> SEQ ID NO 2
<211> LENGTH: 589
<212> TYPE: PRT
<213> ORGANISM: S. cerevisiae
<400> SEQUENCE: 2
Met Ser Gly Val Ser Asn Lys Thr Val Ser Il
#e Asn Gly Trp Tyr Gly
  1 5
# 10
# 15
Met Pro Ile His Leu Leu Arg Glu Glu Gly As
#p Phe Ala Gln Phe Met
                        20
# 25
# 30
Ile Leu Thr Ile Asn Glu Leu Lys Ile Ala Il
#e His Gly Tyr Leu Arg
                35
# 40
# 45
Asn Thr Pro Trp Tyr Asn Met Leu Lys Asp Ty
#r Leu Phe Val Ile Phe
        50
# 55
# 60
Cys Tyr Lys Leu Ile Ser Asn Phe Phe Tyr Le
#u Leu Lys Val Tyr Gly
65
#70
#75
#80
Pro Val Arg Leu Ala Val Arg Thr Tyr Glu Hi
#s Ser Ser Arg Arg Leu
                                85
# 90
# 95
Phe Arg Trp Leu Leu Asp Ser Pro Phe Leu Ar
#g Gly Thr Val Glu Lys
                        100
# 105
# 110
Glu Val Thr Lys Val Lys Gln Ser Ile Glu As
#p Glu Leu Ile Arg Ser
                115
# 120
# 125
Asp Ser Gln Leu Met Asn Phe Pro Gln Leu Pr
#o Ser Asn Gly Ile Pro
        130
# 135
# 140
Gln Asp Asp Val Ile Glu Glu Leu Asn Lys Le
#u Asn Asp Leu Ile Pro
145 1
#50 1
#55 1
#60
His Thr Gln Trp Lys Glu Gly Lys Val Ser Gl
#y Ala Val Tyr His Gly
                                165
# 170
# 175
Gly Asp Asp Leu Ile His Leu Gln Thr Ile Al
#a Tyr Glu Lys Tyr Cys
                        180
# 185
# 190
Val Ala Asn Gln Leu His Pro Asp Val Phe Pr
#o Ala Val Arg Lys Met
                195
# 200
# 205
Glu Ser Glu Val Val Ser Met Val Leu Arg Me
#t Phe Asn Ala Pro Ser
        210
# 215
# 220
Asp Thr Gly Cys Gly Thr Thr Thr Ser Gly Gl
#y Thr Glu Ser Leu Leu
225 2
#30 2
#35 2
#40
Leu Ala Cys Leu Ser Ala Lys Met Tyr Ala Le
#u His His Arg Gly Ile
                                245
# 250
# 255
Thr Glu Pro Glu Ile Ile Ala Pro Val Thr Al
#a His Ala Gly Phe Asp
                        260
# 265
# 270
Lys Ala Ala Tyr Tyr Phe Gly Met Lys Leu Ar
#g His Val Glu Leu Asp
                275
# 280
# 285
Pro Thr Thr Tyr Gln Val Asp Leu Gly Lys Va
#l Lys Lys Phe Ile Asn
        290
# 295
# 300
Lys Asn Thr Ile Leu Leu Val Gly Ser Ala Pr
#o Asn Phe Pro His Gly
305 3
#10 3
#15 3
#20
Ile Ala Asp Asp Ile Glu Gly Leu Gly Lys Il
#e Ala Gln Lys Tyr Lys
                                325
# 330
# 335
Leu Pro Leu His Val Asp Ser Cys Leu Gly Se
#r Phe Ile Val Ser Phe
                        340
# 345
# 350
Met Glu Lys Ala Gly Tyr Lys Asn Leu Pro Le
#u Leu Asp Phe Arg Val
                355
# 360
# 365
Pro Gly Val Thr Ser Ile Ser Cys Asp Thr Hi
#s Lys Tyr Gly Phe Ala
        370
# 375
# 380
Pro Lys Gly Ser Ser Val Ile Met Tyr Arg As
#n Ser Asp Leu Arg Met
385 3
#90 3
#95 4
#00
His Gln Tyr Tyr Val Asn Pro Ala Trp Thr Gl
#y Gly Leu Tyr Gly Ser
                                405
# 410
# 415
Pro Thr Leu Ala Gly Ser Arg Pro Gly Ala Il
#e Val Val Gly Cys Trp
                        420
# 425
# 430
Ala Thr Met Val Asn Met Gly Glu Asn Gly Ty
#r Ile Glu Ser Cys Gln
                435
# 440
# 445
Glu Ile Val Gly Ala Ala Met Lys Phe Lys Ly
#s Tyr Ile Gln Glu Asn
        450
# 455
# 460
Ile Pro Asp Leu Asn Ile Met Gly Asn Pro Ar
#g Tyr Ser Val Ile Ser
465 4
#70 4
#75 4
#80
Phe Ser Ser Lys Thr Leu Asn Ile His Glu Le
#u Ser Asp Arg Leu Ser
                                485
# 490
# 495
Lys Lys Gly Trp His Phe Asn Ala Leu Gln Ly
#s Pro Val Ala Leu His
                        500
# 505
# 510
Met Ala Phe Thr Arg Leu Ser Ala His Val Va
#l Asp Glu Ile Cys Asp
                515
# 520
# 525
Ile Leu Arg Thr Thr Val Gln Glu Leu Lys Se
#r Glu Ser Asn Ser Lys
        530
# 535
# 540
Pro Ser Pro Asp Gly Thr Ser Ala Leu Tyr Gl
#y Val Ala Gly Ser Val
545 5
#50 5
#55 5
#60
Lys Thr Ala Gly Val Ala Asp Lys Leu Ile Va
#l Gly Phe Leu Asp Ala
                                565
# 570
# 575
Leu Tyr Lys Leu Gly Pro Gly Glu Asp Thr Al
#a Thr Lys
                        580
# 585
<210> SEQ ID NO 3
<211> LENGTH: 1629
<212> TYPE: DNA
<213> ORGANISM: C. elegans
<220> FEATURE:
<221> NAME/KEY: CDS
<222> LOCATION: (1)...(1629)
<400> SEQUENCE: 3
atg gat ttt gca ctg gag caa tat cat agt gc
#a aag gat ttg tta ata 48
Met Asp Phe Ala Leu Glu Gln Tyr His Ser Al
#a Lys Asp Leu Leu Ile
  1 5
# 10
# 15
ttt gag ctt cga aag ttc aat cca att gtt ct
#g gtt tct agt act att 96
Phe Glu Leu Arg Lys Phe Asn Pro Ile Val Le
#u Val Ser Ser Thr Ile
                          20
# 25
# 30
gtt gca aca tac gta ctc acc aat ctg aga ca
#t atg cat tta gat gaa 144
Val Ala Thr Tyr Val Leu Thr Asn Leu Arg Hi
#s Met His Leu Asp Glu
                  35
# 40
# 45
atg ggc atc cgg aaa cgt ttg agc act tgg tt
#t ttc acc act gta aag 192
Met Gly Ile Arg Lys Arg Leu Ser Thr Trp Ph
#e Phe Thr Thr Val Lys
          50
# 55
# 60
cgt gtg cct ttc atc agg aaa atg att gac aa
#a caa cta aac gaa gta 240
Arg Val Pro Phe Ile Arg Lys Met Ile Asp Ly
#s Gln Leu Asn Glu Val
  65
# 70
# 75
# 80
aag gac gag ctt gag aaa agt ctg aga att gt
#g gat cga agc acc gaa 288
Lys Asp Glu Leu Glu Lys Ser Leu Arg Ile Va
#l Asp Arg Ser Thr Glu
                                  85
# 90
# 95
tac ttc act aca atc cca agc cat tca gtt gg
#a aga act gaa gta ctt 336
Tyr Phe Thr Thr Ile Pro Ser His Ser Val Gl
#y Arg Thr Glu Val Leu
                        100
# 105
# 110
cgc ctt gct gcc atc tat gat gat ttg gaa gg
#a cca gct ttt ttg gaa 384
Arg Leu Ala Ala Ile Tyr Asp Asp Leu Glu Gl
#y Pro Ala Phe Leu Glu
                115
# 120
# 125
gga aga gta tct gga gca gtc ttc aat aga ga
#a gac gac aag gac gaa 432
Gly Arg Val Ser Gly Ala Val Phe Asn Arg Gl
#u Asp Asp Lys Asp Glu
        130
# 135
# 140
cgg gag atg tat gag gag gtg ttc gga aaa tt
#t gcc tgg acc aac cca 480
Arg Glu Met Tyr Glu Glu Val Phe Gly Lys Ph
#e Ala Trp Thr Asn Pro
145 1
#50 1
#55 1
#60
ctt tgg cca aaa ttg ttc cct gga gtg aga at
#c atg gag gct gaa gtt 528
Leu Trp Pro Lys Leu Phe Pro Gly Val Arg Il
#e Met Glu Ala Glu Val
                                165
# 170
# 175
gtt cgc atg tgt tgt aat atg atg aat gga ga
#t tcg gag aca tgt gga 576
Val Arg Met Cys Cys Asn Met Met Asn Gly As
#p Ser Glu Thr Cys Gly
                        180
# 185
# 190
act atg tca act ggt gga tcc att tca att ct
#t ttg gcg tgc ctg gct 624
Thr Met Ser Thr Gly Gly Ser Ile Ser Ile Le
#u Leu Ala Cys Leu Ala
                195
# 200
# 205
cat cgt aat cgt ctt ttg aaa aga gga gaa aa
#g tac aca gag atg att 672
His Arg Asn Arg Leu Leu Lys Arg Gly Glu Ly
#s Tyr Thr Glu Met Ile
        210
# 215
# 220
gtc cca tca tcc gtc cat gca gcg ttc ttc aa
#a gct gcc gaa tgt ttc 720
Val Pro Ser Ser Val His Ala Ala Phe Phe Ly
#s Ala Ala Glu Cys Phe
225 2
#30 2
#35 2
#40
cgt atc aaa gtt cgc aag att cca gtt gat cc
#t gtt act ttc aaa gta 768
Arg Ile Lys Val Arg Lys Ile Pro Val Asp Pr
#o Val Thr Phe Lys Val
                                245
# 250
# 255
gac ctt gtc aaa atg aaa gcc gca att aac aa
#g aga aca tgt atg tta 816
Asp Leu Val Lys Met Lys Ala Ala Ile Asn Ly
#s Arg Thr Cys Met Leu
                        260
# 265
# 270
gtt gga tct gct cca aac ttt cca ttt gga ac
#t gtt gat gac att gaa 864
Val Gly Ser Ala Pro Asn Phe Pro Phe Gly Th
#r Val Asp Asp Ile Glu
                275
# 280
# 285
gct att gga cag cta gga ctt gaa tat gac at
#c cca gtt cat gtt gat 912
Ala Ile Gly Gln Leu Gly Leu Glu Tyr Asp Il
#e Pro Val His Val Asp
        290
# 295
# 300
gct tgt ctt ggt ggt ttc ctt ctt cca ttc ct
#t gaa gaa gac gag att 960
Ala Cys Leu Gly Gly Phe Leu Leu Pro Phe Le
#u Glu Glu Asp Glu Ile
305 3
#10 3
#15 3
#20
cgc tat gac ttc cgt gtt cct ggt gta tct tc
#g att tct gca gat agt 1008
Arg Tyr Asp Phe Arg Val Pro Gly Val Ser Se
#r Ile Ser Ala Asp Ser
                                325
# 330
# 335
cac aaa tac gga ctc gct cca aag ggg tca tc
#a gtt gtt ctt tat cgc 1056
His Lys Tyr Gly Leu Ala Pro Lys Gly Ser Se
#r Val Val Leu Tyr Arg
                        340
# 345
# 350
aat aag gaa ctt ctt cat aat cag tac ttc tg
#t gat gct gat tgg caa 1104
Asn Lys Glu Leu Leu His Asn Gln Tyr Phe Cy
#s Asp Ala Asp Trp Gln
                355
# 360
# 365
gga ggt atc tat gca tcg gct act atg gaa gg
#a tca cgc gct ggg cac 1152
Gly Gly Ile Tyr Ala Ser Ala Thr Met Glu Gl
#y Ser Arg Ala Gly His
        370
# 375
# 380
aac att gca ctt tgc tgg gcc gca atg ctt ta
#t cac gct cag gaa gga 1200
Asn Ile Ala Leu Cys Trp Ala Ala Met Leu Ty
#r His Ala Gln Glu Gly
385 3
#90 3
#95 4
#00
tac aag gcc aat gct aga aag att gtt gac ac
#t aca aga aag att aga 1248
Tyr Lys Ala Asn Ala Arg Lys Ile Val Asp Th
#r Thr Arg Lys Ile Arg
                                405
# 410
# 415
aat gga ctt tca aac att aag gga atc aaa tt
#a caa ggg cca agt gat 1296
Asn Gly Leu Ser Asn Ile Lys Gly Ile Lys Le
#u Gln Gly Pro Ser Asp
                        420
# 425
# 430
gtt tgt att gtt agc tgg aca acc aat gat gg
#a gtt gaa ctc tac aga 1344
Val Cys Ile Val Ser Trp Thr Thr Asn Asp Gl
#y Val Glu Leu Tyr Arg
                435
# 440
# 445
ttc cat aac ttc atg aag gaa aaa cat tgg ca
#a ctg aat gga ctt caa 1392
Phe His Asn Phe Met Lys Glu Lys His Trp Gl
#n Leu Asn Gly Leu Gln
        450
# 455
# 460
ttc cca gct gga gtt cat atc atg gtc act at
#g aat cat act cat cct 1440
Phe Pro Ala Gly Val His Ile Met Val Thr Me
#t Asn His Thr His Pro
465 4
#70 4
#75 4
#80
gga ctc gct gaa gct ttc gtc gcc gat tgc ag
#a gct gca gtt gag ttt 1488
Gly Leu Ala Glu Ala Phe Val Ala Asp Cys Ar
#g Ala Ala Val Glu Phe
                                485
# 490
# 495
gtc aaa agc cac aaa cca tcg gaa tcc gac aa
#g aca agt gaa gca gcc 1536
Val Lys Ser His Lys Pro Ser Glu Ser Asp Ly
#s Thr Ser Glu Ala Ala
                        500
# 505
# 510
atc tac gga ctt gct caa agt att cca gac cg
#a tcg ctt gtt cac gag 1584
Ile Tyr Gly Leu Ala Gln Ser Ile Pro Asp Ar
#g Ser Leu Val His Glu
                515
# 520
# 525
ttt gct cac agc tat atc gat gct gtt tat gc
#t tta aca gag tga 1629
Phe Ala His Ser Tyr Ile Asp Ala Val Tyr Al
#a Leu Thr Glu *
        530
# 535
# 540
<210> SEQ ID NO 4
<211> LENGTH: 542
<212> TYPE: PRT
<213> ORGANISM: C. elegans
<400> SEQUENCE: 4
Met Asp Phe Ala Leu Glu Gln Tyr His Ser Al
#a Lys Asp Leu Leu Ile
  1 5
# 10
# 15
Phe Glu Leu Arg Lys Phe Asn Pro Ile Val Le
#u Val Ser Ser Thr Ile
                        20
# 25
# 30
Val Ala Thr Tyr Val Leu Thr Asn Leu Arg Hi
#s Met His Leu Asp Glu
                35
# 40
# 45
Met Gly Ile Arg Lys Arg Leu Ser Thr Trp Ph
#e Phe Thr Thr Val Lys
        50
# 55
# 60
Arg Val Pro Phe Ile Arg Lys Met Ile Asp Ly
#s Gln Leu Asn Glu Val
65
#70
#75
#80
Lys Asp Glu Leu Glu Lys Ser Leu Arg Ile Va
#l Asp Arg Ser Thr Glu
                                85
# 90
# 95
Tyr Phe Thr Thr Ile Pro Ser His Ser Val Gl
#y Arg Thr Glu Val Leu
                        100
# 105
# 110
Arg Leu Ala Ala Ile Tyr Asp Asp Leu Glu Gl
#y Pro Ala Phe Leu Glu
                115
# 120
# 125
Gly Arg Val Ser Gly Ala Val Phe Asn Arg Gl
#u Asp Asp Lys Asp Glu
        130
# 135
# 140
Arg Glu Met Tyr Glu Glu Val Phe Gly Lys Ph
#e Ala Trp Thr Asn Pro
145 1
#50 1
#55 1
#60
Leu Trp Pro Lys Leu Phe Pro Gly Val Arg Il
#e Met Glu Ala Glu Val
                                165
# 170
# 175
Val Arg Met Cys Cys Asn Met Met Asn Gly As
#p Ser Glu Thr Cys Gly
                        180
# 185
# 190
Thr Met Ser Thr Gly Gly Ser Ile Ser Ile Le
#u Leu Ala Cys Leu Ala
                195
# 200
# 205
His Arg Asn Arg Leu Leu Lys Arg Gly Glu Ly
#s Tyr Thr Glu Met Ile
        210
# 215
# 220
Val Pro Ser Ser Val His Ala Ala Phe Phe Ly
#s Ala Ala Glu Cys Phe
225 2
#30 2
#35 2
#40
Arg Ile Lys Val Arg Lys Ile Pro Val Asp Pr
#o Val Thr Phe Lys Val
                                245
# 250
# 255
Asp Leu Val Lys Met Lys Ala Ala Ile Asn Ly
#s Arg Thr Cys Met Leu
                        260
# 265
# 270
Val Gly Ser Ala Pro Asn Phe Pro Phe Gly Th
#r Val Asp Asp Ile Glu
                275
# 280
# 285
Ala Ile Gly Gln Leu Gly Leu Glu Tyr Asp Il
#e Pro Val His Val Asp
        290
# 295
# 300
Ala Cys Leu Gly Gly Phe Leu Leu Pro Phe Le
#u Glu Glu Asp Glu Ile
305 3
#10 3
#15 3
#20
Arg Tyr Asp Phe Arg Val Pro Gly Val Ser Se
#r Ile Ser Ala Asp Ser
                                325
# 330
# 335
His Lys Tyr Gly Leu Ala Pro Lys Gly Ser Se
#r Val Val Leu Tyr Arg
                        340
# 345
# 350
Asn Lys Glu Leu Leu His Asn Gln Tyr Phe Cy
#s Asp Ala Asp Trp Gln
                355
# 360
# 365
Gly Gly Ile Tyr Ala Ser Ala Thr Met Glu Gl
#y Ser Arg Ala Gly His
        370
# 375
# 380
Asn Ile Ala Leu Cys Trp Ala Ala Met Leu Ty
#r His Ala Gln Glu Gly
385 3
#90 3
#95 4
#00
Tyr Lys Ala Asn Ala Arg Lys Ile Val Asp Th
#r Thr Arg Lys Ile Arg
                                405
# 410
# 415
Asn Gly Leu Ser Asn Ile Lys Gly Ile Lys Le
#u Gln Gly Pro Ser Asp
                        420
# 425
# 430
Val Cys Ile Val Ser Trp Thr Thr Asn Asp Gl
#y Val Glu Leu Tyr Arg
                435
# 440
# 445
Phe His Asn Phe Met Lys Glu Lys His Trp Gl
#n Leu Asn Gly Leu Gln
        450
# 455
# 460
Phe Pro Ala Gly Val His Ile Met Val Thr Me
#t Asn His Thr His Pro
465 4
#70 4
#75 4
#80
Gly Leu Ala Glu Ala Phe Val Ala Asp Cys Ar
#g Ala Ala Val Glu Phe
                                485
# 490
# 495
Val Lys Ser His Lys Pro Ser Glu Ser Asp Ly
#s Thr Ser Glu Ala Ala
                        500
# 505
# 510
Ile Tyr Gly Leu Ala Gln Ser Ile Pro Asp Ar
#g Ser Leu Val His Glu
                515
# 520
# 525
Phe Ala His Ser Tyr Ile Asp Ala Val Tyr Al
#a Leu Thr Glu
        530
# 535
# 540
<210> SEQ ID NO 5
<211> LENGTH: 1707
<212> TYPE: DNA
<213> ORGANISM: Mus musculus
<220> FEATURE:
<221> NAME/KEY: CDS
<222> LOCATION: (1)...(1707)
<400> SEQUENCE: 5
atg ccc gga acc gac ctc ctc aag ctg aag ga
#c ttc gag cct tat ttg 48
Met Pro Gly Thr Asp Leu Leu Lys Leu Lys As
#p Phe Glu Pro Tyr Leu
  1 5
# 10
# 15
gag att ttg gaa tct tat tcc aca aaa gcc aa
#g aat tat gtg aat gga 96
Glu Ile Leu Glu Ser Tyr Ser Thr Lys Ala Ly
#s Asn Tyr Val Asn Gly
                          20
# 25
# 30
tat tgc acc aaa tat gag ccc tgg cag ctc at
#t gcg tgg agt gtc ctg 144
Tyr Cys Thr Lys Tyr Glu Pro Trp Gln Leu Il
#e Ala Trp Ser Val Leu
                  35
# 40
# 45
tgt act ctg ctg ata gtc tgg gtg tat gag ct
#t atc ttc cag cca gag 192
Cys Thr Leu Leu Ile Val Trp Val Tyr Glu Le
#u Ile Phe Gln Pro Glu
          50
# 55
# 60
agt tta tgg tct cgg ttt aaa aaa aaa tta tt
#t aag ctt atc agg aag 240
Ser Leu Trp Ser Arg Phe Lys Lys Lys Leu Ph
#e Lys Leu Ile Arg Lys
  65
# 70
# 75
# 80
atg cca ttt att gga cgt aag atc gaa caa ca
#g gtg agc aaa gcc aag 288
Met Pro Phe Ile Gly Arg Lys Ile Glu Gln Gl
#n Val Ser Lys Ala Lys
                                  85
# 90
# 95
aag gat ctt gtc aag aac atg cca ttc cta aa
#g gtg gac aag gat tat 336
Lys Asp Leu Val Lys Asn Met Pro Phe Leu Ly
#s Val Asp Lys Asp Tyr
                        100
# 105
# 110
gtg aaa act ctg cct gct cag ggt atg ggc ac
#a gct gag gtt ctg gag 384
Val Lys Thr Leu Pro Ala Gln Gly Met Gly Th
#r Ala Glu Val Leu Glu
                115
# 120
# 125
aga ctc aag gag tac agc tcc atg gat ggt tc
#c tgg caa gaa ggg aaa 432
Arg Leu Lys Glu Tyr Ser Ser Met Asp Gly Se
#r Trp Gln Glu Gly Lys
        130
# 135
# 140
gcc tca gga gct gtg tac aat ggg gaa ccg aa
#g ctc acg gag ctg ctg 480
Ala Ser Gly Ala Val Tyr Asn Gly Glu Pro Ly
#s Leu Thr Glu Leu Leu
145 1
#50 1
#55 1
#60
gtg cag gct tat gga gaa ttc acg tgg agc aa
#t cca ctg cat cca gat 528
Val Gln Ala Tyr Gly Glu Phe Thr Trp Ser As
#n Pro Leu His Pro Asp
                                165
# 170
# 175
atc ttc cct gga ttg cgg aag tta gag gca ga
#a atc gtt agg atg act 576
Ile Phe Pro Gly Leu Arg Lys Leu Glu Ala Gl
#u Ile Val Arg Met Thr
                        180
# 185
# 190
tgt tcc ctc ttc aat ggg gga cca gat tcc tg
#t gga tgt gtg act tct 624
Cys Ser Leu Phe Asn Gly Gly Pro Asp Ser Cy
#s Gly Cys Val Thr Ser
                195
# 200
# 205
ggg gga acg gaa agc atc ctg atg gcc tgc aa
#a gct tac cgg gac ttg 672
Gly Gly Thr Glu Ser Ile Leu Met Ala Cys Ly
#s Ala Tyr Arg Asp Leu
        210
# 215
# 220
gcg tta gag aag ggg atc aaa act cca gaa at
#t gtg gct ccc gag agt 720
Ala Leu Glu Lys Gly Ile Lys Thr Pro Glu Il
#e Val Ala Pro Glu Ser
225 2
#30 2
#35 2
#40
gcc cat gct gca ttc gac aaa gca gct cat ta
#t ttt ggg atg aag att 768
Ala His Ala Ala Phe Asp Lys Ala Ala His Ty
#r Phe Gly Met Lys Ile
                                245
# 250
# 255
gtc cga gtt gca ctg aaa aag aac atg gag gt
#g gat gtg cag gca atg 816
Val Arg Val Ala Leu Lys Lys Asn Met Glu Va
#l Asp Val Gln Ala Met
                        260
# 265
# 270
aag aga gcc atc tcc agg aac aca gct atg ct
#g gtc tgt tct acc cca 864
Lys Arg Ala Ile Ser Arg Asn Thr Ala Met Le
#u Val Cys Ser Thr Pro
                275
# 280
# 285
cag ttt cct cat ggt gtg atg gat cct gtc cc
#c gaa gtg gcc aag tta 912
Gln Phe Pro His Gly Val Met Asp Pro Val Pr
#o Glu Val Ala Lys Leu
        290
# 295
# 300
act gtc aga tat aaa atc cca ctc cat gtg ga
#t gct tgt ctg ggg ggc 960
Thr Val Arg Tyr Lys Ile Pro Leu His Val As
#p Ala Cys Leu Gly Gly
305 3
#10 3
#15 3
#20
ttc ctc att gtc ttc atg gag aaa gca ggg ta
#c cca ctg gag aaa cca 1008
Phe Leu Ile Val Phe Met Glu Lys Ala Gly Ty
#r Pro Leu Glu Lys Pro
                                325
# 330
# 335
ttt gat ttc cgg gtg aaa ggt gtg acc agc at
#t tca gca gat act cat 1056
Phe Asp Phe Arg Val Lys Gly Val Thr Ser Il
#e Ser Ala Asp Thr His
                        340
# 345
# 350
aag tat ggc tat gct cct aaa ggt tca tca gt
#g gtg atg tac tct aac 1104
Lys Tyr Gly Tyr Ala Pro Lys Gly Ser Ser Va
#l Val Met Tyr Ser Asn
                355
# 360
# 365
gag aag tac agg acg tac cag ttc ttt gtt gg
#t gca gac tgg caa ggt 1152
Glu Lys Tyr Arg Thr Tyr Gln Phe Phe Val Gl
#y Ala Asp Trp Gln Gly
        370
# 375
# 380
ggt gtc tac gca tct cca agc ata gct ggc tc
#a cgg cct ggt ggc atc 1200
Gly Val Tyr Ala Ser Pro Ser Ile Ala Gly Se
#r Arg Pro Gly Gly Ile
385 3
#90 3
#95 4
#00
att gca gcc tgt tgg gcg gcc ttg atg cac tt
#c ggt gag aac ggc tat 1248
Ile Ala Ala Cys Trp Ala Ala Leu Met His Ph
#e Gly Glu Asn Gly Tyr
                                405
# 410
# 415
gtt gaa gct acc aaa cag atc atc aaa act gc
#t cgc ttc ctg aag tca 1296
Val Glu Ala Thr Lys Gln Ile Ile Lys Thr Al
#a Arg Phe Leu Lys Ser
                        420
# 425
# 430
gaa ctg gaa aac atc aaa aac atc ttc att tt
#c ggt gat cct caa ttg 1344
Glu Leu Glu Asn Ile Lys Asn Ile Phe Ile Ph
#e Gly Asp Pro Gln Leu
                435
# 440
# 445
tca gtt att gct ctg gga tcc aac gat ttt ga
#c att tac cga cta tct 1392
Ser Val Ile Ala Leu Gly Ser Asn Asp Phe As
#p Ile Tyr Arg Leu Ser
        450
# 455
# 460
aat atg atg tct gct aag ggg tgg aat ttt aa
#c tac ctg cag ttc cca 1440
Asn Met Met Ser Ala Lys Gly Trp Asn Phe As
#n Tyr Leu Gln Phe Pro
465 4
#70 4
#75 4
#80
aga agc att cat ttc tgc att acg tta gta ca
#t act cgg aag cga gtg 1488
Arg Ser Ile His Phe Cys Ile Thr Leu Val Hi
#s Thr Arg Lys Arg Val
                                485
# 490
# 495
gcg atc cag ttc cta aag gat atc cgg gaa tc
#a gtc aca caa atc atg 1536
Ala Ile Gln Phe Leu Lys Asp Ile Arg Glu Se
#r Val Thr Gln Ile Met
                        500
# 505
# 510
aag aat cct aaa gct aag acc aca gga atg gg
#t gcc atc tat ggc atg 1584
Lys Asn Pro Lys Ala Lys Thr Thr Gly Met Gl
#y Ala Ile Tyr Gly Met
                515
# 520
# 525
gcc cag gca acc att gac agg aag ctg gtt gc
#a gaa ata tcc tcc gtc 1632
Ala Gln Ala Thr Ile Asp Arg Lys Leu Val Al
#a Glu Ile Ser Ser Val
        530
# 535
# 540
ttc ttg gac tgc ctt tat act acg gac ccc gt
#g act cag ggc aac cag 1680
Phe Leu Asp Cys Leu Tyr Thr Thr Asp Pro Va
#l Thr Gln Gly Asn Gln
545 5
#50 5
#55 5
#60
atg aac ggt tct cca aag ccc cgc tga
#
# 1707
Met Asn Gly Ser Pro Lys Pro Arg *
                                565
<210> SEQ ID NO 6
<211> LENGTH: 568
<212> TYPE: PRT
<213> ORGANISM: Mus musculus
<400> SEQUENCE: 6
Met Pro Gly Thr Asp Leu Leu Lys Leu Lys As
#p Phe Glu Pro Tyr Leu
  1 5
# 10
# 15
Glu Ile Leu Glu Ser Tyr Ser Thr Lys Ala Ly
#s Asn Tyr Val Asn Gly
                        20
# 25
# 30
Tyr Cys Thr Lys Tyr Glu Pro Trp Gln Leu Il
#e Ala Trp Ser Val Leu
                35
# 40
# 45
Cys Thr Leu Leu Ile Val Trp Val Tyr Glu Le
#u Ile Phe Gln Pro Glu
        50
# 55
# 60
Ser Leu Trp Ser Arg Phe Lys Lys Lys Leu Ph
#e Lys Leu Ile Arg Lys
65
#70
#75
#80
Met Pro Phe Ile Gly Arg Lys Ile Glu Gln Gl
#n Val Ser Lys Ala Lys
                                85
# 90
# 95
Lys Asp Leu Val Lys Asn Met Pro Phe Leu Ly
#s Val Asp Lys Asp Tyr
                        100
# 105
# 110
Val Lys Thr Leu Pro Ala Gln Gly Met Gly Th
#r Ala Glu Val Leu Glu
                115
# 120
# 125
Arg Leu Lys Glu Tyr Ser Ser Met Asp Gly Se
#r Trp Gln Glu Gly Lys
        130
# 135
# 140
Ala Ser Gly Ala Val Tyr Asn Gly Glu Pro Ly
#s Leu Thr Glu Leu Leu
145 1
#50 1
#55 1
#60
Val Gln Ala Tyr Gly Glu Phe Thr Trp Ser As
#n Pro Leu His Pro Asp
                                165
# 170
# 175
Ile Phe Pro Gly Leu Arg Lys Leu Glu Ala Gl
#u Ile Val Arg Met Thr
                        180
# 185
# 190
Cys Ser Leu Phe Asn Gly Gly Pro Asp Ser Cy
#s Gly Cys Val Thr Ser
                195
# 200
# 205
Gly Gly Thr Glu Ser Ile Leu Met Ala Cys Ly
#s Ala Tyr Arg Asp Leu
        210
# 215
# 220
Ala Leu Glu Lys Gly Ile Lys Thr Pro Glu Il
#e Val Ala Pro Glu Ser
225 2
#30 2
#35 2
#40
Ala His Ala Ala Phe Asp Lys Ala Ala His Ty
#r Phe Gly Met Lys Ile
                                245
# 250
# 255
Val Arg Val Ala Leu Lys Lys Asn Met Glu Va
#l Asp Val Gln Ala Met
                        260
# 265
# 270
Lys Arg Ala Ile Ser Arg Asn Thr Ala Met Le
#u Val Cys Ser Thr Pro
                275
# 280
# 285
Gln Phe Pro His Gly Val Met Asp Pro Val Pr
#o Glu Val Ala Lys Leu
        290
# 295
# 300
Thr Val Arg Tyr Lys Ile Pro Leu His Val As
#p Ala Cys Leu Gly Gly
305 3
#10 3
#15 3
#20
Phe Leu Ile Val Phe Met Glu Lys Ala Gly Ty
#r Pro Leu Glu Lys Pro
                                325
# 330
# 335
Phe Asp Phe Arg Val Lys Gly Val Thr Ser Il
#e Ser Ala Asp Thr His
                        340
# 345
# 350
Lys Tyr Gly Tyr Ala Pro Lys Gly Ser Ser Va
#l Val Met Tyr Ser Asn
                355
# 360
# 365
Glu Lys Tyr Arg Thr Tyr Gln Phe Phe Val Gl
#y Ala Asp Trp Gln Gly
        370
# 375
# 380
Gly Val Tyr Ala Ser Pro Ser Ile Ala Gly Se
#r Arg Pro Gly Gly Ile
385 3
#90 3
#95 4
#00
Ile Ala Ala Cys Trp Ala Ala Leu Met His Ph
#e Gly Glu Asn Gly Tyr
                                405
# 410
# 415
Val Glu Ala Thr Lys Gln Ile Ile Lys Thr Al
#a Arg Phe Leu Lys Ser
                        420
# 425
# 430
Glu Leu Glu Asn Ile Lys Asn Ile Phe Ile Ph
#e Gly Asp Pro Gln Leu
                435
# 440
# 445
Ser Val Ile Ala Leu Gly Ser Asn Asp Phe As
#p Ile Tyr Arg Leu Ser
        450
# 455
# 460
Asn Met Met Ser Ala Lys Gly Trp Asn Phe As
#n Tyr Leu Gln Phe Pro
465 4
#70 4
#75 4
#80
Arg Ser Ile His Phe Cys Ile Thr Leu Val Hi
#s Thr Arg Lys Arg Val
                                485
# 490
# 495
Ala Ile Gln Phe Leu Lys Asp Ile Arg Glu Se
#r Val Thr Gln Ile Met
                        500
# 505
# 510
Lys Asn Pro Lys Ala Lys Thr Thr Gly Met Gl
#y Ala Ile Tyr Gly Met
                515
# 520
# 525
Ala Gln Ala Thr Ile Asp Arg Lys Leu Val Al
#a Glu Ile Ser Ser Val
        530
# 535
# 540
Phe Leu Asp Cys Leu Tyr Thr Thr Asp Pro Va
#l Thr Gln Gly Asn Gln
545 5
#50 5
#55 5
#60
Met Asn Gly Ser Pro Lys Pro Arg
                                565
<210> SEQ ID NO 7
<211> LENGTH: 1707
<212> TYPE: DNA
<213> ORGANISM: Homo sapiens
<220> FEATURE:
<221> NAME/KEY: CDS
<222> LOCATION: (1)...(1707)
<400> SEQUENCE: 7
atg cct agc aca gac ctt ctg atg ttg aag gc
#c ttt gag ccc tac tta 48
Met Pro Ser Thr Asp Leu Leu Met Leu Lys Al
#a Phe Glu Pro Tyr Leu
  1 5
# 10
# 15
gag att ttg gaa gta tac tcc aca aaa gcc aa
#g aat tat gta aat gga 96
Glu Ile Leu Glu Val Tyr Ser Thr Lys Ala Ly
#s Asn Tyr Val Asn Gly
                          20
# 25
# 30
cat tgc acc aag tat gag ccc tgg cag cta at
#t gca tgg agt gtc gtg 144
His Cys Thr Lys Tyr Glu Pro Trp Gln Leu Il
#e Ala Trp Ser Val Val
                  35
# 40
# 45
tgg acc ctg ctg ata gtc tgg gga tat gag tt
#t gtc ttc cag cca gag 192
Trp Thr Leu Leu Ile Val Trp Gly Tyr Glu Ph
#e Val Phe Gln Pro Glu
          50
# 55
# 60
agt tta tgg tca agg ttt aaa aag aaa tgt tt
#t aag ctc acc agg aag 240
Ser Leu Trp Ser Arg Phe Lys Lys Lys Cys Ph
#e Lys Leu Thr Arg Lys
  65
# 70
# 75
# 80
atg ccc att att ggt cgt aag att caa gac aa
#g ttg aac aag acc aag 288
Met Pro Ile Ile Gly Arg Lys Ile Gln Asp Ly
#s Leu Asn Lys Thr Lys
                                  85
# 90
# 95
gat gat att agc aag aac atg tca ttc ctg aa
#a gtg gac aaa gag tat 336
Asp Asp Ile Ser Lys Asn Met Ser Phe Leu Ly
#s Val Asp Lys Glu Tyr
                        100
# 105
# 110
gtg aaa gct tta ccc tcc cag ggt ctg agc tc
#a tct gct gtt ttg gag 384
Val Lys Ala Leu Pro Ser Gln Gly Leu Ser Se
#r Ser Ala Val Leu Glu
                115
# 120
# 125
aaa ctt aag gag tac agc tct atg gac gcc tt
#c tgg caa gag ggg aga 432
Lys Leu Lys Glu Tyr Ser Ser Met Asp Ala Ph
#e Trp Gln Glu Gly Arg
        130
# 135
# 140
gcc tct gga aca gtg tac agt ggg gag gag aa
#g ctc act gag ctc ctt 480
Ala Ser Gly Thr Val Tyr Ser Gly Glu Glu Ly
#s Leu Thr Glu Leu Leu
145 1
#50 1
#55 1
#60
gtg aag gct tat gga gat ttt gca tgg agt aa
#c ccc ctg cat cca gat 528
Val Lys Ala Tyr Gly Asp Phe Ala Trp Ser As
#n Pro Leu His Pro Asp
                                165
# 170
# 175
atc ttc cca gga cta cgc aag ata gag gca ga
#a att gtg agg ata gct 576
Ile Phe Pro Gly Leu Arg Lys Ile Glu Ala Gl
#u Ile Val Arg Ile Ala
                        180
# 185
# 190
tgt tcc ctg ttc aat ggg gga cca gat tcg tg
#t gga tgt gtg act tct 624
Cys Ser Leu Phe Asn Gly Gly Pro Asp Ser Cy
#s Gly Cys Val Thr Ser
                195
# 200
# 205
ggg gga aca gaa agc ata ctc atg gcc tgc aa
#a gca tgt cgg gat ctg 672
Gly Gly Thr Glu Ser Ile Leu Met Ala Cys Ly
#s Ala Cys Arg Asp Leu
        210
# 215
# 220
gcc ttt gag aag ggg atc aaa act cca gaa at
#t gtg gct ccc caa agt 720
Ala Phe Glu Lys Gly Ile Lys Thr Pro Glu Il
#e Val Ala Pro Gln Ser
225 2
#30 2
#35 2
#40
gcc cat gct gca ttt aac aaa gca gcc agt ta
#c ttt ggg atg aag att 768
Ala His Ala Ala Phe Asn Lys Ala Ala Ser Ty
#r Phe Gly Met Lys Ile
                                245
# 250
# 255
gtg cgg gtc cca ttg acg aag atg atg gag gt
#g gat gtg agg gca atg 816
Val Arg Val Pro Leu Thr Lys Met Met Glu Va
#l Asp Val Arg Ala Met
                        260
# 265
# 270
aga aga gct atc tcc agg aac act gcc atg ct
#c gtc tgt tct acc cca 864
Arg Arg Ala Ile Ser Arg Asn Thr Ala Met Le
#u Val Cys Ser Thr Pro
                275
# 280
# 285
cag ttt cct cat ggt gta ata gat cct gtc cc
#t gaa gtg gcc aag ctg 912
Gln Phe Pro His Gly Val Ile Asp Pro Val Pr
#o Glu Val Ala Lys Leu
        290
# 295
# 300
gct gtc aaa tac aaa ata ccc ctt cat gtc ga
#c gct tgt ctg gga ggc 960
Ala Val Lys Tyr Lys Ile Pro Leu His Val As
#p Ala Cys Leu Gly Gly
305 3
#10 3
#15 3
#20
ttc ctc atc gtc ttt atg gag aaa gca gga ta
#c cca ctg gag cac cca 1008
Phe Leu Ile Val Phe Met Glu Lys Ala Gly Ty
#r Pro Leu Glu His Pro
                                325
# 330
# 335
ttt gat ttc cgg gtg aaa ggt gta acc agc at
#t tca gct gac acc cat 1056
Phe Asp Phe Arg Val Lys Gly Val Thr Ser Il
#e Ser Ala Asp Thr His
                        340
# 345
# 350
aag tat ggc tat gcc cca aaa ggc tca tca tt
#g gtg ttg tat agt gac 1104
Lys Tyr Gly Tyr Ala Pro Lys Gly Ser Ser Le
#u Val Leu Tyr Ser Asp
                355
# 360
# 365
aag aag tac agg aac tat cag ttc ttc gtc ga
#t aca gat tgg cag ggt 1152
Lys Lys Tyr Arg Asn Tyr Gln Phe Phe Val As
#p Thr Asp Trp Gln Gly
        370
# 375
# 380
ggc atc tat gct tcc cca acc atc gca ggc tc
#a cgg cct ggt ggc att 1200
Gly Ile Tyr Ala Ser Pro Thr Ile Ala Gly Se
#r Arg Pro Gly Gly Ile
385 3
#90 3
#95 4
#00
agc gca gcc tgt tgg gct gcc ttg atg cac tt
#c ggt gag aac ggc tat 1248
Ser Ala Ala Cys Trp Ala Ala Leu Met His Ph
#e Gly Glu Asn Gly Tyr
                                405
# 410
# 415
gtt gaa gct acc aaa cag atc atc aaa act gc
#t cgc ttc ctc aag tca 1296
Val Glu Ala Thr Lys Gln Ile Ile Lys Thr Al
#a Arg Phe Leu Lys Ser
                        420
# 425
# 430
gaa ctg gaa aat atc aaa ggc atc ttt gtt tt
#t ggg aat ccc caa ttg 1344
Glu Leu Glu Asn Ile Lys Gly Ile Phe Val Ph
#e Gly Asn Pro Gln Leu
                435
# 440
# 445
tca ctc att gct ctg gga tcc cgt gat ttt ga
#c atc tac cga cta tca 1392
Ser Leu Ile Ala Leu Gly Ser Arg Asp Phe As
#p Ile Tyr Arg Leu Ser
        450
# 455
# 460
aac ctg atg act gct aag ggg tgg aac ttg aa
#c cag ttg cag ttc cca 1440
Asn Leu Met Thr Ala Lys Gly Trp Asn Leu As
#n Gln Leu Gln Phe Pro
465 4
#70 4
#75 4
#80
ccc agt att cat ttc tgc atc aca tta cta ca
#c gcc cgg aaa cga gta 1488
Pro Ser Ile His Phe Cys Ile Thr Leu Leu Hi
#s Ala Arg Lys Arg Val
                                485
# 490
# 495
gct ata caa ttc cta aag gac att cga gaa tc
#t gtc act caa atc atg 1536
Ala Ile Gln Phe Leu Lys Asp Ile Arg Glu Se
#r Val Thr Gln Ile Met
                        500
# 505
# 510
aag aat cct aaa gcg aag acc aca gga atg gg
#t gcc atc tat gcc atg 1584
Lys Asn Pro Lys Ala Lys Thr Thr Gly Met Gl
#y Ala Ile Tyr Ala Met
                515
# 520
# 525
gcc cag aca act gtt gac agg aat atg gtt gc
#a gaa ttg tcc tca gtc 1632
Ala Gln Thr Thr Val Asp Arg Asn Met Val Al
#a Glu Leu Ser Ser Val
        530
# 535
# 540
ttc ttg gac agc ttg tac agc acc gac act gt
#c acc cag ggc agc cag 1680
Phe Leu Asp Ser Leu Tyr Ser Thr Asp Thr Va
#l Thr Gln Gly Ser Gln
545 5
#50 5
#55 5
#60
atg aat ggt tct cca aaa ccc cac tga
#
# 1707
Met Asn Gly Ser Pro Lys Pro His *
                                565
<210> SEQ ID NO 8
<211> LENGTH: 568
<212> TYPE: PRT
<213> ORGANISM: Homo sapiens
<400> SEQUENCE: 8
Met Pro Ser Thr Asp Leu Leu Met Leu Lys Al
#a Phe Glu Pro Tyr Leu
  1 5
# 10
# 15
Glu Ile Leu Glu Val Tyr Ser Thr Lys Ala Ly
#s Asn Tyr Val Asn Gly
                        20
# 25
# 30
His Cys Thr Lys Tyr Glu Pro Trp Gln Leu Il
#e Ala Trp Ser Val Val
                35
# 40
# 45
Trp Thr Leu Leu Ile Val Trp Gly Tyr Glu Ph
#e Val Phe Gln Pro Glu
        50
# 55
# 60
Ser Leu Trp Ser Arg Phe Lys Lys Lys Cys Ph
#e Lys Leu Thr Arg Lys
65
#70
#75
#80
Met Pro Ile Ile Gly Arg Lys Ile Gln Asp Ly
#s Leu Asn Lys Thr Lys
                                85
# 90
# 95
Asp Asp Ile Ser Lys Asn Met Ser Phe Leu Ly
#s Val Asp Lys Glu Tyr
                        100
# 105
# 110
Val Lys Ala Leu Pro Ser Gln Gly Leu Ser Se
#r Ser Ala Val Leu Glu
                115
# 120
# 125
Lys Leu Lys Glu Tyr Ser Ser Met Asp Ala Ph
#e Trp Gln Glu Gly Arg
        130
# 135
# 140
Ala Ser Gly Thr Val Tyr Ser Gly Glu Glu Ly
#s Leu Thr Glu Leu Leu
145 1
#50 1
#55 1
#60
Val Lys Ala Tyr Gly Asp Phe Ala Trp Ser As
#n Pro Leu His Pro Asp
                                165
# 170
# 175
Ile Phe Pro Gly Leu Arg Lys Ile Glu Ala Gl
#u Ile Val Arg Ile Ala
                        180
# 185
# 190
Cys Ser Leu Phe Asn Gly Gly Pro Asp Ser Cy
#s Gly Cys Val Thr Ser
                195
# 200
# 205
Gly Gly Thr Glu Ser Ile Leu Met Ala Cys Ly
#s Ala Cys Arg Asp Leu
        210
# 215
# 220
Ala Phe Glu Lys Gly Ile Lys Thr Pro Glu Il
#e Val Ala Pro Gln Ser
225 2
#30 2
#35 2
#40
Ala His Ala Ala Phe Asn Lys Ala Ala Ser Ty
#r Phe Gly Met Lys Ile
                                245
# 250
# 255
Val Arg Val Pro Leu Thr Lys Met Met Glu Va
#l Asp Val Arg Ala Met
                        260
# 265
# 270
Arg Arg Ala Ile Ser Arg Asn Thr Ala Met Le
#u Val Cys Ser Thr Pro
                275
# 280
# 285
Gln Phe Pro His Gly Val Ile Asp Pro Val Pr
#o Glu Val Ala Lys Leu
        290
# 295
# 300
Ala Val Lys Tyr Lys Ile Pro Leu His Val As
#p Ala Cys Leu Gly Gly
305 3
#10 3
#15 3
#20
Phe Leu Ile Val Phe Met Glu Lys Ala Gly Ty
#r Pro Leu Glu His Pro
                                325
# 330
# 335
Phe Asp Phe Arg Val Lys Gly Val Thr Ser Il
#e Ser Ala Asp Thr His
                        340
# 345
# 350
Lys Tyr Gly Tyr Ala Pro Lys Gly Ser Ser Le
#u Val Leu Tyr Ser Asp
                355
# 360
# 365
Lys Lys Tyr Arg Asn Tyr Gln Phe Phe Val As
#p Thr Asp Trp Gln Gly
        370
# 375
# 380
Gly Ile Tyr Ala Ser Pro Thr Ile Ala Gly Se
#r Arg Pro Gly Gly Ile
385 3
#90 3
#95 4
#00
Ser Ala Ala Cys Trp Ala Ala Leu Met His Ph
#e Gly Glu Asn Gly Tyr
                                405
# 410
# 415
Val Glu Ala Thr Lys Gln Ile Ile Lys Thr Al
#a Arg Phe Leu Lys Ser
                        420
# 425
# 430
Glu Leu Glu Asn Ile Lys Gly Ile Phe Val Ph
#e Gly Asn Pro Gln Leu
                435
# 440
# 445
Ser Leu Ile Ala Leu Gly Ser Arg Asp Phe As
#p Ile Tyr Arg Leu Ser
        450
# 455
# 460
Asn Leu Met Thr Ala Lys Gly Trp Asn Leu As
#n Gln Leu Gln Phe Pro
465 4
#70 4
#75 4
#80
Pro Ser Ile His Phe Cys Ile Thr Leu Leu Hi
#s Ala Arg Lys Arg Val
                                485
# 490
# 495
Ala Ile Gln Phe Leu Lys Asp Ile Arg Glu Se
#r Val Thr Gln Ile Met
                        500
# 505
# 510
Lys Asn Pro Lys Ala Lys Thr Thr Gly Met Gl
#y Ala Ile Tyr Ala Met
                515
# 520
# 525
Ala Gln Thr Thr Val Asp Arg Asn Met Val Al
#a Glu Leu Ser Ser Val
        530
# 535
# 540
Phe Leu Asp Ser Leu Tyr Ser Thr Asp Thr Va
#l Thr Gln Gly Ser Gln
545 5
#50 5
#55 5
#60
Met Asn Gly Ser Pro Lys Pro His
                                565
<210> SEQ ID NO 9
<211> LENGTH: 1467
<212> TYPE: DNA
<213> ORGANISM: Homo sapiens
<220> FEATURE:
<221> NAME/KEY: CDS
<222> LOCATION: (1)...(1467)
<400> SEQUENCE: 9
atg cct agc aca gac ctt ctg atg ttg aag gc
#c ttt gag ccc tac tta 48
Met Pro Ser Thr Asp Leu Leu Met Leu Lys Al
#a Phe Glu Pro Tyr Leu
  1 5
# 10
# 15
gag att ttg gaa gta tac tcc aca aaa gcc aa
#g aat tat gta aat gga 96
Glu Ile Leu Glu Val Tyr Ser Thr Lys Ala Ly
#s Asn Tyr Val Asn Gly
                          20
# 25
# 30
cat tgc acc aag tat gag ccc tgg cag cta at
#t gca tgg agt gtc gtg 144
His Cys Thr Lys Tyr Glu Pro Trp Gln Leu Il
#e Ala Trp Ser Val Val
                  35
# 40
# 45
tgg acc ctg ctg ata gtc tgg gga tat gag tt
#t gtc ttc cag cca gag 192
Trp Thr Leu Leu Ile Val Trp Gly Tyr Glu Ph
#e Val Phe Gln Pro Glu
          50
# 55
# 60
agt tta tgg tca agg ttt aaa aag aaa tgt tt
#t aag ctc acc agg aag 240
Ser Leu Trp Ser Arg Phe Lys Lys Lys Cys Ph
#e Lys Leu Thr Arg Lys
  65
# 70
# 75
# 80
atg ccc att att ggt cgt aag att caa gac aa
#g ttg aac aag acc aag 288
Met Pro Ile Ile Gly Arg Lys Ile Gln Asp Ly
#s Leu Asn Lys Thr Lys
                                  85
# 90
# 95
gat gat att agc aag aac atg tca ttc ctg aa
#a gtg gac aaa gag tat 336
Asp Asp Ile Ser Lys Asn Met Ser Phe Leu Ly
#s Val Asp Lys Glu Tyr
                        100
# 105
# 110
gtg aaa gct tta ccc tcc cag ggt ctg agc tc
#a tct gct gtt ttg gag 384
Val Lys Ala Leu Pro Ser Gln Gly Leu Ser Se
#r Ser Ala Val Leu Glu
                115
# 120
# 125
aaa ctt aag gag tac agc tct atg gac gcc tt
#c tgg caa gag ggg aga 432
Lys Leu Lys Glu Tyr Ser Ser Met Asp Ala Ph
#e Trp Gln Glu Gly Arg
        130
# 135
# 140
gcc tct gga aca gtg tac agt ggg gag gag aa
#g ctc act gag ctc ctt 480
Ala Ser Gly Thr Val Tyr Ser Gly Glu Glu Ly
#s Leu Thr Glu Leu Leu
145 1
#50 1
#55 1
#60
gtg aag gct tat gga gat ttt gca tgg agt aa
#c ccc ctg cat cca gat 528
Val Lys Ala Tyr Gly Asp Phe Ala Trp Ser As
#n Pro Leu His Pro Asp
                                165
# 170
# 175
atc ttc cca gga cta cgc aag ata gag gca ga
#a att gtg agg ata gct 576
Ile Phe Pro Gly Leu Arg Lys Ile Glu Ala Gl
#u Ile Val Arg Ile Ala
                        180
# 185
# 190
tgt tcc ctg ttc aat ggg gga cca gat tcg tg
#t gga tgt gtg act tct 624
Cys Ser Leu Phe Asn Gly Gly Pro Asp Ser Cy
#s Gly Cys Val Thr Ser
                195
# 200
# 205
ggg gga aca gaa agc ata ctc atg gcc tgc aa
#a gca tgt cgg gat ctg 672
Gly Gly Thr Glu Ser Ile Leu Met Ala Cys Ly
#s Ala Cys Arg Asp Leu
        210
# 215
# 220
gcc ttt gag aag ggg atc aaa act cca gaa at
#t gtg gct ccc caa agt 720
Ala Phe Glu Lys Gly Ile Lys Thr Pro Glu Il
#e Val Ala Pro Gln Ser
225 2
#30 2
#35 2
#40
gcc cat gct gca ttt aac aaa gca gcc agt ta
#c ttt ggg atg aag att 768
Ala His Ala Ala Phe Asn Lys Ala Ala Ser Ty
#r Phe Gly Met Lys Ile
                                245
# 250
# 255
gtg cgg gtc cca ttg acg aag atg atg gag gt
#g gat gtg agg gca atg 816
Val Arg Val Pro Leu Thr Lys Met Met Glu Va
#l Asp Val Arg Ala Met
                        260
# 265
# 270
aga aga gct atc tcc agg aac act gcc atg ct
#c gtc tgt tct acc cca 864
Arg Arg Ala Ile Ser Arg Asn Thr Ala Met Le
#u Val Cys Ser Thr Pro
                275
# 280
# 285
cag ttt cct cat ggt gta ata gat cct gtc cc
#t gaa gtg gcc aag ctg 912
Gln Phe Pro His Gly Val Ile Asp Pro Val Pr
#o Glu Val Ala Lys Leu
        290
# 295
# 300
gct gtc aaa tac aaa ata ccc ctt cat gtc ga
#c gct tgt ctg gga ggc 960
Ala Val Lys Tyr Lys Ile Pro Leu His Val As
#p Ala Cys Leu Gly Gly
305 3
#10 3
#15 3
#20
ttc ctc atc gtc ttt atg gag aaa gca gga ta
#c cca ctg gag cac cca 1008
Phe Leu Ile Val Phe Met Glu Lys Ala Gly Ty
#r Pro Leu Glu His Pro
                                325
# 330
# 335
ttt gat ttc cgg gtg aaa ggt gta acc agc at
#t tca gct gac acc cat 1056
Phe Asp Phe Arg Val Lys Gly Val Thr Ser Il
#e Ser Ala Asp Thr His
                        340
# 345
# 350
aag ctg gaa aat atc aaa ggc atc ttt gtt tt
#t ggg aat ccc caa ttg 1104
Lys Leu Glu Asn Ile Lys Gly Ile Phe Val Ph
#e Gly Asn Pro Gln Leu
                355
# 360
# 365
tca ctc att gct ctg gga tcc cgt gat ttt ga
#c atc tac cga cta tca 1152
Ser Leu Ile Ala Leu Gly Ser Arg Asp Phe As
#p Ile Tyr Arg Leu Ser
        370
# 375
# 380
aac ctg atg act gct aag ggg tgg aac ttg aa
#c cag ttg cag ttc cca 1200
Asn Leu Met Thr Ala Lys Gly Trp Asn Leu As
#n Gln Leu Gln Phe Pro
385 3
#90 3
#95 4
#00
ccc agt att cat ttc tgc atc aca tta cta ca
#c gcc cgg aaa cga gta 1248
Pro Ser Ile His Phe Cys Ile Thr Leu Leu Hi
#s Ala Arg Lys Arg Val
                                405
# 410
# 415
gct ata caa ttc cta aag gac att cga gaa tc
#t gtc act caa atc atg 1296
Ala Ile Gln Phe Leu Lys Asp Ile Arg Glu Se
#r Val Thr Gln Ile Met
                        420
# 425
# 430
aag aat cct aaa gcg aag acc aca gga atg gg
#t gcc atc tat gcc atg 1344
Lys Asn Pro Lys Ala Lys Thr Thr Gly Met Gl
#y Ala Ile Tyr Ala Met
                435
# 440
# 445
gcc cag aca act gtt gac agg aat atg gtt gc
#a gaa ttg tcc tca gtc 1392
Ala Gln Thr Thr Val Asp Arg Asn Met Val Al
#a Glu Leu Ser Ser Val
        450
# 455
# 460
ttc ttg gac agc ttg tac agc acc gac act gt
#c acc cag ggc agc cag 1440
Phe Leu Asp Ser Leu Tyr Ser Thr Asp Thr Va
#l Thr Gln Gly Ser Gln
465 4
#70 4
#75 4
#80
atg aat ggt tct cca aaa ccc cac tga
#
# 1467
Met Asn Gly Ser Pro Lys Pro His *
                                485
<210> SEQ ID NO 10
<211> LENGTH: 488
<212> TYPE: PRT
<213> ORGANISM: Homo sapiens
<400> SEQUENCE: 10
Met Pro Ser Thr Asp Leu Leu Met Leu Lys Al
#a Phe Glu Pro Tyr Leu
  1 5
# 10
# 15
Glu Ile Leu Glu Val Tyr Ser Thr Lys Ala Ly
#s Asn Tyr Val Asn Gly
                        20
# 25
# 30
His Cys Thr Lys Tyr Glu Pro Trp Gln Leu Il
#e Ala Trp Ser Val Val
                35
# 40
# 45
Trp Thr Leu Leu Ile Val Trp Gly Tyr Glu Ph
#e Val Phe Gln Pro Glu
        50
# 55
# 60
Ser Leu Trp Ser Arg Phe Lys Lys Lys Cys Ph
#e Lys Leu Thr Arg Lys
65
#70
#75
#80
Met Pro Ile Ile Gly Arg Lys Ile Gln Asp Ly
#s Leu Asn Lys Thr Lys
                                85
# 90
# 95
Asp Asp Ile Ser Lys Asn Met Ser Phe Leu Ly
#s Val Asp Lys Glu Tyr
                        100
# 105
# 110
Val Lys Ala Leu Pro Ser Gln Gly Leu Ser Se
#r Ser Ala Val Leu Glu
                115
# 120
# 125
Lys Leu Lys Glu Tyr Ser Ser Met Asp Ala Ph
#e Trp Gln Glu Gly Arg
        130
# 135
# 140
Ala Ser Gly Thr Val Tyr Ser Gly Glu Glu Ly
#s Leu Thr Glu Leu Leu
145 1
#50 1
#55 1
#60
Val Lys Ala Tyr Gly Asp Phe Ala Trp Ser As
#n Pro Leu His Pro Asp
                                165
# 170
# 175
Ile Phe Pro Gly Leu Arg Lys Ile Glu Ala Gl
#u Ile Val Arg Ile Ala
                        180
# 185
# 190
Cys Ser Leu Phe Asn Gly Gly Pro Asp Ser Cy
#s Gly Cys Val Thr Ser
                195
# 200
# 205
Gly Gly Thr Glu Ser Ile Leu Met Ala Cys Ly
#s Ala Cys Arg Asp Leu
        210
# 215
# 220
Ala Phe Glu Lys Gly Ile Lys Thr Pro Glu Il
#e Val Ala Pro Gln Ser
225 2
#30 2
#35 2
#40
Ala His Ala Ala Phe Asn Lys Ala Ala Ser Ty
#r Phe Gly Met Lys Ile
                                245
# 250
# 255
Val Arg Val Pro Leu Thr Lys Met Met Glu Va
#l Asp Val Arg Ala Met
                        260
# 265
# 270
Arg Arg Ala Ile Ser Arg Asn Thr Ala Met Le
#u Val Cys Ser Thr Pro
                275
# 280
# 285
Gln Phe Pro His Gly Val Ile Asp Pro Val Pr
#o Glu Val Ala Lys Leu
        290
# 295
# 300
Ala Val Lys Tyr Lys Ile Pro Leu His Val As
#p Ala Cys Leu Gly Gly
305 3
#10 3
#15 3
#20
Phe Leu Ile Val Phe Met Glu Lys Ala Gly Ty
#r Pro Leu Glu His Pro
                                325
# 330
# 335
Phe Asp Phe Arg Val Lys Gly Val Thr Ser Il
#e Ser Ala Asp Thr His
                        340
# 345
# 350
Lys Leu Glu Asn Ile Lys Gly Ile Phe Val Ph
#e Gly Asn Pro Gln Leu
                355
# 360
# 365
Ser Leu Ile Ala Leu Gly Ser Arg Asp Phe As
#p Ile Tyr Arg Leu Ser
        370
# 375
# 380
Asn Leu Met Thr Ala Lys Gly Trp Asn Leu As
#n Gln Leu Gln Phe Pro
385 3
#90 3
#95 4
#00
Pro Ser Ile His Phe Cys Ile Thr Leu Leu Hi
#s Ala Arg Lys Arg Val
                                405
# 410
# 415
Ala Ile Gln Phe Leu Lys Asp Ile Arg Glu Se
#r Val Thr Gln Ile Met
                        420
# 425
# 430
Lys Asn Pro Lys Ala Lys Thr Thr Gly Met Gl
#y Ala Ile Tyr Ala Met
                435
# 440
# 445
Ala Gln Thr Thr Val Asp Arg Asn Met Val Al
#a Glu Leu Ser Ser Val
        450
# 455
# 460
Phe Leu Asp Ser Leu Tyr Ser Thr Asp Thr Va
#l Thr Gln Gly Ser Gln
465 4
#70 4
#75 4
#80
Met Asn Gly Ser Pro Lys Pro His
                                485
<210> SEQ ID NO 11
<211> LENGTH: 552
<212> TYPE: PRT
<213> ORGANISM: C. elegans
<400> SEQUENCE: 11
Met Asp Ser Val Lys His Thr Thr Glu Ile Il
#e Val Asp Leu Thr Lys
  1 5
# 10
# 15
Met His Tyr His Met Ile Asn Asp Arg Leu Se
#r Arg Tyr Asp Pro Val
                        20
# 25
# 30
Val Leu Val Leu Ala Ala Phe Gly Gly Thr Le
#u Val Tyr Thr Lys Val
                35
# 40
# 45
Val His Leu Tyr Arg Lys Ser Glu Asp Pro Il
#e Leu Lys Arg Met Gly
        50
# 55
# 60
Ala Tyr Val Phe Ser Leu Leu Arg Lys Leu Pr
#o Ala Val Arg Asp Lys
65
#70
#75
#80
Ile Glu Lys Glu Leu Ala Ala Glu Lys Pro Ly
#s Leu Ile Glu Ser Ile
                                85
# 90
# 95
His Lys Asp Asp Lys Asp Lys Gln Phe Ile Se
#r Thr Leu Pro Ile Ala
                        100
# 105
# 110
Pro Leu Ser Gln Asp Ser Ile Met Glu Leu Al
#a Lys Lys Tyr Glu Asp
                115
# 120
# 125
Tyr Asn Thr Phe Asn Ile Asp Gly Gly Arg Va
#l Ser Gly Ala Val Tyr
        130
# 135
# 140
Thr Asp Arg His Ala Glu His Ile Asn Leu Le
#u Gly Lys Ile Tyr Glu
145 1
#50 1
#55 1
#60
Lys Tyr Ala Phe Ser Asn Pro Leu His Pro As
#p Val Phe Pro Gly Ala
                                165
# 170
# 175
Arg Lys Met Glu Ala Glu Leu Ile Arg Met Va
#l Leu Asn Leu Tyr Asn
                        180
# 185
# 190
Gly Pro Glu Asp Ser Ser Gly Ser Val Thr Se
#r Gly Gly Thr Glu Ser
                195
# 200
# 205
Ile Ile Met Ala Cys Phe Ser Tyr Arg Asn Ar
#g Ala His Ser Leu Gly
        210
# 215
# 220
Ile Glu His Pro Val Ile Leu Ala Cys Lys Th
#r Ala His Ala Ala Phe
225 2
#30 2
#35 2
#40
Asp Lys Ala Ala His Leu Cys Gly Met Arg Le
#u Arg His Val Pro Val
                                245
# 250
# 255
Asp Ser Asp Asn Arg Val Asp Leu Lys Glu Me
#t Glu Arg Leu Ile Asp
                        260
# 265
# 270
Ser Asn Val Cys Met Leu Val Gly Ser Ala Pr
#o Asn Phe Pro Ser Gly
                275
# 280
# 285
Thr Ile Asp Pro Ile Pro Glu Ile Ala Lys Le
#u Gly Lys Lys Tyr Gly
        290
# 295
# 300
Ile Pro Val His Val Asp Ala Cys Leu Gly Gl
#y Phe Met Ile Pro Phe
305 3
#10 3
#15 3
#20
Met Asn Asp Ala Gly Tyr Leu Ile Pro Val Ph
#e Asp Phe Arg Asn Pro
                                325
# 330
# 335
Gly Val Thr Ser Ile Ser Cys Asp Thr His Ly
#s Tyr Gly Cys Thr Pro
                        340
# 345
# 350
Lys Gly Ser Ser Ile Val Met Tyr Arg Ser Ly
#s Glu Leu His His Phe
                355
# 360
# 365
Gln Tyr Phe Ser Val Ala Asp Trp Cys Gly Gl
#y Ile Tyr Ala Thr Pro
        370
# 375
# 380
Thr Ile Ala Gly Ser Arg Ala Gly Ala Asn Th
#r Ala Val Ala Trp Ala
385 3
#90 3
#95 4
#00
Thr Leu Leu Ser Phe Gly Arg Asp Glu Tyr Va
#l Arg Arg Cys Ala Gln
                                405
# 410
# 415
Ile Val Lys His Thr Arg Met Leu Ala Glu Ly
#s Ile Glu Lys Ile Lys
                        420
# 425
# 430
Trp Ile Lys Pro Tyr Gly Lys Ser Asp Val Se
#r Leu Val Ala Phe Ser
                435
# 440
# 445
Gly Asn Gly Val Asn Ile Tyr Glu Val Ser As
#p Lys Met Met Lys Leu
        450
# 455
# 460
Gly Trp Asn Leu Asn Thr Leu Gln Asn Pro Al
#a Ala Ile His Ile Cys
465 4
#70 4
#75 4
#80
Leu Thr Ile Asn Gln Ala Asn Glu Glu Val Va
#l Asn Ala Phe Ala Val
                                485
# 490
# 495
Asp Leu Glu Lys Ile Cys Glu Glu Leu Ala Al
#a Lys Gly Glu Gln Lys
                        500
# 505
# 510
Ala Asp Ser Gly Met Ala Ala Met Tyr Gly Me
#t Ala Ala Gln Val Pro
                515
# 520
# 525
Lys Ser Val Val Asp Glu Val Ile Ala Leu Ty
#r Ile Asp Ala Thr Tyr
        530
# 535
# 540
Ser Ala Pro Pro Ser Thr Ser Asn
545 5
#50
<210> SEQ ID NO 12
<211> LENGTH: 3162
<212> TYPE: DNA
<213> ORGANISM: C. elegans
<400> SEQUENCE: 12
atggattcgg ttaagcacac aaccgaaatt attgtcgact tgacaaaaat gc
#actatcac 60
atgataaatg ataggtgaat tttaaacaaa aattagatat ttggaaatta ct
#aattcaag 120
attttcagac tttctcggta tgatccggtt gttctagtgt tggccgcttt tg
#ggggtacc 180
cttgtctata caaaagtcgt ccatttgtac cgaaaaagcg aggatccaat tt
#tgaaacgg 240
caagtgtttt cttgcgaatt ttagaaatat caaaatgaaa ttttcagcat gg
#gagcttat 300
gtattctcac ttcttcgaaa acttccagct gttcgggata aaatcgaaaa ag
#agctggct 360
gctgagaagc caaagcttat tgaatcgatt cataaggatg ataaggacaa gc
#aattcatt 420
tccagtttgt ttgaacattt attaattaac caattcatta attctatttt tc
#agctcttc 480
ccatcgctcc attatctcag gactcaatta tggaactggc gaaaaaatat ga
#ggattaca 540
acacatttaa cattgacgga ggacgagtat ctggagcggt ttatactgat cg
#tcatgctg 600
aacacattaa tttgcttgga aaggtttaga aattctagaa tttttcaaaa tc
#ttagctct 660
caaatatatt ctcttgtaaa tagctacata gtatatcctg tagggaagct tt
#gaatccaa 720
ttcagatcag gggcgacaaa cgattttttc cggcaaatcg gcaaatcgcc gg
#aatggaaa 780
tttcctgcaa atcggcaaat tgccggaatg gaaatttcct gcaagttggc aa
#attgacgg 840
aattgaaatt tccggcaaac cgacaaattt ccgtaattaa aatttcctgc aa
#accggcga 900
attggcggaa ttgaaatttc ctgcaaaccg gcaaattgcc gtaattgaaa tt
#tcctgcaa 960
accggcaaat tgccggaatt gaaatttccg gcaaaccggc aaatcggctg aa
#ttgaaatt 1020
tcctgcaaac cggcaaattg cggtaattga aatttcctgc aaaccggtca gt
#tgccgatt 1080
tgcctttgcc tgaaaaacgg cgattgccag aaatattcgg caaattgtgg tt
#ttgcacat 1140
ttttctggaa atttcaggca aaattgtacg catcctatga atatccctat ta
#acatcttt 1200
tttgaaaagt cagtaaatta tatgaaaata tctaaagaaa acggggaaaa ta
#tttcaaag 1260
aggcacagtt ttatgtgttt ccgtcatcta aatagtccct ctaaacattt cc
#ggcaaatc 1320
tgatatccgg caaacggcaa atcgggatat tgccggaatt taaaatttgc cg
#aacttgtc 1380
gacaaaaaaa atgcgccttg aatccgattc agatattcaa aaattgaatt tt
#ggacgttt 1440
tagaaatcat ttagtttgtc aattttcaag aaatttctag aaaattggat gg
#tttccgcc 1500
aagaaatatt agctacatga aaataatttt gaaactagac atttcttaaa at
#aaaaattg 1560
ccatctttta tatccagatt tacgaaaagt atgcgttctc gaatcccctc ca
#ccctgacg 1620
tctttccggg agctcgtaaa atggaggcag aacttattcg aatggttctg aa
#cctgtata 1680
atggaccaga agattctagt ggaagtgtaa cttctggtgg tactgaaagt at
#tattatgg 1740
catgcttttc gtatcggtaa gcatttattc aactcttaaa attcaatttt gc
#aaactcta 1800
cagaaatcgt gcacactctc ttggcattga acatccagtt attttggcat gt
#aaaacagc 1860
tcacgcggca tttgataagg ccgcccatct atgcggaatg cgtcttcgcc ac
#gttccagt 1920
tgattcggat aatcgtgtcg atttaaaaga aatggagaga ctaattgatt cg
#aatgtttg 1980
tatgttggtt ggctcagcgc ctaacttccc atcaggcaca attgatccaa tt
#ccggaaat 2040
tgctaaggta ctggaaattc ccgcctcaat atcgcggaaa aaatagagaa at
#gactgaac 2100
aaaattacat tgtgagcggg aactctaatt gaattcagca aaaatacgat ac
#ttttttct 2160
aacttaaaat aatttttaaa aaaactcaca gatgctagtc caaaaaatgg cc
#ttttttga 2220
ttacttaatc gaacgtttac actttcagct cggcaaaaag tatggaatcc cg
#gtccacgt 2280
ggacgcatgt cttggtggat tcatgattcc atttatgaat gacgccggat ac
#ctgattcc 2340
tgtattcgat ttcagaaatc ccggtgttac atctatttcg tgtgatactc at
#aaggttgg 2400
atacagttct atccattttt ttccttcaat tcaaaatctt tcagtacgga tg
#cacaccga 2460
aaggttcatc gattgtcatg tatcgttcca aggaacttca tcacttccag ta
#tttctcgg 2520
ttgccgattg gtgtggaggc atctatgcca ccccgactat tgcaggtttg aa
#gaatgttt 2580
tagtagcttc aatagaatca aagagatccc ttaggatccc gagctggagc ca
#acactgcc 2640
gtcgcctggg ccacactttt atccttcggt cgagacgaat atgttcgaag at
#gtgctcaa 2700
attgtgaagc atacacgaat gctggccgag aaaattgaga aaatcaaatg ga
#tcaagcct 2760
tatggaaaat cggatgtttc attggtggcg ttctccggaa atggtgtgaa ta
#tctacgaa 2820
gtttctgaca aaatgatgaa gctcggatgg aatttgaaca ctctgcagaa tc
#cagcggcg 2880
tatgtttatc aattttatga gttatcagct tgctaaattt tttgtttcag aa
#tccacatt 2940
tgtttgacaa tcaatcaagc gaacgaggaa gttgtgaatg cgttcgccgt cg
#accttgag 3000
aagatttgtg aagaactcgc tgcaaaaggt gaacaaaaag ctgacagtgg aa
#tggctgcg 3060
atgtatggaa tggctgcgca agtaccaaaa tcagtagtgg acgaggttat cg
#ctctgtac 3120
attgacgcaa cttattcagc tccaccttca acttctaatt aa
#
#3162
<210> SEQ ID NO 13
<211> LENGTH: 34
<212> TYPE: DNA
<213> ORGANISM: Artificial Sequence
<220> FEATURE:
<223> OTHER INFORMATION: primer
<400> SEQUENCE: 13
gaggaattca tggattcggt taagcacaca accg
#
# 34
<210> SEQ ID NO 14
<211> LENGTH: 33
<212> TYPE: DNA
<213> ORGANISM: Artificial Sequence
<220> FEATURE:
<223> OTHER INFORMATION: primer
<400> SEQUENCE: 14
agcctcgagt taattagaag ttgaaggtgg agc
#
# 33
<210> SEQ ID NO 15
<211> LENGTH: 1638
<212> TYPE: DNA
<213> ORGANISM: Drosophila melanogaster
<400> SEQUENCE: 15
atgcgtccgt tctccggcag cgattgcctt aagcccgtca ccgagggcat ca
#accgggcg 60
ttcggcgcca aggagccctg gcaggtggcc accatcacgg ccaccacggt gc
#tgggaggc 120
gtctggctct ggactgtgat ctgccaggat gaaaatcttt acattcgtgg ca
#agcgtcag 180
ttctttaagt ttgccaagaa gattccagcc gtgcgtcgtc aggtggagac tg
#aattggcc 240
aaggccaaaa acgacttcga gacggaaatc aaaaagagca acgcccacct ta
#cctactcg 300
gaaactctgc ccgagaaggg actcagcaag gaggagatcc tccgactggt gg
#atgagcac 360
ctgaagactg gtcactacaa ctggcgtgat ggtcgtgtat ctggcgcggt ct
#acggctac 420
aagcctgatc tggtggagct cgtcactgaa gtgtacggca aggcctccta ca
#ccaatccc 480
ttgcacgcag atcttttccc gggagtttgc aaaatggagg cggaggtagt gc
#gcatggca 540
tgcaacctgt tccatggaaa ctcagccagc tgtggaacca tgaccaccgg cg
#gcaccgaa 600
tccattgtaa tggccatgaa ggcgtacagg gatttcgcta gagagtacaa gg
#gaatcacc 660
aggccaaaca tcgtggtgcc taagacggtc cacgcggcct tcgacaaggg cg
#gtcagtac 720
tttaatatcc acgtgcgatc cgtggatgta gatccggaga cctacgaagt gg
#acattaag 780
aagttcaaac gtgccattaa caggaacacg attctgctgg ttgggtctgc tc
#cgaacttc 840
ccctatggaa ccatcgatga catcgaagct atcgccgctt tgggcgttaa gt
#acgacatt 900
cccgtgcacg tggacgcctg cctgggcagc tttgtggtgg ccttggtccg ca
#acgccggc 960
tataagctgc gtcccttcga ctttgaggtc aagggagtga ccagtatctc cg
#ctgatacc 1020
cacaagtatg gtttcgcgcc caagggatca tcggtgatcc tttactcgga ca
#agaagtac 1080
aaggaccatc agttcactgt gactactgac tggcctggcg gcgtgtatgg tt
#ctcccaca 1140
gtcaacggtt cccgtgccgg aggtattatc gccgcctgct gggctaccat ga
#tgagcttt 1200
ggctatgatg gttatctgga agccactaag cgcattgtgg atacggcgcg ct
#atatcgag 1260
aggggcgttc gcgacatcga tggcatcttt atctttggca agccagctac tt
#cagtgatt 1320
gccctgggtt ccaatgtgtt tgacattttc cggctatcgg attcgctgtg ca
#aactgggc 1380
tggaacctaa atgcgctgca gtttccatct ggtatccacc tgtgcgtgac gg
#acatgcac 1440
acacagcccg gagtcgcgga taaattcatt gccgatgtgc gcagctgtac gg
#cggagatc 1500
atgaaggatc ccggccagcc cgtcgttgga aagatggctc tttacggcat gg
#cacagagc 1560
atacccgacc gttcggtgat cggagaagtg actcgcctat tcctgcactc ca
#tgtactac 1620
actcccagcc agaaatag
#
#
#1638
<210> SEQ ID NO 16
<211> LENGTH: 545
<212> TYPE: PRT
<213> ORGANISM: Drosophila melanogaster
<400> SEQUENCE: 16
Met Arg Pro Phe Ser Gly Ser Asp Cys Leu Ly
#s Pro Val Thr Glu Gly
  1 5
# 10
# 15
Ile Asn Arg Ala Phe Gly Ala Lys Glu Pro Tr
#p Gln Val Ala Thr Ile
                        20
# 25
# 30
Thr Ala Thr Thr Val Leu Gly Gly Val Trp Le
#u Trp Thr Val Ile Cys
                35
# 40
# 45
Gln Asp Glu Asn Leu Tyr Ile Arg Gly Lys Ar
#g Gln Phe Phe Lys Phe
        50
# 55
# 60
Ala Lys Lys Ile Pro Ala Val Arg Arg Gln Va
#l Glu Thr Glu Leu Ala
65
#70
#75
#80
Lys Ala Lys Asn Asp Phe Glu Thr Glu Ile Ly
#s Lys Ser Asn Ala His
                                85
# 90
# 95
Leu Thr Tyr Ser Glu Thr Leu Pro Glu Lys Gl
#y Leu Ser Lys Glu Glu
                        100
# 105
# 110
Ile Leu Arg Leu Val Asp Glu His Leu Lys Th
#r Gly His Tyr Asn Trp
                115
# 120
# 125
Arg Asp Gly Arg Val Ser Gly Ala Val Tyr Gl
#y Tyr Lys Pro Asp Leu
        130
# 135
# 140
Val Glu Leu Val Thr Glu Val Tyr Gly Lys Al
#a Ser Tyr Thr Asn Pro
145 1
#50 1
#55 1
#60
Leu His Ala Asp Leu Phe Pro Gly Val Cys Ly
#s Met Glu Ala Glu Val
                                165
# 170
# 175
Val Arg Met Ala Cys Asn Leu Phe His Gly As
#n Ser Ala Ser Cys Gly
                        180
# 185
# 190
Thr Met Thr Thr Gly Gly Thr Glu Ser Ile Va
#l Met Ala Met Lys Ala
                195
# 200
# 205
Tyr Arg Asp Phe Ala Arg Glu Tyr Lys Gly Il
#e Thr Arg Pro Asn Ile
        210
# 215
# 220
Val Val Pro Lys Thr Val His Ala Ala Phe As
#p Lys Gly Gly Gln Tyr
225 2
#30 2
#35 2
#40
Phe Asn Ile His Val Arg Ser Val Asp Val As
#p Pro Glu Thr Tyr Glu
                                245
# 250
# 255
Val Asp Ile Lys Lys Phe Lys Arg Ala Ile As
#n Arg Asn Thr Ile Leu
                        260
# 265
# 270
Leu Val Gly Ser Ala Pro Asn Phe Pro Tyr Gl
#y Thr Ile Asp Asp Ile
                275
# 280
# 285
Glu Ala Ile Ala Ala Leu Gly Val Lys Tyr As
#p Ile Pro Val His Val
        290
# 295
# 300
Asp Ala Cys Leu Gly Ser Phe Val Val Ala Le
#u Val Arg Asn Ala Gly
305 3
#10 3
#15 3
#20
Tyr Lys Leu Arg Pro Phe Asp Phe Glu Val Ly
#s Gly Val Thr Ser Ile
                                325
# 330
# 335
Ser Ala Asp Thr His Lys Tyr Gly Phe Ala Pr
#o Lys Gly Ser Ser Val
                        340
# 345
# 350
Ile Leu Tyr Ser Asp Lys Lys Tyr Lys Asp Hi
#s Gln Phe Thr Val Thr
                355
# 360
# 365
Thr Asp Trp Pro Gly Gly Val Tyr Gly Ser Pr
#o Thr Val Asn Gly Ser
        370
# 375
# 380
Arg Ala Gly Gly Ile Ile Ala Ala Cys Trp Al
#a Thr Met Met Ser Phe
385 3
#90 3
#95 4
#00
Gly Tyr Asp Gly Tyr Leu Glu Ala Thr Lys Ar
#g Ile Val Asp Thr Ala
                                405
# 410
# 415
Arg Tyr Ile Glu Arg Gly Val Arg Asp Ile As
#p Gly Ile Phe Ile Phe
                        420
# 425
# 430
Gly Lys Pro Ala Thr Ser Val Ile Ala Leu Gl
#y Ser Asn Val Phe Asp
                435
# 440
# 445
Ile Phe Arg Leu Ser Asp Ser Leu Cys Lys Le
#u Gly Trp Asn Leu Asn
        450
# 455
# 460
Ala Leu Gln Phe Pro Ser Gly Ile His Leu Cy
#s Val Thr Asp Met His
465 4
#70 4
#75 4
#80
Thr Gln Pro Gly Val Ala Asp Lys Phe Ile Al
#a Asp Val Arg Ser Cys
                                485
# 490
# 495
Thr Ala Glu Ile Met Lys Asp Pro Gly Gln Pr
#o Val Val Gly Lys Met
                        500
# 505
# 510
Ala Leu Tyr Gly Met Ala Gln Ser Ile Pro As
#p Arg Ser Val Ile Gly
                515
# 520
# 525
Glu Val Thr Arg Leu Phe Leu His Ser Met Ty
#r Tyr Thr Pro Ser Gln
        530
# 535
# 540
Lys
545
<210> SEQ ID NO 17
<211> LENGTH: 1707
<212> TYPE: DNA
<213> ORGANISM: Homo sapiens
<220> FEATURE:
<221> NAME/KEY: CDS
<222> LOCATION: (1)...(1707)
<400> SEQUENCE: 17
atg cct agc aca gac ctt ctg atg ttg aag gc
#c ttt gag ccc tac tta 48
Met Pro Ser Thr Asp Leu Leu Met Leu Lys Al
#a Phe Glu Pro Tyr Leu
  1 5
# 10
# 15
gag att ttg gaa gta tac tcc aca aaa gcc aa
#g aat tat gta aat gga 96
Glu Ile Leu Glu Val Tyr Ser Thr Lys Ala Ly
#s Asn Tyr Val Asn Gly
                          20
# 25
# 30
cat tgc acc aag tat gag ccc tgg cag cta at
#t gca tgg agt gtc gtg 144
His Cys Thr Lys Tyr Glu Pro Trp Gln Leu Il
#e Ala Trp Ser Val Val
                  35
# 40
# 45
tgg acc ctg ctg ata gtc tgg gga tat gag tt
#t gtc ttc cag cca gag 192
Trp Thr Leu Leu Ile Val Trp Gly Tyr Glu Ph
#e Val Phe Gln Pro Glu
          50
# 55
# 60
agt tta tgg tca agg ttt aaa aag aaa tgt tt
#t aag ctc acc agg aag 240
Ser Leu Trp Ser Arg Phe Lys Lys Lys Cys Ph
#e Lys Leu Thr Arg Lys
  65
# 70
# 75
# 80
atg ccc att att ggt cgt aag att caa gac aa
#g ttg aac aag acc aag 288
Met Pro Ile Ile Gly Arg Lys Ile Gln Asp Ly
#s Leu Asn Lys Thr Lys
                                  85
# 90
# 95
gat gat att agc aag aac atg tca ttc ctg aa
#a gtg gac aaa gag tat 336
Asp Asp Ile Ser Lys Asn Met Ser Phe Leu Ly
#s Val Asp Lys Glu Tyr
                        100
# 105
# 110
gtg aaa gct tta ccc tcc cag ggt ctg agc tc
#a tct gct gtt ttg gag 384
Val Lys Ala Leu Pro Ser Gln Gly Leu Ser Se
#r Ser Ala Val Leu Glu
                115
# 120
# 125
aaa ctt aag gag tac agc tct atg gac gcc tt
#c tgg caa gag ggg aga 432
Lys Leu Lys Glu Tyr Ser Ser Met Asp Ala Ph
#e Trp Gln Glu Gly Arg
        130
# 135
# 140
gcc tct gga aca gtg tac agt ggg gag gag aa
#g ctc act gag ctc ctt 480
Ala Ser Gly Thr Val Tyr Ser Gly Glu Glu Ly
#s Leu Thr Glu Leu Leu
145 1
#50 1
#55 1
#60
gtg aag gct tat gga gat ttt gca tgg agt aa
#c ccc ctg cat cca gat 528
Val Lys Ala Tyr Gly Asp Phe Ala Trp Ser As
#n Pro Leu His Pro Asp
                                165
# 170
# 175
atc ttc cca gga cta cgc aag ata gag gca ga
#a att gtg agg ata gct 576
Ile Phe Pro Gly Leu Arg Lys Ile Glu Ala Gl
#u Ile Val Arg Ile Ala
                        180
# 185
# 190
tgt tcc ctg ttc aat ggg gga cca gat tcg tg
#t gga tgt gtg act tct 624
Cys Ser Leu Phe Asn Gly Gly Pro Asp Ser Cy
#s Gly Cys Val Thr Ser
                195
# 200
# 205
ggg gga aca gaa agc ata ctc atg gcc tgc aa
#a gca tat cgg gat ctg 672
Gly Gly Thr Glu Ser Ile Leu Met Ala Cys Ly
#s Ala Tyr Arg Asp Leu
        210
# 215
# 220
gcc ttt gag aag ggg atc aaa act cca gaa at
#t gtg gct ccc caa agt 720
Ala Phe Glu Lys Gly Ile Lys Thr Pro Glu Il
#e Val Ala Pro Gln Ser
225 2
#30 2
#35 2
#40
gcc cat gct gca ttt aac aaa gca gcc agt ta
#c ttt ggg atg aag att 768
Ala His Ala Ala Phe Asn Lys Ala Ala Ser Ty
#r Phe Gly Met Lys Ile
                                245
# 250
# 255
gtg cgg gtc cca ttg acg aag atg atg gag gt
#g gat gtg agg gca atg 816
Val Arg Val Pro Leu Thr Lys Met Met Glu Va
#l Asp Val Arg Ala Met
                        260
# 265
# 270
aga aga gct atc tcc agg aac act gcc atg ct
#c gtc tgt tct acc cca 864
Arg Arg Ala Ile Ser Arg Asn Thr Ala Met Le
#u Val Cys Ser Thr Pro
                275
# 280
# 285
cag ttt cct cat ggt gta ata gat cct gtc cc
#t gaa gtg gcc aag ctg 912
Gln Phe Pro His Gly Val Ile Asp Pro Val Pr
#o Glu Val Ala Lys Leu
        290
# 295
# 300
gct gtc aaa tac aaa ata ccc ctt cat gtc ga
#c gct tgt ctg gga ggc 960
Ala Val Lys Tyr Lys Ile Pro Leu His Val As
#p Ala Cys Leu Gly Gly
305 3
#10 3
#15 3
#20
ttc ctc atc gtc ttt atg gag aaa gca gga ta
#c cca ctg gag cac cca 1008
Phe Leu Ile Val Phe Met Glu Lys Ala Gly Ty
#r Pro Leu Glu His Pro
                                325
# 330
# 335
ttt gat ttc cgg gtg aaa ggt gta acc agc at
#t tca gct gac acc cat 1056
Phe Asp Phe Arg Val Lys Gly Val Thr Ser Il
#e Ser Ala Asp Thr His
                        340
# 345
# 350
aag tat ggc tat gcc cca aaa ggc tca tca tt
#g gtg ttg tat agt gac 1104
Lys Tyr Gly Tyr Ala Pro Lys Gly Ser Ser Le
#u Val Leu Tyr Ser Asp
                355
# 360
# 365
aag aag tac agg aac tat cag ttc ttc gtc ga
#t aca gat tgg cag ggt 1152
Lys Lys Tyr Arg Asn Tyr Gln Phe Phe Val As
#p Thr Asp Trp Gln Gly
        370
# 375
# 380
ggc atc tat gct tcc cca acc atc gca ggc tc
#a cgg cct ggt ggc att 1200
Gly Ile Tyr Ala Ser Pro Thr Ile Ala Gly Se
#r Arg Pro Gly Gly Ile
385 3
#90 3
#95 4
#00
agc gca gcc tgt tgg gct gcc ttg atg cac tt
#c ggt gag aac ggc tat 1248
Ser Ala Ala Cys Trp Ala Ala Leu Met His Ph
#e Gly Glu Asn Gly Tyr
                                405
# 410
# 415
gtt gaa gct acc aaa cag atc atc aaa act gc
#t cgc ttc ctc aag tca 1296
Val Glu Ala Thr Lys Gln Ile Ile Lys Thr Al
#a Arg Phe Leu Lys Ser
                        420
# 425
# 430
gaa ctg gaa aat atc aaa ggc atc ttt gtt tt
#t ggg aat ccc caa ttg 1344
Glu Leu Glu Asn Ile Lys Gly Ile Phe Val Ph
#e Gly Asn Pro Gln Leu
                435
# 440
# 445
tca gtc att gct ctg gga tcc cgt gat ttt ga
#c atc tac cga cta tca 1392
Ser Val Ile Ala Leu Gly Ser Arg Asp Phe As
#p Ile Tyr Arg Leu Ser
        450
# 455
# 460
aac ctg atg act gct aag ggg tgg aac ttg aa
#c cag ttg cag ttc cca 1440
Asn Leu Met Thr Ala Lys Gly Trp Asn Leu As
#n Gln Leu Gln Phe Pro
465 4
#70 4
#75 4
#80
ccc agt att cat ttc tgc atc aca tta cta ca
#c gcc cgg aaa cga gta 1488
Pro Ser Ile His Phe Cys Ile Thr Leu Leu Hi
#s Ala Arg Lys Arg Val
                                485
# 490
# 495
gct ata caa ttc cta aag gac att cga gaa tc
#t gtc act caa atc atg 1536
Ala Ile Gln Phe Leu Lys Asp Ile Arg Glu Se
#r Val Thr Gln Ile Met
                        500
# 505
# 510
aag aat cct aaa gcg aag acc aca gga atg gg
#t gcc atc tat ggc atg 1584
Lys Asn Pro Lys Ala Lys Thr Thr Gly Met Gl
#y Ala Ile Tyr Gly Met
                515
# 520
# 525
gcc cag aca act gtt gac agg aat atg gtt gc
#a gaa ttg tcc tca gtc 1632
Ala Gln Thr Thr Val Asp Arg Asn Met Val Al
#a Glu Leu Ser Ser Val
        530
# 535
# 540
ttc ttg gac agc ttg tac agc acc gac act gt
#c acc cag ggc agc cag 1680
Phe Leu Asp Ser Leu Tyr Ser Thr Asp Thr Va
#l Thr Gln Gly Ser Gln
545 5
#50 5
#55 5
#60
atg aat ggt tct cca aaa ccc cac tga
#
# 1707
Met Asn Gly Ser Pro Lys Pro His *
                                565
<210> SEQ ID NO 18
<211> LENGTH: 568
<212> TYPE: PRT
<213> ORGANISM: Homo sapiens
<400> SEQUENCE: 18
Met Pro Ser Thr Asp Leu Leu Met Leu Lys Al
#a Phe Glu Pro Tyr Leu
  1 5
# 10
# 15
Glu Ile Leu Glu Val Tyr Ser Thr Lys Ala Ly
#s Asn Tyr Val Asn Gly
                        20
# 25
# 30
His Cys Thr Lys Tyr Glu Pro Trp Gln Leu Il
#e Ala Trp Ser Val Val
                35
# 40
# 45
Trp Thr Leu Leu Ile Val Trp Gly Tyr Glu Ph
#e Val Phe Gln Pro Glu
        50
# 55
# 60
Ser Leu Trp Ser Arg Phe Lys Lys Lys Cys Ph
#e Lys Leu Thr Arg Lys
65
#70
#75
#80
Met Pro Ile Ile Gly Arg Lys Ile Gln Asp Ly
#s Leu Asn Lys Thr Lys
                                85
# 90
# 95
Asp Asp Ile Ser Lys Asn Met Ser Phe Leu Ly
#s Val Asp Lys Glu Tyr
                        100
# 105
# 110
Val Lys Ala Leu Pro Ser Gln Gly Leu Ser Se
#r Ser Ala Val Leu Glu
                115
# 120
# 125
Lys Leu Lys Glu Tyr Ser Ser Met Asp Ala Ph
#e Trp Gln Glu Gly Arg
        130
# 135
# 140
Ala Ser Gly Thr Val Tyr Ser Gly Glu Glu Ly
#s Leu Thr Glu Leu Leu
145 1
#50 1
#55 1
#60
Val Lys Ala Tyr Gly Asp Phe Ala Trp Ser As
#n Pro Leu His Pro Asp
                                165
# 170
# 175
Ile Phe Pro Gly Leu Arg Lys Ile Glu Ala Gl
#u Ile Val Arg Ile Ala
                        180
# 185
# 190
Cys Ser Leu Phe Asn Gly Gly Pro Asp Ser Cy
#s Gly Cys Val Thr Ser
                195
# 200
# 205
Gly Gly Thr Glu Ser Ile Leu Met Ala Cys Ly
#s Ala Tyr Arg Asp Leu
        210
# 215
# 220
Ala Phe Glu Lys Gly Ile Lys Thr Pro Glu Il
#e Val Ala Pro Gln Ser
225 2
#30 2
#35 2
#40
Ala His Ala Ala Phe Asn Lys Ala Ala Ser Ty
#r Phe Gly Met Lys Ile
                                245
# 250
# 255
Val Arg Val Pro Leu Thr Lys Met Met Glu Va
#l Asp Val Arg Ala Met
                        260
# 265
# 270
Arg Arg Ala Ile Ser Arg Asn Thr Ala Met Le
#u Val Cys Ser Thr Pro
                275
# 280
# 285
Gln Phe Pro His Gly Val Ile Asp Pro Val Pr
#o Glu Val Ala Lys Leu
        290
# 295
# 300
Ala Val Lys Tyr Lys Ile Pro Leu His Val As
#p Ala Cys Leu Gly Gly
305 3
#10 3
#15 3
#20
Phe Leu Ile Val Phe Met Glu Lys Ala Gly Ty
#r Pro Leu Glu His Pro
                                325
# 330
# 335
Phe Asp Phe Arg Val Lys Gly Val Thr Ser Il
#e Ser Ala Asp Thr His
                        340
# 345
# 350
Lys Tyr Gly Tyr Ala Pro Lys Gly Ser Ser Le
#u Val Leu Tyr Ser Asp
                355
# 360
# 365
Lys Lys Tyr Arg Asn Tyr Gln Phe Phe Val As
#p Thr Asp Trp Gln Gly
        370
# 375
# 380
Gly Ile Tyr Ala Ser Pro Thr Ile Ala Gly Se
#r Arg Pro Gly Gly Ile
385 3
#90 3
#95 4
#00
Ser Ala Ala Cys Trp Ala Ala Leu Met His Ph
#e Gly Glu Asn Gly Tyr
                                405
# 410
# 415
Val Glu Ala Thr Lys Gln Ile Ile Lys Thr Al
#a Arg Phe Leu Lys Ser
                        420
# 425
# 430
Glu Leu Glu Asn Ile Lys Gly Ile Phe Val Ph
#e Gly Asn Pro Gln Leu
                435
# 440
# 445
Ser Val Ile Ala Leu Gly Ser Arg Asp Phe As
#p Ile Tyr Arg Leu Ser
        450
# 455
# 460
Asn Leu Met Thr Ala Lys Gly Trp Asn Leu As
#n Gln Leu Gln Phe Pro
465 4
#70 4
#75 4
#80
Pro Ser Ile His Phe Cys Ile Thr Leu Leu Hi
#s Ala Arg Lys Arg Val
                                485
# 490
# 495
Ala Ile Gln Phe Leu Lys Asp Ile Arg Glu Se
#r Val Thr Gln Ile Met
                        500
# 505
# 510
Lys Asn Pro Lys Ala Lys Thr Thr Gly Met Gl
#y Ala Ile Tyr Gly Met
                515
# 520
# 525
Ala Gln Thr Thr Val Asp Arg Asn Met Val Al
#a Glu Leu Ser Ser Val
        530
# 535
# 540
Phe Leu Asp Ser Leu Tyr Ser Thr Asp Thr Va
#l Thr Gln Gly Ser Gln
545 5
#50 5
#55 5
#60
Met Asn Gly Ser Pro Lys Pro His
                                565
<210> SEQ ID NO 19
<211> LENGTH: 490
<212> TYPE: PRT
<213> ORGANISM: Drosophila melanogaster
<400> SEQUENCE: 19
Phe Arg Ser Ser Asn Asp Tyr Gly Val Asn Le
#u Gln Thr Ala Glu Met
  1 5
# 10
# 15
Trp His His Thr Ile Arg Lys His Lys Arg Gl
#y Asn Gly Ser Ser Ser
                        20
# 25
# 30
Pro Ala Asp Cys Gly Lys Gln Leu Leu Ile Le
#u Leu Asn Pro Lys Ser
                35
# 40
# 45
Gly Ser Gly Lys Gly Arg Glu Leu Phe Gln Ly
#s Gln Val Ala Pro Leu
        50
# 55
# 60
Leu Thr Glu Ala Glu Val Gln Tyr Asp Leu Gl
#n Ile Thr Thr His Pro
65
#70
#75
#80
Gln Tyr Ala Lys Glu Phe Val Arg Thr Arg Ar
#g Asp Leu Leu Thr Arg
                                85
# 90
# 95
Tyr Ser Gly Ile Val Val Ala Ser Gly Asp Gl
#y Leu Phe Tyr Glu Val
                        100
# 105
# 110
Leu Asn Gly Leu Met Glu Arg Met Asp Trp Ar
#g Arg Ala Cys Arg Glu
                115
# 120
# 125
Leu Pro Leu Gly Ile Ile Pro Cys Gly Ser Gl
#y Asn Gly Leu Ala Lys
        130
# 135
# 140
Ser Val Ala His His Cys Asn Glu Pro Tyr Gl
#u Pro Lys Pro Ile Leu
145 1
#50 1
#55 1
#60
His Ala Thr Leu Thr Cys Met Ala Gly Lys Se
#r Thr Pro Met Asp Val
                                165
# 170
# 175
Val Arg Val Glu Leu Ala Thr Arg Asp Lys Hi
#s Phe Val Met Tyr Ser
                        180
# 185
# 190
Phe Leu Ser Val Gly Trp Gly Leu Ile Ala As
#p Ile Asp Ile Glu Ser
                195
# 200
# 205
Glu Arg Leu Arg Ser Ile Gly Ala Gln Arg Ph
#e Thr Leu Trp Ala Ile
        210
# 215
# 220
Lys Arg Leu Ile Gly Leu Arg Ser Tyr Lys Gl
#y Arg Val Ser Tyr Leu
225 2
#30 2
#35 2
#40
Leu Gly Lys Gly Lys Lys Glu Pro Pro Val Gl
#u Ala Ala Arg Glu Leu
                                245
# 250
# 255
Pro Ala Glu Ser Thr Ala Ala Gly Ile Arg Se
#r Ser Leu Pro Leu Asn
                        260
# 265
# 270
Ala Gly Glu Phe His Asp Leu Pro Glu Glu Gl
#u Glu Gly Glu Ala Val
                275
# 280
# 285
Leu Asp Gly Glu Gln Phe Ala Asp Ala Ile Se
#r Leu Asp Arg Ser Val
        290
# 295
# 300
Tyr Arg Gln His Ala Asp Ser Trp His Ser Al
#a Met Ser Arg Arg Thr
305 3
#10 3
#15 3
#20
Ala Tyr Tyr Ser Leu Gly Gly Pro Ser Met Ar
#g Ser Asn Arg Ser Arg
                                325
# 330
# 335
Met Ser Ile Ser Gln Arg Ile Glu Ala Ala As
#n Ala Glu Phe Ala Glu
                        340
# 345
# 350
Arg Val Pro Thr Gly Thr Ile Pro Pro Leu Gl
#n Met Pro Leu Leu Ser
                355
# 360
# 365
Ser Asp Gly Trp Ile Cys Glu Asp Gly Asp Ph
#e Val Met Val His Ala
        370
# 375
# 380
Ala Tyr Thr Thr His Leu Ser Ser Asp Val Ph
#e Phe Ala Pro Glu Ser
385 3
#90 3
#95 4
#00
Arg Leu Asp Asp Gly Leu Ile Tyr Leu Val Il
#e Ile Arg Arg Gly Val
                                405
# 410
# 415
Ser Arg His Gln Leu Leu Asn Phe Met Leu As
#n Leu Asn Ala Gly Thr
                        420
# 425
# 430
His Leu Pro Ile Gly Glu Asp Pro Phe Ile Ly
#s Val Val Pro Cys Arg
                435
# 440
# 445
Ala Phe Arg Ile Glu Pro Ser Ser Ser Asp Gl
#y Ile Leu Val Val Asp
        450
# 455
# 460
Gly Glu Arg Val Glu Tyr Gly Pro Ile Gln Al
#a Glu Val Met Pro Gly
465 4
#70 4
#75 4
#80
Leu Ile Asn Val Met Thr Thr Ser Gly Gln
                                485
# 490
<210> SEQ ID NO 20
<211> LENGTH: 524
<212> TYPE: PRT
<213> ORGANISM: Drosophila melanogaster
<400> SEQUENCE: 20
Phe Arg Ser Phe Asp Thr Phe Glu Asp Asn Me
#t Arg Glu Ala Asp Arg
  1 5
# 10
# 15
Trp Tyr Arg Ser Leu Arg Trp Gln Leu His Ar
#g Thr Leu Glu Glu Ile
                        20
# 25
# 30
Phe Val Ala Pro Thr Val Asp Glu Arg Arg Ar
#g Arg Val Leu Val Leu
                35
# 40
# 45
Leu Asn Pro Lys Ser Gly Ser Gly Asp Ala Ar
#g Glu Val Phe Asn Met
        50
# 55
# 60
His Val Thr Pro Val Leu Asn Glu Ala Glu Va
#l Pro Tyr Asp Leu Tyr
65
#70
#75
#80
Val Thr Lys His Ser Asn Phe Ala Ile Glu Ph
#e Leu Ser Thr Arg Cys
                                85
# 90
# 95
Leu Asp Ala Trp Cys Cys Val Val Ala Val Gl
#y Gly Asp Gly Leu Phe
                        100
# 105
# 110
His Glu Ile Val Asn Gly Leu Leu Gln Arg Gl
#n Asp Trp Ala His Val
                115
# 120
# 125
Leu Pro His Leu Ala Leu Gly Ile Ile Pro Cy
#s Gly Ser Gly Asn Gly
        130
# 135
# 140
Leu Ala Arg Ser Ile Ala His Cys Tyr Asn Ly
#s Pro Val Leu Gly Ala
145 1
#50 1
#55 1
#60
Ala Leu Thr Val Ile Ser Gly Arg Ser Ser Pr
#o Met Asp Val Val Arg
                                165
# 170
# 175
Val Gln Leu Gln Ser Arg Ser Leu Tyr Ser Ph
#e Leu Ser Ile Gly Trp
                        180
# 185
# 190
Gly Leu Ile Ser Asp Val Asp Ile Glu Ser Gl
#u Arg Ile Arg Met Leu
                195
# 200
# 205
Gly Tyr Gln Arg Phe Thr Val Trp Thr Leu Ty
#r Arg Leu Val Asn Leu
        210
# 215
# 220
Arg Thr Tyr Asn Gly Arg Ile Ser Tyr Leu Le
#u Thr Asp His Glu Val
225 2
#30 2
#35 2
#40
Ser Ser Thr His Ser Ala Thr Gly Tyr Ala Al
#a Gln Arg Arg Met Gln
                                245
# 250
# 255
Ser Ser Arg Ser Cys Asn Thr His Ile Asp Me
#t Leu Asn Gly Pro Ala
                        260
# 265
# 270
Pro Ile Tyr His Ser Ser Ala Glu Tyr Leu Pr
#o Gln Glu Phe Ala Asp
                275
# 280
# 285
Val Ile Ser Leu Glu Thr Ser Ile Asn Gln Se
#r Phe Arg Ser Arg Cys
        290
# 295
# 300
Asp Ser Trp Leu Ser Gly Gly Ser Arg Arg Se
#r Phe Tyr Tyr Ser Ile
305 3
#10 3
#15 3
#20
Ser Glu Ser Ile Tyr His Ser Leu Ala Asp Gl
#u Ser Glu Phe Ala Gly
                                325
# 330
# 335
Leu Ala Ala Ala Ser Leu Glu Asn Arg Gln Gl
#n Asn Tyr Gly Pro Ala
                        340
# 345
# 350
Ser Glu Leu Pro Asp Leu Asn Glu Pro Leu Se
#r Glu Asp Gln Gly Trp
                355
# 360
# 365
Leu Val Glu Glu Gly Glu Phe Val Met Met Hi
#s Ala Val Tyr Gln Thr
        370
# 375
# 380
His Leu Gly Ile Asp Cys His Phe Ala Pro Ly
#s Ala Gln Leu Asn Asp
385 3
#90 3
#95 4
#00
Gly Thr Ile Tyr Leu Ile Leu Ile Arg Ala Gl
#y Ile Ser Arg Pro His
                                405
# 410
# 415
Leu Leu Ser Phe Leu Tyr Asn Met Ser Ser Gl
#y Thr His Leu Pro Glu
                        420
# 425
# 430
Ser His Asp Asp His Val Lys Val Leu Pro Va
#l Arg Ala Phe Arg Leu
                435
# 440
# 445
Glu Pro Tyr Asp Asn His Gly Ile Ile Thr Va
#l Asp Gly Glu Arg Val
        450
# 455
# 460
Glu Phe Gly Pro Leu Gln Ala Glu Val Leu Pr
#o Gly Ile Ala Arg Val
465 4
#70 4
#75 4
#80
Met Val Pro Asn Val Ser Thr Phe Arg Phe Gl
#n Ser Ala Thr Leu Gln
                                485
# 490
# 495
His Gly Ile Pro Val Cys Ile Pro Val Arg Ly
#s Arg Phe Val Leu Tyr
                        500
# 505
# 510
Asn Met Ser Ser Glu Glu Leu Ala Pro Ile As
#n Glu
                515
# 520
<210> SEQ ID NO 21
<211> LENGTH: 368
<212> TYPE: PRT
<213> ORGANISM: Homo sapiens
<400> SEQUENCE: 21
Val Leu Val Leu Leu Asn Pro Arg Gly Gly Ly
#s Gly Lys Ala Leu Gln
  1 5
# 10
# 15
Leu Phe Arg Ser His Val Gln Pro Leu Leu Al
#a Glu Ala Glu Ile Ser
                        20
# 25
# 30
Phe Thr Leu Met Leu Thr Glu Arg Arg Asn Hi
#s Ala Arg Glu Leu Val
                35
# 40
# 45
Arg Ser Glu Glu Leu Gly Arg Trp Asp Ala Le
#u Val Val Met Ser Gly
        50
# 55
# 60
Asp Gly Leu Met His Glu Val Val Asn Gly Le
#u Met Glu Arg Pro Asp
65
#70
#75
#80
Trp Glu Thr Ala Ile Gln Lys Pro Leu Cys Se
#r Leu Pro Ala Gly Ser
                                85
# 90
# 95
Gly Asn Ala Leu Ala Ala Ser Leu Asn His Ty
#r Ala Gly Tyr Glu Gln
                        100
# 105
# 110
Val Thr Asn Glu Asp Leu Leu Thr Asn Cys Th
#r Leu Leu Leu Cys Arg
                115
# 120
# 125
Arg Leu Leu Ser Pro Met Asn Leu Leu Ser Le
#u His Thr Ala Ser Gly
        130
# 135
# 140
Leu Arg Leu Phe Ser Val Leu Ser Leu Ala Tr
#p Gly Phe Ile Ala Asp
145 1
#50 1
#55 1
#60
Val Asp Leu Glu Ser Glu Lys Tyr Arg Arg Le
#u Gly Glu Met Arg Phe
                                165
# 170
# 175
Thr Leu Gly Thr Phe Leu Arg Leu Ala Ala Le
#u Arg Thr Tyr Arg Gly
                        180
# 185
# 190
Arg Leu Ala Tyr Leu Pro Val Gly Arg Val Gl
#y Ser Lys Thr Pro Ala
                195
# 200
# 205
Ser Pro Val Val Val Gln Gln Gly Pro Val As
#p Ala His Leu Val Pro
        210
# 215
# 220
Leu Glu Glu Pro Val Pro Ser His Trp Thr Va
#l Val Pro Asp Glu Asp
225 2
#30 2
#35 2
#40
Phe Val Leu Val Leu Ala Leu Leu His Ser Hi
#s Leu Gly Ser Glu Met
                                245
# 250
# 255
Phe Ala Ala Pro Met Gly Arg Cys Ala Ala Gl
#y Val Met His Leu Phe
                        260
# 265
# 270
Tyr Val Arg Ala Gly Val Ser Arg Ala Met Le
#u Leu Arg Leu Phe Leu
                275
# 280
# 285
Ala Met Glu Lys Gly Arg His Met Glu Tyr Gl
#u Cys Pro Tyr Leu Val
        290
# 295
# 300
Tyr Val Pro Val Val Ala Phe Arg Leu Glu Pr
#o Lys Asp Gly Lys Gly
305 3
#10 3
#15 3
#20
Val Phe Ala Val Asp Gly Glu Leu Met Val Se
#r Glu Ala Val Gln Gly
                                325
# 330
# 335
Gln Val His Pro Asn Tyr Phe Trp Met Val Se
#r Gly Cys Val Glu Pro
                        340
# 345
# 350
Pro Pro Ser Trp Lys Pro Gln Gln Met Pro Pr
#o Pro Glu Glu Pro Leu
                355
# 360
# 365
<210> SEQ ID NO 22
<211> LENGTH: 1152
<212> TYPE: DNA
<213> ORGANISM: Homo sapiens
<400> SEQUENCE: 22
atggatccag cgggcggccc ccggggcgtg ctcccgcggc cctgccgcgt gc
#tggtgctg 60
ctgaacccgc gcggcggcaa gggcaaggcc ttgcagctct tccggagtca cg
#tgcagccc 120
cttttggctg aggctgaaat ctccttcacg ctgatgctca ctgagcggcg ga
#accacgcg 180
cgggagctgg tgcggtcgga ggagctgggc cgctgggacg ctctggtggt ca
#tgtctgga 240
gacgggctga tgcacgaggt ggtgaacggg ctcatggagc ggcctgactg gg
#agaccgcc 300
atccagaagc ccctgtgtag cctcccagca ggctctggca acgcgctggc ag
#cttccttg 360
aaccattatg ctggctatga gcaggtcacc aatgaagacc tcctgaccaa ct
#gcacgcta 420
ttgctgtgcc gccggctgct gtcacccatg aacctgctgt ctctgcacac gg
#cttcgggg 480
ctgcgcctct tctctgtgct cagcctggcc tggggcttca ttgctgatgt gg
#acctagag 540
agtgagaagt atcggcgtct gggggagatg cgcttcactc tgggcacctt cc
#tgcgtctg 600
gcagccctgc gcacctaccg cggccgactg gcctacctcc ctgtaggaag ag
#tgggttcc 660
aagacacctg cctcccccgt tgtggtccag cagggcccgg tagatgcaca cc
#ttgtgcca 720
ctggaggagc cagtgccctc tcactggaca gtggtgcccg acgaggactt tg
#tgctagtc 780
ctggcactgc tgcactcgca cctgggcagt gagatgtttg ctgcacccat gg
#gccgctgt 840
gcagctggcg tcatgcatct gttctacgtg cgggcgggag tgtctcgtgc ca
#tgctgctg 900
cgcctcttcc tggccatgga gaagggcagg catatggagt atgaatgccc ct
#acttggta 960
tatgtgcccg tggtcgcctt ccgcttggag cccaaggatg ggaaaggtgt gt
#ttgcagtg 1020
gatggggaat tgatggttag cgaggccgtg cagggccagg tgcacccaaa ct
#acttctgg 1080
atggtcagtg gttgcgtgga gcccccgccc agctggaagc cccagcagat gc
#caccgcca 1140
gaagagccct ta
#
#
# 1152
<210> SEQ ID NO 23
<211> LENGTH: 1707
<212> TYPE: DNA
<213> ORGANISM: Homo sapiens
<400> SEQUENCE: 23
atgcctagca cagaccttct gatgttgaag gcctttgagc cctacttaga ga
#ttttggaa 60
gtatactcca caaaagccaa gaattatgta aatggacatt gcaccaagta tg
#agccctgg 120
cagctaattg catggagtgt cgtgtggacc ctgctgatag tctggggata tg
#agtttgtc 180
ttccagccag agagtttatg gtcaaggttt aaaaagaaat gttttaagct ca
#ccaggaag 240
atgcccatta ttggtcgtaa gattcaagac aagttgaaca agaccaagga tg
#atattagc 300
aagaacatgt cattcctgaa agtggacaaa gagtatgtga aagctttacc ct
#cccagggt 360
ctgagctcat ctgctgtttt ggagaaactt aaggagtaca gctctatgga cg
#ccttctgg 420
caagagggga gagcctctgg aacagtgtac agtggggagg agaagctcac tg
#agctcctt 480
gtgaaggctt atggagattt tgcatggagt aaccccctgc atccagatat ct
#tcccagga 540
ctacgcaaga tagaggcaga aattgtgagg atagcttgtt ccctgttcaa tg
#ggggacca 600
gattcgtgtg gatgtgtgac ttctggggga acagaaagca tactgatggc ct
#gcaaagca 660
tatcgggatc tggcctttga gaaggggatc aaaactccag aaattgtggc tc
#cccaaagt 720
gcccatgctg catttaacaa agcagccagt tactttggga tgaagattgt gc
#gggtccca 780
ttgacgaaga tgatggaggt ggatgtgcgg gcaatgagaa gagctatctc ca
#ggaacact 840
gccatgctcg tctgttctac cccacagttt cctcatggtg taatagatcc tg
#tccctgaa 900
gtggccaagc tggctgtcaa atacaaaata ccccttcatg tcgacgcttg tc
#tgggaggc 960
ttcctcatcg tctttatgga gaaagcagga tacccactgg agcacccatt tg
#atttccgg 1020
gtgaaaggtg taaccagcat ttcagctgac acccataagt atggctatgc cc
#caaaaggc 1080
tcatcattgg tgttgtatag tgacaagaag tacaggaact atcagttctt cg
#tcgataca 1140
gattggcagg gtggcatcta tgcttcccca accatcgcag gctcacggcc tg
#gtggcatt 1200
agcgcagcct gttgggctgc cttgatgcac ttcggtgaga acggctatgt tg
#aagctacc 1260
aaacagatca tcaaaactgc tcgcttcctc aagtcagaac tggaaaatat ca
#aaggcatc 1320
tttgtttttg ggaatcccca attgtcagtc attgctctgg gatcccgtga tt
#ttgacatc 1380
taccgactat caaacctgat gactgctaag gggtggaact tgaaccagtt gc
#agttccca 1440
cccagtattc atttctgcat cacattacta cacgcccgga aacgagtagc ta
#tacaattc 1500
ctaaaggaca ttcgagaatc tgtcactcaa atcatgaaga atcctaaagc ga
#agaccaca 1560
ggaatgggtg ccatctatgg catggcccag acaactgttg acaggaatat gg
#ttgcagaa 1620
ttgtcctcag tcttcttgga cagcttgtac agcaccgaca ctgtcaccca gg
#gcagccag 1680
atgaatggtt ctccaaaacc ccactga
#
# 1707
<210> SEQ ID NO 24
<211> LENGTH: 2629
<212> TYPE: DNA
<213> ORGANISM: Drosophila melanogaster
<400> SEQUENCE: 24
agtacgaatt gtcgtgtcaa gcgcacagga agcgatcgca acccggatcg ga
#ttggatcg 60
gccggatcga gccaccatat gtactatata caaacacaca tatatatata ga
#tatatcgc 120
agtctttacg gtgaacagcg tcgatcgcat gtggacaaac aattaaatac aa
#ggtcaaag 180
aagcgctaaa aagtgaagtt aagtcgaaac aaacacgaag ataccaaaga ag
#atatgacg 240
gccaacacag ggacgtcggg tgaaaatctg atgggaaatg gtggaaagac gc
#atgaaccc 300
tccacgccca cttcggatac ggaagtggcc agcggcacgc caacggaact ga
#gcgaaatc 360
ttcttcgtgg acaatagccg gcgcaagcag agcatcaaaa tccaggtgaa ac
#tatgcccg 420
gaaggcgttt acctgcgacg cgaaaccgag gaagatgatc acatcaatga gc
#agctgatc 480
aggatcgatg atatcatagg atcacgctac ggacggcgtt tgaagaaacg ag
#cccgaggc 540
ggtctaaact cctgccgcaa tccaaatgtt ccgggccagg aggcggattc gg
#aaccggat 600
agcgataata gcgcctattt gtacatctat gcatatttga agaaggagaa ac
#cgttgcga 660
cgtgtccaaa cgctccggat tctacgcttt cgttcgagca atgactacgg ag
#tgaatcta 720
cagaccgccg agatgtggca tcatacgatt cgaaagcaca agcgtggcaa tg
#gcagcagt 780
tcgcccgccg attgtggcaa acagttgctc atcctactga atccgaaatc cg
#gttcgggc 840
aaagggcgtg agctcttcca gaaacaggtg gcacctttgc tgacggaagc ag
#aggtgcaa 900
tacgatctcc agatcaccac acatccgcag tatgccaagg agttcgtgcg ga
#ccagaagg 960
gatctgctga cacgctattc gggcattgtg gttgcctccg gcgatggtct at
#tctacgaa 1020
gtgctcaatg ggctaatgga acgcatggat tggcgccgag cctgcaggga gc
#taccgctt 1080
ggcattatac catgtggttc cgggaatggt ctggccaaaa gtgtggccca tc
#attgcaat 1140
gaaccgtacg aaccgaagcc cattctccac gccaccttga cctgcatggc gg
#gcaaaagt 1200
acacccatgg atgtggtcag agtggagctg gcgacgcggg acaagcactt tg
#tgatgtac 1260
tccttcctgt cggtgggctg gggtctgata gccgacatcg atatagagag cg
#agcgattg 1320
agatcgattg gagcgcaaag gtttacgctg tgggccatca agcgattgat cg
#ggctgcgc 1380
agctacaaag gccgagtgtc ctatctactg ggcaagggca agaaggaacc ac
#cagtggaa 1440
gcggctcgag agttgcctgc agaatcaacg gctgcaggaa tccgctcatc tc
#tgcctctg 1500
aatgccgggg aattccatga tctacccgag gaggaggagg gggaggcggt ct
#tggatgga 1560
gaacagttcg ccgatgccat atctttggat cgttcggttt accgccagca tg
#ccgacagt 1620
tggcactcgg ccatgtccag gcgaacggca tattactccc tgggcggacc ca
#gtatgcga 1680
tccaatcgca gccggatgag cattagccag cggatcgagg cagcaaatgc gg
#aattcgct 1740
gagagggtgc caacgggcac cattccacca ttacagatgc cactgctcag ca
#gcgatggt 1800
tggatctgcg aggatggtga ctttgtgatg gtccatgccg cctataccac cc
#atctctcc 1860
tccgatgtct tctttgcgcc cgaatcccgt ctggacgatg gcctcatcta cc
#tggtgatc 1920
atccggagag gcgttagtcg ccatcagctg ctcaatttca tgctgaacct aa
#acgcaggc 1980
acccatctgc ccatcggcga ggatccgttc atcaaggtgg tgccttgtcg gg
#cattccgc 2040
atcgagccga gcagctccga tggcatcctg gtggtggacg gcgagcgggt gg
#aatatgga 2100
cccattcagg cggaggttat gcccggcctg atcaatgtga tgaccaccag tg
#ggcagtag 2160
tgaaatatta aatccgtgtg ctttgcaaag ccttaatcat aacgagatac at
#tcgaagaa 2220
tgtcaacatt caatagctac tttagatcaa acttagttta taagaagttg cc
#tttccaat 2280
taataaccct tactaaattc ataacttttt tatatctgct aggaactttt tc
#caaagttt 2340
acgcattttg acattattag gtggtaaagt tcacaaaatt tctattatct ct
#gtttctat 2400
tgttagtgaa aacccattct catggctctg aaacgcaatc tgtaattttt cc
#atcagccc 2460
atcaaaaaaa aaaaaggaac ttcacactat gcacttaagt agttggttat aa
#gttgtttt 2520
ctattatttt tttttttttt aatcggtcga tagtgttaat tattttaaga ac
#catacgta 2580
gacaaataat aaaccaaaat gctataaaaa atgttaaaaa aaaaaaaaa
# 2629
<210> SEQ ID NO 25
<211> LENGTH: 2609
<212> TYPE: DNA
<213> ORGANISM: Drosophila melanogaster
<400> SEQUENCE: 25
ctgccaccga cggtaacact gcggctatga ttggatgata agcgatccat aa
#aagcctgg 60
acacaaaaga cctgaaacat gctgcttggc agccgaatct ccagtcgaaa tg
#agcgaatc 120
tcttgataag accaccagcc cgagctcggc cagttccagg gccacgcccc cg
#ggaacgca 180
ggatgcggat gagggccacg acgtgagcga taccttctac acgagccagc gc
#aagaaggg 240
cagccacgta tttcgggtgc gccttgacgc cacaggattc accctgcagc gg
#gagtcgcc 300
gggcggtagc attgttaagg agcaacatgt ccgcatatcg gacattgtgg gt
#gcccgctg 360
catgcggccc aagaagagcc ggcgcctggc gatgtcgggc gcctgtgcgt gc
#agctccgg 420
taatcccaat tcgccagcca tctcggcgtc cggcgatcac catcgccctg cc
#accacacc 480
aagcaaatgc agcaccaata gtcgggataa tattccttcg gatggcggcg at
#gtcagcgc 540
gtttctctac gtttttgcct atgttctgaa gaagaggagc ctgcggtcgg ag
#ttgcaccg 600
ggagcgaacg gtgctcactc tgcgcttccg gtcgttcgac accttcgagg ac
#aacatgag 660
ggaggcggat cgttggtaca gatcccttcg ctggcagttg catcgcacgc tg
#gaggagat 720
ctttgtggcg ccgacggtgg atgagcgacg ccgtcgagtg cttgtgctgt tg
#aatcccaa 780
atccggttcc ggtgacgctc gtgaggtctt caacatgcac gtgacgccgg tg
#ctcaacga 840
ggccgaggtg ccctacgacc tgtatgtaac caagcattcc aactttgcca tc
#gagttctt 900
gagcaccagg tgcctggacg cctggtgctg cgtggtggct gtcggcggag ac
#ggtctctt 960
ccacgagata gtcaatggac tgctgcagcg ccaggactgg gcccacgtcc tg
#cctcatct 1020
ggcactggga atcattcctt gcggctccgg aaatggattg gcccgctcca tt
#gcccattg 1080
ttacaacaag ccagtgctag gagctgctct gaccgtaatc agtggacgca gt
#tcacccat 1140
ggacgtggtc cgggtgcagc tgcagagtcg ctccctctac tccttcctgt cc
#atcggctg 1200
gggtctgatc tcggacgtgg acatcgagag cgagcgcatt cgcatgttgg gc
#taccagcg 1260
cttcaccgtg tggaccctct accgtctggt gaatctgcgc acctacaacg gc
#cgaatcag 1320
ctatcttctg acggaccatg aggtgtcctc aacccatagc gctaccggtt at
#gctgccca 1380
gcggagaatg cagagcagcc gtagctgcaa cacgcacatc gacatgctaa at
#gggccggc 1440
gcccatctat cattccagtg ccgagtacct gccacaggag tttgcggacg tg
#atctccct 1500
ggagacgtcc atcaatcagt cgttccgctc gaggtgcgac agctggttgt cg
#gggggatc 1560
gcggcgcagc ttttactatt ccatatcgga gagcatctac cacagtctgg cg
#gatgagag 1620
cgagttcgcc ggcctggcgg ccgcctcgct ggaaaaccgg cagcagaact ac
#ggtccggc 1680
aagcgagctg ccggatctga acgaaccgct gtccgaggat cagggttggc tg
#gtggagga 1740
gggcgagttc gtcatgatgc acgccgttta ccagacccat ctgggcatcg ac
#tgtcattt 1800
tgcgcccaag gcccagctga acgacggcac catctacctg atcctcatac gc
#gccggcat 1860
cagccgcccg cacctgctga gcttcctcta caacatgagc tccggcactc ac
#ctgccgga 1920
gtcgcacgac gaccatgtga aggtgctgcc agtgcgagca ttccgcctgg ag
#ccctacga 1980
caatcacggc atcatcacgg tcgacggcga gcgcgtcgag ttcgggcccc tc
#caagctga 2040
ggtcctgccg ggcatagccc gcgtcatggt gcccaagtag gaggagctta ct
#gaagacca 2100
tcaagcaata gaaatctcaa ttttggaatc tctgcatttg tagctaatac tt
#agggtccc 2160
aggtggccga tatgagagag ttgtgcattc tacatatttc gtgtttttgt gg
#cctgcttc 2220
tgccaaccaa tcatgtattg ttaacatttt aaacacaata acagctattt cc
#gaaatatc 2280
taacatgttt gtttataaaa cgtgtgccat atgaagtgca cgtgaattta tt
#tttatctc 2340
ggctgttcaa aatagcgatg aaagtcttat ttattttgtt cttttttttt tt
#aaactgtg 2400
taacgaaatg agatatatat tcaaaatgtt taaagatgaa tacaaataaa tc
#ttcatgaa 2460
ttcaaaaatc ttagaaagta acagtgtaag taacagagct aaatcattta ca
#attccata 2520
ttttaaagta ggaagttaag aatataacat ctttgagctt gaaataaaaa at
#aaaaatgt 2580
taactaaaaa aaaaaaaaaa aaaaaaaaa
#
# 2609
<210> SEQ ID NO 26
<211> LENGTH: 2043
<212> TYPE: DNA
<213> ORGANISM: Drosophila melanogaster
<400> SEQUENCE: 26
gtcactctaa gccgcaatga gtttgtacga ttaaaagttt atgtctattc gc
#gtttttcg 60
aagctttccc gattcccgta gctgtcccac tgtacagctt gccacacgat gc
#gtccgttc 120
tccggcagcg attgccttaa gcccgtcacc gagggcatca accgggcgtt cg
#gcgccaag 180
gagccttggc aggtcgccac catcacggcc accacggtgc tgggaggcgt ct
#ggctctgg 240
actgtgatct gccaggatga aaatctttac attcgtggca agcgtcagtt ct
#ttaagttt 300
gccaagaaga ttccagccgt gcgtcgtcag gtggagactg aattggccaa gg
#ccaaaaac 360
gacttcgaga cggaaatcaa aaagagcaac gcccacctta cctactcgga aa
#ctctgccc 420
gagaagggac tcagcaagga ggagatcctc cgactggtgg atgagcacct ga
#agactggt 480
cactacaact ggcgtgatgg tcgtgtatct ggcgcggtct acggctacaa gc
#ctgatctg 540
gtggagctcg tcactgaagt gtacggcaag gcctcctaca ccaatccctt gc
#acgcagat 600
cttttcccgg gagtttgcaa aatggaggcg gaggtagtgc gcatggcatg ca
#acctgttc 660
catggaaact cagccagctg tggaaccatg accaccggcg gcaccgaatc ca
#ttgtaatg 720
gccatgaagg cgtacaggga tttcgctaga gagtacaagg gaatcaccag gc
#caaacatc 780
gtggtgccta agacggtcca cgcggccttc gacaagggtg gtcagtactt ta
#atatccac 840
gtgcgatccg tggatgtaga tccggagacc tacgaagtgg acattaagaa gt
#tcaaacgt 900
gccattaata ggaacacgat tctgctggtt gggtctgctc caaacttccc ct
#atggaacc 960
atagatgata tcgaagctat cgccgctttg ggcgttaagt acgacattcc cg
#tgcacgtg 1020
gacgcctgcc tgggcagctt tgtggtggcc ttggtccgca acgccggcta ta
#agctgcgt 1080
cccttcgact ttgaggtcaa gggagtgacc agtatctccg ctgataccca ca
#agtatggt 1140
ttcgcgccca agggatcatc ggtgatcctt tactcggaca agaagtacaa gg
#accatcag 1200
ttcactgtga ctactgactg gcctggcggc gtgtatggtt ctcccacagt ca
#acggttcc 1260
cgtgccggag gtattatcgc cgcctgctgg gctaccatga tgagctttgg ct
#atgatggt 1320
tatctggaag ccactaagcg cattgtggat acggcgcgct atatcgagag gg
#gcgttcgc 1380
gacatcgatg gcatctttat ctttggcaag ccagctactt cagtgattgc cc
#tgggttcc 1440
aatgtgtttg acattttccg gctatcggat tcgctgtgca aactgggctg ga
#acctaaat 1500
gcgctgcagt ttccatctgg tatccacctg tgcgtgacgg acatgcacac ac
#agcccgga 1560
gtcgcggata aattcattgc cgatgtgcgc agctgtacgg cggagatcat ga
#aggatccc 1620
ggccagcccg tcgttggaaa gatggctctc tacggcatgg cacagagcat ac
#ccgaccgt 1680
tcggtgatcg gagaagtgac tcgcctattc ctgcactcca tgtactacac tc
#ccagccag 1740
aaatagacac ctggagcaat ccccgttctc ttcgcccacc ccacggagct aa
#tgcatttc 1800
ctgtgctgta tttaaaccac caaaacaccc cgtcgttaaa ccttcctcaa gc
#aatttata 1860
ttaggatgca attagtgctg taatcgaggg tacaaaacgt cgttctacgc ga
#aaatctat 1920
ctacctatgt tcatcccatt tgtcaacatt cgtcgctcta agagccatgt ta
#ttaaagtg 1980
tttttctgtg taacttgcta gtgaaataat aatataatat taatcaaaaa aa
#aaaaaaaa 2040
aaa
#
#
# 2043
<210> SEQ ID NO 27
<211> LENGTH: 2043
<212> TYPE: DNA
<213> ORGANISM: Drosophila melanogaster
<400> SEQUENCE: 27
gtcactctaa gccgcaatga gtttgtacga ttaaaagttt atgtctattc gc
#gtttttcg 60
aagctttccc gattcccgta gctgtcccac tgtacagctt gccacacgat gc
#gtccgttc 120
tccggcagcg attgccttaa gcccgtcacc gagggcatca accgggcgtt cg
#gcgccaag 180
gagccttggc aggtcgccac catcacggcc accacggtgc tgggaggcgt ct
#ggctctgg 240
actgtgatct gccaggatga aaatctttac attcgtggca agcgtcagtt ct
#ttaagttt 300
gccaagaaga ttccagccgt gcgtcgtcag gtggagactg aattggccaa gg
#ccaaaaac 360
gacttcgaga cggaaatcaa aaagagcaac gcccacctta cctactcgga aa
#ctctgccc 420
gagaagggac tcagcaagga ggagatcctc cgactggtgg atgagcacct ga
#agactggt 480
cactacaact ggcgtgatgg tcgtgtatct ggcgcggtct acggctacaa gc
#ctgatctg 540
gtggagctcg tcactgaagt gtacggcaag gcctcctaca ccaatccctt gc
#acgcagat 600
cttttcccgg gagtttgcaa aatggaggcg gaggtagtgc gcatggcatg ca
#acctgttc 660
catggaaact cagccagctg tggaaccatg accaccggcg gcaccgaatc ca
#ttgtaatg 720
gccatgaagg cgtacaggga tttcgctaga gagtacaagg gaatcaccag gc
#caaacatc 780
gtggtgccta agacggtcca cgcggccttc gacaagggtg gtcagtactt ta
#atatccac 840
gtgcgatccg tggatgtaga tccggagacc tacgaagtgg acattaagaa gt
#tcaaacgt 900
gccattaata ggaacacgat tctgctggtt gggtctgctc caaacttccc ct
#atggaacc 960
atagatgata tcgaagctat cgccgctttg ggcgttaagt acgacattcc cg
#tgcacgtg 1020
gacgcctgcc tgggcagctt tgtggtggcc ttggtccgca acgccggcta ta
#agctgcgt 1080
cccttcgact ttgaggtcaa gggagtgacc agtatctccg ctgataccca ca
#agtatggt 1140
ttcgcgccca agggatcatc ggtgatcctt tactcggaca agaagtacaa gg
#accatcag 1200
ttcactgtga ctactgactg gcctggcggc gtgtatggtt ctcccacagt ca
#acggttcc 1260
cgtgccggag gtattatcgc cgcctgctgg gctaccatga tgagctttgg ct
#atgatggt 1320
tatctggaag ccactaagcg cattgtggat acggcgcgct atatcgagag gg
#gcgttcgc 1380
gacatcgatg gcatctttat ctttggcaag ccagctactt cagtgattgc cc
#tgggttcc 1440
aatgtgtttg acattttccg gctatcggat tcgctgtgca aactgggctg ga
#acctaaat 1500
gcgctgcagt ttccatctgg tatccacctg tgcgtgacgg acatgcacac ac
#agcccgga 1560
gtcgcggata aattcattgc cgatgtgcgc agctgtacgg cggagatcat ga
#aggatccc 1620
ggccagcccg tcgttggaaa gatggctctc tacggcatgg cacagagcat ac
#ccgaccgt 1680
tcggtgatcg gagaagtgac tcgcctattc ctgcactcca tgtactacac tc
#ccagccag 1740
aaatagacac ctggagcaat ccccgttctc ttcgcccacc ccacggagct aa
#tgcatttc 1800
ctgtgctgta tttaaaccac caaaacaccc cgtcgttaaa ccttcctcaa gc
#aatttata 1860
ttaggatgca attagtgctg taatcgaggg tacaaaacgt cgttctacgc ga
#aaatctat 1920
ctacctatgt tcatcccatt tgtcaacatt cgtcgctcta agagccatgt ta
#ttaaagtg 1980
tttttctgtg taacttgcta gtgaaataat aatataatat taatcaaaaa aa
#aaaaaaaa 2040
aaa
#
#
# 2043
<210> SEQ ID NO 28
<211> LENGTH: 641
<212> TYPE: PRT
<213> ORGANISM: Drosophlia melanogaster
<400> SEQUENCE: 28
Met Thr Ala Asn Thr Gly Thr Ser Gly Glu As
#n Leu Met Gly Asn Gly
  1 5
# 10
# 15
Gly Lys Thr His Glu Pro Ser Thr Pro Thr Se
#r Asp Thr Glu Val Ala
                        20
# 25
# 30
Ser Gly Thr Pro Thr Glu Leu Ser Glu Ile Ph
#e Phe Val Asp Asn Ser
                35
# 40
# 45
Arg Arg Lys Gln Ser Ile Lys Ile Gln Val Ly
#s Leu Cys Pro Glu Gly
        50
# 55
# 60
Val Tyr Leu Arg Arg Glu Thr Glu Glu Asp As
#p His Ile Asn Glu Gln
65
#70
#75
#80
Leu Ile Arg Ile Asp Asp Ile Ile Gly Ser Ar
#g Tyr Gly Arg Arg Leu
                                85
# 90
# 95
Lys Lys Arg Ala Arg Gly Gly Leu Asn Ser Cy
#s Arg Asn Pro Asn Val
                        100
# 105
# 110
Pro Gly Gln Glu Ala Asp Ser Glu Pro Asp Se
#r Asp Asn Ser Ala Tyr
                115
# 120
# 125
Leu Tyr Ile Tyr Ala Tyr Leu Lys Lys Glu Ly
#s Pro Leu Arg Arg Val
        130
# 135
# 140
Gln Thr Leu Arg Ile Leu Arg Phe Arg Ser Se
#r Asn Asp Tyr Gly Val
145 1
#50 1
#55 1
#60
Asn Leu Gln Thr Ala Glu Met Trp His His Th
#r Ile Arg Lys His Lys
                                165
# 170
# 175
Arg Gly Asn Gly Ser Ser Ser Pro Ala Asp Cy
#s Gly Lys Gln Leu Leu
                        180
# 185
# 190
Ile Leu Leu Asn Pro Lys Ser Gly Ser Gly Ly
#s Gly Arg Glu Leu Phe
                195
# 200
# 205
Gln Lys Gln Val Ala Pro Leu Leu Thr Glu Al
#a Glu Val Gln Tyr Asp
        210
# 215
# 220
Leu Gln Ile Thr Thr His Pro Gln Tyr Ala Ly
#s Glu Phe Val Arg Thr
225 2
#30 2
#35 2
#40
Arg Arg Asp Leu Leu Thr Arg Tyr Ser Gly Il
#e Val Val Ala Ser Gly
                                245
# 250
# 255
Asp Gly Leu Phe Tyr Glu Val Leu Asn Gly Le
#u Met Glu Arg Met Asp
                        260
# 265
# 270
Trp Arg Arg Ala Cys Arg Glu Leu Pro Leu Gl
#y Ile Ile Pro Cys Gly
                275
# 280
# 285
Ser Gly Asn Gly Leu Ala Lys Ser Val Ala Hi
#s His Cys Asn Glu Pro
        290
# 295
# 300
Tyr Glu Pro Lys Pro Ile Leu His Ala Thr Le
#u Thr Cys Met Ala Gly
305 3
#10 3
#15 3
#20
Lys Ser Thr Pro Met Asp Val Val Arg Val Gl
#u Leu Ala Thr Arg Asp
                                325
# 330
# 335
Lys His Phe Val Met Tyr Ser Phe Leu Ser Va
#l Gly Trp Gly Leu Ile
                        340
# 345
# 350
Ala Asp Ile Asp Ile Glu Ser Glu Arg Leu Ar
#g Ser Ile Gly Ala Gln
                355
# 360
# 365
Arg Phe Thr Leu Trp Ala Ile Lys Arg Leu Il
#e Gly Leu Arg Ser Tyr
        370
# 375
# 380
Lys Gly Arg Val Ser Tyr Leu Leu Gly Lys Gl
#y Lys Lys Glu Pro Pro
385 3
#90 3
#95 4
#00
Val Glu Ala Ala Arg Glu Leu Pro Ala Glu Se
#r Thr Ala Ala Gly Ile
                                405
# 410
# 415
Arg Ser Ser Leu Pro Leu Asn Ala Gly Glu Ph
#e His Asp Leu Pro Glu
                        420
# 425
# 430
Glu Glu Glu Gly Glu Ala Val Leu Asp Gly Gl
#u Gln Phe Ala Asp Ala
                435
# 440
# 445
Ile Ser Leu Asp Arg Ser Val Tyr Arg Gln Hi
#s Ala Asp Ser Trp His
        450
# 455
# 460
Ser Ala Met Ser Arg Arg Thr Ala Tyr Tyr Se
#r Leu Gly Gly Pro Ser
465 4
#70 4
#75 4
#80
Met Arg Ser Asn Arg Ser Arg Met Ser Ile Se
#r Gln Arg Ile Glu Ala
                                485
# 490
# 495
Ala Asn Ala Glu Phe Ala Glu Arg Val Pro Th
#r Gly Thr Ile Pro Pro
                        500
# 505
# 510
Leu Gln Met Pro Leu Leu Ser Ser Asp Gly Tr
#p Ile Cys Glu Asp Gly
                515
# 520
# 525
Asp Phe Val Met Val His Ala Ala Tyr Thr Th
#r His Leu Ser Ser Asp
        530
# 535
# 540
Val Phe Phe Ala Pro Glu Ser Arg Leu Asp As
#p Gly Leu Ile Tyr Leu
545 5
#50 5
#55 5
#60
Val Ile Ile Arg Arg Gly Val Ser Arg His Gl
#n Leu Leu Asn Phe Met
                                565
# 570
# 575
Leu Asn Leu Asn Ala Gly Thr His Leu Pro Il
#e Gly Glu Asp Pro Phe
                        580
# 585
# 590
Ile Lys Val Val Pro Cys Arg Ala Phe Arg Il
#e Glu Pro Ser Ser Ser
                595
# 600
# 605
Asp Gly Ile Leu Val Val Asp Gly Glu Arg Va
#l Glu Tyr Gly Pro Ile
        610
# 615
# 620
Gln Ala Glu Val Met Pro Gly Leu Ile Asn Va
#l Met Thr Thr Ser Gly
625 6
#30 6
#35 6
#40
Gln
<210> SEQ ID NO 29
<211> LENGTH: 907
<212> TYPE: PRT
<213> ORGANISM: Drosophila melanogaster
<400> SEQUENCE: 29
Met Ser Glu Ser Leu Asp Lys Thr Thr Ser Pr
#o Ser Ser Ala Ser Ser
  1 5
# 10
# 15
Arg Ala Thr Pro Pro Gly Thr Gln Asp Ala As
#p Glu Gly His Asp Val
                        20
# 25
# 30
Ser Asp Thr Phe Tyr Thr Ser Gln Arg Lys Ly
#s Gly Ser His Val Phe
                35
# 40
# 45
Arg Val Arg Leu Asp Ala Thr Gly Phe Thr Le
#u Gln Arg Glu Ser Pro
        50
# 55
# 60
Gly Gly Ser Ile Val Lys Glu Gln His Val Ar
#g Ile Ser Asp Ile Val
65
#70
#75
#80
Gly Ala Arg Cys Met Arg Pro Lys Lys Ser Ar
#g Arg Leu Ala Met Ser
                                85
# 90
# 95
Gly Ala Cys Ala Cys Ser Ser Gly Asn Pro As
#n Ser Pro Ala Ile Ser
                        100
# 105
# 110
Ala Ser Gly Asp His His Arg Pro Ala Thr Th
#r Pro Ser Lys Cys Ser
                115
# 120
# 125
Thr Asn Ser Arg Asp Asn Ile Pro Ser Asp Gl
#y Gly Asp Val Ser Ala
        130
# 135
# 140
Phe Leu Tyr Val Phe Ala Tyr Val Leu Lys Ly
#s Arg Ser Leu Arg Ser
145 1
#50 1
#55 1
#60
Glu Leu His Arg Glu Arg Thr Val Leu Thr Le
#u Arg Phe Arg Ser Phe
                                165
# 170
# 175
Asp Thr Phe Glu Asp Asn Met Arg Glu Ala As
#p Arg Trp Tyr Arg Ser
                        180
# 185
# 190
Leu Arg Trp Gln Leu His Arg Thr Leu Glu Gl
#u Ile Phe Val Ala Pro
                195
# 200
# 205
Thr Val Asp Glu Arg Arg Arg Arg Val Leu Va
#l Leu Leu Asn Pro Lys
        210
# 215
# 220
Ser Gly Ser Gly Asp Ala Arg Glu Val Phe As
#n Met His Val Thr Pro
225 2
#30 2
#35 2
#40
Val Leu Asn Glu Ala Glu Val Pro Tyr Asp Le
#u Tyr Val Thr Lys His
                                245
# 250
# 255
Ser Asn Phe Ala Ile Glu Phe Leu Ser Thr Ar
#g Cys Leu Asp Ala Trp
                        260
# 265
# 270
Cys Cys Val Val Ala Val Gly Gly Asp Gly Le
#u Phe His Glu Ile Val
                275
# 280
# 285
Asn Gly Leu Leu Gln Arg Gln Asp Trp Ala Hi
#s Val Leu Pro His Leu
        290
# 295
# 300
Ala Leu Gly Ile Ile Pro Cys Gly Ser Gly As
#n Gly Leu Ala Arg Ser
305 3
#10 3
#15 3
#20
Ile Ala His Cys Tyr Asn Lys Pro Val Leu Gl
#y Ala Ala Leu Thr Val
                                325
# 330
# 335
Ile Ser Gly Arg Ser Ser Pro Met Asp Val Va
#l Arg Val Gln Leu Gln
                        340
# 345
# 350
Ser Arg Ser Leu Tyr Ser Phe Leu Ser Ile Gl
#y Trp Gly Leu Ile Ser
                355
# 360
# 365
Asp Val Asp Ile Glu Ser Glu Arg Ile Arg Me
#t Leu Gly Tyr Gln Arg
        370
# 375
# 380
Phe Thr Val Trp Thr Leu Tyr Arg Leu Val As
#n Leu Arg Thr Tyr Asn
385 3
#90 3
#95 4
#00
Gly Arg Ile Ser Tyr Leu Leu Thr Asp His Gl
#u Val Ser Ser Thr His
                                405
# 410
# 415
Ser Ala Thr Gly Tyr Ala Ala Gln Arg Arg Me
#t Gln Ser Ser Arg Ser
                        420
# 425
# 430
Cys Asn Thr His Ile Asp Met Leu Asn Gly Pr
#o Ala Pro Ile Tyr His
                435
# 440
# 445
Ser Ser Ala Glu Tyr Leu Pro Gln Glu Phe Al
#a Asp Val Ile Ser Leu
        450
# 455
# 460
Glu Thr Ser Ile Asn Gln Ser Phe Arg Ser Ar
#g Cys Asp Ser Trp Leu
465 4
#70 4
#75 4
#80
Ser Gly Gly Ser Arg Arg Ser Phe Tyr Tyr Se
#r Ile Ser Glu Ser Ile
                                485
# 490
# 495
Tyr His Ser Leu Ala Asp Glu Ser Glu Phe Al
#a Gly Leu Ala Ala Ala
                        500
# 505
# 510
Ser Leu Glu Asn Arg Gln Gln Asn Tyr Gly Pr
#o Ala Ser Glu Leu Pro
                515
# 520
# 525
Asp Leu Asn Glu Pro Leu Ser Glu Asp Gln Gl
#y Trp Leu Val Glu Glu
        530
# 535
# 540
Gly Glu Phe Val Met Met His Ala Val Tyr Gl
#n Thr His Leu Gly Ile
545 5
#50 5
#55 5
#60
Asp Cys His Phe Ala Pro Lys Ala Gln Leu As
#n Asp Gly Thr Ile Tyr
                                565
# 570
# 575
Leu Ile Leu Ile Arg Ala Gly Ile Ser Arg Pr
#o His Leu Leu Ser Phe
                        580
# 585
# 590
Leu Tyr Asn Met Ser Ser Gly Thr His Leu Pr
#o Glu Ser His Asp Asp
                595
# 600
# 605
His Val Lys Val Leu Pro Val Arg Ala Phe Ar
#g Leu Glu Pro Tyr Asp
        610
# 615
# 620
Asn His Gly Ile Ile Thr Val Asp Gly Glu Ar
#g Val Glu Phe Gly Pro
625 6
#30 6
#35 6
#40
Leu Gln Ala Glu Val Leu Pro Gly Ile Ala Ar
#g Val Met Val Pro Asn
                                645
# 650
# 655
Val Ser Thr Phe Arg Phe Gln Ser Ala Thr Le
#u Gln His Gly Ile Pro
                        660
# 665
# 670
Val Cys Ile Pro Val Arg Lys Arg Phe Val Le
#u Tyr Asn Met Ser Ser
                675
# 680
# 685
Glu Glu Leu Ala Pro Ile Asn Glu Gln Asp Ph
#e Lys Asp Leu Lys Glu
        690
# 695
# 700
Arg Met Lys Leu Ile Val Glu Ala Asp Pro Ly
#s Gln Tyr His Asn Asp
705 7
#10 7
#15 7
#20
Phe Ser Leu Arg Arg Tyr Leu Arg Ala Phe Ly
#s Thr Thr Asp Asp Ala
                                725
# 730
# 735
Phe Gln Ala Ile Leu Lys Thr Asn Lys Trp Ar
#g Glu Thr Tyr Gly Val
                        740
# 745
# 750
Asp Lys Leu Ser Glu Met Asp Arg Ser Gln Le
#u Asp Lys Lys Ala Arg
                755
# 760
# 765
Leu Leu Arg His Arg Asp Cys Ile Gly Arg Pr
#o Val Ile Tyr Ile Pro
        770
# 775
# 780
Ala Lys Asn His Ser Ser Glu Arg Asp Ile As
#p Glu Leu Thr Arg Phe
785 7
#90 7
#95 8
#00
Ile Val Tyr Asn Leu Glu Glu Ala Cys Lys Ly
#s Cys Phe Glu Glu Val
                                805
# 810
# 815
Thr Asp Arg Leu Cys Ile Val Phe Asp Leu Al
#a Glu Phe Ser Thr Ser
                        820
# 825
# 830
Cys Met Asp Tyr Gln Leu Val Gln Asn Leu Il
#e Trp Leu Leu Gly Lys
                835
# 840
# 845
His Phe Pro Glu Arg Leu Gly Val Cys Leu Il
#e Ile Asn Ser Pro Gly
        850
# 855
# 860
Leu Phe Ser Thr Ile Trp Pro Ala Ile Arg Va
#l Leu Leu Asp Asp Asn
865 8
#70 8
#75 8
#80
Thr Ala Lys Lys Val Lys Phe Val Ala Asp Gl
#u Ala Glu Leu Cys Gln
                                885
# 890
# 895
Tyr Leu Ile Pro Asp Ile Leu Pro Thr Asp Me
#t
                        900
# 905
<210> SEQ ID NO 30
<211> LENGTH: 1875
<212> TYPE: DNA
<213> ORGANISM: Saccharomyces cerevisiaie
<400> SEQUENCE: 30
atggtggtgc agaaaaaact tagggctatc ttgaccgatg aaggtgtatt ga
#tcaaatcg 60
caatcacacc atatgttcaa taagcatggt caactcagaa gcggagattc tt
#tatccttg 120
ttgagctgct tgtcctgtct ggatgatgga actttgagct ctgatggagg tt
#cttttgat 180
gaggatgatt ccctggaact gttgcctctt aatactacca ttccgttcaa ca
#gaattttg 240
aacgcaaaat atgtgaatgt cggtcagaaa ggcttcaata atggcaaaat tt
#cttcgaat 300
ccttttcaaa cggaaaatct gagttcttcg tctgaaaatg acgacgttga ga
#atcatagt 360
ttgagcaatg ataaggctcc tgtaagcgaa tcacagtcat ttcccaaaaa ag
#acaagtgg 420
gatacaaaaa cgaacactgt gaaggtgtct cccgatgatt cacaggataa ct
#caccatct 480
ttagggataa aagataatca acagttaatt gagttaactt ttgctgtacc ca
#agggccat 540
gatgttatac cacaaaaatt aaccttgtta atagatcacg tttctaggaa at
#cgagagca 600
aataccggag aggagaacat ttcttctggt actgtggaag aaatcctgga aa
#aaagttat 660
gaaaattcca agagaaacag atcgatatta gtcattatta atccccacgg tg
#gtaaaggt 720
actgctaaaa atttattcct gacaaaagca aggccaatac tagtggaaag tg
#gctgcaaa 780
atagaaattg catacacaaa atatgcccgt cacgccatcg atattgccaa ag
#atttagat 840
atcagcaaat acgataccat tgcatgtgcc tcgggtgatg gtattccata cg
#aagtaatt 900
aatgggcttt atagaagacc cgacagagtg gatgcgttca ataaactagc cg
#taactcag 960
ctaccttgcg gttcaggaaa tgctatgagc atttcatgtc attggacaaa ta
#acccatcg 1020
tacgccgctc tgtgccttgt caaatccatt gaaacaagaa tagacttaat gt
#gttgttcc 1080
caaccttctt atatgaacga atggccaaga ttatcctttt tgagtcagac gt
#acggcgtt 1140
attgcagaat ctgatattaa cactgaattc atcagatgga tgggtcccgt ta
#ggtttaat 1200
ttgggtgtag cattcaacat tatccaaggt aagaaatatc cctgcgaagt tt
#tcgtcaaa 1260
tatgctgcca aatctaaaaa ggaattaaaa gttcatttct tagaaaataa ag
#ataaaaac 1320
aaaggatgtt taaccttcga accaaatcct agcccaaact cttcgccgga tt
#tactatct 1380
aaaaacaata tcaacaacag tacaaaagat gaactttcac cgaattttct ca
#acgaggac 1440
aactttaaat taaagtatcc gatgacggaa ccagtaccta gagactggga ga
#aaatggat 1500
tcagagctaa ctgataactt aacaatcttt tacacaggga aaatgccgta ta
#ttgctaag 1560
gacaccaaat tttttcccgc tgctttacca gcggatggta ccattgattt ag
#tcataacg 1620
gatgcaagaa tcccagtgac aagaatgaca ccaattttat tatccttgga ta
#aaggttct 1680
catgtattag agccagaagt tattcactca aaaatattgg cttataagat ta
#taccaaaa 1740
gtggagtcag gtttattttc agtggatggt gaaaagtttc ctttggaacc ct
#tgcaagtg 1800
gaaataatgc ccatgttatg caagacgttg ctaaggaatg gtagatatat cg
#atacagag 1860
tttgaatcca tgtag
#
#
# 1875
<210> SEQ ID NO 31
<211> LENGTH: 624
<212> TYPE: PRT
<213> ORGANISM: Saccharomyces cerevisiaie
<400> SEQUENCE: 31
Met Val Val Gln Lys Lys Leu Arg Ala Ile Le
#u Thr Asp Glu Gly Val
  1 5
# 10
# 15
Leu Ile Lys Ser Gln Ser His His Met Phe As
#n Lys His Gly Gln Leu
                        20
# 25
# 30
Arg Ser Gly Asp Ser Leu Ser Leu Leu Ser Cy
#s Leu Ser Cys Leu Asp
                35
# 40
# 45
Asp Gly Thr Leu Ser Ser Asp Gly Gly Ser Ph
#e Asp Glu Asp Asp Ser
        50
# 55
# 60
Leu Glu Leu Leu Pro Leu Asn Thr Thr Ile Pr
#o Phe Asn Arg Ile Leu
65
#70
#75
#80
Asn Ala Lys Tyr Val Asn Val Gly Gln Lys Gl
#y Phe Asn Asn Gly Lys
                                85
# 90
# 95
Ile Ser Ser Asn Pro Phe Gln Thr Glu Asn Le
#u Ser Ser Ser Ser Glu
                        100
# 105
# 110
Asn Asp Asp Val Glu Asn His Ser Leu Ser As
#n Asp Lys Ala Pro Val
                115
# 120
# 125
Ser Glu Ser Gln Ser Phe Pro Lys Lys Asp Ly
#s Trp Asp Thr Lys Thr
        130
# 135
# 140
Asn Thr Val Lys Val Ser Pro Asp Asp Ser Gl
#n Asp Asn Ser Pro Ser
145 1
#50 1
#55 1
#60
Leu Gly Ile Lys Asp Asn Gln Gln Leu Ile Gl
#u Leu Thr Phe Ala Val
                                165
# 170
# 175
Pro Lys Gly His Asp Val Ile Pro Gln Lys Le
#u Thr Leu Leu Ile Asp
                        180
# 185
# 190
His Val Ser Arg Lys Ser Arg Ala Asn Thr Gl
#y Glu Glu Asn Ile Ser
                195
# 200
# 205
Ser Gly Thr Val Glu Glu Ile Leu Glu Lys Se
#r Tyr Glu Asn Ser Lys
        210
# 215
# 220
Arg Asn Arg Ser Ile Leu Val Ile Ile Asn Pr
#o His Gly Gly Lys Gly
225 2
#30 2
#35 2
#40
Thr Ala Lys Asn Leu Phe Leu Thr Lys Ala Ar
#g Pro Ile Leu Val Glu
                                245
# 250
# 255
Ser Gly Cys Lys Ile Glu Ile Ala Tyr Thr Ly
#s Tyr Ala Arg His Ala
                        260
# 265
# 270
Ile Asp Ile Ala Lys Asp Leu Asp Ile Ser Ly
#s Tyr Asp Thr Ile Ala
                275
# 280
# 285
Cys Ala Ser Gly Asp Gly Ile Pro Tyr Glu Va
#l Ile Asn Gly Leu Tyr
        290
# 295
# 300
Arg Arg Pro Asp Arg Val Asp Ala Phe Asn Ly
#s Leu Ala Val Thr Gln
305 3
#10 3
#15 3
#20
Leu Pro Cys Gly Ser Gly Asn Ala Met Ser Il
#e Ser Cys His Trp Thr
                                325
# 330
# 335
Asn Asn Pro Ser Tyr Ala Ala Leu Cys Leu Va
#l Lys Ser Ile Glu Thr
                        340
# 345
# 350
Arg Ile Asp Leu Met Cys Cys Ser Gln Pro Se
#r Tyr Met Asn Glu Trp
                355
# 360
# 365
Pro Arg Leu Ser Phe Leu Ser Gln Thr Tyr Gl
#y Val Ile Ala Glu Ser
        370
# 375
# 380
Asp Ile Asn Thr Glu Phe Ile Arg Trp Met Gl
#y Pro Val Arg Phe Asn
385 3
#90 3
#95 4
#00
Leu Gly Val Ala Phe Asn Ile Ile Gln Gly Ly
#s Lys Tyr Pro Cys Glu
                                405
# 410
# 415
Val Phe Val Lys Tyr Ala Ala Lys Ser Lys Ly
#s Glu Leu Lys Val His
                        420
# 425
# 430
Phe Leu Glu Asn Lys Asp Lys Asn Lys Gly Cy
#s Leu Thr Phe Glu Pro
                435
# 440
# 445
Asn Pro Ser Pro Asn Ser Ser Pro Asp Leu Le
#u Ser Lys Asn Asn Ile
        450
# 455
# 460
Asn Asn Ser Thr Lys Asp Glu Leu Ser Pro As
#n Phe Leu Asn Glu Asp
465 4
#70 4
#75 4
#80
Asn Phe Lys Leu Lys Tyr Pro Met Thr Glu Pr
#o Val Pro Arg Asp Trp
                                485
# 490
# 495
Glu Lys Met Asp Ser Glu Leu Thr Asp Asn Le
#u Thr Ile Phe Tyr Thr
                        500
# 505
# 510
Gly Lys Met Pro Tyr Ile Ala Lys Asp Thr Ly
#s Phe Phe Pro Ala Ala
                515
# 520
# 525
Leu Pro Ala Asp Gly Thr Ile Asp Leu Val Il
#e Thr Asp Ala Arg Ile
        530
# 535
# 540
Pro Val Thr Arg Met Thr Pro Ile Leu Leu Se
#r Leu Asp Lys Gly Ser
545 5
#50 5
#55 5
#60
His Val Leu Glu Pro Glu Val Ile His Ser Ly
#s Ile Leu Ala Tyr Lys
                                565
# 570
# 575
Ile Ile Pro Lys Val Glu Ser Gly Leu Phe Se
#r Val Asp Gly Glu Lys
                        580
# 585
# 590
Phe Pro Leu Glu Pro Leu Gln Val Glu Ile Me
#t Pro Met Leu Cys Lys
                595
# 600
# 605
Thr Leu Leu Arg Asn Gly Arg Tyr Ile Asp Th
#r Glu Phe Glu Ser Met
        610
# 615
# 620
<210> SEQ ID NO 32
<211> LENGTH: 2064
<212> TYPE: DNA
<213> ORGANISM: Saccharomyces cerevisiaie
<400> SEQUENCE: 32
atgactttga aaccttcaaa gagacgtaag ggcaggtctc gccattccag ga
#agaagcaa 60
ataacgtcag cgatactgac tgaggaagga ataatgatca aggcaaaacc at
#caagtcct 120
tacacatacg caaatagaat ggcagataaa cgaagtcgca gcagcattga ca
#acatcagt 180
agaactagct ttcaaagcaa catcagtaga actagctttc aaagcaacag tg
#ataacaac 240
agtatatttg aaacggcttc actaattagc tgtgttacct gtttaagcga ta
#ctgataca 300
atagacagat cggaaacatc gacaacggat acaagtaaag atgatctttc tg
#ctaatcca 360
aaacttcatt atccttcggt gaatggtcaa ttgccagcaa acaccgttat cc
#cctatgga 420
cgaattctgg atgccagata cattgaaaag gaacctctgc attattatga tg
#ccaattca 480
tcacccagtt cacctttaag cagctcaatg agtaacatta gtgaaaagtg tg
#atcttgat 540
gaattagagt cttcccaaaa aaaagaaagg aagggcaact cgctatcgcg ag
#gaagtaac 600
agtagtagta gcctcctgac ttccagatct ccttttacga aactagtaga gg
#ttatattt 660
gctaggccaa gacggcatga cgttgtaccc aaaagggttt cattgtatat tg
#actataaa 720
ccccattcat cttctcactt aaaagaagaa gatgacttgg ttgaggagat tt
#taaagaga 780
agctacaaaa acactagaag gaacaaatcc atatttgtga tcattaatcc gt
#ttggtggt 840
aaaggtaagg cgaaaaaact gtttatgaca aaggcaaagc cgttactatt ag
#caagtcgg 900
tgttccatag aagtggttta tacaaaatac cctggtcatg ctatagagat cg
#cgcgggaa 960
atggatattg acaaatatga cactattgct tgcgcttcgg gagatggcat tc
#ctcatgag 1020
gtgatcaatg ggttatacca aaggcctgat catgtcaaag cattcaacaa ta
#tcgccatt 1080
acagaaattc catgcggatc aggtaacgca atgagcgtat cctgccactg ga
#caaacaat 1140
ccttcgtact caactttatg cttaattaaa tcgatagaga ctagaattga tt
#tgatgtgt 1200
tgttcgcagc cttcttatgc aagagagcac ccaaagttat catttttaag tc
#aaacatat 1260
ggtctcattg cagaaactga cataaacact gaatttatta gatggatggg ac
#ctgcaagg 1320
tttgaattgg gtgtagcctt caatatcata caaaaaaaaa aatatccttg tg
#agatatat 1380
gtaaagtatg ctgccaaatc aaaaaacgag ttaaaaaatc attacctgga ac
#acaaaaat 1440
aaagggtcgt tagaattcca gcatattact atgaacaaag ataacgagga tt
#gtgataat 1500
tacaattacg aaaatgaata cgaaaccgaa aacgaagatg aagatgaaga tg
#cggatgcg 1560
gatgacgaag actcccactt gatatctcgt gatctggcag attctagtgc tg
#atcaaatt 1620
aaagaggaag atttcaaaat aaaatatcca ttagatgaag gtatccctag tg
#actgggaa 1680
agattggatc ctaatatttc gaacaaccta ggtatcttct atacgggtaa aa
#tgccatat 1740
gtggctgctg acactaaatt ctttccggca gcgcttcctt cagatggtac aa
#tggatatg 1800
gttatcaccg atgcaagaac ctcgttgacg aggatggcac caatcctgct gg
#gactagat 1860
aagggttccc atgttttaca accggaagtc ttacactcta aaattttggc at
#acaagata 1920
ataccaaagc tagggaacgg cttgttctct gtcgatggcg agaaatttcc tc
#tagagccc 1980
cttcaagtcg aaattatgcc acgcttatgc aagacgttac tgagaaatgg cc
#gttatgtg 2040
gacacagatt tcgattctat gtga
#
# 2064
<210> SEQ ID NO 33
<211> LENGTH: 687
<212> TYPE: PRT
<213> ORGANISM: Saccharomyces cerevisiaie
<400> SEQUENCE: 33
Met Thr Leu Lys Pro Ser Lys Arg Arg Lys Gl
#y Arg Ser Arg His Ser
  1 5
# 10
# 15
Arg Lys Lys Gln Ile Thr Ser Ala Ile Leu Th
#r Glu Glu Gly Ile Met
                        20
# 25
# 30
Ile Lys Ala Lys Pro Ser Ser Pro Tyr Thr Ty
#r Ala Asn Arg Met Ala
                35
# 40
# 45
Asp Lys Arg Ser Arg Ser Ser Ile Asp Asn Il
#e Ser Arg Thr Ser Phe
        50
# 55
# 60
Gln Ser Asn Ile Ser Arg Thr Ser Phe Gln Se
#r Asn Ser Asp Asn Asn
65
#70
#75
#80
Ser Ile Phe Glu Thr Ala Ser Leu Ile Ser Cy
#s Val Thr Cys Leu Ser
                                85
# 90
# 95
Asp Thr Asp Thr Ile Asp Arg Ser Glu Thr Se
#r Thr Thr Asp Thr Ser
                        100
# 105
# 110
Lys Asp Asp Leu Ser Ala Asn Pro Lys Leu Hi
#s Tyr Pro Ser Val Asn
                115
# 120
# 125
Gly Gln Leu Pro Ala Asn Thr Val Ile Pro Ty
#r Gly Arg Ile Leu Asp
        130
# 135
# 140
Ala Arg Tyr Ile Glu Lys Glu Pro Leu His Ty
#r Tyr Asp Ala Asn Ser
145 1
#50 1
#55 1
#60
Ser Pro Ser Ser Pro Leu Ser Ser Ser Met Se
#r Asn Ile Ser Glu Lys
                                165
# 170
# 175
Cys Asp Leu Asp Glu Leu Glu Ser Ser Gln Ly
#s Lys Glu Arg Lys Gly
                        180
# 185
# 190
Asn Ser Leu Ser Arg Gly Ser Asn Ser Ser Se
#r Ser Leu Leu Thr Ser
                195
# 200
# 205
Arg Ser Pro Phe Thr Lys Leu Val Glu Val Il
#e Phe Ala Arg Pro Arg
        210
# 215
# 220
Arg His Asp Val Val Pro Lys Arg Val Ser Le
#u Tyr Ile Asp Tyr Lys
225 2
#30 2
#35 2
#40
Pro His Ser Ser Ser His Leu Lys Glu Glu As
#p Asp Leu Val Glu Glu
                                245
# 250
# 255
Ile Leu Lys Arg Ser Tyr Lys Asn Thr Arg Ar
#g Asn Lys Ser Ile Phe
                        260
# 265
# 270
Val Ile Ile Asn Pro Phe Gly Gly Lys Gly Ly
#s Ala Lys Lys Leu Phe
                275
# 280
# 285
Met Thr Lys Ala Lys Pro Leu Leu Leu Ala Se
#r Arg Cys Ser Ile Glu
        290
# 295
# 300
Val Val Tyr Thr Lys Tyr Pro Gly His Ala Il
#e Glu Ile Ala Arg Glu
305 3
#10 3
#15 3
#20
Met Asp Ile Asp Lys Tyr Asp Thr Ile Ala Cy
#s Ala Ser Gly Asp Gly
                                325
# 330
# 335
Ile Pro His Glu Val Ile Asn Gly Leu Tyr Gl
#n Arg Pro Asp His Val
                        340
# 345
# 350
Lys Ala Phe Asn Asn Ile Ala Ile Thr Glu Il
#e Pro Cys Gly Ser Gly
                355
# 360
# 365
Asn Ala Met Ser Val Ser Cys His Trp Thr As
#n Asn Pro Ser Tyr Ser
        370
# 375
# 380
Thr Leu Cys Leu Ile Lys Ser Ile Glu Thr Ar
#g Ile Asp Leu Met Cys
385 3
#90 3
#95 4
#00
Cys Ser Gln Pro Ser Tyr Ala Arg Glu His Pr
#o Lys Leu Ser Phe Leu
                                405
# 410
# 415
Ser Gln Thr Tyr Gly Leu Ile Ala Glu Thr As
#p Ile Asn Thr Glu Phe
                        420
# 425
# 430
Ile Arg Trp Met Gly Pro Ala Arg Phe Glu Le
#u Gly Val Ala Phe Asn
                435
# 440
# 445
Ile Ile Gln Lys Lys Lys Tyr Pro Cys Glu Il
#e Tyr Val Lys Tyr Ala
        450
# 455
# 460
Ala Lys Ser Lys Asn Glu Leu Lys Asn His Ty
#r Leu Glu His Lys Asn
465 4
#70 4
#75 4
#80
Lys Gly Ser Leu Glu Phe Gln His Ile Thr Me
#t Asn Lys Asp Asn Glu
                                485
# 490
# 495
Asp Cys Asp Asn Tyr Asn Tyr Glu Asn Glu Ty
#r Glu Thr Glu Asn Glu
                        500
# 505
# 510
Asp Glu Asp Glu Asp Ala Asp Ala Asp Asp Gl
#u Asp Ser His Leu Ile
                515
# 520
# 525
Ser Arg Asp Leu Ala Asp Ser Ser Ala Asp Gl
#n Ile Lys Glu Glu Asp
        530
# 535
# 540
Phe Lys Ile Lys Tyr Pro Leu Asp Glu Gly Il
#e Pro Ser Asp Trp Glu
545 5
#50 5
#55 5
#60
Arg Leu Asp Pro Asn Ile Ser Asn Asn Leu Gl
#y Ile Phe Tyr Thr Gly
                                565
# 570
# 575
Lys Met Pro Tyr Val Ala Ala Asp Thr Lys Ph
#e Phe Pro Ala Ala Leu
                        580
# 585
# 590
Pro Ser Asp Gly Thr Met Asp Met Val Ile Th
#r Asp Ala Arg Thr Ser
                595
# 600
# 605
Leu Thr Arg Met Ala Pro Ile Leu Leu Gly Le
#u Asp Lys Gly Ser His
        610
# 615
# 620
Val Leu Gln Pro Glu Val Leu His Ser Lys Il
#e Leu Ala Tyr Lys Ile
625 6
#30 6
#35 6
#40
Ile Pro Lys Leu Gly Asn Gly Leu Phe Ser Va
#l Asp Gly Glu Lys Phe
                                645
# 650
# 655
Pro Leu Glu Pro Leu Gln Val Glu Ile Met Pr
#o Arg Leu Cys Lys Thr
                        660
# 665
# 670
Leu Leu Arg Asn Gly Arg Tyr Val Asp Thr As
#p Phe Asp Ser Met
                675
# 680
# 685
<210> SEQ ID NO 34
<211> LENGTH: 1230
<212> TYPE: DNA
<213> ORGANISM: Saccharomyces cerevisiaie
<400> SEQUENCE: 34
atggtagatg gactgaatac ctcgaacatt aggaaaagag ccaggactct ct
#ctaacccc 60
aatgactttc aagagcctaa ttacttgctg gatcccggta atcatccctc ag
#atcatttt 120
cgaactcgaa tgtccaaatt tcggtttaat attagagaga agctgttagt gt
#ttaccaac 180
aatcaatcat tcacattaag ccgctggcaa aagaagtacc gttctgcgtt ta
#atgatctc 240
tactttactt atacttcctt aatgggatcg cataccttct atgttctgtg tt
#tacctatg 300
cccgtgtggt ttggatattt tgaaacaaca aaagatatgg tttatatctt gg
#gatattct 360
atctacttga gtggtttttt taaagattac tggtgcttgc ccaggcctag ag
#cacctcca 420
ttacatcgaa ttacgttaag tgaatataca acgaaggaat atggtgctcc aa
#gctcccat 480
acagcaaatg caacaggagt gagtctcttg tttctctaca acatctggag ga
#tgcaagaa 540
tcttctgtca tggtccaact attgttgtca tgtgtggttt tattttatta ta
#tgactttg 600
gttttcggta gaatatactg tgggatgcat ggcatattag atttagtaag cg
#gtgggctc 660
attggaatag tgtgtttcat tgttaggatg tatttcaagt acaggtttcc gg
#gtttacgc 720
attgaggagc attggtggtt tcctttgttt agtgtgggat ggggtcttct tc
#ttttgttt 780
aaacatgtta agcccgtaga cgaatgtcct tgcttccaag atagtgttgc gt
#tcatgggc 840
gttgtgtcag gtattgaatg ctgtgattgg ttgggcaaag tgtttggagt ca
#ccctggtg 900
tacaatttgg aacctaactg tggctggcgg ttaaccttag ccaggctgct gg
#tgggccta 960
ccgtgcgttg ttatctggaa gtacgtgatc agcaaaccga tgatctacac gt
#tattgatc 1020
aaagttttcc atctgaagga tgacagaaac gttgcggcaa gaaaaagact gg
#aggccacg 1080
cacaaagaag gtgcaagcaa gtacgaatgt ccattatata ttggagagcc ca
#agattgac 1140
attctaggta gatttattat ctatgctggc gttccattca ccgttgtaat gt
#gcagcccc 1200
gtcctatttt ccctcttaaa tatagcataa
#
# 1230
<210> SEQ ID NO 35
<211> LENGTH: 409
<212> TYPE: PRT
<213> ORGANISM: Saccharomyces cerevisiaie
<400> SEQUENCE: 35
Met Val Asp Gly Leu Asn Thr Ser Asn Ile Ar
#g Lys Arg Ala Arg Thr
  1 5
# 10
# 15
Leu Ser Asn Pro Asn Asp Phe Gln Glu Pro As
#n Tyr Leu Leu Asp Pro
                        20
# 25
# 30
Gly Asn His Pro Ser Asp His Phe Arg Thr Ar
#g Met Ser Lys Phe Arg
                35
# 40
# 45
Phe Asn Ile Arg Glu Lys Leu Leu Val Phe Th
#r Asn Asn Gln Ser Phe
        50
# 55
# 60
Thr Leu Ser Arg Trp Gln Lys Lys Tyr Arg Se
#r Ala Phe Asn Asp Leu
65
#70
#75
#80
Tyr Phe Thr Tyr Thr Ser Leu Met Gly Ser Hi
#s Thr Phe Tyr Val Leu
                                85
# 90
# 95
Cys Leu Pro Met Pro Val Trp Phe Gly Tyr Ph
#e Glu Thr Thr Lys Asp
                        100
# 105
# 110
Met Val Tyr Ile Leu Gly Tyr Ser Ile Tyr Le
#u Ser Gly Phe Phe Lys
                115
# 120
# 125
Asp Tyr Trp Cys Leu Pro Arg Pro Arg Ala Pr
#o Pro Leu His Arg Ile
        130
# 135
# 140
Thr Leu Ser Glu Tyr Thr Thr Lys Glu Tyr Gl
#y Ala Pro Ser Ser His
145 1
#50 1
#55 1
#60
Thr Ala Asn Ala Thr Gly Val Ser Leu Leu Ph
#e Leu Tyr Asn Ile Trp
                                165
# 170
# 175
Arg Met Gln Glu Ser Ser Val Met Val Gln Le
#u Leu Leu Ser Cys Val
                        180
# 185
# 190
Val Leu Phe Tyr Tyr Met Thr Leu Val Phe Gl
#y Arg Ile Tyr Cys Gly
                195
# 200
# 205
Met His Gly Ile Leu Asp Leu Val Ser Gly Gl
#y Leu Ile Gly Ile Val
        210
# 215
# 220
Cys Phe Ile Val Arg Met Tyr Phe Lys Tyr Ar
#g Phe Pro Gly Leu Arg
225 2
#30 2
#35 2
#40
Ile Glu Glu His Trp Trp Phe Pro Leu Phe Se
#r Val Gly Trp Gly Leu
                                245
# 250
# 255
Leu Leu Leu Phe Lys His Val Lys Pro Val As
#p Glu Cys Pro Cys Phe
                        260
# 265
# 270
Gln Asp Ser Val Ala Phe Met Gly Val Val Se
#r Gly Ile Glu Cys Cys
                275
# 280
# 285
Asp Trp Leu Gly Lys Val Phe Gly Val Thr Le
#u Val Tyr Asn Leu Glu
        290
# 295
# 300
Pro Asn Cys Gly Trp Arg Leu Thr Leu Ala Ar
#g Leu Leu Val Gly Leu
305 3
#10 3
#15 3
#20
Pro Cys Val Val Ile Trp Lys Tyr Val Ile Se
#r Lys Pro Met Ile Tyr
                                325
# 330
# 335
Thr Leu Leu Ile Lys Val Phe His Leu Lys As
#p Asp Arg Asn Val Ala
                        340
# 345
# 350
Ala Arg Lys Arg Leu Glu Ala Thr His Lys Gl
#u Gly Ala Ser Lys Tyr
                355
# 360
# 365
Glu Cys Pro Leu Tyr Ile Gly Glu Pro Lys Il
#e Asp Ile Leu Gly Arg
        370
# 375
# 380
Phe Ile Ile Tyr Ala Gly Val Pro Phe Thr Va
#l Val Met Cys Ser Pro
385 3
#90 3
#95 4
#00
Val Leu Phe Ser Leu Leu Asn Ile Ala
                                405
<210> SEQ ID NO 36
<211> LENGTH: 1215
<212> TYPE: DNA
<213> ORGANISM: Saccharomyces cerevisiaie
<400> SEQUENCE: 36
atgaccatta ttcagacggt tactgaattg ggtgttaccg aggacaccat ta
#aggttcag 60
atggctcctt ccggcggcaa acacctattg gcagatcccg gtaatcatcc ag
#cggagcat 120
tttgagagcc agatgtcgtg gctaaggttt caaacaaggc agtatctaac ta
#gattcaca 180
gacaaccaat cagatttcgt acattcttta caaaaaaagc acagaacgcc tt
#ttagagac 240
gtttatttca aatacacttc gcttatgggt tcccacatgt tttatgttat cg
#tgcttccc 300
atgcctgtgt ggcttggata ccgcgattta acacgggaca tgatctacgt tc
#ttggttat 360
tcaatttatt tgagtggcta cttaaaggat tattggtgcc taccaaggcc aa
#aatcaccg 420
ccagttgaca gaatcacact aagtgaatac actacgaaag aatatggtgc ac
#ccagttca 480
cattctgcta acgctactgc ggtaagtcta ttattctttt ggagaatatg tt
#tatctgac 540
acactggtat ggccaacaaa gcttctttta ctgagtctgg tgatatttta ct
#acttaacc 600
ctggtttttg gtagagttta ctgcggtatg catggtatgc tggatttatt ta
#gcggcgcc 660
gcagttggag ctatctgttt ttttataaga atctgggtgg tgcatgcttt ac
#gaaatttc 720
cagattggtg aacatctctg gtttcccctt ttgagtgtag catggggctt gt
#ttattctg 780
tttaaccacg tcaggcccat tgatgaatgt ccttgtttcg aagatagcgt ag
#cgttcatt 840
ggcgtagtca gtgggctgga ttgcagcgac tggttaaccg aaagatacgg at
#ggaacctt 900
gtatgtagta ggtacgcatc atgtggttct aaggtgttct tgaggcctct gg
#taggtgtc 960
gcttctgtga ttgtttggaa agacgtcatt agcaagacag ctgtctacac gc
#tgttaatt 1020
aaactactca gatttcacga tgatagaagc gaaaaggttc atttccataa cg
#agacaagt 1080
gaagaagaag agtgtttatt gtacagcggt gtttccaaag tggaaatcgt cg
#gaaggttt 1140
ctcatatacg caggtatacc tacaaccgtc tttttgctat gcccagtttt tt
#tcacttgg 1200
acaaacttaa ggtag
#
#
# 1215
<210> SEQ ID NO 37
<211> LENGTH: 404
<212> TYPE: PRT
<213> ORGANISM: Sacchromyces cerevisiaie
<400> SEQUENCE: 37
Met Thr Ile Ile Gln Thr Val Thr Glu Leu Gl
#y Val Thr Glu Asp Thr
  1 5
# 10
# 15
Ile Lys Val Gln Met Ala Pro Ser Gly Gly Ly
#s His Leu Leu Ala Asp
                        20
# 25
# 30
Pro Gly Asn His Pro Ala Glu His Phe Glu Se
#r Gln Met Ser Trp Leu
                35
# 40
# 45
Arg Phe Gln Thr Arg Gln Tyr Leu Thr Arg Ph
#e Thr Asp Asn Gln Ser
        50
# 55
# 60
Asp Phe Val His Ser Leu Gln Lys Lys His Ar
#g Thr Pro Phe Arg Asp
65
#70
#75
#80
Val Tyr Phe Lys Tyr Thr Ser Leu Met Gly Se
#r His Met Phe Tyr Val
                                85
# 90
# 95
Ile Val Leu Pro Met Pro Val Trp Leu Gly Ty
#r Arg Asp Leu Thr Arg
                        100
# 105
# 110
Asp Met Ile Tyr Val Leu Gly Tyr Ser Ile Ty
#r Leu Ser Gly Tyr Leu
                115
# 120
# 125
Lys Asp Tyr Trp Cys Leu Pro Arg Pro Lys Se
#r Pro Pro Val Asp Arg
        130
# 135
# 140
Ile Thr Leu Ser Glu Tyr Thr Thr Lys Glu Ty
#r Gly Ala Pro Ser Ser
145 1
#50 1
#55 1
#60
His Ser Ala Asn Ala Thr Ala Val Ser Leu Le
#u Phe Phe Trp Arg Ile
                                165
# 170
# 175
Cys Leu Ser Asp Thr Leu Val Trp Pro Thr Ly
#s Leu Leu Leu Leu Ser
                        180
# 185
# 190
Leu Val Ile Phe Tyr Tyr Leu Thr Leu Val Ph
#e Gly Arg Val Tyr Cys
                195
# 200
# 205
Gly Met His Gly Met Leu Asp Leu Phe Ser Gl
#y Ala Ala Val Gly Ala
        210
# 215
# 220
Ile Cys Phe Phe Ile Arg Ile Trp Val Val Hi
#s Ala Leu Arg Asn Phe
225 2
#30 2
#35 2
#40
Gln Ile Gly Glu His Leu Trp Phe Pro Leu Le
#u Ser Val Ala Trp Gly
                                245
# 250
# 255
Leu Phe Ile Leu Phe Asn His Val Arg Pro Il
#e Asp Glu Cys Pro Cys
                        260
# 265
# 270
Phe Glu Asp Ser Val Ala Phe Ile Gly Val Va
#l Ser Gly Leu Asp Cys
                275
# 280
# 285
Ser Asp Trp Leu Thr Glu Arg Tyr Gly Trp As
#n Leu Val Cys Ser Arg
        290
# 295
# 300
Tyr Ala Ser Cys Gly Ser Lys Val Phe Leu Ar
#g Pro Leu Val Gly Val
305 3
#10 3
#15 3
#20
Ala Ser Val Ile Val Trp Lys Asp Val Ile Se
#r Lys Thr Ala Val Tyr
                                325
# 330
# 335
Thr Leu Leu Ile Lys Leu Leu Arg Phe His As
#p Asp Arg Ser Glu Lys
                        340
# 345
# 350
Val His Phe His Asn Glu Thr Ser Glu Glu Gl
#u Glu Cys Leu Leu Tyr
                355
# 360
# 365
Ser Gly Val Ser Lys Val Glu Ile Val Gly Ar
#g Phe Leu Ile Tyr Ala
        370
# 375
# 380
Gly Ile Pro Thr Thr Val Phe Leu Leu Cys Pr
#o Val Phe Phe Thr Trp
385 3
#90 3
#95 4
#00
Thr Asn Leu Arg
<210> SEQ ID NO 38
<211> LENGTH: 1050
<212> TYPE: DNA
<213> ORGANISM: Saccharomyces cerevisiaie
<400> SEQUENCE: 38
atgaacgtaa catcgaatgc aactgcagcc ggttcctttc cactagcatt tg
#gtctcaag 60
acctcatttg ggtttatgca ctatgccaag gcccctgcca ttaatttacg cc
#ccaaggaa 120
tccttgctgc cggaaatgag tgatggtgtg ctggccttgg ttgcgccggt tg
#ttgcctac 180
tgggcgttgt ctggtatatt ccatgtaata gacactttcc atctggctga ga
#agtacaga 240
attcatccga gcgaagaggt tgccaagagg aacaaggcgt cgagaatgca tg
#ttttcctt 300
gaagtgattc tacaacatat catacagacc attgttggcc ttatctttat gc
#acttcgag 360
ccgatctaca tgactgggtt tgaagaaaat gccatgtgga agcttcgtgc ag
#accttcct 420
cggattattc cagatgccgc tatttattac ggctatatgt acggaatgtc cg
#ctttgaag 480
atctttgcag gctttttatt cgttgataca tggcaatact ttttgcatag at
#tgatgcat 540
atgaataaga ccttatacaa atggttccac tctgttcatc atgaactata cg
#tgccatat 600
gcttacggtg ctcttttcaa caatcctgtt gagggcttct tgttagatac tt
#tgggaacc 660
ggtattgcca tgacgttaac tcatttgact cacagagagc aaatcattct tt
#ttaccttt 720
gccaccatga agactgtcga tgaccactgt gggtatgctt tgccacttga cc
#cattccaa 780
tggcttttcc ctaataacgc tgtctatcac gatatccacc accagcaatt tg
#gtatcaag 840
acgaactttg ctcaaccatt tttcactttc tgggacaatt tgttccaaac ta
#actttaaa 900
gggtttgaag aatatcaaaa gaagcaaaga cgtgtcacca tcgacaagta ca
#aagagttt 960
ttgcaagaga gagaattgga aaagaaggag aaactcaaaa acttcaaagc ta
#tgaatgct 1020
gctgaaaatg aagtaaagaa agagaaataa
#
# 1050
<210> SEQ ID NO 39
<211> LENGTH: 349
<212> TYPE: PRT
<213> ORGANISM: Saccharomyces cerevisiaie
<400> SEQUENCE: 39
Met Asn Val Thr Ser Asn Ala Thr Ala Ala Gl
#y Ser Phe Pro Leu Ala
  1 5
# 10
# 15
Phe Gly Leu Lys Thr Ser Phe Gly Phe Met Hi
#s Tyr Ala Lys Ala Pro
                        20
# 25
# 30
Ala Ile Asn Leu Arg Pro Lys Glu Ser Leu Le
#u Pro Glu Met Ser Asp
                35
# 40
# 45
Gly Val Leu Ala Leu Val Ala Pro Val Val Al
#a Tyr Trp Ala Leu Ser
        50
# 55
# 60
Gly Ile Phe His Val Ile Asp Thr Phe His Le
#u Ala Glu Lys Tyr Arg
65
#70
#75
#80
Ile His Pro Ser Glu Glu Val Ala Lys Arg As
#n Lys Ala Ser Arg Met
                                85
# 90
# 95
His Val Phe Leu Glu Val Ile Leu Gln His Il
#e Ile Gln Thr Ile Val
                        100
# 105
# 110
Gly Leu Ile Phe Met His Phe Glu Pro Ile Ty
#r Met Thr Gly Phe Glu
                115
# 120
# 125
Glu Asn Ala Met Trp Lys Leu Arg Ala Asp Le
#u Pro Arg Ile Ile Pro
        130
# 135
# 140
Asp Ala Ala Ile Tyr Tyr Gly Tyr Met Tyr Gl
#y Met Ser Ala Leu Lys
145 1
#50 1
#55 1
#60
Ile Phe Ala Gly Phe Leu Phe Val Asp Thr Tr
#p Gln Tyr Phe Leu His
                                165
# 170
# 175
Arg Leu Met His Met Asn Lys Thr Leu Tyr Ly
#s Trp Phe His Ser Val
                        180
# 185
# 190
His His Glu Leu Tyr Val Pro Tyr Ala Tyr Gl
#y Ala Leu Phe Asn Asn
                195
# 200
# 205
Pro Val Glu Gly Phe Leu Leu Asp Thr Leu Gl
#y Thr Gly Ile Ala Met
        210
# 215
# 220
Thr Leu Thr His Leu Thr His Arg Glu Gln Il
#e Ile Leu Phe Thr Phe
225 2
#30 2
#35 2
#40
Ala Thr Met Lys Thr Val Asp Asp His Cys Gl
#y Tyr Ala Leu Pro Leu
                                245
# 250
# 255
Asp Pro Phe Gln Trp Leu Phe Pro Asn Asn Al
#a Val Tyr His Asp Ile
                        260
# 265
# 270
His His Gln Gln Phe Gly Ile Lys Thr Asn Ph
#e Ala Gln Pro Phe Phe
                275
# 280
# 285
Thr Phe Trp Asp Asn Leu Phe Gln Thr Asn Ph
#e Lys Gly Phe Glu Glu
        290
# 295
# 300
Tyr Gln Lys Lys Gln Arg Arg Val Thr Ile As
#p Lys Tyr Lys Glu Phe
305 3
#10 3
#15 3
#20
Leu Gln Glu Arg Glu Leu Glu Lys Lys Glu Ly
#s Leu Lys Asn Phe Lys
                                325
# 330
# 335
Ala Met Asn Ala Ala Glu Asn Glu Val Lys Ly
#s Glu Lys
                        340
# 345
<210> SEQ ID NO 40
<211> LENGTH: 32
<212> TYPE: DNA
<213> ORGANISM: Artificial Sequence
<220> FEATURE:
<223> OTHER INFORMATION: PCR primer
<400> SEQUENCE: 40
atattaagct tatggatcca gcgggcggcc cc
#
# 32
<210> SEQ ID NO 41
<211> LENGTH: 30
<212> TYPE: DNA
<213> ORGANISM: Artificial Sequence
<220> FEATURE:
<223> OTHER INFORMATION: PCR primer
<400> SEQUENCE: 41
agatctagat cataagggct cttctggcgg
#
# 30
```

What is claimed is:

1. A method for identifying an agent that modulates sphingolipid metabolism, comprising:
(a) culturing a mutant yeast strain with sphingosine in the absence and presence of a candidate agent under conditions and for a time sufficient to observe in said mutant yeast strain an altered level of either (i) at least one sphingolipid intermediate, or (ii) activity of at least one component of a sphingolipid pathway, wherein:
the mutant yeast strain comprises a null allele of at least one gene encoding a component of a sphingolipid pathway that results in an altered activity level of at least one sphingolipid pathway component, and wherein said mutant strain of yeast has been genetically altered to express at least one nonendogenous sphingolipid pathway component and wherein the mutant yeast strain exhibits growth inhibition in the presence of sphingosine; and
(b) comparing growth of the mutant yeast strain in the presence of the candidate agent to the growth of the mutant yeast strain in the absence of the candidate agent, wherein an increase in growth of the mutant yeast strain in the presence of the agent indicates the agent modulates sphingolipid metabolism.

2. The method of claim 1 wherein said at least one gene comprises DPL1.

3. The method of claim 1 wherein said at least one gene comprises DPL1 and LCB4.

4. The method of claim 3 wherein said at least one nonendogenous sphingolipid pathway component comprises human SPHK1.

5. The method of claim 4 wherein the altered level of the sphingolipid intermediate in the presence of the candidate agent comprises a decrease in at least one LCBP.

6. The method of claim 5 wherein said at least one LCBP comprises sphingosine-1-phosphate.

7. The method of claim 4 wherein the altered level of the activity of the sphingolipid pathway component in the presence of the candidate agent comprises a decrease in the human SPHK1 activity.

8. A method for identifying an inhibitor of SK, comprising:
(a) culturing a null mutant yeast strain with sphingosine in the absence and presence of a candidate agent under conditions and for a time sufficient to observe in said mutant yeast strain an altered ability of said yeast mutant strain to grow, wherein:
the mutant yeast strain comprises a null allele of at least DPL1 and LCB4, and wherein said mutant strain of yeast has been genetically altered to express nonendogenous SK and wherein the mutant yeast strain exhibits growth inhibition in the presence of sphingosine; and
(b) comparing growth of the mutant yeast strain in the presence of the candidate agent to the growth of the mutant yeast strain in the absence of the candidate agent, wherein an increase in growth of said mutant yeast strain in the presence of the agent indicates the agent is an inhibitor of SK.

9. A method for identifying an inhibitor of SK, comprising:
(a) culturing a null mutant yeast strain with an inducer in the absence and presence of a candidate agent under conditions and for a time sufficient to observe altered growth in said mutant yeast strain, wherein:
the mutant yeast strain comprises a null allele of at least DPL1, LCB4, and SPP, and wherein said mutant strain of yeast has been genetically altered to express nonendogenous SK under the control of a promoter that is induced by the inducer and wherein the mutant yeast strain exhibits growth inhibition in the presence of the inducer; and
(b) comparing growth of the mutant yeast strain in the presence of the candidate agent to the growth of the mutant yeast strain in the absence of the candidate agent, wherein an increase in growth of said mutant yeast strain in the presence of the candidate agent indicates the candidate agent is an inhibitor of SK.

10. The method of claim 9 wherein said inducible promoter is a galactose-inducible promoter.

11. The method of claim 10 wherein said inducer is galactose.

12. A method for identifying an agent that modulates sphingolipid metabolism, comprising:
(a) culturing a mutant yeast strain with sphingosine in the absence and presence of a candidate agent under conditions and for a time sufficient to observe in said mutant yeast strain an altered level of either (i) at least one sphingolipid intermediate, or (ii) activity of at least one component of a sphingolipid pathway, wherein:
the mutant yeast strain comprises a null allele of at least one gene encoding a component of a sphingolipid pathway that results in an altered activity level of at least one sphingolipid pathway component, and wherein said mutant strain of yeast has been genetically altered to express at least one nonendogenous sphingolipid pathway component and wherein the mutant yeast strain exhibits growth inhibition in the presence of sphingosine; and
(b) comparing the level of either (i) or (ii) in the mutant yeast strain cultured in the presence of the candidate agent to the level of either (i) or (ii) in the mutant yeast strain cultured in the absence of the candidate agent, wherein an altered level in the presence of the agent indicates the agent modulates sphingolipid metabolism.

13. The method of claim 12 wherein said altered level of said at least one sphingolipid intermediate comprises a decrease in S-1-P.

14. The method of claim 13 wherein said altered level of said activity of at least one component of a sphingolipid pathway comprises a decrease in the activity of said at least one nonendogenous sphingolipid pathway component.

15. The method of claim 14 wherein said at least one nonendogenous sphingolipid pathway component comprises human SPHK1.

16. A method for identifying an agent that modulates sphingolipid metabolism, comprising:
(a) culturing a null mutant yeast strain with sphingosine in the absence and presence of a candidate agent under conditions and for a time sufficient to observe altered growth of said mutant yeast strain, wherein:
the mutant yeast strain comprises a null allele of at least one gene encoding a component of a sphingolipid pathway that results in an altered activity level of at least one sphingolipid pathway component, and wherein said mutant strain of yeast has been genetically altered to express at least one nonendogenous sphingolipid pathway component, and wherein the mutant yeast strain exhibits growth inhibition in the presence of sphingosine; and
(b) comparing growth of the mutant yeast strain in the presence of the candidate agent to growth of the mutant yeast strain in the absence of the candidate agent, wherein an increase in growth of said mutant yeast strain in the presence of the candidate agent indicates the agent modulates sphingolipid metabolism.

17. The method of claim 16 wherein said at least one gene comprises DPL1.

18. The method of claim 16 wherein said at least one gene comprises DPL1 and LCB4.

19. The method of claim 16 wherein said at least one nonendogenous sphingolipid pathway component comprises human SPHK1.

20. A method for identifying an agent that modulates sphingolipid metabolism, comprising:
(a) culturing a mutant yeast strain with an inducer in the absence and presence of a candidate agent under conditions and for a time sufficient to observe in said mutant yeast strain an altered level of either (i) at least one sphingolipid intermediate, or (ii) activity of at least one component of a sphingolipid pathway, wherein:
the mutant yeast strain comprises a null allele of at least one gene encoding a component of a sphingolipid pathway that results in an altered activity level of at least one sphingolipid pathway component, and wherein said mutant strain of yeast has been genetically altered to express at least one nonendogenous sphingolipid pathway component under the control of a promoter that is induced by the inducer and wherein the mutant yeast strain exhibits growth inhibition in the presence of the inducer; and
(b) comparing the level of either (i) or (ii) in the mutant yeast strain cultured in the presence of the candidate agent to the level of either (i) or (ii) in the mutant yeast strain cultured in the absence of the candidate agent, wherein an altered level in the presence of the agent indicates the agent modulates sphingolipid metabolism.

21. The method of claim 20 wherein said altered level of said at least one sphingolipid intermediate comprises a decrease in LCBPs.

22. The method of claim 20 wherein said altered level of said activity of at least one component of a sphingolipid pathway comprises a decrease in the activity of said at least one nonendogenous sphingolipid pathway component.

23. The method of claim 22 wherein said at least one nonendogenous sphingolipid pathway component comprises human SPHK1.

24. The method of claim 20 wherein said at least one gene comprises DPL1.

25. The method of claim 20 wherein said at least one gene comprises DPL1 and LCB4.

26. The method of claim 20 wherein said at least one gene comprises DPL1, LCB4, and YSR2.

27. A method for identifying an agent that modulates sphingolipid metabolism, comprising:
(a) culturing a null mutant yeast strain with an inducer in the absence and presence of a candidate agent under conditions and for a time sufficient to observe altered growth of said mutant yeast strain, wherein:
the mutant yeast strain comprises a null allele of at least one gene encoding a component of a sphingolipid pathway that results in an altered activity level of at least one sphingolipid pathway component, and wherein said mutant strain of yeast has been genetically altered to express at least one nonendogenous sphingolipid pathway component under the control of a promoter that is induced by the inducer and wherein the mutant yeast strain exhibits growth inhibition in the presence of the inducer; and
(b) comparing growth of the mutant yeast strain in the presence of the candidate agent to growth of the mutant yeast strain in the absence of the candidate agent, wherein an increase in growth of said mutant yeast strain in the presence of the candidate agent indicates the agent modulates sphingolipid metabolism.

28. The method of claim 27 wherein said at least one gene comprises DPL1.

29. The method of claim 27 wherein said at least one gene comprises DPL1 and LCB4.

30. The method of claim 27 wherein said at least one gene comprises DPL1, LCB4, and YSR2.

31. The method of claim 30 wherein said at least one nonendogenous sphingolipid pathway component comprises human SPHK1.
